(19) United States
(12) Patent Application Publication
(10) Pub. No.: US 2025/0382292 A1
(43) Pub. Date: Dec. 18, 2025

(54) COMPOUNDS FOR TREATING LIGHT CHAIN AMYLOIDOSIS

(71) Applicant: Protego Biopharma, Inc., San Diego, CA (US)

(72) Inventors: Bo QIN, La Jolla, CA (US); Huang QIU, San Diego, CA (US); Hank Michael James PETRASSI, La Jolla, CA (US); Virginia Heather Sharron GRANT, San Diego, CA (US); Steven J. WILKENS, San Diego, CA (US); Justyna Maria SOSNA, San Diego, CA (US)

(21) Appl. No.: 19/239,698

(22) Filed: Jun. 16, 2025

Related U.S. Application Data
(60) Provisional application No. 63/660,919, filed on Jun. 17, 2024.

(51) Int. Cl. C07D 471/04; A61K 31/444; A61K 31/4545; A61K 45/06; C07D 519/00

(52) U.S. Cl. CPC C07D 471/04; A61K 31/444; A61K 31/4545; A61K 45/06; C07D 519/00

(57) ABSTRACT

Provided herein are compounds and pharmaceutically acceptable derivatives thereof for use in compositions and methods of treating light chain amyloidosis. Also provided are methods of degrading immunoglobulin light chains using the compounds and compositions provided herein.

### CROSS-REFERENCE TO RELATED APPLICATIONS

The present application claims priority from application No. 63/660,919, filed on Jun. 17, 2024, which is incorporated herein by reference in its entirety for all purposes.

### FIELD

Provided herein are compounds for use in compositions and methods of treating light chain amyloidosis (AL). In one embodiment, the compounds are proteolysis targeting chimeras (PROTACs).

### BACKGROUND

PROTACs are heterobifunctional compounds that contain two distinct moieties optionally covalently linked by a chemical bond or a divalent chemical linker. PROTACs are sometimes referred to as degraders. One of the moieties in a PROTAC binds a target protein (i.e., a protein whose activity and/or expression is desired to be diminished), while the other moiety is a ligand that recruits an E3 ligase (e.g., cereblon), sometimes referred to as a ubiquitin E3 ligase ligand or binder. To induce degradation, PROTACs are believed to induce formation of a ternary complex between the target protein, the PROTAC compound, and an E3 ligase ligand. The PROTAC thus brings the target protein into close proximity to an E3 ligase, which leads to (poly)ubiquitination of the surface of the target protein making it a neosubstrate for proteosomal degradation. By choosing an appropriate protein-targeting moiety, PROTACs can be designed to be highly specific for the targeted protein. The choice of divalent chemical linker and E3 ligase ligand also affects the activity and/or specificity of PROTACs.

A degradation approach for a target protein can have potential advantages compared to, e.g., small molecule stabilization or inhibition of the target protein. One potential advantage is that the duration of effect of a heterobifunctional compound is generally based on the resynthesis rate of the target protein. Another potential advantage is that many heterobifunctional compounds are believed to be released from the ubiquitinated target protein-E3 ligase complex and made available for formation of further ternary complexes; this is sometimes referred to as “catalytic” turnover of the heterobifunctional compound. Degradation of a target protein can also be advantageous over small molecule stabilization or inhibition in some cases, as degradation can impair a scaffolding function of a target protein, whereas a small molecule might not. It is also generally believed that for formation of a ternary complex, high affinity to the target protein is not always required. A degradation approach can also include other methods of bivalency including but not limited to LYTACS, ASGPR mediated degradation, ABtac, Gluetac and others well known to those of skill in the art. See, e.g., WO 2017/184995; WO 2019/144117; WO 2020/163823; WO 2021/078301; WO 2021/146536; WO 2021/007307; WO 2021/222114; WO 2021/078301; WO 2022/169780; WO 2023/044046; Chamberlain and Hamann, Nature Chemical Biology 15.10 (2019): 937-944; Li and Song, Journal of Hematology & Oncology 13 (2020): 1-14; Wu, et al. Nature Structural & Molecular Biology 27.7 (2020): 605-614; Dong, et al., Journal of Medicinal Chemistry 64.15 (2021): 10606-10620; Yang, et al., Targeted Oncology 16.1 (2021): 1-12; Lv, et al., Nature Communications 12.1 (2021): 6896.

Alternatively, PROTACs contain a moiety that activates the N-degron pathway (i.e., the N-end rule pathway) of ubiquitination and thus proteasome degradation, and a moiety that targets a protein of interest. See, e.g., Pan, et al. Nature 2021, 600(7888), 334-338; Kim, et al. Int. J. Mol. Sci. 2021, 22, 8323; Zhang, et al. J. Biol. Chem. 2023, 299(8), 104994. Briefly, the lifespan of a protein depends on the character of its N-terminal residue. N-terminal residues that destabilize a protein are termed N-degrons, classified as type 1 or type 2. Type 1 N-degrons contain positively charged amino acids such as Arg, Lys, and His, and type 2 N-degrons include hydrophobic residues such as Phe, Trp, Tyr, Leu, and Ile. In this type of PROTAC, a small molecule that carries an N-terminus that ends with one of these amino acids (Arg, Lys, His, Leu, Ile, Phe, Tyr or Trp) can serve as an N-terminal degradation signal sequence (N-degron).

Light chain (LC) amyloidosis (AL amyloidosis) is a progressive and often fatal degenerative disease caused by monoclonal plasma cell proliferation, resulting in an abnormal free light chain (FLC) ratio and conformational changes within involved immunoglobulin light chains (iFLC) after secretion by clonal plasma cells that result in organ toxicity, e.g., cardiomyopathy, nephrotic syndrome and end-stage renal failure. Organ damage remains the major source of mortality and morbidity. Lambda light chains are more often amyloidogenic than the kappa light chains (˜80%). The light chain conformational changes also often lead to light chain aggregation, which may also drive proteotoxicity in some post-mitotic tissues. The pathologic mechanisms of disease leading to organ toxicity include both toxicity of amyloidogenic LC and mass effects of deposits, both modulated by misfolded LC concentration.

Light chain amyloidosis patients are treated today by targeting the cancer component of this disease (proliferating clonal plasma cells) employing chemotherapy cocktails typically involving proteasome inhibitors (and, when possible, stem cell transplants), which ideally eliminate the clonal plasma cells secreting full-length light chains. However, complete clonal plasma cell eradication is achieved in only 30-40% of the patients and most eventually relapse. Restoration of organ function in treated patients is highly variable and often incomplete, resulting in poor outcomes. The current hematologic response criteria for AL amyloidosis define complete response (CR) as no evidence of monoclonal protein based on serum and urine immunofixation, as well as achieving a normal FLC ratio. The response criteria do not take the levels of iFLC into consideration. It has been shown that increased levels of iFLCs at the time of normal FLC ratio and complete or very good partial hematological response are associated with inferior incomes, i.e., lower organ response and lower overall survival. However, even low levels of amyloidogenic monoclonal FLC can result in organ dysfunction. Moreover, light chain amyloidosis patients exhibiting cardiac involvement are often too sick to tolerate chemotherapy and die within a year of diagnosis.

Thus, there is a need for additional treatments of light chain amyloidosis.

### SUMMARY

Provided herein are compounds for use in compositions and methods of treating AL. In one embodiment, the compounds are PROTACs that target immunoglobulin light chains. In one embodiment, the compounds for use in the compositions and methods provided herein have Formula I:

E-L-X
where E is an E3 ligase ligand or a moiety that activates the N-degron pathway; L is a bond or a divalent chemical linker; and X is a moiety that stabilizes immunoglobulin light chains.

In one embodiment, provided herein is a method of treating light chain amyloidosis by administering to a subject a compound or composition provided herein. In another embodiment, provided is a method of degrading immunoglobulin light chains by contacting the immunoglobulin light chains or a composition containing the immunoglobulin light chains with a compound provided herein. In another embodiment, provided herein is a method of stabilizing immunoglobulin light chains by contacting the immunoglobulin light chains with a compound provided herein. In one embodiment, the immunoglobulin light chains are stabilized in a native conformation thereof. In certain embodiments, the immunoglobulin light chains are dimers. In another embodiment, provided herein is a method of preventing or lessening immunoglobulin light chain misfolding and/or endoproteolysis by contacting the immunoglobulin light chains with a compound provided herein. In another embodiment, provided is a method of maintenance therapy upon recurrence of light chain amyloidosis following primary treatment by administering to a subject a compound or composition provided herein. In another embodiment, provided is a method of combination therapy using a compound or composition provided herein in combination with one or more additional active agents that treat light chain amyloidosis, deplete clonal plasma cells, stabilize immunoglobulin light chains, prevent or lessen immunoglobulin light chain misfolding and/or endoproteolysis, promote clearance of fibrils, or that are effective in maintenance therapy upon recurrence of light chain amyloidosis following primary treatment.

### DETAILED DESCRIPTION

### I. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C=C(OR) where R is alkyl, alkenyl, alkynyl, aryl, aralkyl and cycloalkyl. Pharmaceutically acceptable enol esters include, but are not limited to, derivatives of formula C=C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating AL.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e., C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e., C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R‘- and —R’C(O)₂—.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbornanyl, bicyclo[2.2.2]octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein. The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, ═NR′, =N—OR′, —NR′R″, —SR′, halo, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR′″, —NR—C(NR′R″)=NR′″, —S(O)R′, —S(O)₂R′, —S(O)₂NR′R″, —NRSO₂R′, —NRSO2NR′R″, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R′″ and R′″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R′″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR′″, —NR—C(NR′R″)=NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R′″ and R′″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′″ and R′″ groups when more than one of these groups is present.

Two of the substituent moieties on adjacent atoms of an aryl or heteroaryl ring may optionally form a ring of the formula -Q′—C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(CR″R′″)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R′″ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds provided herein, whether radioactive or not, are encompassed within the scope of the present disclosure.

### II. Compounds for Use in Compositions and Methods

In one embodiment, provided herein is a compound for use in the compositions and methods provided herein having Formula IIa, IIb or IIc:

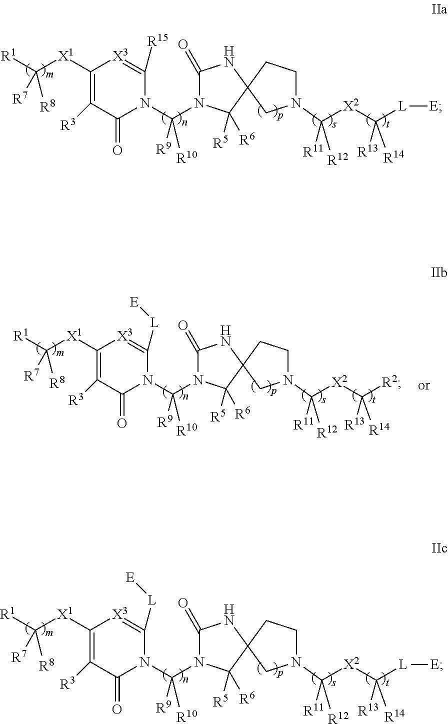

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl, heteroaryl or heterocycloalkyl;
R² is aryl or heteroaryl;
R³ to R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, s and t are each 0, X² is a bond and the compound for use in the compositions and methods provided herein has Formula IId, IIe or IIf:

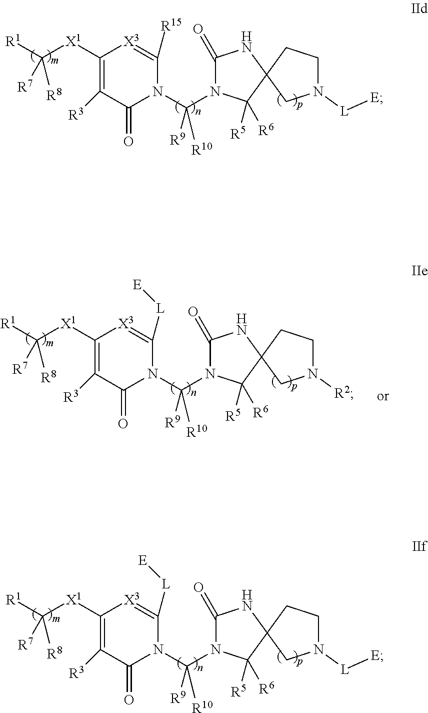

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

In another embodiment, p is 2, s and t are each 0, X² is a bond and the compound for use in the compositions and methods provided herein has Formula IIg, IIh or IIi:

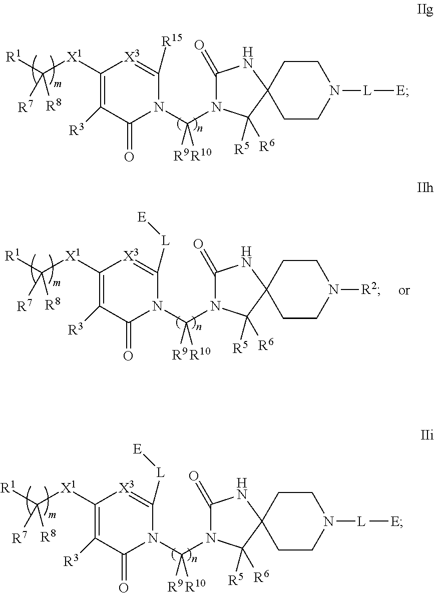

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

In another embodiment, each of R³ to R⁶, R⁹ and R¹⁰ are H, p is 2, s and t are each 0, X² is a bond and the compound for use in the compositions and methods provided herein has Formula IIj, IIk or IIm:

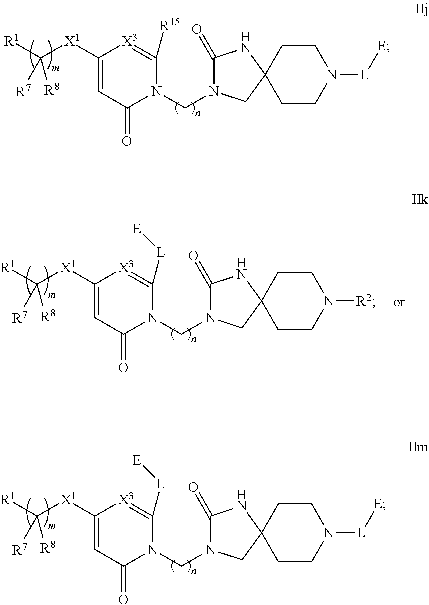

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR¹⁶ or CO;
X³ is N or CH;
R¹ is aryl, heteroaryl or heterocycloalkyl;
R² is heteroaryl;
R⁷ and R⁸ are each independently H, alkyl or aralkyl, or together with the carbon atom to which they are attached form cycloalkyl; and
R¹⁵ and R¹⁶ are each independently H or alkyl.

In another embodiment, R¹ in Formulae II, IIa, IIb or IIc is aryl or heterocycloalkyl. In another embodiment, R¹ in Formulae II, IIa, IIb or IIc is aryl. In another embodiment, R¹ in Formulae II, IIa, IIb or IIc is heterocycloalkyl.

In another embodiment, X¹ is a bond, each of R³ to R⁶, R⁹ and R¹⁰ are H, p is 2, m, s and t are each 0, X² is a bond and the compound for use in the compositions and methods provided herein has Formula IIn, IIo or IIp:

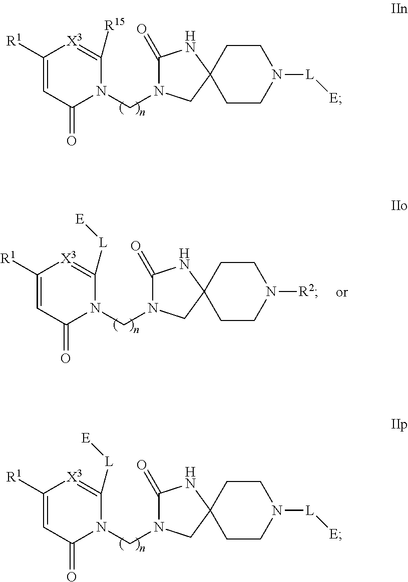

or a pharmaceutically acceptable derivative thereof, wherein
E is an E3 ligase ligand or a moiety that activates the N-degron pathway;
L is a bond or a divalent chemical linker;
n is 2 or 3;
X³ is N or CH;
R¹ is heterocycloalkyl, optionally substituted with 1-3 substituents each independently selected from alkyl and aryl;
R² is heteroaryl; and
R¹⁵ is alkyl.

In another embodiment, the compound for use in the compositions and methods provided herein has Formula IIq, IIr or IIs:

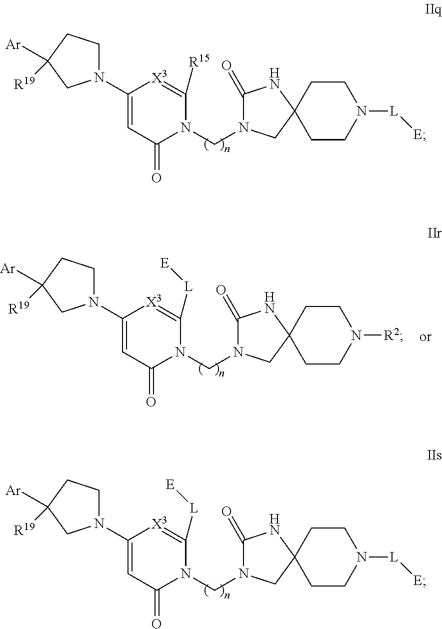

or a pharmaceutically acceptable derivative thereof, wherein
E is an E3 ligase ligand or a moiety that activates the N-degron pathway;
L is a bond or a divalent chemical linker;
n is 2 or 3;
X³ is N or CH;
Ar is aryl;
R² is heteroaryl;
R¹⁵ is alkyl; and
R¹⁹ is alkyl.

In another embodiment, R¹⁹ is lower alkyl. In another embodiment, R¹⁹ is methyl or ethyl. In another embodiment, R¹⁹ is methyl. In another embodiment, R¹⁹ is ethyl.

In another embodiment, the compound for use in the compositions and methods provided herein has the formula:

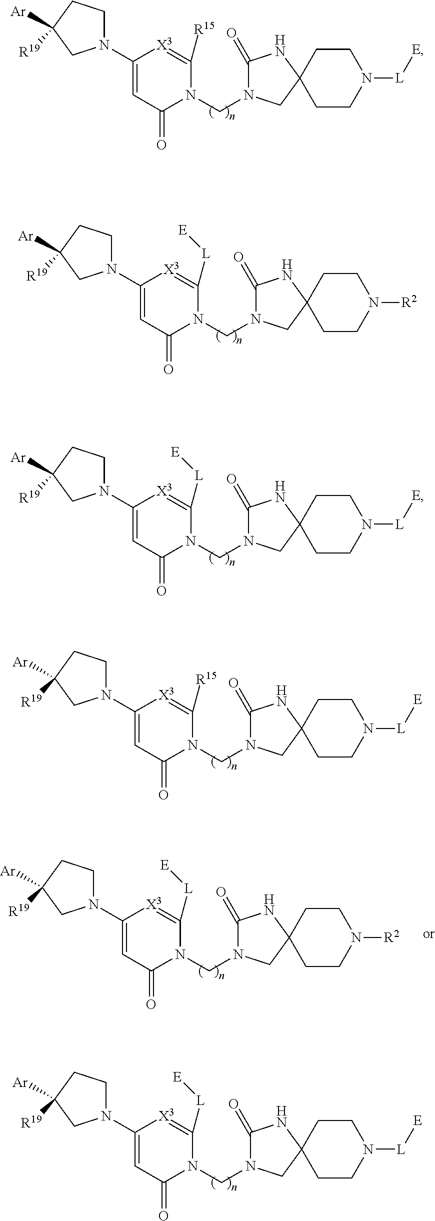

where the variables areas defined herein.

In another embodiment, the compound for use in the compositions and methods provided herein has Formula IIt, IIu or IIv:

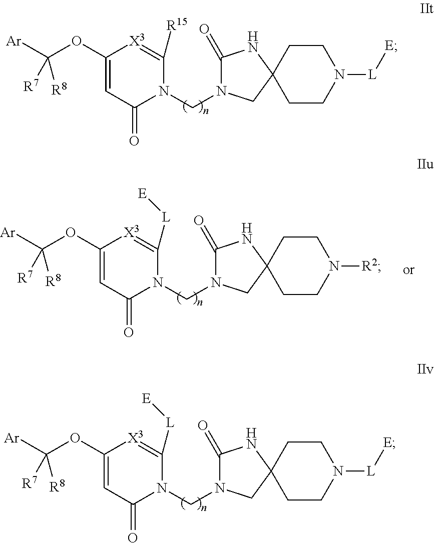

or a pharmaceutically acceptable derivative thereof, wherein
E is an E3 ligase ligand or a moiety that activates the N-degron pathway;
L is a bond or a divalent chemical linker;
n is 2 or 3;
X³ is N or CH;
Ar is aryl;
R² is heteroaryl;
R¹⁵ is alkyl; and
R⁷ and R⁸ are each independently H or alkyl, or together with the carbon atom to which they are attached form cycloalkyl.

In another embodiment, R⁷ is alkyl and R⁸ is H. In another embodiment, R⁷ is methyl and R⁸ is H. In another embodiment, R⁷ and R⁸ together with the carbon atom to which they are attached form cyclopropyl.

In another embodiment, the compound for use in the compositions and methods provided herein has the formula:

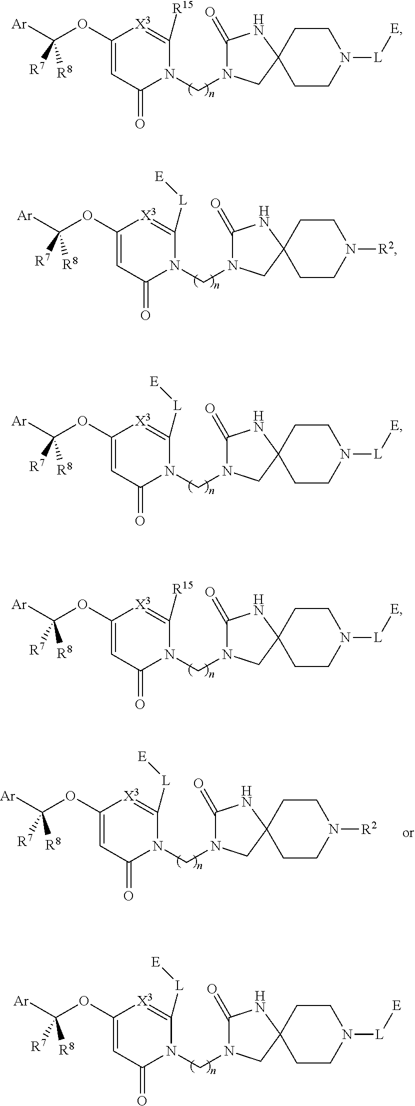

where the variables are as defined herein.

In another embodiment, p is 1, s and t are each 0, X² is a bond and the compound for use in the compositions and methods provided herein has Formula IIw, IIx or IIy:

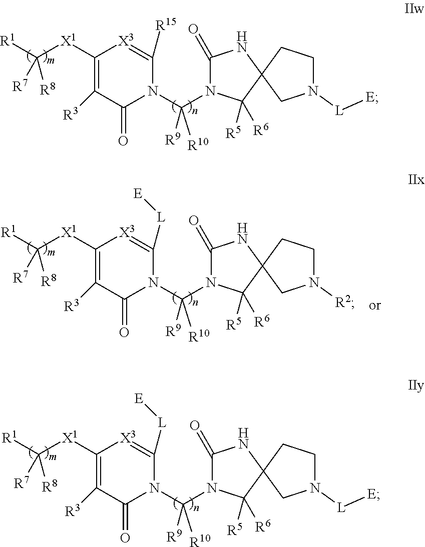

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

In another embodiment, each of R³ to R⁶, R⁹ and R¹⁰ are H, p is 1, s and t are each 0, X² is a bond and the compound for use in the compositions and methods provided herein has Formula IIaa, IIbb or IIcc:

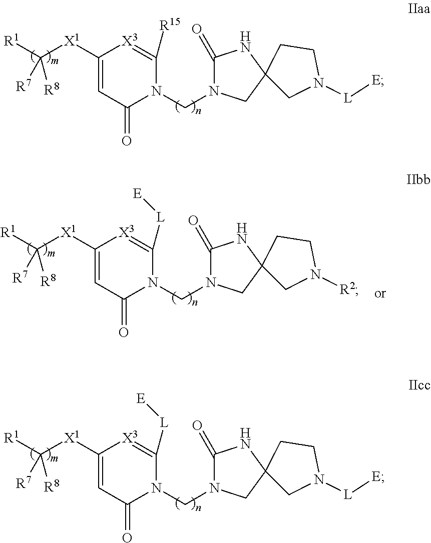

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR¹⁶ or CO;
X³ is N or CH;
R¹ is aryl, heteroaryl or heterocycloalkyl;
R² is heteroaryl;
R⁷ and R⁸ are each independently H, alkyl or aralkyl, or together with the carbon atom to which they are attached form cycloalkyl; and
R¹⁵ and R¹⁶ are each independently H or alkyl.

In another embodiment, R¹ in Formulae IIg or IIh is aryl or heterocycloalkyl. In another embodiment, R¹ in Formulae IIg or IIh is aryl. In another embodiment, R¹ in Formulae IIg or IIh is heterocycloalkyl.

In another embodiment, X¹ is a bond, each of R³ to R⁶, R⁹ and R¹⁰ are H, p is 1, m, s and t are each 0, X² is a bond and the compound for use in the compositions and methods provided herein has Formula IIdd, IIee or IIff.

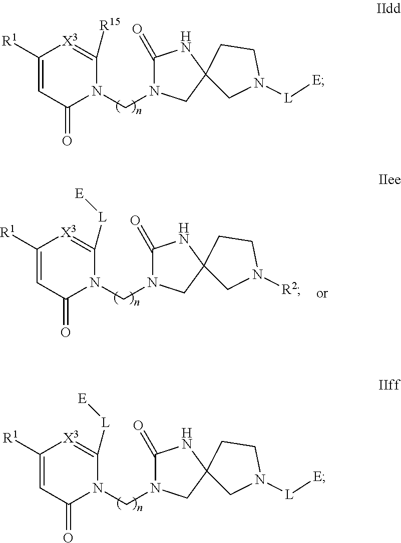

or a pharmaceutically acceptable derivative thereof, wherein
E is an E3 ligase ligand or a moiety that activates the N-degron pathway;
L is a bond or a divalent chemical linker;
n is 2 or 3;
X³ is N or CH;
R¹ is heterocycloalkyl, optionally substituted with 1-3 substituents each independently selected from alkyl and aryl;
R² is heteroaryl; and
R¹⁵ is alkyl.

In another embodiment, the compound for use in the compositions and methods provided herein has Formula IIgg, IIhh or IIii:

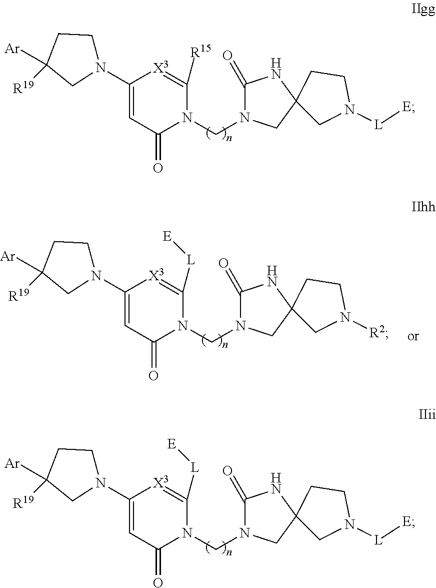

or a pharmaceutically acceptable derivative thereof, wherein
E is an E3 ligase ligand or a moiety that activates the N-degron pathway;
L is a bond or a divalent chemical linker;
n is 2 or 3;
X³ is N or CH;
Ar is aryl;
R² is heteroaryl;
R¹⁵ is alkyl; and
R¹⁹ is alkyl.

In another embodiment, R¹⁹ is lower alkyl. In another embodiment, R¹⁹ is methyl or ethyl. In another embodiment, R¹⁹ is methyl. In another embodiment, R¹⁹ is ethyl.

In another embodiment, the compound for use in the compositions and methods provided herein has the formula:

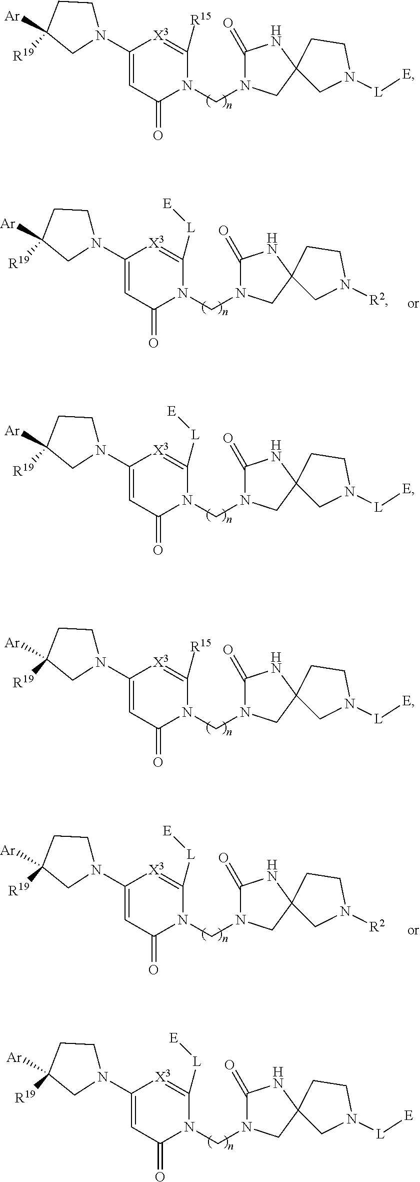

where the variables are as defined herein.

In another embodiment, the compound for use in the compositions and methods provided herein has Formula IIjj, IIkk or IImm:

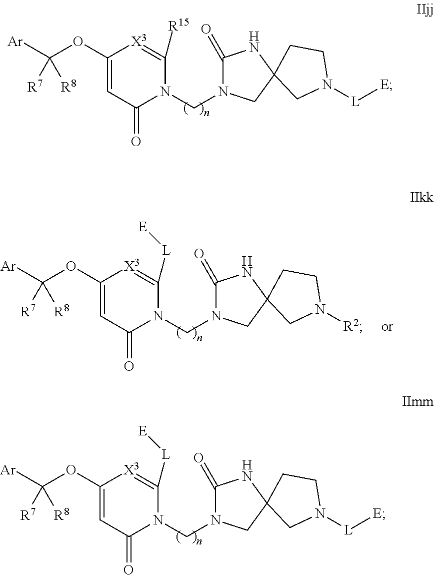

or a pharmaceutically acceptable derivative thereof, wherein
E is an E3 ligase ligand or a moiety that activates the N-degron pathway;
L is a bond or a divalent chemical linker;
n is 2 or 3;
X³ is N or CH;
Ar is aryl;
R² is heteroaryl;
R¹⁵ is alkyl; and
R⁷ and R⁸ are each independently H or alkyl, or together with the carbon atom to which they are attached form cycloalkyl.

In another embodiment, R⁷ is alkyl and R⁸ is H. In another embodiment, R⁷ is methyl and R⁸ is H. In another embodiment, R⁷ and R⁸ together with the carbon atom to which they are attached form cyclopropyl.

In another embodiment, the compound for use in the compositions and methods provided herein has the formula:

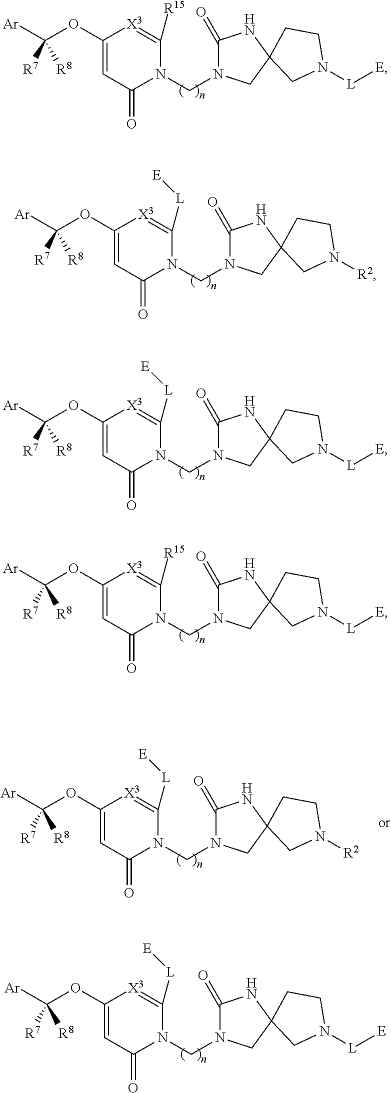

where the variables are as defined herein.

In another embodiment, n is 2. In another embodiment, n is 3.

In another embodiment, X³ is N. In another embodiment, X³ is CH.

In another embodiment, Ar is unsubstituted aryl. In another embodiment, Ar is optionally substituted phenyl. In another embodiment, Ar is unsubstituted phenyl.

In another embodiment, R″ is lower alkyl. In another embodiment, R″ is methyl.

In one embodiment, provided herein is a compound for use in the compositions and methods provided herein having Formula IInn, IIoo or IIpp:

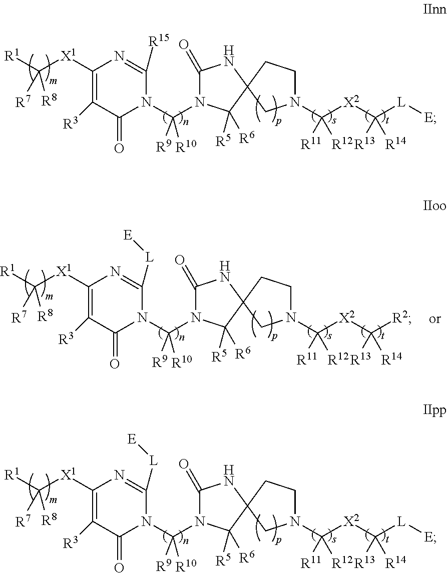

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl, heteroaryl or heterocycloalkyl;
R² is aryl or heteroaryl;
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula IInn, IIoo or IIpp, or a pharmaceutically acceptable derivative thereof, wherein:
E is an E3 ligase ligand or a moiety that activates the N-degron pathway;
L is a bond or a divalent chemical linker;
n is 2 or 3;
p is 2;
m is 0 or 1;
s and t are each 0;
X¹ is a bond or 0;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula IInn, IIoo or IIpp, or a pharmaceutically acceptable derivative thereof, wherein:
E is an E3 ligase ligand or a moiety that activates the N-degron pathway;
L is a bond or a divalent chemical linker;
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁸ to R¹⁴ are each independently H; and
R⁷ and R¹⁵ are each independently alkyl.

In another embodiment, the compounds provided herein for use in the compositions and methods provided herein have Formula IInn, IIoo or IIpp, or a pharmaceutically acceptable derivative thereof, wherein:
E is an E3 ligase ligand or a moiety that activates the N-degron pathway;
L is a bond or a divalent chemical linker;
n is 2 or 3;
p is 1;
m is 0 or 1;
s and t are each 0;
X¹ is a bond or 0;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula IInn, IIoo or IIpp, or a pharmaceutically acceptable derivative thereof, wherein:
E is an E3 ligase ligand or a moiety that activates the N-degron pathway;
L is a bond or a divalent chemical linker;
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁸ to R¹⁴ are each independently H; and
R⁷ and R¹⁵ are each independently alkyl.

In another embodiment, provided herein is a compound for use in the compositions and methods provided herein having Formula IIqq, IIrr or IIss:

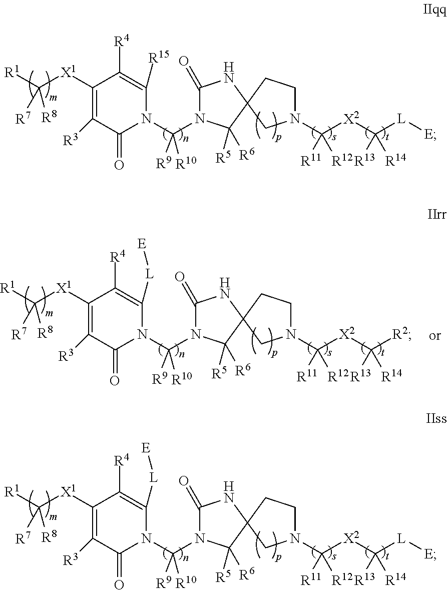

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl, heteroaryl or heterocycloalkyl;
R² is aryl or heteroaryl;
R³ to R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula IIqq, IIrr or IIss, or a pharmaceutically acceptable derivative thereof, wherein:
E is an E3 ligase ligand or a moiety that activates the N-degron pathway;
L is a bond or a divalent chemical linker;
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁶ and R⁹ to R¹⁵ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula IIqq, IIrr or IIss, or a pharmaceutically acceptable derivative thereof, wherein:
E is an E3 ligase ligand or a moiety that activates the N-degron pathway;
L is a bond or a divalent chemical linker;
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁵, R⁶ and R⁸ to R¹⁴ are each independently H; and
R⁷ and R¹⁵ are each independently alkyl.

In another embodiment, the compounds provided herein for use in the compositions and methods provided herein have Formula IIqq, IIrr or IIss, or a pharmaceutically acceptable derivative thereof, wherein:
E is an E3 ligase ligand or a moiety that activates the N-degron pathway;
L is a bond or a divalent chemical linker;
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁶ and R⁹ to R¹⁵ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula IIqq, IIrr or IIss, or a pharmaceutically acceptable derivative thereof, wherein:
E is an E3 ligase ligand or a moiety that activates the N-degron pathway;
L is a bond or a divalent chemical linker;
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁵, R⁶ and R⁸ to R¹⁴ are each independently H; and
R⁷ and R¹⁵ are each independently alkyl.

In another embodiment, s and t are each 0, X² is a bond, X³ is CR⁴ and the compounds for use in the compositions and methods provided herein have Formula IItt, IIuu or IIvv:

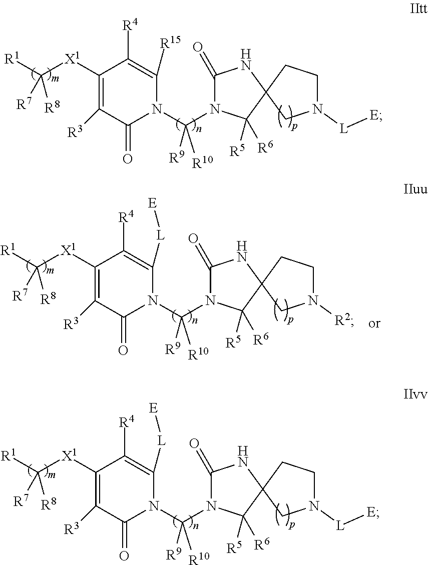

wherein:
E is an E3 ligase ligand or a moiety that activates the N-degron pathway;
L is a bond or a divalent chemical linker;
n is an integer from 1-4;
p is an integer from 1-3;
m is an integer from 0-3;
X¹ is a bond, O, NR¹⁶ or CO;
R¹ is aryl or heterocycloalkyl;
R² is heteroaryl; and
R³ to R¹⁰, R¹⁵ and R¹⁶ are each independently H or alkyl.

In another embodiment, s and t are each 0, X² is a bond, X³ is N and the compounds for use in the compositions and methods provided herein have Formula IIww, IIxx or IIyy:

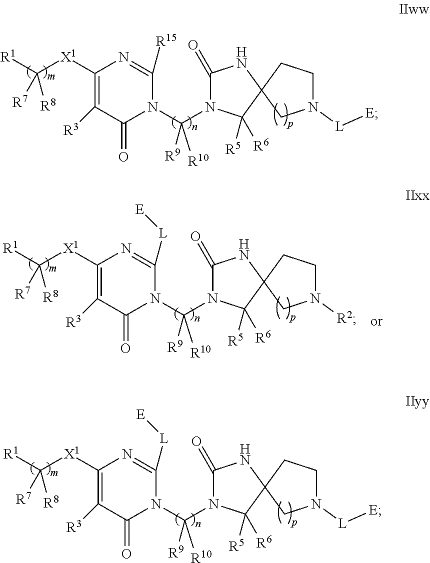

wherein:
E is an E3 ligase ligand or a moiety that activates the N-degron pathway;
L is a bond or a divalent chemical linker;
n is an integer from 1-4;
p is an integer from 1-3;
m is an integer from 0-3;
X¹ is a bond, O, NR¹⁶ or CO;
R¹ is aryl or heterocycloalkyl;
R² is heteroaryl; and
R³, R⁵ to R¹⁰, R¹⁵ and R¹⁶ are each independently H or alkyl.

In another embodiment, the compounds for use in the compositions and methods provided herein have Formula IItt, IIuu, IIvv, IIww, IIxx or IIyy, wherein:
E is an E3 ligase ligand or a moiety that activates the N-degron pathway;
L is a bond or a divalent chemical linker;
n is 2 or 3;
p is 2;
m is 0 or 1;
X¹ is a bond or O;
R¹ is aryl or heterocycloalkyl, each optionally substituted with 1-3 substituents each independently selected from alkyl and aryl;
R² is heteroaryl; and
R³, R⁵ to R¹⁰, R¹⁵ and R¹⁶ are each independently H or alkyl.

In another embodiment, the compounds for use in the compositions and methods provided herein have Formula IItt, IIuu, IIvv, IIww, IIxx or IIyy, wherein:
E is an E3 ligase ligand or a moiety that activates the N-degron pathway;
L is a bond or a divalent chemical linker;
n is 2 or 3;
p is 1;
m is 0 or 1;
X¹ is a bond or O;
R¹ is aryl or heterocycloalkyl, each optionally substituted with 1-3 substituents each independently selected from alkyl and aryl;
R² is heteroaryl; and
R³, R⁵ to R¹⁰, R¹⁵ and R¹⁶ are each independently H or alkyl.

In one embodiment, the compounds provided herein for use in the compositions and methods provided herein are selected from:

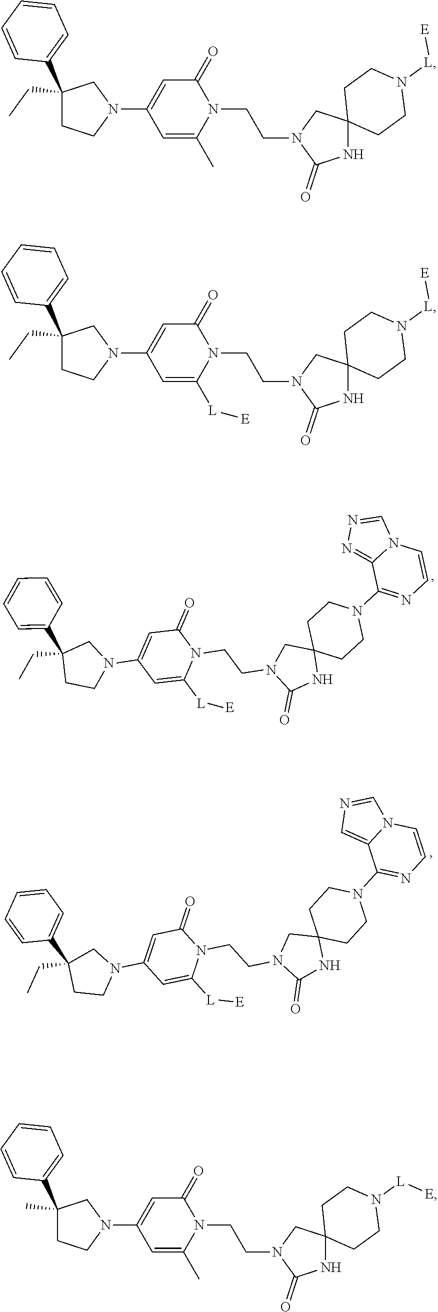

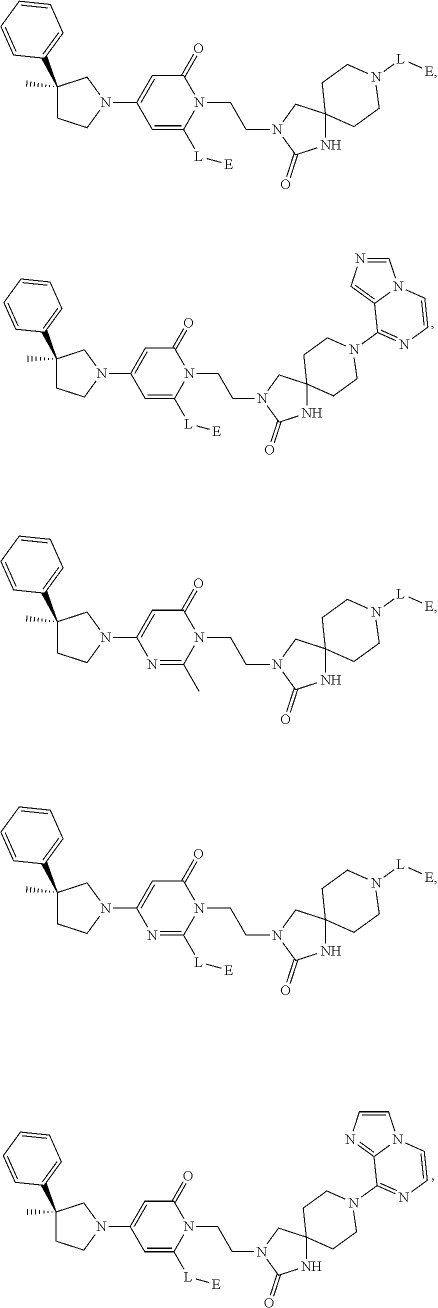

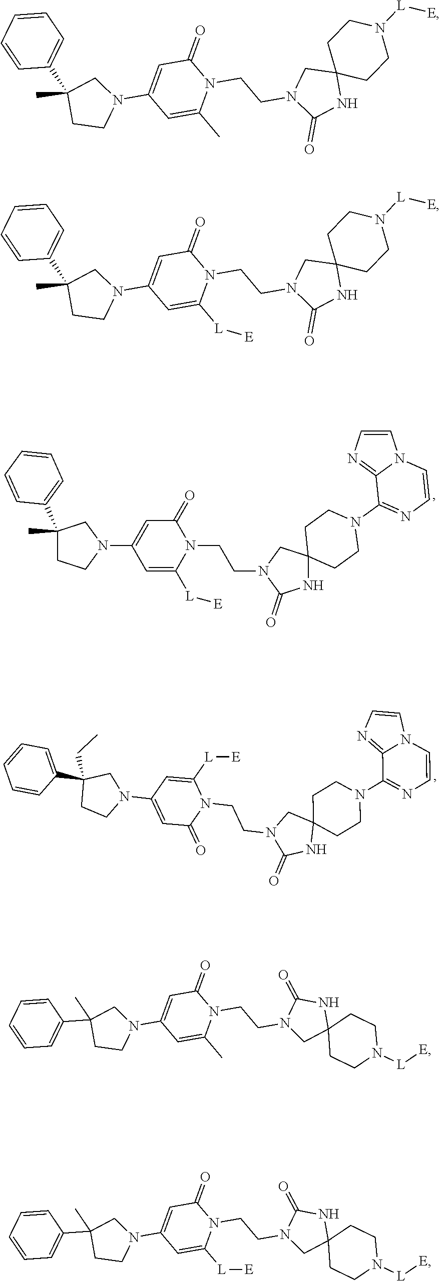

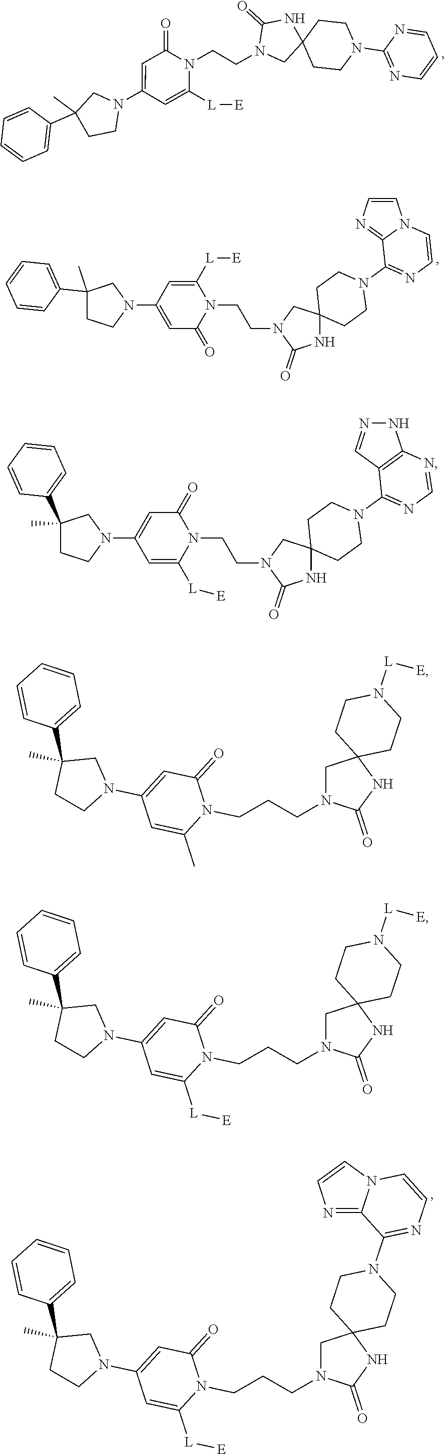

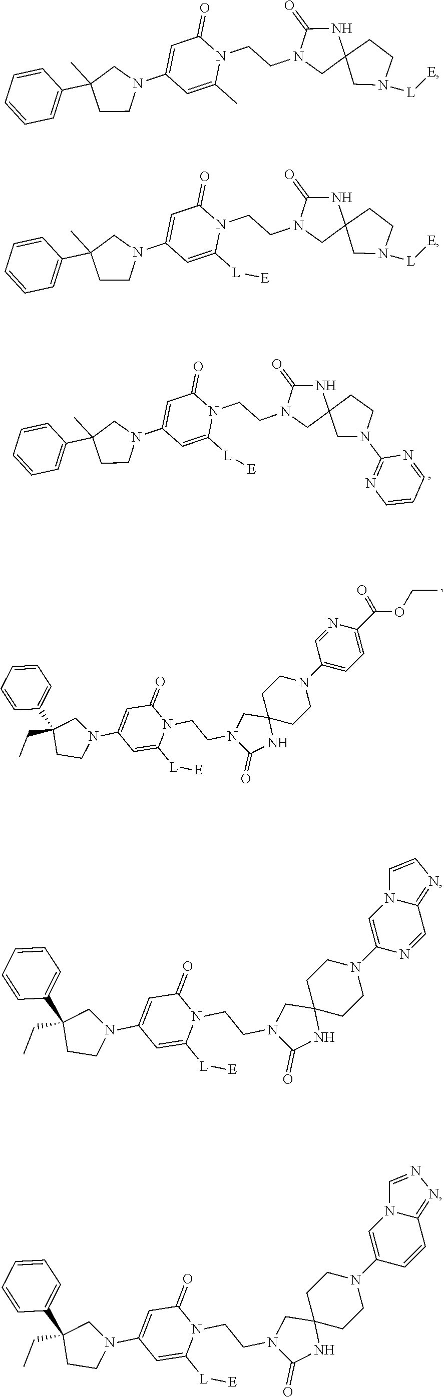

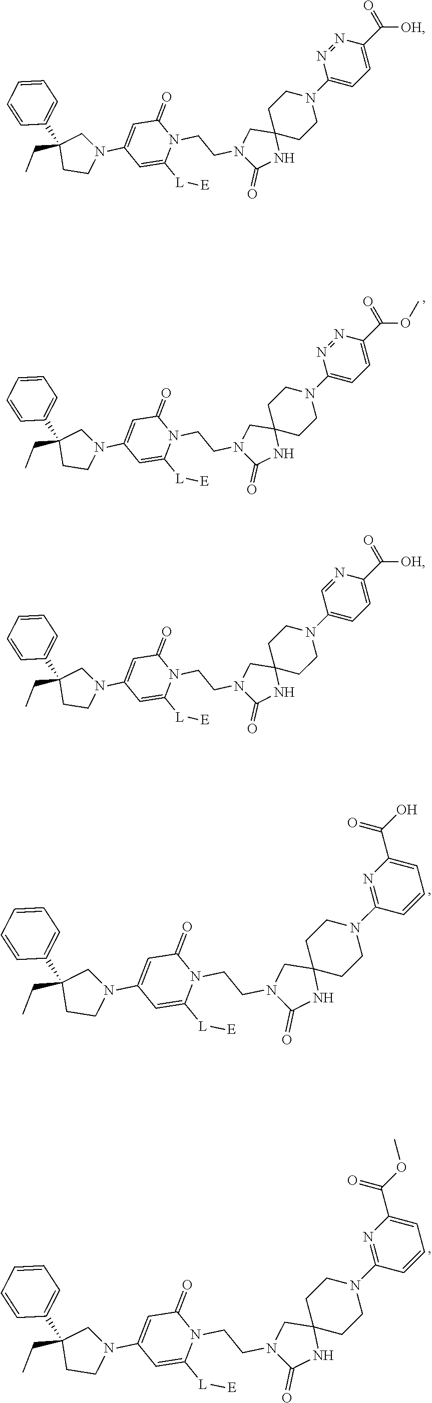

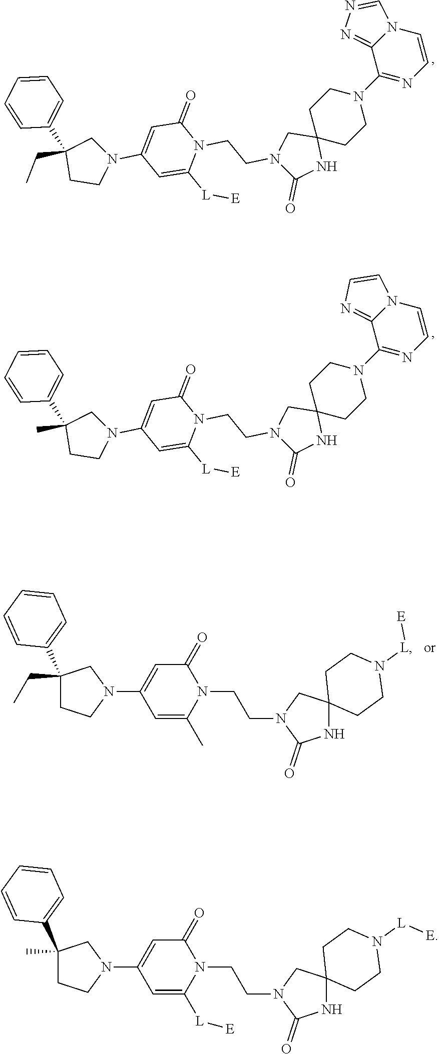

In another embodiment, provided herein is a compound for use in the compositions and methods provided herein having Formula IIIa, IIIb or IIc:

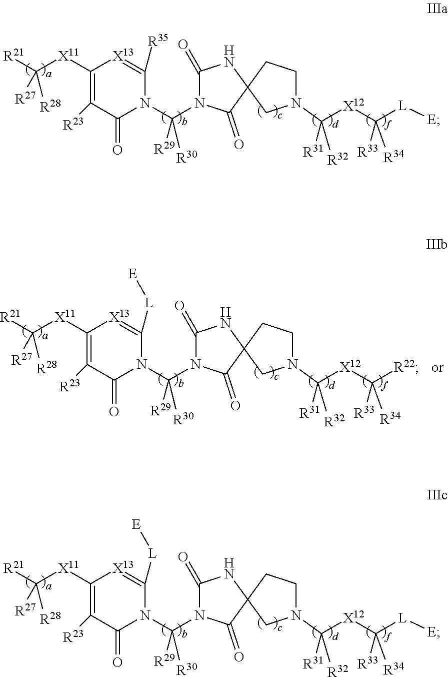

or a pharmaceutically acceptable derivative thereof, wherein:
E is an E3 ligase ligand or a moiety that activates the N-degron pathway;
L is a bond or a divalent chemical linker;
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
X¹³ is N or CR²⁴;
R²¹ is aryl, heteroaryl or heterocycloalkyl;
R²² is aryl or heteroaryl;
R²³ to R²⁶ and R²⁹ to R³⁵ are each independently H, alkyl or aralkyl; and
R²⁷ and R²⁸ are each independently H, alkyl or aralkyl, or together with the carbon atom to which they are attached form cycloalkyl.

In another embodiment, d and f are each 0, X¹² is a bond and the compound for use in the compositions and methods provided herein has Formula IIId, IIIe or IIIf.

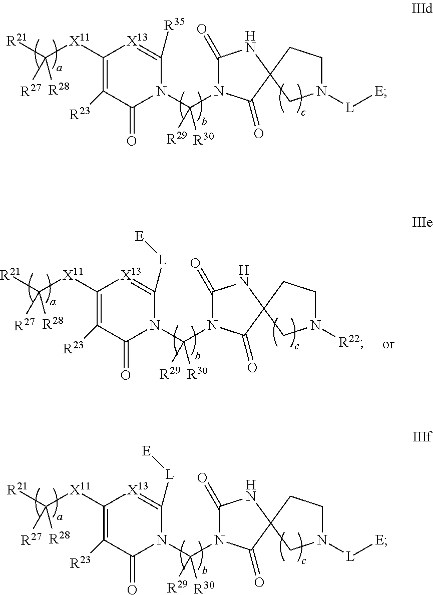

or a pharmaceutically acceptable derivative thereof, wherein:
E is an E3 ligase ligand or a moiety that activates the N-degron pathway;
L is a bond or a divalent chemical linker;
b is an integer from 1-4;
c is an integer from 1-3;
a is an integer from 0-3;
X¹¹ is a bond, O, NR²⁵ or CO;
X¹³ is N or CR²⁴;
R²¹ is aryl, heteroaryl or heterocycloalkyl;
R²² is heteroaryl;
R²³ to R²⁵, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

In another embodiment, c is 2, d and f are each 0, X¹² is a bond and the compound for use in the compositions and methods provided herein has Formula IIIg, IIIh or IIIi:

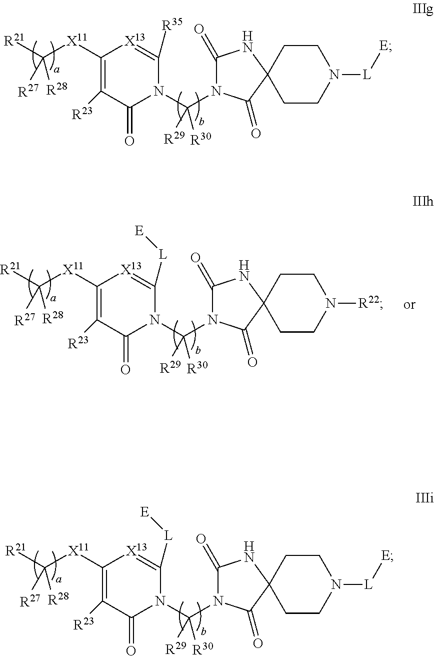

or a pharmaceutically acceptable derivative thereof, wherein:
E is an E3 ligase ligand or a moiety that activates the N-degron pathway;
L is a bond or a divalent chemical linker;
b is an integer from 1-4;
a is an integer from 0-3;
X¹¹ is a bond, O, NR²⁵ or CO;
X¹³ is N or CR²⁴;
R²¹ is aryl, heteroaryl or heterocycloalkyl;
R²² is heteroaryl;
R²³ to R²⁵, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

In another embodiment, each of R²³, R²⁹ and R³⁰ are H, c is 2, d and f are each 0, X¹² is a bond and the compound for use in the compositions and methods provided herein has Formula IIIj, IIIk or IIIm:

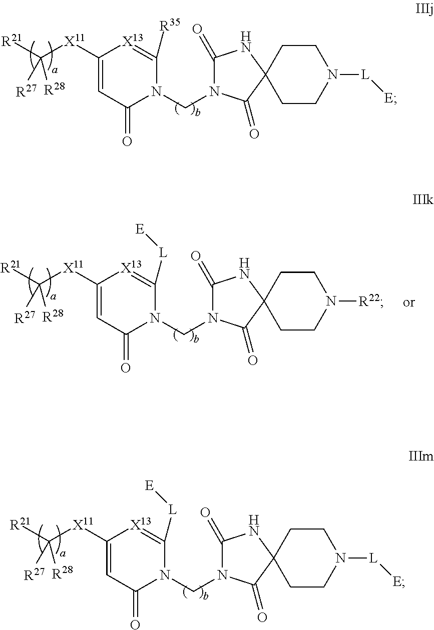

or a pharmaceutically acceptable derivative thereof, wherein:
E is an E3 ligase ligand or a moiety that activates the N-degron pathway;
L is a bond or a divalent chemical linker;
b is an integer from 1-4;
a is an integer from 0-3;
X¹¹ is a bond, O, NR²⁵ or CO;
X¹³ is N or CH;
R²¹ is aryl, heteroaryl or heterocycloalkyl;
R²² is heteroaryl;
R²⁵ and R³⁵ are each independently H or alkyl; and
R²⁷ and R²⁸ are each independently H, alkyl or aralkyl, or together with the carbon atom to which they are attached form cycloalkyl.

In another embodiment, R²¹ in Formulae IIIa, IIIb, IIIc, IIId, IIIe, IIIf, IIIg, IIIh, IIIi, IIIj, IIIk or IIIm is aryl or heterocycloalkyl. In another embodiment, R²¹ in Formulae IIIa, IIIhb, IIIc, IIId, IIIe, IIIf, IIIg, IIIh, IIIi, IIIj, IIIk or IIIm is aryl. In another embodiment, R²¹ in Formulae IIIa, IIIh, IIIc, IIId, IIIe, IIIf, IIIg, IIIh, IIIi, IIIj, IIIk or IIIm is heterocycloalkyl.

In another embodiment, X¹¹ is a bond, each of R²³, R²⁹ and R³⁰ are H, c is 2, a, d and f are each 0, X¹² is a bond and the compound for use in the compositions and methods provided herein has Formula IIIn, IIIo or IIIp:

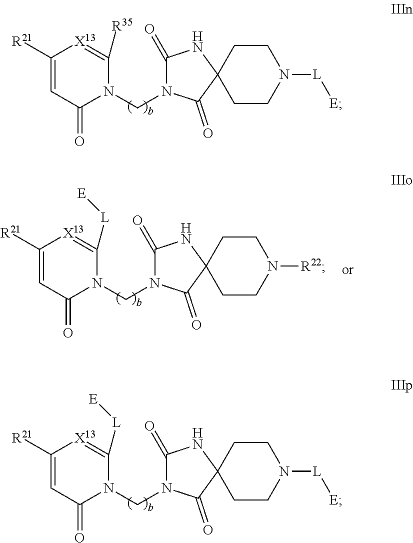

or a pharmaceutically acceptable derivative thereof, wherein:
E is an E3 ligase ligand or a moiety that activates the N-degron pathway;
L is a bond or a divalent chemical linker;
b is 2 or 3;
X¹³ is N or CH;
R²¹ is heterocycloalkyl, optionally substituted with 1-3 substituents each independently selected from alkyl and aryl;
R²² is heteroaryl; and
R³⁵ is alkyl.

In another embodiment, the compound for use in the compositions and methods provided herein has Formula IIIq, IIIr or IIIs:

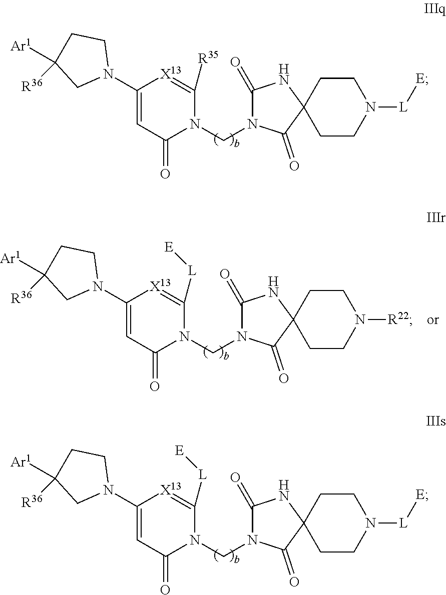

or a pharmaceutically acceptable derivative thereof, wherein:
E is an E3 ligase ligand or a moiety that activates the N-degron pathway;
L is a bond or a divalent chemical linker;
b is 2 or 3;
X¹³ is N or CH;
Ar¹ is aryl;
R²² is heteroaryl;
R³⁵ is alkyl; and
R³⁶ is alkyl.

In another embodiment, R³⁶ is lower alkyl. In another embodiment, R³⁶ is methyl or ethyl. In another embodiment, R³⁶ is methyl. In another embodiment, R³⁶ is ethyl.

In another embodiment, the compound for use in the compositions and methods provided herein has the formula:

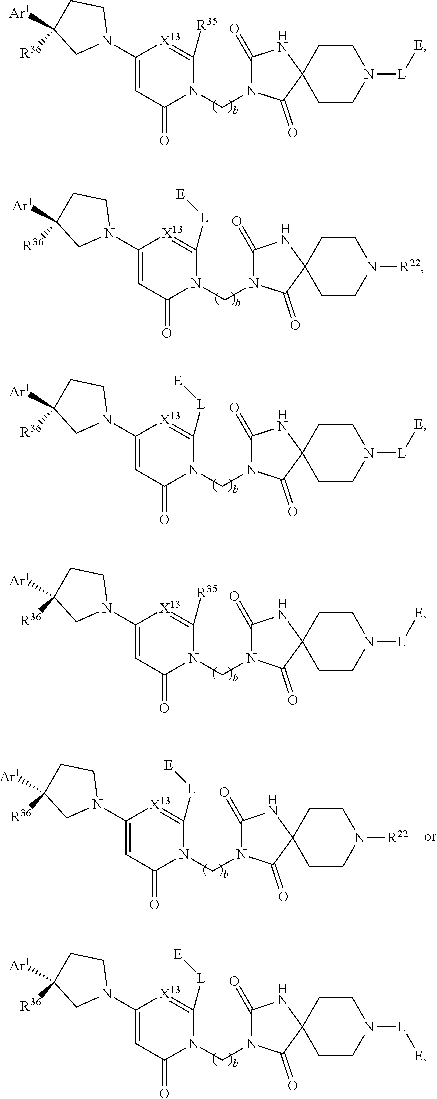

where the variables are as defined herein.

In another embodiment, the compound for use in the compositions and methods provided herein has Formula IIIt, IIIu or IIIy:

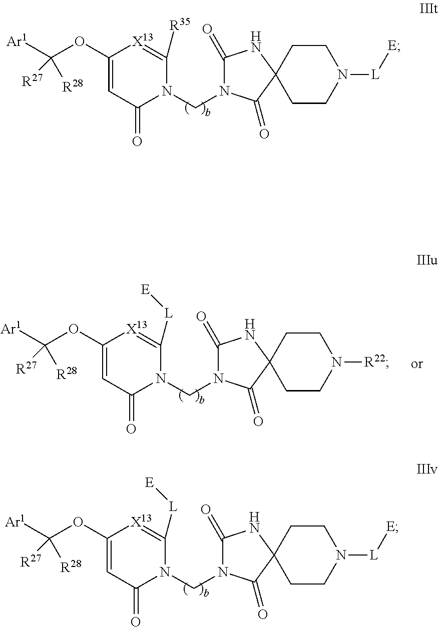

or a pharmaceutically acceptable derivative thereof, wherein:
E is an E3 ligase ligand or a moiety that activates the N-degron pathway;
L is a bond or a divalent chemical linker;
b is 2 or 3;
X¹³ is N or CH;
Ar¹ is aryl;
R²² is heteroaryl;
R³⁵ is alkyl; and
R²⁷ and R²⁸ are each independently H, alkyl or aralkyl, or together with the carbon atom to which they are attached form cycloalkyl.

In another embodiment, R²⁷ is alkyl and R²⁸ is H. In another embodiment, R²⁷ is methyl and R²⁸ is H. In another embodiment, R²⁷ and R²⁸ together with the carbon atom to which they are attached form cyclopropyl.

In another embodiment, the compound for use in the compositions and methods provided herein has the formula:

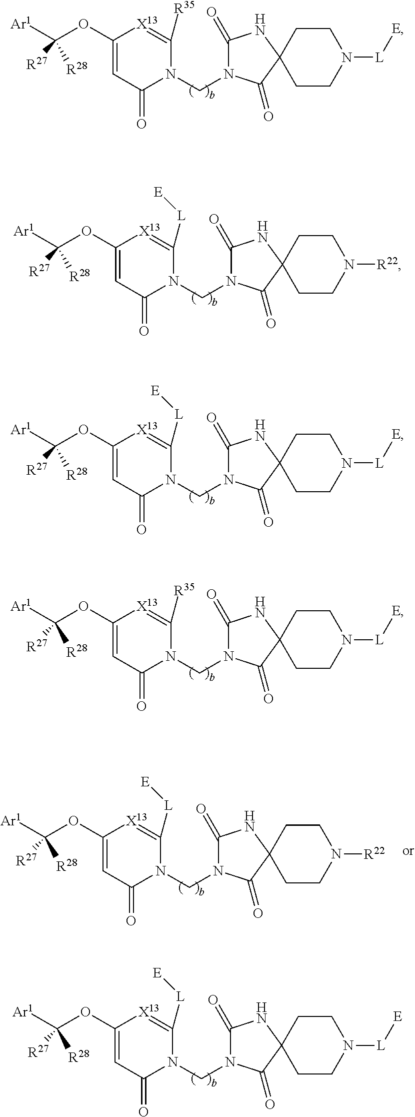

where the variables are as defined herein.

In another embodiment, c is 1, d and f are each 0, X¹² is a bond and the compound for use in the compositions and methods provided herein has Formula IIIw, IIIx or IIIy:

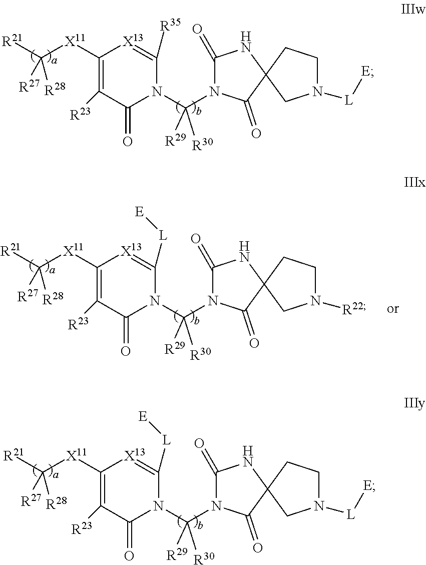

or a pharmaceutically acceptable derivative thereof, wherein:
E is an E3 ligase ligand or a moiety that activates the N-degron pathway;
L is a bond or a divalent chemical linker;
b is an integer from 1-4;
a is an integer from 0-3;
X¹¹ is a bond, O, NR²⁵ or CO;
X¹³ is N or CR²⁴.
R²¹ is aryl, heteroaryl or heterocycloalkyl;
R²² is heteroaryl;
R²³ to R²⁵, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

In another embodiment, each of R²³, R²⁹ and R³⁰ are H, c is 1, d and f are each 0, X¹² is a bond and the compound for use in the compositions and methods provided herein has Formula IIIaa, IIIbb or IIIcc:

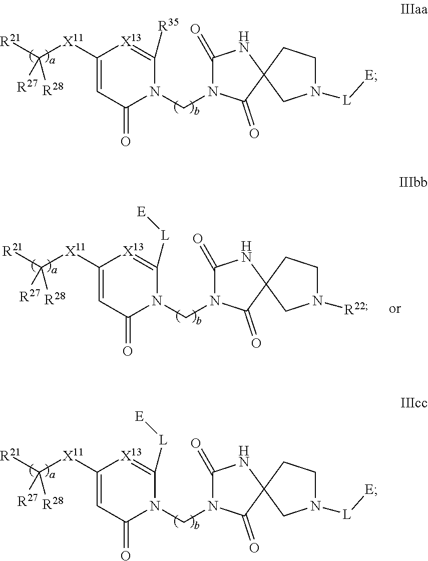

or a pharmaceutically acceptable derivative thereof, wherein:
E is an E3 ligase ligand or a moiety that activates the N-degron pathway;
L is a bond or a divalent chemical linker;
b is an integer from 1-4;
a is an integer from 0-3;
X¹¹ is a bond, O, NR²⁵ or CO;
X¹³ is N or CH;
R²¹ is aryl, heteroaryl or heterocycloalkyl;
R²² is heteroaryl;
R²⁵ and R³⁵ are each independently H or alkyl; and
R²⁷ and R²⁸ are each independently H, alkyl or aralkyl, or together with the carbon atom to which they are attached form cycloalkyl.

In another embodiment, R²¹ in Formulae IIIw, IIIx, IIIy, IIIaa, IIIbb or IIIcc is aryl or heterocycloalkyl. In another embodiment, R²¹ in Formulae IIIw, IIIx, IIIy, IIIaa, IIIbb or IIIcc is aryl. In another embodiment, R²¹ in Formulae IIIw, IIIx, IIIy, IIIaa, IIIbb or IIIcc is heterocycloalkyl.

In another embodiment, X¹¹ is a bond, each of R²³, R²⁹ and R³⁰ are H, c is 2, a, d and f are each 0, X¹² is a bond and the compound for use in the compositions and methods provided herein has Formula IIIdd, IIIee or IIIff:

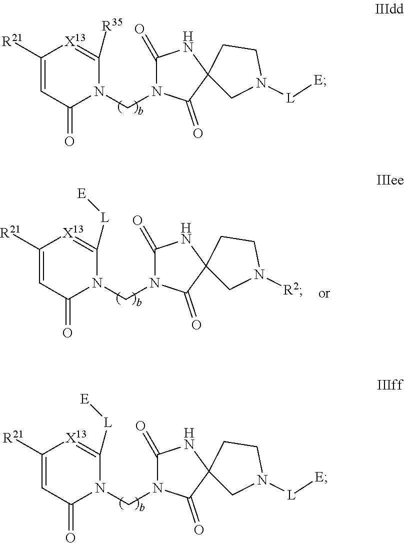

or a pharmaceutically acceptable derivative thereof, wherein:
E is an E3 ligase ligand or a moiety that activates the N-degron pathway;
L is a bond or a divalent chemical linker;
b is 2 or 3;
X¹³ is N or CH;
R²¹ is heterocycloalkyl, optionally substituted with 1-3 substituents each independently selected from alkyl and aryl;
R²² is heteroaryl; and
R³⁵ is alkyl.

In another embodiment, the compound for use in the compositions and methods provided herein has Formula IIIgg, IIIhh or IIIii:

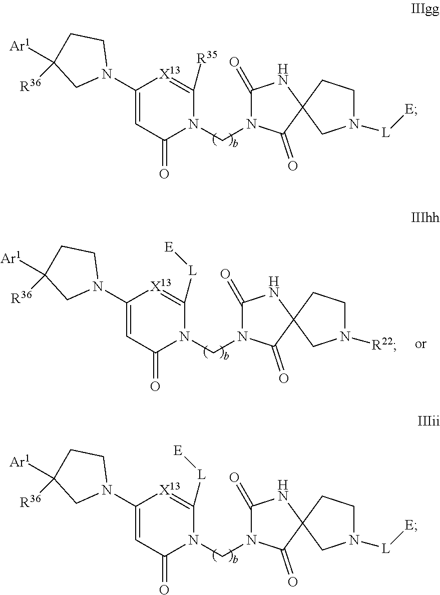

or a pharmaceutically acceptable derivative thereof, wherein:
E is an E3 ligase ligand or a moiety that activates the N-degron pathway;
L is a bond or a divalent chemical linker;
b is 2 or 3;
X¹³ is N or CH;
Ar¹ is aryl;
R²² is heteroaryl;
R³⁵ is alkyl; and
R³⁶ is alkyl.

In another embodiment, R³⁶ is lower alkyl. In another embodiment, R³⁶ is methyl or ethyl. In another embodiment, R³⁶ is methyl. In another embodiment, R³⁶ is ethyl.

In another embodiment, the compound for use in the compositions and methods provided herein has the formula:

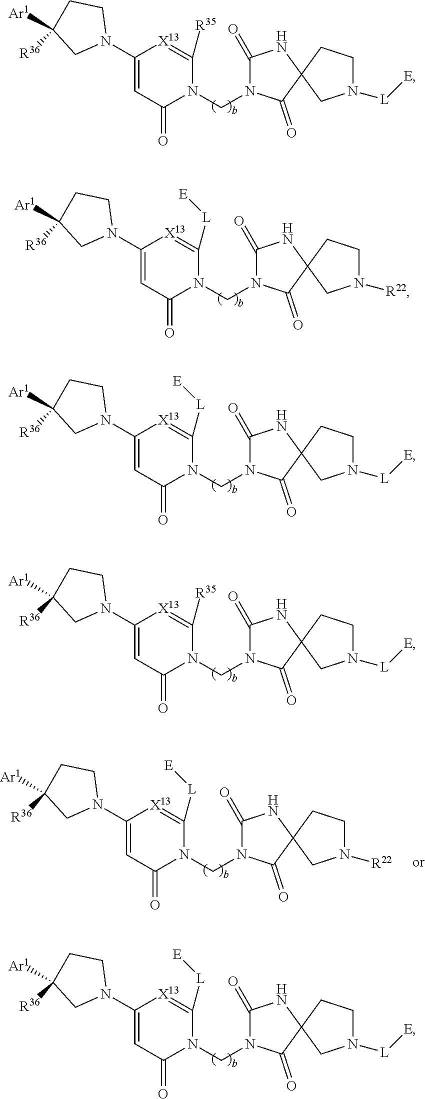

where the variables are as defined herein.

In another embodiment, the compound for use in the compositions and methods provided herein has Formula IIIjj, IIIkk or IIImm:

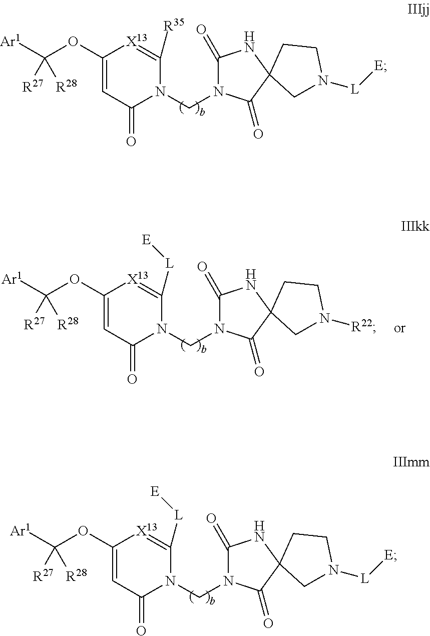

or a pharmaceutically acceptable derivative thereof, wherein:
E is an E3 ligase ligand or a moiety that activates the N-degron pathway;
L is a bond or a divalent chemical linker;
b is 2 or 3;
X¹³ is N or CH;
Ar¹ is aryl;
R²² is heteroaryl;
R³⁵ is alkyl; and
R²⁷ and R²⁸ are each independently H, alkyl or aralkyl, or together with the carbon atom to which they are attached form cycloalkyl.

In another embodiment, R²⁷ is alkyl and R²⁸ is H. In another embodiment, R²⁷ is methyl and R²⁸ is H. In another embodiment, R²⁷ and R²⁸ together with the carbon atom to which they are attached form cyclopropyl.

In another embodiment, the compound for use in the compositions and methods provided herein has the formula:

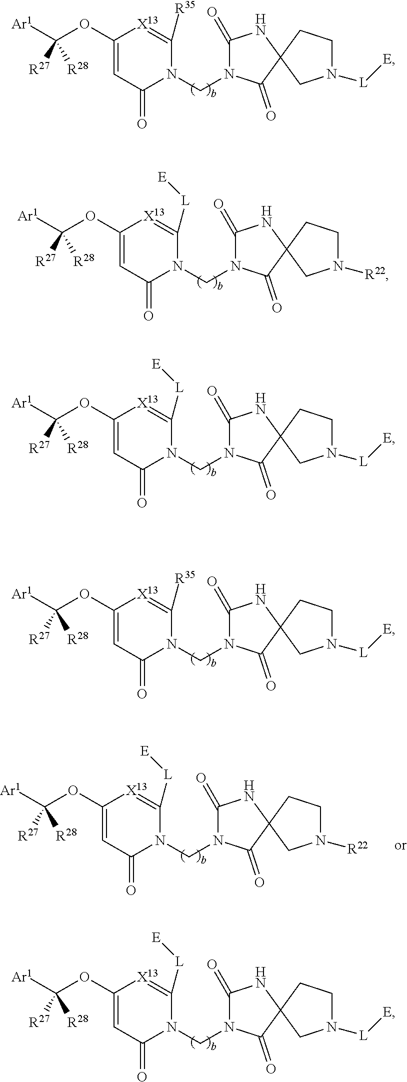

where the variables are as defined herein.

In another embodiment, b is 2. In another embodiment, b is 3.

In another embodiment, X¹³ is N. In another embodiment, X¹³ is CH.

In another embodiment, Ar¹ is unsubstituted aryl. In another embodiment, Ar¹ is optionally substituted phenyl. In another embodiment, Ar¹ is unsubstituted phenyl.

In another embodiment, R²² is bicyclic or monocyclic heteroaryl. In another embodiment, R²² is triazolopyrazinyl, imidazopyrazinyl, pyrazolopyrimidinyl, pyridyl, pyridazinyl or pyrimidinyl. In another embodiment, R²² is triazolopyrazinyl, imidazopyrazinyl, pyrazolopyrimidinyl or pyrimidinyl. In another embodiment, R²² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl or 2-pyrimidinyl. In another embodiment, R²² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl, 2-pyridyl, 3-pyridyl, 3-pyridazinyl or 2-pyrimidinyl. In another embodiment, R²² is [1,2,4]-triazolo[4,3-a]pyrazin-8-yl, imidazo[1,5-a]pyrazin-8-yl, 5-carboxyimidazo[1,5-a]pyrazin-8-yl, imidazo[1,2-a]pyrazin-8-yl, 1H-pyrazolo[3,4-d]pyrimidin-4-yl, 6-carboxy-3-pyridyl, 6-carboxy-2-pyridyl, 6-ethoxycarbonyl-2-pyridyl, 6-ethoxycarbonyl-3-pyridyl, 6-carboxy-3-pyridazinyl, 6-ethoxycarbonyl-3-pyridazinyl or 2-pyrimidinyl.

In another embodiment, R³⁵ is lower alkyl. In another embodiment, R³⁵ is methyl.

In one embodiment, provided herein is a compound for use in the compositions and methods provided herein having Formula IIInn, IIIoo or IIIpp:

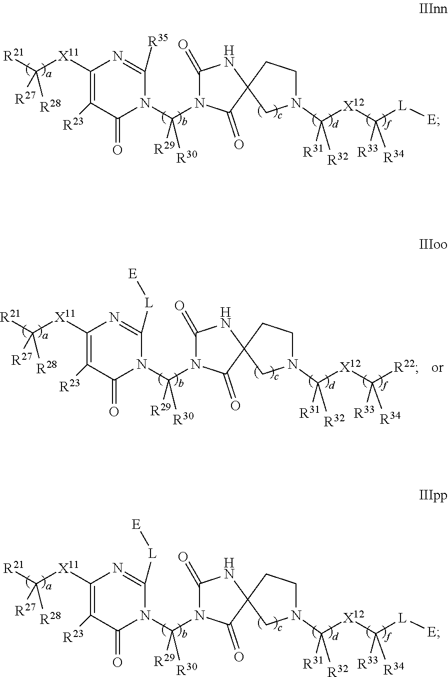

or a pharmaceutically acceptable derivative thereof, wherein
E is an E3 ligase ligand or a moiety that activates the N-degron pathway;
L is a bond or a divalent chemical linker;
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
R²¹ is aryl, heteroaryl or heterocycloalkyl;
R²² is aryl or heteroaryl;
R²³, R²⁵, R²⁶ and R²⁹ to R³⁵ are each independently H, alkyl or aralkyl; and
R²⁷ and R²⁸ are each independently H, alkyl or aralkyl, or together with the carbon atom to which they are attached form cycloalkyl.

In another embodiment, the compounds provided herein for use in the compositions and methods provided herein have Formula IIInn, IIIoo or IIIpp, or a pharmaceutically acceptable derivative thereof, wherein:
E is an E3 ligase ligand or a moiety that activates the N-degron pathway;
L is a bond or a divalent chemical linker;
b is 2 or 3;
c is 2;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

In another embodiment, the compounds provided herein for use in the compositions and methods provided herein have Formula IIInn, IIIoo or IIIpp, or a pharmaceutically acceptable derivative thereof, wherein:
E is an E3 ligase ligand or a moiety that activates the N-degron pathway;
L is a bond or a divalent chemical linker;
b is 2 or 3;
c is 2;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³ and R²⁸ to R³⁰ are each independently H; and
R²⁷ and R³⁵ are each independently alkyl.

In another embodiment, the compounds provided herein for use in the compositions and methods provided herein have Formula IIInn, IIIoo or IIIpp, or a pharmaceutically acceptable derivative thereof, wherein:
E is an E3 ligase ligand or a moiety that activates the N-degron pathway;
L is a bond or a divalent chemical linker;
b is 2 or 3;
c is 1;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

In another embodiment, the compounds provided herein for use in the compositions and methods provided herein have Formula IIInn, IIIoo or IIIpp, or a pharmaceutically acceptable derivative thereof, wherein:
E is an E3 ligase ligand or a moiety that activates the N-degron pathway;
L is a bond or a divalent chemical linker;
b is 2 or 3;
c is 1;
a is 0 or 1;
d and f are each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³ and R²⁸ to R³⁰ are each independently H; and
R²⁷ and R³⁵ are each independently alkyl.

In another embodiment, provided herein is a compound for use in the compositions and methods provided herein having Formula IIIqq, IIIrr or IIIss:

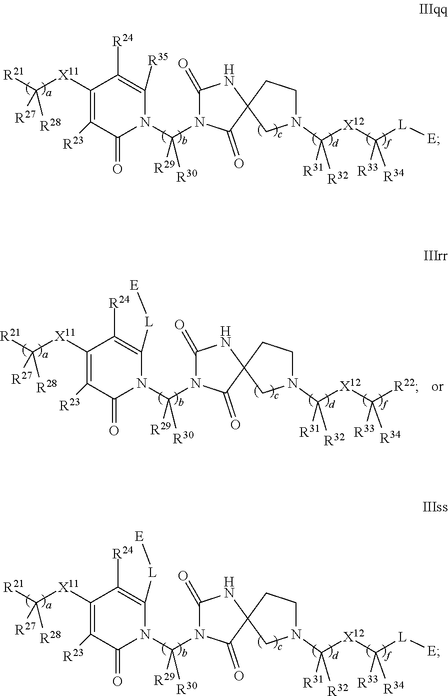

or a pharmaceutically acceptable derivative thereof, wherein
E is an E3 ligase ligand or a moiety that activates the N-degron pathway;
L is a bond or a divalent chemical linker;
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
R²¹ is aryl, heteroaryl or heterocycloalkyl;
R²² is aryl or heteroaryl;
R²³ to R²⁶ and R²⁹ to R³⁵ are each independently H, alkyl or aralkyl; and
R²⁷ and R²⁸ are each independently H, alkyl or aralkyl, or together with the carbon atom to which they are attached form cycloalkyl.

In another embodiment, the compounds provided herein for use in the compositions and methods provided herein have Formula IIIqq, IIIrr or IIIss, or a pharmaceutically acceptable derivative thereof, wherein:
E is an E3 ligase ligand or a moiety that activates the N-degron pathway;
L is a bond or a divalent chemical linker;
b is 2 or 3;
c is 2;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³, R²⁴, R²⁹, R³⁰, and R″ are each independently H, alkyl or aralkyl; and
R²⁷ and R²⁸ are each independently H, alkyl or aralkyl, or together with the carbon atom to which they are attached form cycloalkyl.

In another embodiment, the compounds provided herein for use in the compositions and methods provided herein have Formula IIIqq, IIIrr or IIIss, or a pharmaceutically acceptable derivative thereof, wherein:
E is an E3 ligase ligand or a moiety that activates the N-degron pathway;
L is a bond or a divalent chemical linker;
b is 2 or 3;
c is 2;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³, R²⁴ and R²⁸ to R³⁰ are each independently H; and
R²⁷ and R³⁵ are each independently alkyl.

In another embodiment, the compounds provided herein for use in the compositions and methods provided herein have Formula IIIqq, IIIrr or IIIss, or a pharmaceutically acceptable derivative thereof, wherein:
E is an E3 ligase ligand or a moiety that activates the N-degron pathway;
L is a bond or a divalent chemical linker;
b is 2 or 3;
c is 1;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³, R²⁴, R²⁹, R³⁰, and R″ are each independently H, alkyl or aralkyl; and
R²⁷ and R²⁸ are each independently H, alkyl or aralkyl, or together with the carbon atom to which they are attached form cycloalkyl.

In another embodiment, the compounds provided herein for use in the compositions and methods provided herein have Formula IIIqq, IIIrr or IIIss, or a pharmaceutically acceptable derivative thereof, wherein:
E is an E3 ligase ligand or a moiety that activates the N-degron pathway;
L is a bond or a divalent chemical linker;
b is 2 or 3;
c is 1;
a is 0 or 1;
d and f are each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³, R²⁴ and R²⁸ to R³⁰ are each independently H; and
R²⁷ and R³⁵ are each independently alkyl.

In another embodiment, the compound for use in the compositions and methods provided herein has Formula IIItt, IIIuu or IIIvv:

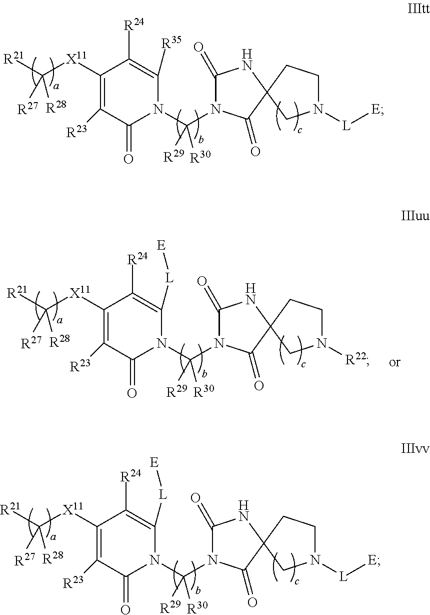

wherein:
E is an E3 ligase ligand or a moiety that activates the N-degron pathway;
L is a bond or a divalent chemical linker;
b is an integer from 1-4;
c is an integer from 1-3;
a is an integer from 0-3;
X¹¹ is a bond, O, NR²⁵ or CO;
R²¹ is aryl or heterocycloalkyl;
R²² is heteroaryl; and
R²³ to R²⁵, R²⁷ to R³⁰ and R″ are each independently H or alkyl.

In another embodiment, the compound for use in the compositions and methods provided herein has Formula IIIww, IIIxx or IIIyy:

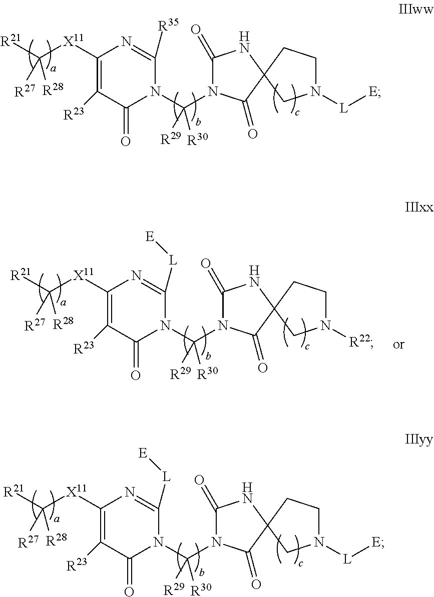

wherein:
E is an E3 ligase ligand or a moiety that activates the N-degron pathway;
L is a bond or a divalent chemical linker;
b is an integer from 1-4;
c is an integer from 1-3;
a is an integer from 0-3;
X¹¹ is a bond, O, NR²⁵ or CO;
R²¹ is aryl or heterocycloalkyl;
R²² is heteroaryl; and
R²³, R²⁵, R²⁷ to R³⁰ and R³⁵ are each independently H or alkyl.

In another embodiment, he compounds for use in the compositions and methods provided herein have Formula IIItt, IIIuu, IIIvv, IIIwww, IIIxx or IIIyy, wherein:
E is an E3 ligase ligand or a moiety that activates the N-degron pathway;
L is a bond or a divalent chemical linker;
b is 2 or 3;
c is 2;
a is 0 or 1;
X¹¹ is a bond or 0;
R²¹ is aryl or heterocycloalkyl, each optionally substituted with 1-3 substituents each independently selected from alkyl and aryl;
R²² is heteroaryl; and
R²³, R²⁵, R²⁷ to R³⁰ and R³⁵ are each independently H or alkyl.

In another embodiment, he compounds for use in the compositions and methods provided herein have Formula IIItt, IIIuu, IIIvv, IIIwww, IIIxx or IIIyy, wherein:
E is an E3 ligase ligand or a moiety that activates the N-degron pathway;
L is a bond or a divalent chemical linker;
b is 2 or 3;
c is 1;
a is 0 or 1;
X¹¹ is a bond or 0;
R²¹ is aryl or heterocycloalkyl, each optionally substituted with 1-3 substituents each independently selected from alkyl and aryl;
R²² is heteroaryl; and
R²³, R²⁵, R²⁷ to R³⁰ and R³⁵ are each independently H or alkyl.

In one embodiment, the compounds provided herein for use in the compositions and methods provided herein are selected from:

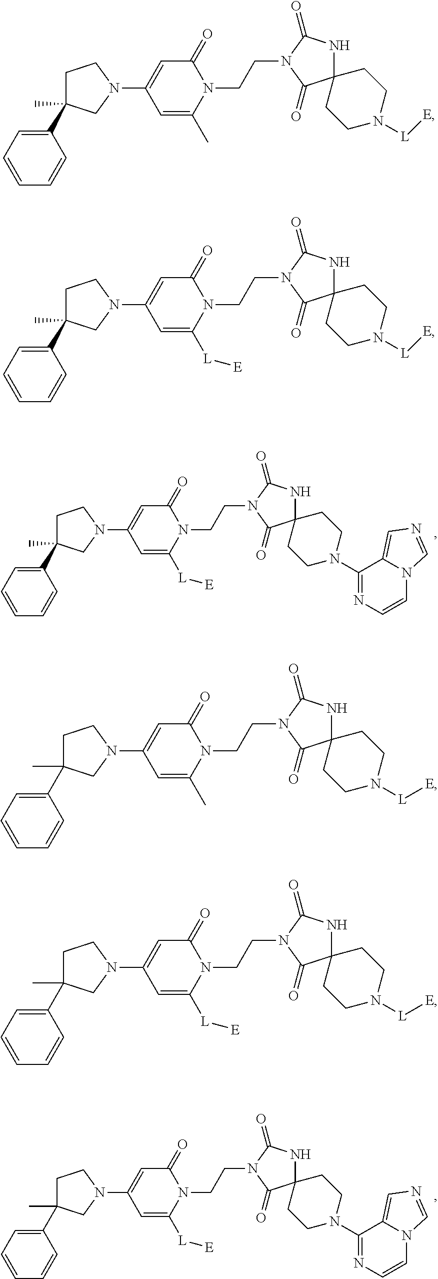

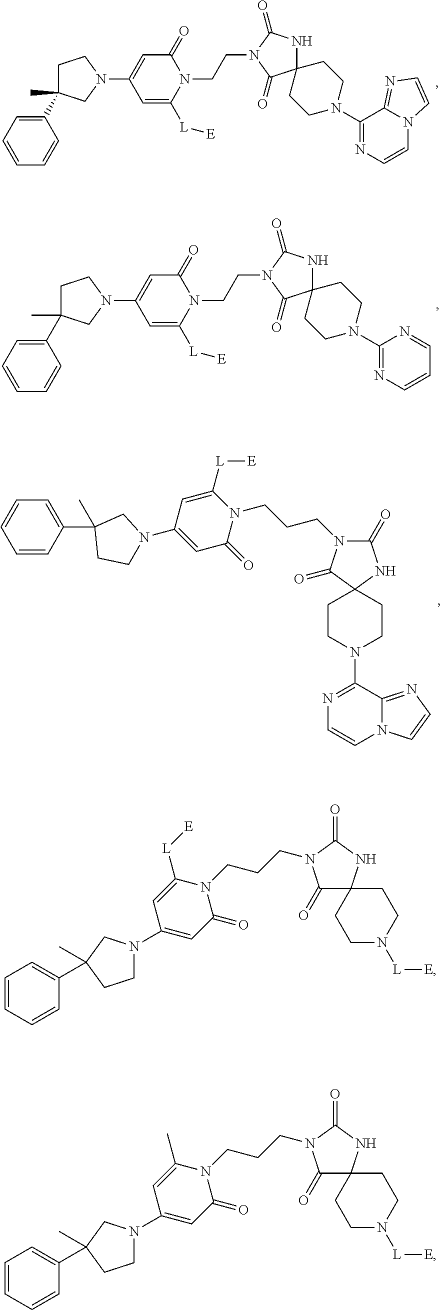

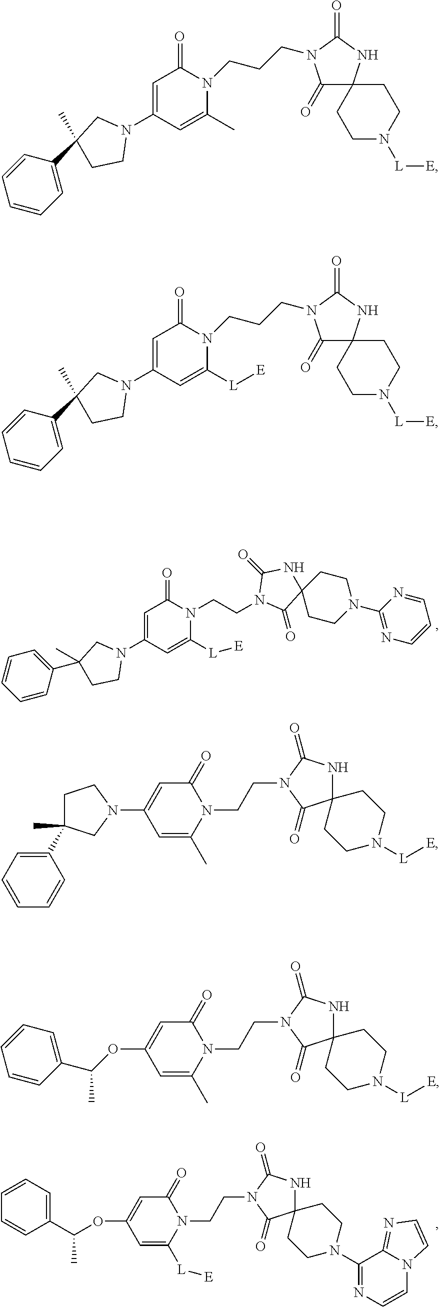

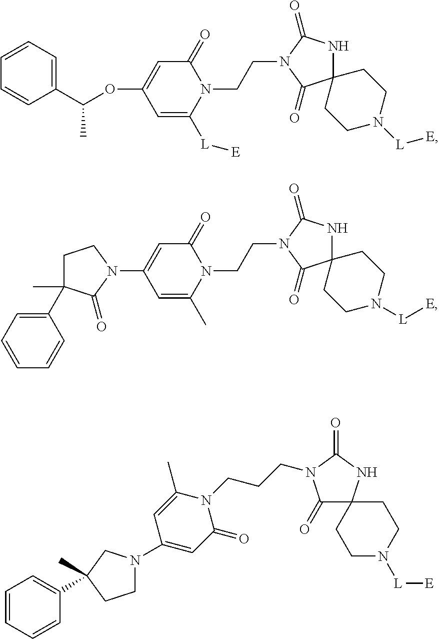

In another embodiment, provided herein is a compound for use in the compositions and methods provided herein having Formula IVa or IVb:

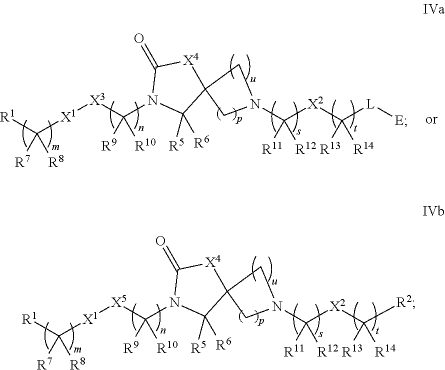

or a pharmaceutically acceptable derivative thereof, wherein
E is an E3 ligase ligand or a moiety that activates the N-degron pathway;
L is a bond or a divalent chemical linker;
n is an integer from 1-4;
p and u are each independently an integer from 1-3;
m, s and t are each independently an integer from 0-3;
X¹ is a bond, O, NR³ or CO;
X² is a bond, CONR⁴, SO₂NR⁴, CO or SO₂;
X³ is bicyclic heteroarylene optionally substituted with -L-E;
X⁴ is (CH₂)₁₋₂, O(CH₂)₀₋₁ or NH(CH₂)₀₋₁;
X⁵ is bicyclic heteroarylene substituted with -L-E;
R¹ is aryl, heteroaryl or heterocycloalkyl;
R² is cycloalkyl, heterocyclyl, aryl or heteroaryl;
R³ to R⁶, R¹¹ and R¹² are each independently H, alkyl or aralkyl;
R⁷ and R⁸ are each independently H, alkyl or aralkyl, or together with the carbon atom to which they are attached form cycloalkyl;
R⁹ and R¹⁰ are each independently H, OH, alkoxy, alkyl or aralkyl, or R⁹ and R¹⁰ together form oxo; and
R¹³ and R¹⁴ are each independently H, alkyl or aralkyl, or together with the carbon atom to which they are attached form cycloalkyl.

In another embodiment, X⁴ is CH₂, O or NH. In another embodiment, X⁴ is CH₂. In another embodiment, X⁴ is O. In another embodiment, X⁴ is NH.

In another embodiment, u is 1 or 2. In another embodiment, u is 1. In another embodiment, u is 2.

In one embodiment, provided herein is a compound for use in the compositions and methods provided herein having Formula IVc or IVd:

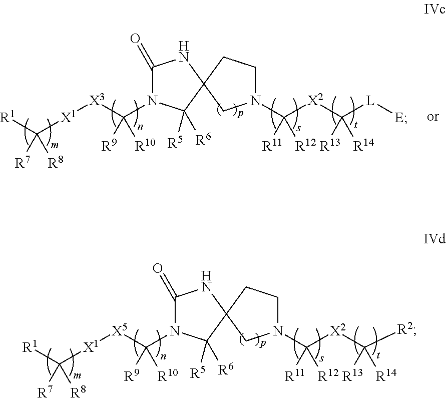

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m, s and t are each independently an integer from 0-3;
X¹ is a bond, O, NR³ or CO;
X² is a bond, CONR⁴, SO₂NR⁴, CO or SO₂;
X³ is bicyclic heteroarylene optionally substituted with -L-E;
X⁵ is bicyclic heteroarylene substituted with -L-E;
R¹ is aryl, heteroaryl or heterocycloalkyl;
R² is cycloalkyl, heterocyclyl, aryl or heteroaryl;
R³ to R⁶, R¹¹ and R¹² are each independently H, alkyl or aralkyl;
R⁷ and R⁸ are each independently H, alkyl or aralkyl, or together with the carbon atom to which they are attached form cycloalkyl;
R⁹ and R¹⁰ are each independently H, OH, alkoxy, alkyl or aralkyl, or R⁹ and R¹⁰ together form oxo; and
R¹³ and R¹⁴ are each independently H, alkyl or aralkyl, or together with the carbon atom to which they are attached form cycloalkyl.

In one embodiment, provided herein is a compound for use in the compositions and methods provided herein having Formula IVc or IVd,
or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m, s and t are each independently an integer from 0-3;
X¹ is a bond, O, NR³ or CO;
X² is a bond, CONR⁴, SO₂NR⁴, CO or SO₂;
X³ is bicyclic heteroarylene optionally substituted with -L-E;
X⁵ is bicyclic heteroarylene substituted with -L-E;
R¹ is aryl, heteroaryl or heterocycloalkyl;
R² is aryl or heteroaryl;
R³ to R⁶, R¹¹ and R¹² are each independently H, alkyl or aralkyl;
R⁷ and R⁸ are each independently H, alkyl or aralkyl, or together with the carbon atom to which they are attached form cycloalkyl;
R⁹ and R¹⁰ are each independently H, OH, alkoxy, alkyl or aralkyl, or R⁹ and R¹⁰ together form oxo; and
R¹³ and R¹⁴ are each independently H, alkyl or aralkyl, or together with the carbon atom to which they are attached form cycloalkyl.

In another embodiment, s and t are each 0, X² is a bond and the compound for use in the compositions and methods provided herein has Formula IVe or IVf:

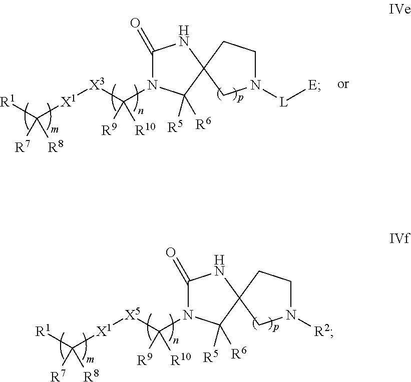

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m is an integer from 0-3;
X¹ is a bond, O, NR³ or CO;
X³ is bicyclic heteroarylene optionally substituted with -L-E;
X⁵ is bicyclic heteroarylene substituted with -L-E;
R¹ is aryl, heteroaryl or heterocycloalkyl;
R² is heteroaryl;
R³, R⁵, R⁶, R¹¹ and R¹⁰ are each independently H, alkyl or aralkyl;
R⁷ and R⁸ are each independently H, alkyl or aralkyl, or together with the carbon atom to which they are attached form cycloalkyl; and
R⁹ and R¹⁰ are each independently H, OH, alkoxy, alkyl or aralkyl, or R⁹ and R¹⁰ together form oxo.

In another embodiment, p and u are each 2, s and t are each 0, X² is a bond and the compound for use in the compositions and methods provided herein has Formula IVg or IVh:

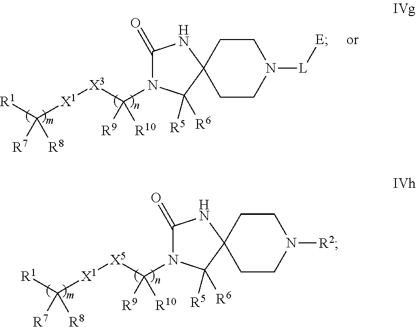

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR³ or CO.
X³ is bicyclic heteroarylene optionally substituted with -L-E;
X⁵ is bicyclic heteroarylene substituted with -L-E;
R¹ is aryl, heteroaryl or heterocycloalkyl;
R² is heteroaryl;
R³, R⁵ and R⁶ are each independently H, alkyl or aralkyl;
R⁷ and R⁸ are each independently H, alkyl or aralkyl, or together with the carbon atom to which they are attached form cycloalkyl; and
R⁹ and R¹⁰ are each independently H, OH, alkoxy, alkyl or aralkyl, or R⁹ and R¹⁰ together form oxo.

In another embodiment, each of R⁵, R⁶, R⁹ and R¹⁰ are H, p is 2, s and t are each 0, X² is a bond and the compound for use in the compositions and methods provided herein has Formula IVi or IVj:

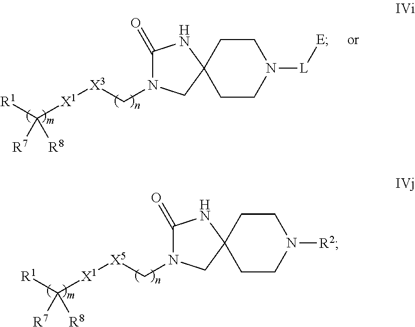

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR³ or CO;
X³ is bicyclic heteroarylene optionally substituted with -L-E;
X⁵ is bicyclic heteroarylene substituted with -L-E;
R¹ is aryl, heteroaryl or heterocycloalkyl;
R² is heteroaryl;
R³ is H or alkyl; and
R⁷ and R³ are each independently H or alkyl, or together with the carbon atom to which they are attached form cycloalkyl.

In another embodiment, X¹ is a bond, each of R³ to R⁶, R⁹ and R¹⁰ are H, p is 2, m, s and t are each 0, X² is a bond and the compound for use in the compositions and methods provided herein has Formula IVk or IVm:

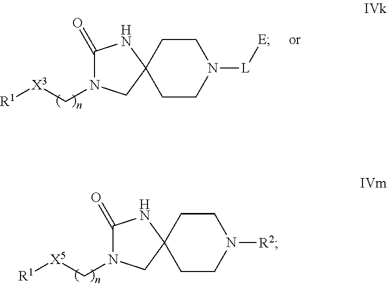

or a pharmaceutically acceptable derivative thereof, wherein
E is an E3 ligase ligand or a moiety that activates the N-degron pathway;
L is a bond or a divalent chemical linker;
n is 2 or 3;
X³ is bicyclic heteroarylene optionally substituted with -L-E;
X⁵ is bicyclic heteroarylene substituted with -L-E;
R¹ is heterocycloalkyl, optionally substituted with 1-3 substituents each independently selected from alkyl and aryl; and
R² is heteroaryl.

In another embodiment, the compound for use in the compositions and methods provided herein has Formula IVn or IVo:

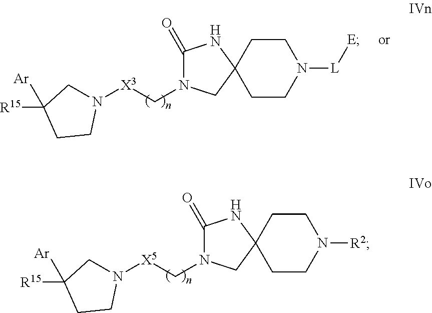

or a pharmaceutically acceptable derivative thereof, wherein
E is an E3 ligase ligand or a moiety that activates the N-degron pathway;
L is a bond or a divalent chemical linker;
n is 2 or 3;
X³ is bicyclic heteroarylene optionally substituted with -L-E;
X⁵ is bicyclic heteroarylene substituted with -L-E;
Ar is aryl;
R² is heteroaryl; and
R¹⁵ is alkyl.

In another embodiment, the compound for use in the compositions and methods provided herein has the formula:

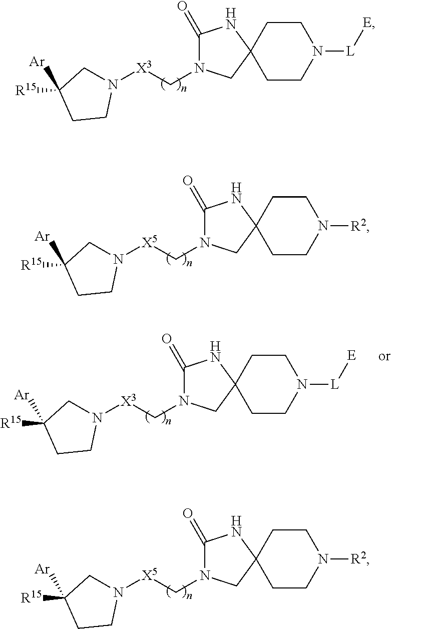

where the variables are as defined herein.

In another embodiment, the compound for use in the compositions and methods provided herein has Formula IVp or IVq:

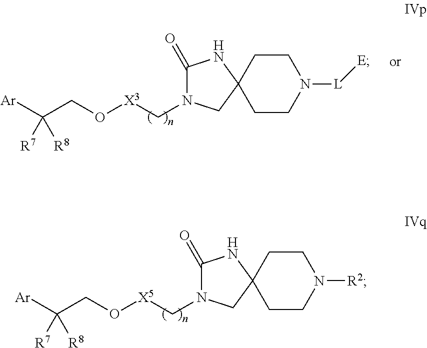

or a pharmaceutically acceptable derivative thereof, wherein
E is an E3 ligase ligand or a moiety that activates the N-degron pathway;
L is a bond or a divalent chemical linker;
n is 2 or 3;
X³ is bicyclic heteroarylene optionally substituted with -L-E;
X⁵ is bicyclic heteroarylene substituted with -L-E;
Ar is aryl;
R² is heteroaryl; and
R⁷ and R⁸ are each independently H or alkyl, or together with the carbon atom to which they are attached form cycloalkyl.

In another embodiment, the compound for use in the compositions and methods provided herein has the formula:

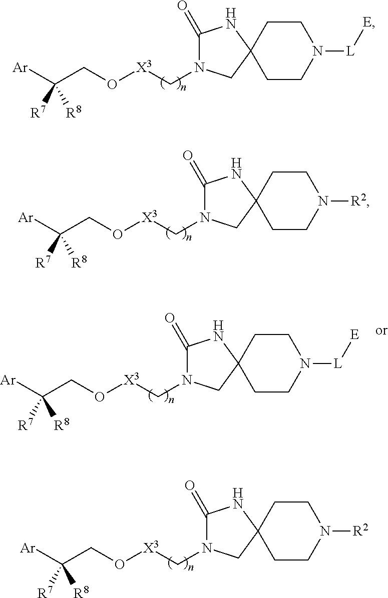

where the variables are as defined herein.

In another embodiment, p and u are is 1, s and t are each 0, X² is a bond and the compound for use in the compositions and methods provided herein has Formula IVr or IVs:

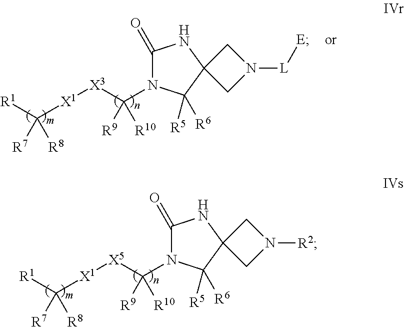

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR³ or CO;
X³ is bicyclic heteroarylene optionally substituted with -L-E;
X⁵ is bicyclic heteroarylene substituted with -L-E;
R¹ is aryl, heteroaryl or heterocycloalkyl;
R² is heteroaryl;
R³, R⁵ and R⁶ are each independently H, alkyl or aralkyl;
R⁷ and R⁸ are each independently H, alkyl or aralkyl, or together with the carbon atom to which they are attached form cycloalkyl; and
R⁹ and R¹⁰ are each independently H, OH, alkoxy, alkyl or aralkyl, or R⁹ and R¹⁰ together form oxo.

In another embodiment, each of R⁵, R⁶, R⁹ and R¹⁰ are H, p and u are each 1, s and t are each 0, X² is a bond and the compound for use in the compositions and methods provided herein has Formula IVt or IVu:

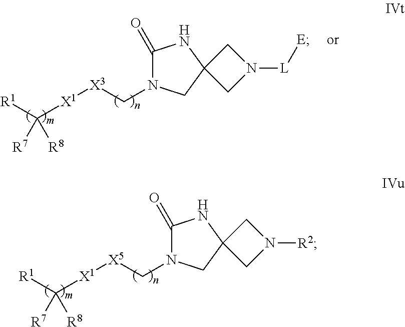

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR³ or CO;
X³ is bicyclic heteroarylene optionally substituted with -L-E;
X⁵ is bicyclic heteroarylene substituted with -L-E;
R¹ is aryl, heteroaryl or heterocycloalkyl;
R² is heteroaryl;
R³ is H or alkyl; and
R⁷ and R⁸ are each independently H or alkyl, or together with the carbon atom to which they are attached form cycloalkyl.

In another embodiment, R¹ in Formulae IVa, IVb, IVc, IVd, IVe, IVf, IVg, IVh, IVi, IVj, IVr, IVs, IVt or IVu is aryl or heterocycloalkyl. In another embodiment, R¹ in Formulae IVa, IVb, IVc, IVd, IVe, IVf, IVg, IVh, IVi, IVj, IVr, IVs, IVt or IVu is aryl. In another embodiment, R¹ in Formulae IVa, IVb, IVc, IVd, IVe, IVf, IVg, IVh, IVi, IVj, IVr, IVs, IVt or IVu is heterocycloalkyl.

In another embodiment, X¹ is a bond, each of R³ to R⁶, R⁹ and R¹⁰ are H, p and u are each 1, m, s and t are each 0, X² is a bond and the compound for use in the compositions and methods provided herein has Formula IVv or IVw:

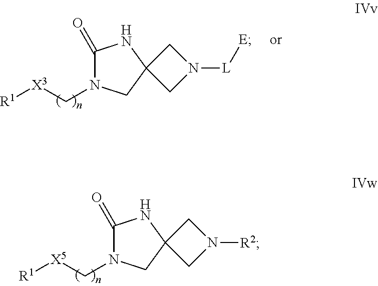

or a pharmaceutically acceptable derivative thereof, wherein
E is an E3 ligase ligand or a moiety that activates the N-degron pathway;
L is a bond or a divalent chemical linker;
n is 2 or 3;
X³ is bicyclic heteroarylene optionally substituted with -L-E; and
X⁵ is bicyclic heteroarylene substituted with -L-E;
R¹ is heterocycloalkyl, optionally substituted with 1-3 substituents each independently selected from alkyl and aryl; and
R² is heteroaryl.

In another embodiment, the compound for use in the compositions and methods provided herein has Formula IVx or IVy:

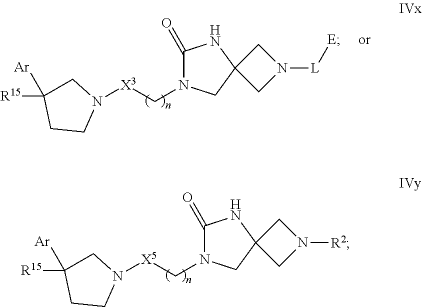

or a pharmaceutically acceptable derivative thereof, wherein
E is an E3 ligase ligand or a moiety that activates the N-degron pathway;
L is a bond or a divalent chemical linker;
n is 2 or 3;
X³ is bicyclic heteroarylene optionally substituted with -L-E;
X⁵ is bicyclic heteroarylene substituted with -L-E;
Ar is aryl;
R² is heteroaryl; and
R¹⁵ is alkyl.

In another embodiment, R¹⁵ is lower alkyl. In another embodiment, R¹⁵ is methyl or ethyl. In another embodiment, R¹⁵ is methyl. In another embodiment, R¹⁵ is ethyl.

In another embodiment, the compound for use in the compositions and methods provided herein has the formula:

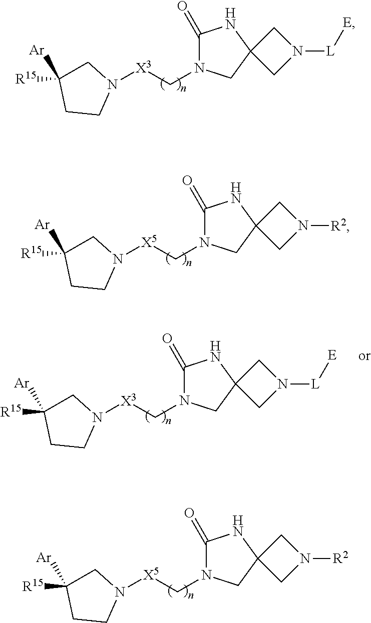

where the variables are as defined herein.

In another embodiment, the compound for use in the compositions and methods provided herein has Formula IVaa or IVbb:

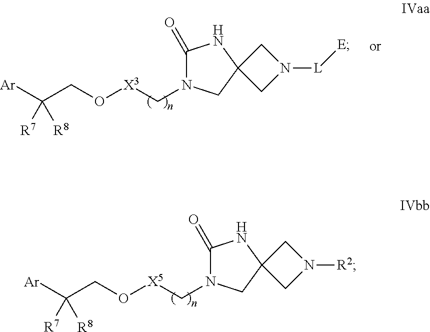

or a pharmaceutically acceptable derivative thereof, wherein
E is an E3 ligase ligand or a moiety that activates the N-degron pathway;
L is a bond or a divalent chemical linker;
n is 2 or 3;
X³ is bicyclic heteroarylene optionally substituted with -L-E;
X⁵ is bicyclic heteroarylene substituted with -L-E;
Ar is aryl;
R² is heteroaryl; and
R⁷ and R⁸ are each independently H or alkyl, or together with the carbon atom to which they are attached form cycloalkyl.

In another embodiment, R⁷ is alkyl and R⁸ is H. In another embodiment, R⁷ is methyl and R⁸ is H. In another embodiment, R⁷ and R⁸ together with the carbon atom to which they are attached form cyclopropyl.

In another embodiment, the compound for use in the compositions and methods provided herein has the formula:

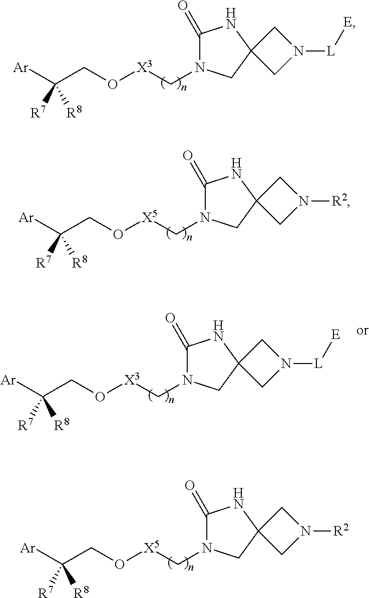

where the variables are as defined herein.

In another embodiment, the compound for use in the compositions and methods provided herein has any of the following formulae where the variables are as defined herein:

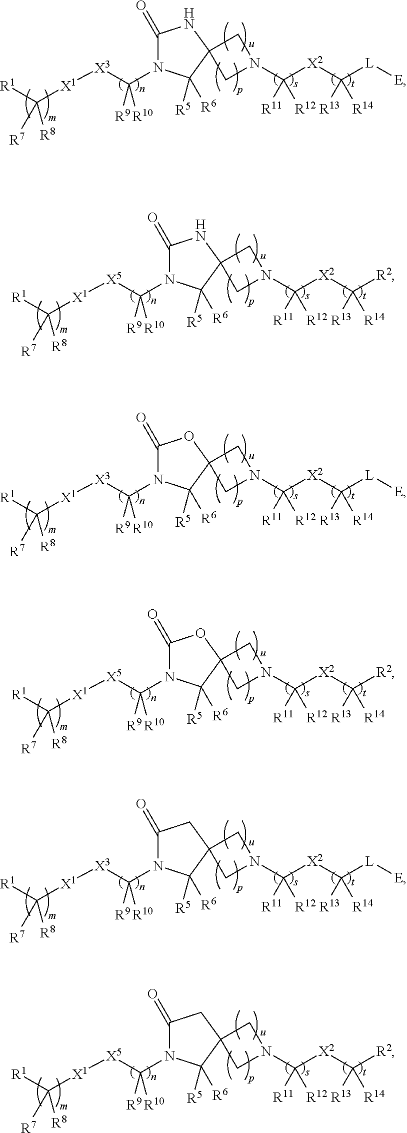

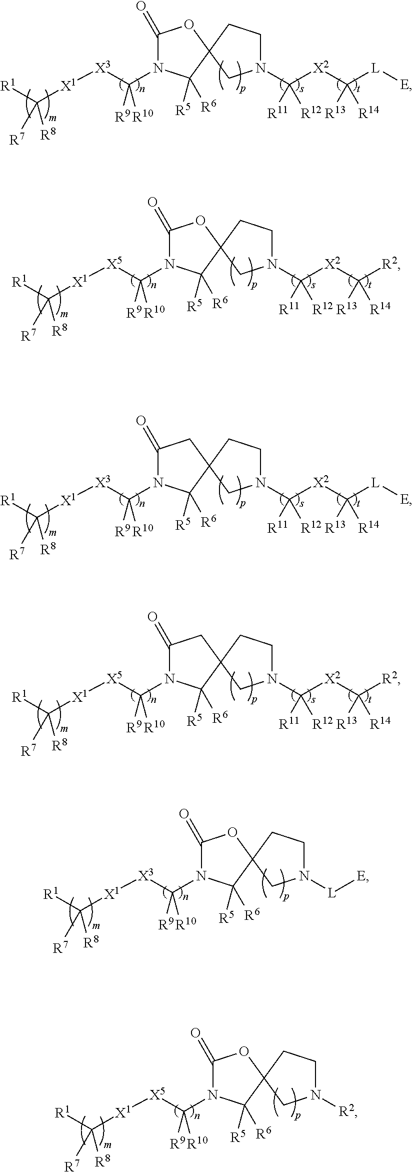

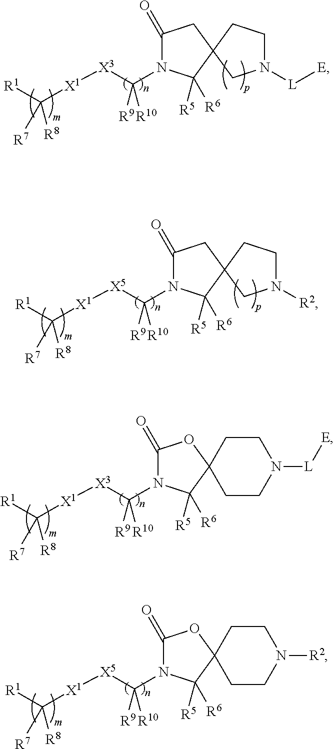

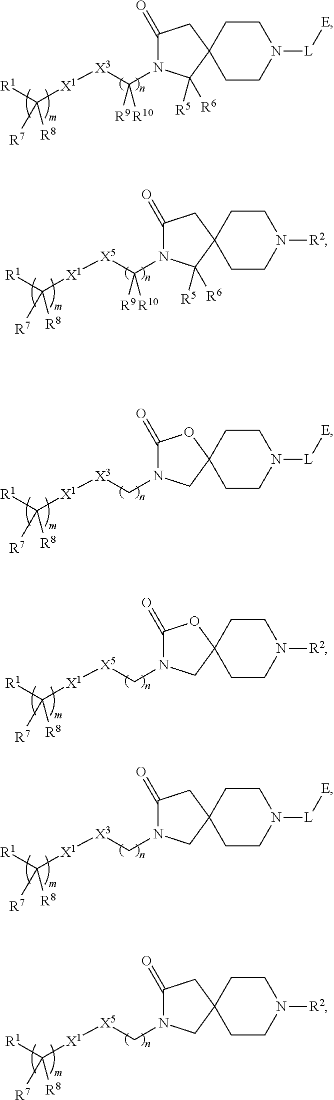

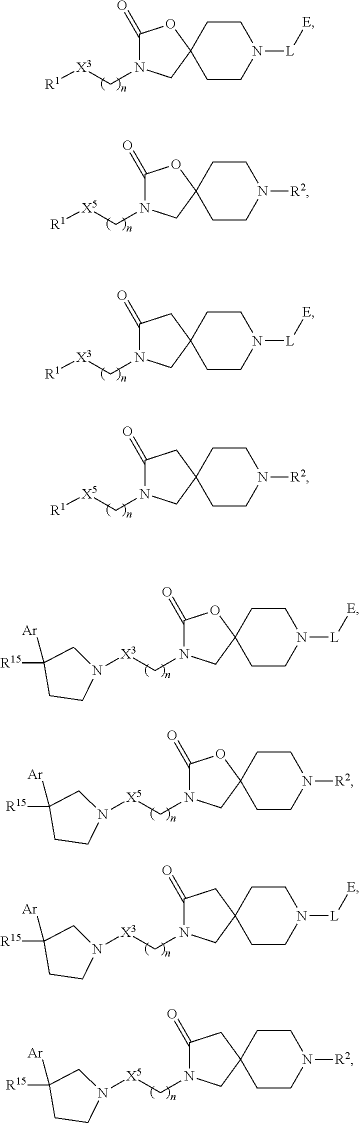

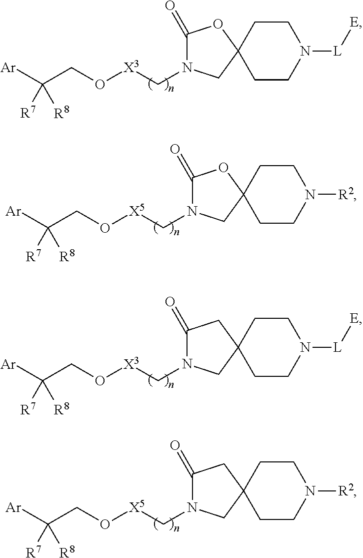

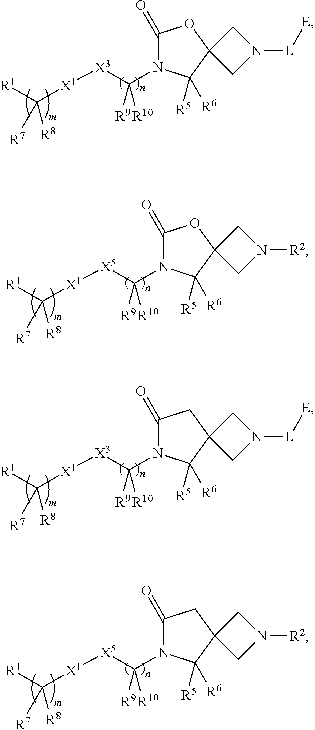

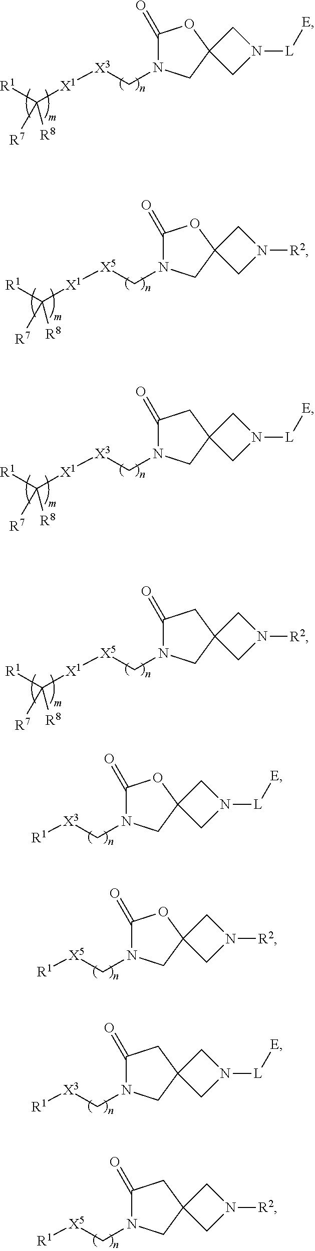

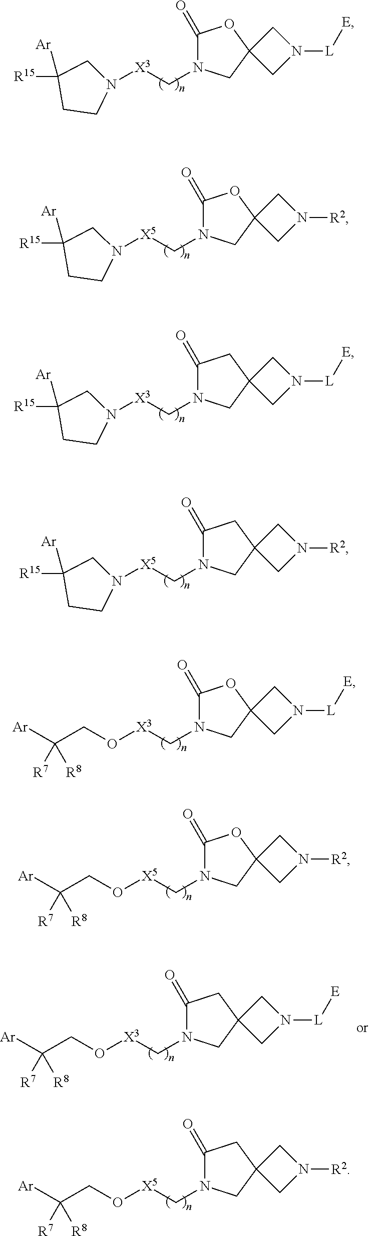

In another embodiment, n is 2. In another embodiment, n is 3.

In another embodiment, X³ is imidazopyridinylene or triazolopyridinylene, each optionally substituted with -L-E, alkyl, haloalkyl, hydroxyalkyl or halo. In another embodiment, X³ is imidazopyridinylene or triazolopyridinylene, each optionally substituted with -L-E, methyl, halomethyl, hydroxymethyl or halo. In another embodiment, X³ is triazolopyridinylene, optionally substituted with -L-E, methyl, halomethyl, hydroxymethyl or halo. In another embodiment, X³ is imidazopyridinylene, optionally substituted with -L-E, methyl, halomethyl, hydroxymethyl or halo. In another embodiment, X³ is 3-methylimidazo[1,2-a]pyridinyl-2,7-ene or 8-methyl-[1,2,4]triazolo[1,5-a]pyridinyl-2,6-ene, each optionally substituted with -L-E. In another embodiment, X³ has the structure:

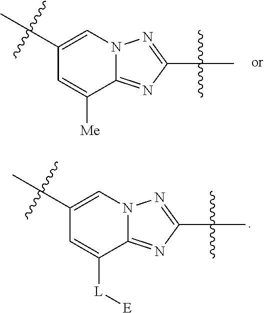

In another embodiment, X³ has the structure:

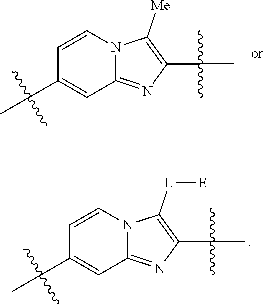

In one embodiment, provided herein is a compound for use in the compositions and methods provided herein having Formula IVn, IVo or IVp:

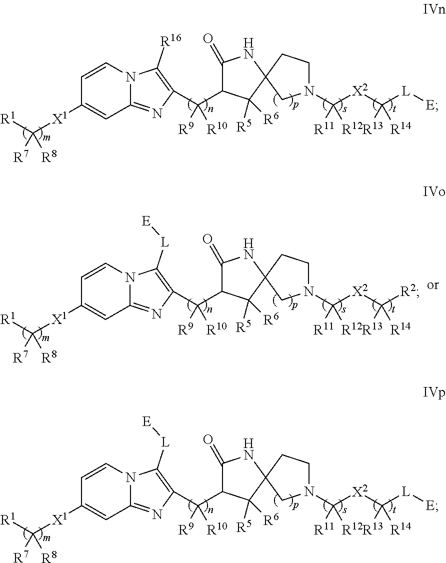

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m, s and t are each independently an integer from 0-3;
X¹ is a bond, O, NR³ or CO;
X² is a bond, CONR⁴, SO₂NR⁴, CO or SO₂;
R¹ is aryl or heterocycloalkyl;
R² is aryl or heteroaryl;
R³ to R⁶, R¹¹ and R¹² are each independently H, alkyl or aralkyl;
R⁷ and R⁸ are each independently H, alkyl or aralkyl, or together with the carbon atom to which they are attached form cycloalkyl;
R⁹ and R¹⁰ are each independently H, OH, alkoxy, alkyl or aralkyl, or R⁹ and R¹⁰ together form oxo;
R¹³ and R¹⁴ are each independently H, alkyl or aralkyl, or together with the carbon atom to which they are attached form cycloalkyl; and
R¹⁶ is H, alkyl, haloalkyl, hydroxyalkyl or halo.

In another embodiment, the compounds provided herein for use in the compositions and methods provided herein have Formula IVn, IVo or IVp, or a pharmaceutically acceptable derivative thereof, wherein:
E is an E3 ligase ligand or a moiety that activates the N-degron pathway;
L is a bond or a divalent chemical linker;
n is 2 or 3;
p is 2;
m is 0 or 2;
s and t are each 0;
X¹ is a bond or O;
X² is a bond;
R¹ is aryl or heterocycloalkyl, each optionally substituted with 1-3 substituents each independently selected from alkyl and phenyl;
R³ to R⁶, R¹¹ and R¹² are each independently H, alkyl or aralkyl;
R⁷ and R⁸ are each independently H, alkyl or aralkyl, or together with the carbon atom to which they are attached form cycloalkyl;
R⁹ and R¹⁰ are each independently H, OH, alkoxy, alkyl or aralkyl, or R⁹ and R¹⁰ together form oxo;
R¹³ and R¹⁴ are each independently H, alkyl or aralkyl, or together with the carbon atom to which they are attached form cycloalkyl; and
R¹⁶ is H, alkyl, haloalkyl, hydroxyalkyl or halo.

In another embodiment, the compounds provided herein for use in the compositions and methods provided herein have Formula IVn, IVo or IVp, or a pharmaceutically acceptable derivative thereof, wherein:
E is an E3 ligase ligand or a moiety that activates the N-degron pathway;
L is a bond or a divalent chemical linker;
n is 2 or 3;
p is 2;
m is 0 or 2;
s and t are each 0;
X¹ is a bond or O;
X² is a bond;
R¹ is aryl or heterocycloalkyl, each optionally substituted with 1-3 substituents each independently selected from alkyl and phenyl;
R³ to R⁶ and R⁸ to R¹⁴ are each independently H; and
R⁷ and R¹⁶ are each independently alkyl.

In another embodiment, provided herein is a compound for use in the compositions and methods provided herein having Formula IVq, IVr or IVs:

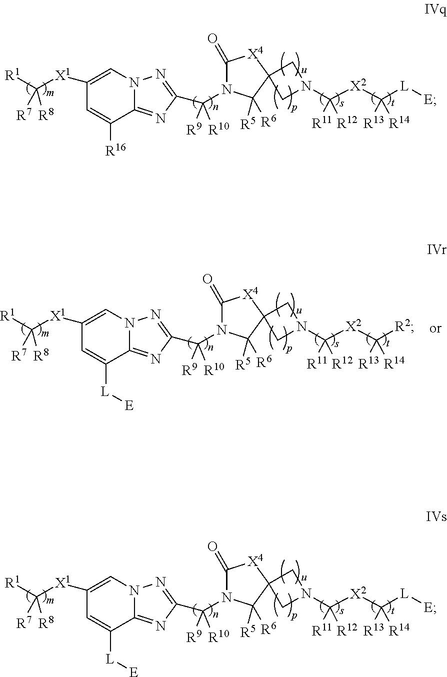

or a pharmaceutically acceptable derivative thereof, wherein
E is an E3 ligase ligand or a moiety that activates the N-degron pathway;
L is a bond or a divalent chemical linker;
n is an integer from 1-4;
p and u are each independently an integer from 1-3;
m, s and t are each independently an integer from 0-3;
X¹ is a bond, O, NR₃ or CO;
X² is a bond, CONR⁴, SO₂NR⁴, CO or SO₂;
X⁴ is (CH₂)₁₋₂, O(CH₂)₀₋₁ or NH(CH₂)₀₋₁;
R¹ is aryl or heterocycloalkyl;
R² is cycloalkyl, heterocyclyl, aryl or heteroaryl;
R³ to R⁶, R¹¹ and R¹² are each independently H, alkyl or aralkyl;
R⁷ and R⁸ are each independently H, alkyl or aralkyl, or together with the carbon atom to which they are attached form cycloalkyl;
R⁹ and R¹⁰ are each independently H, OH, alkoxy, alkyl or aralkyl, or R⁹ and R¹⁰ together form oxo;
R¹³ and R¹⁴ are each independently H, alkyl or aralkyl, or together with the carbon atom to which they are attached form cycloalkyl; and
R¹⁶ is H, alkyl, haloalkyl, hydroxyalkyl or halo.

In another embodiment, provided herein is a compound having one of the following formulae where the variables are as defined herein:

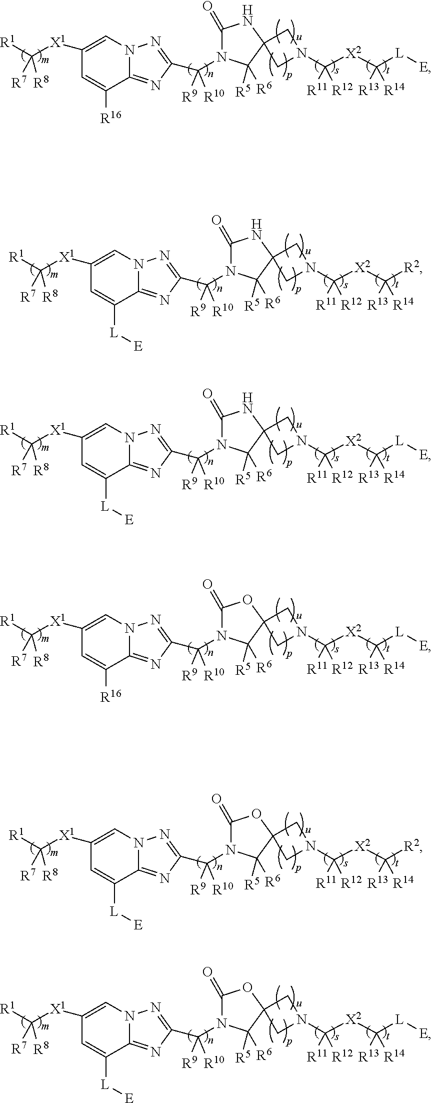

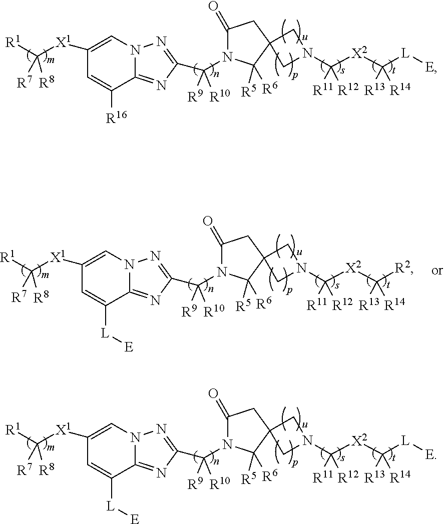

In another embodiment, provided herein is a compound for use in the compositions and methods provided herein having Formula IVt, IVu or IVv:

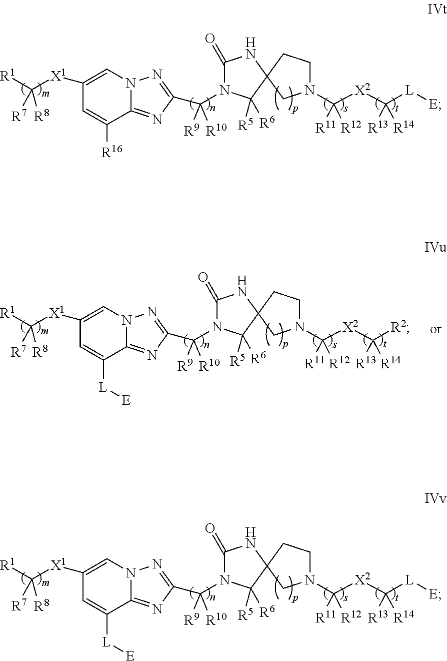

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m, s and t are each independently an integer from 0-3;
X¹ is a bond, O, NR³ or CO;
X² is a bond, CONR⁴, SO₂NR⁴, CO or SO₂;
R¹ is aryl or heterocycloalkyl;
R² is aryl or heteroaryl;
R³ to R⁶, R¹¹ and R¹² are each independently H, alkyl or aralkyl;
R⁷ and R⁸ are each independently H, alkyl or aralkyl, or together with the carbon atom to which they are attached form cycloalkyl;
R⁹ and R¹⁰ are each independently H, OH, alkoxy, alkyl or aralkyl, or R⁹ and R¹⁰ together form oxo;
R¹³ and R¹⁴ are each independently H, alkyl or aralkyl, or together with the carbon atom to which they are attached form cycloalkyl; and
R¹⁶ is H, alkyl, haloalkyl, hydroxyalkyl or halo.

In another embodiment, the compounds provided herein for use in the compositions and methods provided herein have Formula IVt, IVu or IVv, or a pharmaceutically acceptable derivative thereof, wherein:
E is an E3 ligase ligand or a moiety that activates the N-degron pathway;
L is a bond or a divalent chemical linker;
n is 2 or 3;
p is 2;
m is 0 or 2;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁶, R¹¹ and R¹² are each independently H, alkyl or aralkyl;
R⁷ and R⁸ are each independently H, alkyl or aralkyl, or together with the carbon atom to which they are attached form cycloalkyl;
R⁹ and R¹⁰ are each independently H, OH, alkoxy, alkyl or aralkyl, or R⁹ and R¹⁰ together form oxo;
R¹³ and R¹⁴ are each independently H, alkyl or aralkyl, or together with the carbon atom to which they are attached form cycloalkyl; and
R¹⁶ is H, alkyl, haloalkyl, hydroxyalkyl or halo.

In another embodiment, the compounds provided herein for use in the compositions and methods provided herein have Formula IVt, IVu or IVv, or a pharmaceutically acceptable derivative thereof, wherein:
E is an E3 ligase ligand or a moiety that activates the N-degron pathway;
L is a bond or a divalent chemical linker;
n is 2 or 3;
p is 2;
m is 0 or 2;
s and t are each 0;
X¹ is a bond or 0;
X² is a bond;
R¹ is aryl or heterocycloalkyl, each optionally substituted with 1-3 substituents each independently selected from alkyl and phenyl;
R² is heteroaryl;
R³ to R⁶ and R⁸ to R¹⁴ are each independently H; and
R⁷ and R¹⁶ are each independently alkyl.

In another embodiment, provided is a compound having one of the following formulae where the variables are as defined herein:

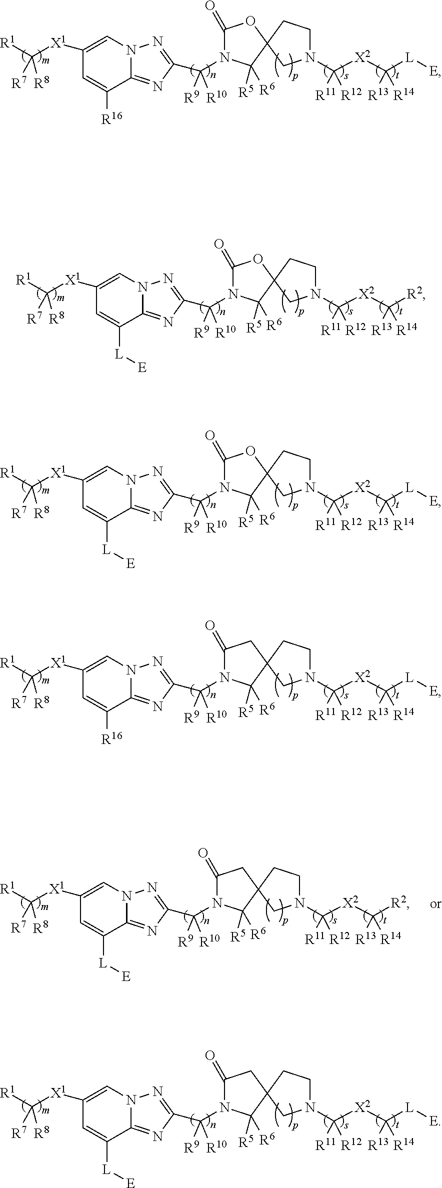

In another embodiment, the compounds for use in the compositions and methods provided herein have Formula IVx, IVy or IVz:

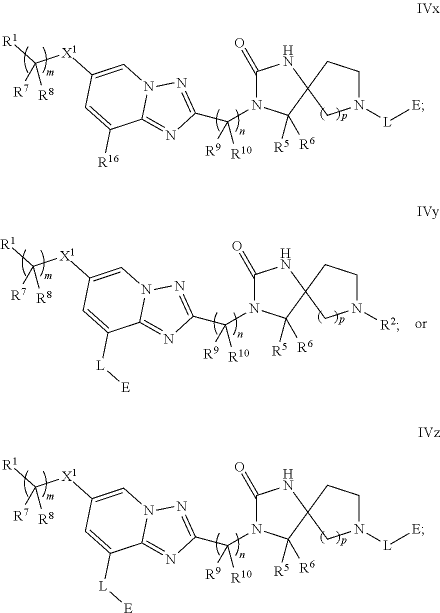

wherein:
E is an E3 ligase ligand or a moiety that activates the N-degron pathway;
L is a bond or a divalent chemical linker;
n is an integer from 1-4;
p is an integer from 1-3;
m is an integer from 0-3;
X¹ is a bond, O, NR³ or CO;
R¹ is aryl, heteroaryl or heterocycloalkyl;
R² is heteroaryl;
R³, R⁵, R⁶, R⁹, R¹⁰ and R¹⁶ are each independently H or alkyl; and
R⁷ and R⁸ are each independently H or alkyl, or together with the carbon atom to which they are attached form cycloalkyl.

In another embodiment, the compounds for use in the compositions and methods provided herein have one of the following formulae where the variables are as defined herein:

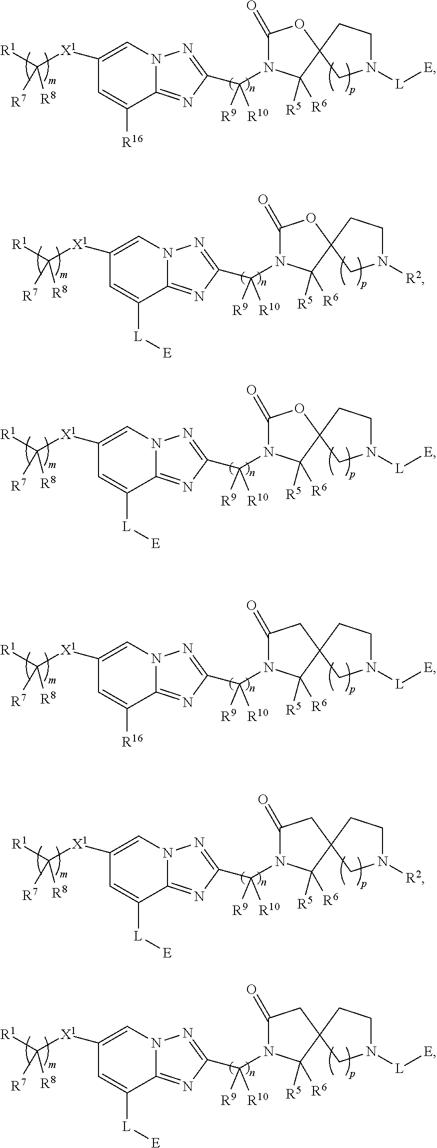

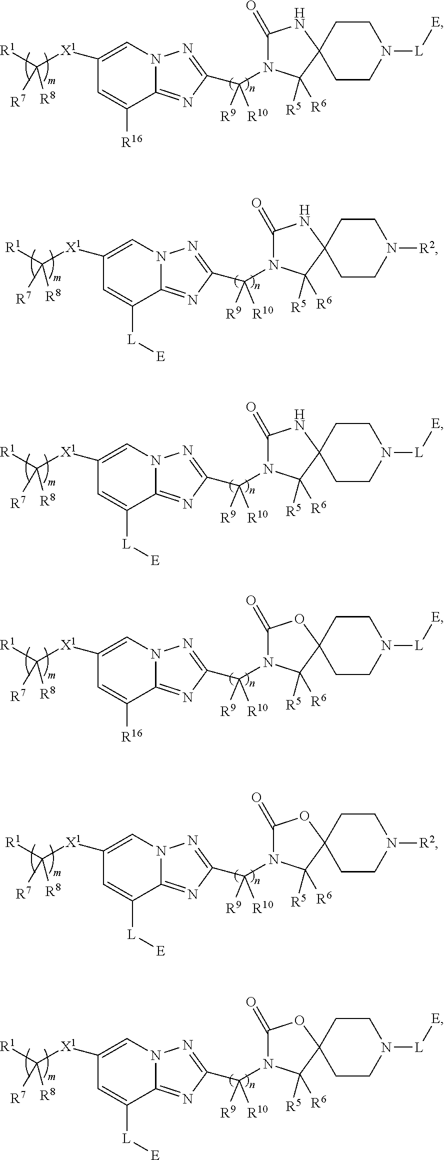

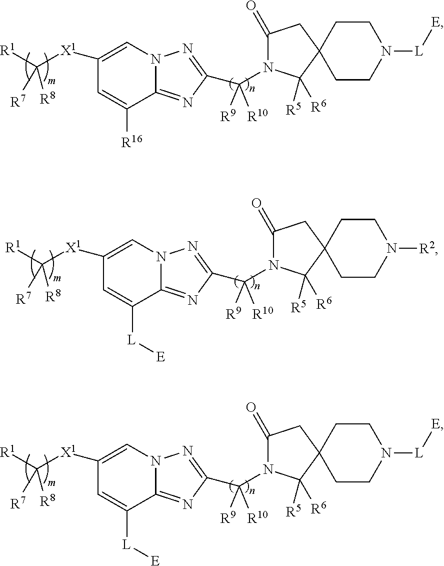

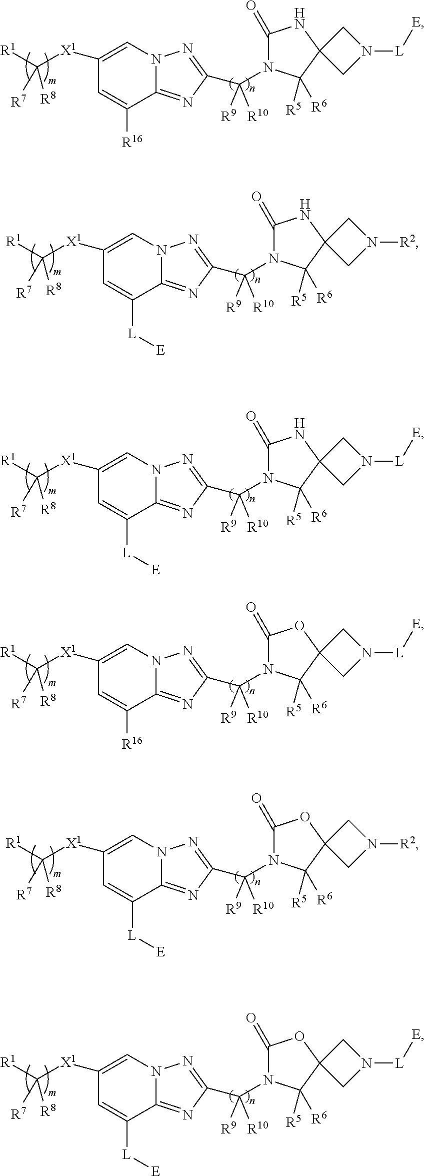

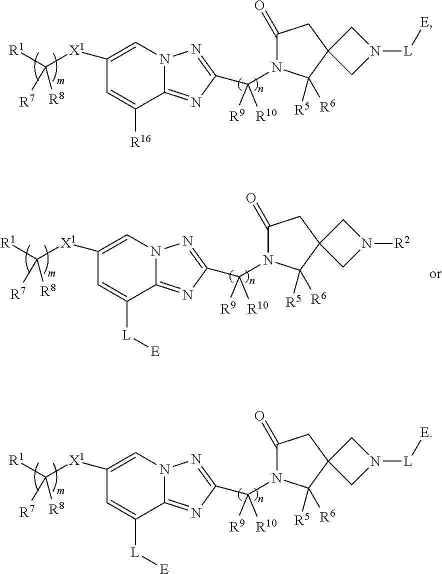

In another embodiment, the compounds for use in the compositions and methods provided herein have Formula IVaa, IVbb or IVcc:

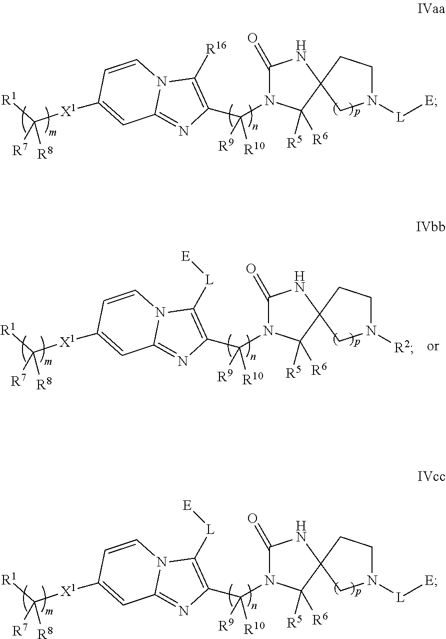

wherein:
E is an E3 ligase ligand or a moiety that activates the N-degron pathway;
L is a bond or a divalent chemical linker;
n is an integer from 1-4;
p is an integer from 1-3;
m is an integer from 0-3;
X¹ is a bond, O, NR³ or CO;
R¹ is aryl, heteroaryl or heterocycloalkyl;
R² is heteroaryl;
R³, R⁵, R⁶, R⁹, R¹⁰ and R¹⁶ are each independently H or alkyl; and
R⁷ and R⁸ are each independently H or alkyl, or together with the carbon atom to which they are attached form cycloalkyl.

In another embodiment, he compounds for use in the compositions and methods provided herein have Formula IVx, IVy, IVz, IVaa, IVbb or IVcc, wherein:
E is an E3 ligase ligand or a moiety that activates the N-degron pathway;
L is a bond or a divalent chemical linker;
n is 2 or 3;
p is 2;
m is 0 or 2;
X¹ is a bond or 0;
R¹ is aryl or heterocycloalkyl, each optionally substituted with 1-3 substituents each independently selected from alkyl and aryl;
R² is heteroaryl;
R⁵, R⁶, R⁹, R¹⁰ and R¹⁶ are each independently H or alkyl; and
R⁷ and R⁸ are each independently H or alkyl, or together with the carbon atom to which they are attached form cycloalkyl.

In another embodiment, the compound has any of Formulae IVa to IVcc, where R⁹ and R¹⁰ are each independently H, alkyl or aralkyl. In another embodiment, the compound has any of Formulae IVa to IVcc where R⁹ and R¹⁰ are each independently H, OH, OMe or methyl, or R⁹ and R¹⁰ together form oxo. In another embodiment, the compound has any of Formulae IVa to IVcc where R⁹ and R¹⁰ are each independently H, OH, OMe or methyl.

In another embodiment, the compound has any of Formulae IVa to IVcc, where R¹³ and R⁴ are each independently H, alkyl or aralkyl.

In another embodiment, the compound has any of Formulae IVa to IVcc, where R⁹ and R¹⁰ are each independently H, alkyl or aralkyl; and R¹³ and R¹⁴ are each independently H, alkyl or aralkyl.

In another embodiment, Ar is unsubstituted aryl. In another embodiment, Ar is optionally substituted phenyl. In another embodiment, Ar is unsubstituted phenyl.

In another embodiment, R² is heterocyclyl, aryl or heteroaryl. In another embodiment, R² is aryl or heteroaryl. In another embodiment, R² is bicyclic or monocyclic heteroaryl. In another embodiment, R² is piperidinyl, pyridyl, isoxazolyl, triazolyl, tetrazolyl, pyridazinyl, pyrimidinyl, thiadiazolyl, pyrazinyl, pyrazolyl, thiazolyl, oxadiazolyl, triazolopyrazinyl or imidazopyrazinyl, each optionally substituted with C(O)Me, Me, OH, COOH, CF₃, F, Cl, CN, NH₂, oxo, S(O)(NH)Me, SO₂Me, COOEt, CONHMe, COOMe or OMe. In another embodiment, R² is triazolopyrazinyl or imidazopyrazinyl. In another embodiment, R² is [1,2,4]-triazolo[4,3-a]pyrazin-8-yl, imidazo[1,5-a]pyrazin-8-yl or imidazo[1,2-a]pyrazin-8-yl.

In another embodiment, provided herein is a compound for use in the compositions and methods provided herein having Formula Va or Vb:

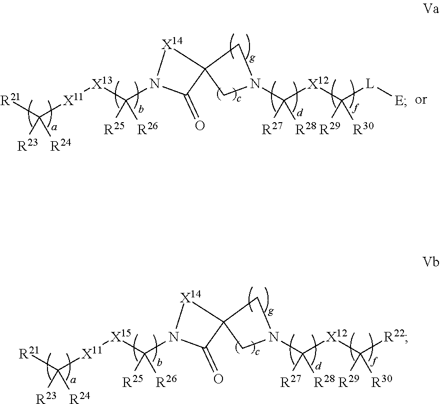

or a pharmaceutically acceptable derivative thereof, wherein:
E is an E3 ligase ligand or a moiety that activates the N-degron pathway;
L is a bond or a divalent chemical linker;
b is an integer from 1-4;
c and g are each independently an integer from 1-3;
a, d and f are each independently an integer from 0-3;
X¹¹ is a bond, O, NR³¹ or CO;
X¹² is a bond, CONR³², SO₂NR³², CO or SO₂;
X¹³ is bicyclic heteroarylene optionally substituted with -L-E;
X¹⁴ is (CH₂)₂₋₃, C(O)NH(CH₂)₀₋₁ or C(O)(CH₂)₁₋₂;
X¹⁵ is bicyclic heteroarylene substituted with -L-E;
R²¹ is aryl, heteroaryl or heterocycloalkyl;
R²² is cycloalkyl, heterocyclyl, aryl or heteroaryl;
R²³ and R²⁴ are each independently H, alkyl or aralkyl, or together with the carbon atom to which they are attached form cycloalkyl; and
R²⁵ and R²⁶ are each independently H, OH, alkoxy, alkyl or aralkyl, or R²⁵ and R²⁶ together form oxo;
R²⁹ and R³⁰ are each independently H, alkyl or aralkyl, or together with the carbon atom to which they are attached form cycloalkyl; and
R²⁷, R²⁸, R³¹ and R³² are each independently H, alkyl or aralkyl.

In one embodiment, provided herein is a compound for use in the compositions and methods provided herein having Formula Vc or Vd:

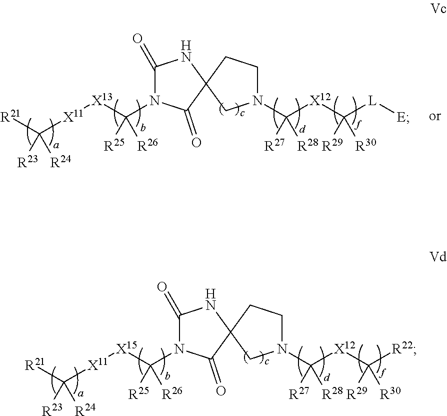

or a pharmaceutically acceptable derivative thereof, wherein:
E is an E3 ligase ligand or a moiety that activates the N-degron pathway;
L is a bond or a divalent chemical linker;
b is an integer from 1-4;
c is an integer from 1-3;
a, d and f are each independently an integer from 0-3;
X¹¹ is a bond, O, NR³¹ or CO;
X¹² is a bond, CONR³², SO₂NR³², CO or SO₂;
X¹³ is bicyclic heteroarylene optionally substituted with -L-E;
X¹⁵ is bicyclic heteroarylene substituted with -L-E;
R²¹ is aryl, heteroaryl or heterocycloalkyl;
R²² is cycloalkyl, heterocyclyl, aryl or heteroaryl;
R²³ and R²⁴ are each independently H, alkyl or aralkyl, or together with the carbon atom to which they are attached form cycloalkyl; and
R²⁵ and R²⁶ are each independently H, OH, alkoxy, alkyl or aralkyl, or R²⁵ and R²⁶ together form oxo;
R²⁹ and R³⁰ are each independently H, alkyl or aralkyl, or together with the carbon atom to which they are attached form cycloalkyl; and
R²⁷, R²⁸, R³¹ and R³² are each independently H, alkyl or aralkyl.

In one embodiment, provided herein is a compound for use in the compositions and methods provided herein having Formula Vc or Vd, or a pharmaceutically acceptable derivative thereof, wherein:
E is an E3 ligase ligand or a moiety that activates the N-degron pathway;
L is a bond or a divalent chemical linker;
b is an integer from 1-4;
c is an integer from 1-3;
a, d and f are each independently an integer from 0-3;
X¹¹ is a bond, O, NR³¹ or CO;
X¹² is a bond, CONR³², SO₂NR³², CO or SO₂;
X¹³ is bicyclic heteroarylene optionally substituted with -L-E;
X¹⁵ is bicyclic heteroarylene substituted with -L-E;
R²¹ is aryl, heteroaryl or heterocycloalkyl;
R²² is aryl or heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo;
R²⁹ and R³⁰ are each independently H, alkyl or aralkyl, or together with the carbon atom to which they are attached form cycloalkyl; and
R²⁷, R²⁸, R³¹ and R³² are each independently H, alkyl or aralkyl.

In another embodiment, d and f are each 0, X¹² is a bond and the compound for use in the compositions and methods provided herein have Formula Ve or Vf:

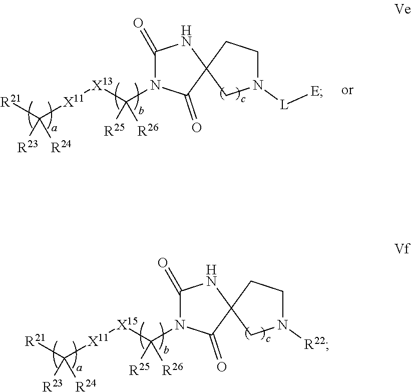

or a pharmaceutically acceptable derivative thereof, wherein:
E is an E3 ligase ligand or a moiety that activates the N-degron pathway;
L is a bond or a divalent chemical linker;
b is an integer from 1-4;
c is an integer from 1-3;
a is an integer from 0-3;
X¹¹ is a bond, O, NR³¹ or CO;
X¹³ is bicyclic heteroarylene optionally substituted with -L-E;
X¹ is bicyclic heteroarylene substituted with -L-E;
R²¹ is aryl, heteroaryl or heterocycloalkyl;
R²² is heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo; and
R³¹ is H, alkyl or aralkyl.

In another embodiment, c and g are each 2, d and f are each 0, X¹² is a bond and the compound for use in the compositions and methods provided herein have Formula Vg or Vh:

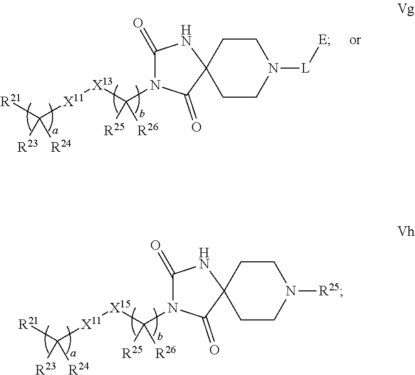

or a pharmaceutically acceptable derivative thereof, wherein:
E is an E3 ligase ligand or a moiety that activates the N-degron pathway;
L is a bond or a divalent chemical linker;
b is an integer from 1-4;
a is an integer from 0-3;
X¹¹ is a bond, O, NR³¹ or CO;
X¹³ is bicyclic heteroarylene optionally substituted with -L-E;
X¹⁵ is bicyclic heteroarylene substituted with -L-E;
R²¹ is aryl, heteroaryl or heterocycloalkyl;
R²² is heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo; and
R³¹ is H, alkyl or aralkyl.

In another embodiment, the compound for use in the compositions and methods provided herein has Formula Vi or Vj:

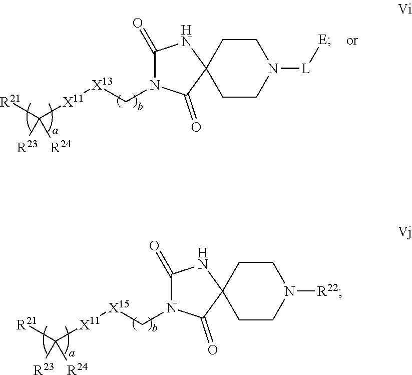

or a pharmaceutically acceptable derivative thereof, wherein:
E is an E3 ligase ligand or a moiety that activates the N-degron pathway;
L is a bond or a divalent chemical linker;
b is an integer from 1-4;
a is an integer from 0-3;
X¹¹ is a bond, O, NR³¹ or CO;
X¹³ is bicyclic heteroarylene optionally substituted with -L-E;
X¹⁵ is bicyclic heteroarylene substituted with -L-E;
R²¹ is aryl, heteroaryl or heterocycloalkyl;
R²² is heteroaryl;
R²³ and R²⁴ are each independently H or alkyl, or together with the carbon atom to which they are attached form cycloalkyl; and
R²⁵, R²⁶ and R³¹ are each independently H or alkyl.

In another embodiment, R²¹ in Formulae Va to Vj is aryl or heterocycloalkyl. In another embodiment, R²¹ in Formulae Va to Vj is aryl. In another embodiment, R²¹ in Formulae Va to Vj is heterocycloalkyl.

In another embodiment, the compound for use in the compositions and methods provided herein has Formula Vk or Vm:

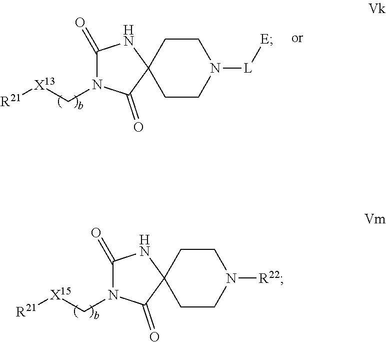

or a pharmaceutically acceptable derivative thereof, wherein:
E is an E3 ligase ligand or a moiety that activates the N-degron pathway;
L is a bond or a divalent chemical linker;
b is 2 or 3;
X¹³ is bicyclic heteroarylene optionally substituted with -L-E;
X¹⁵ is bicyclic heteroarylene substituted with -L-E;
R²¹ is heterocycloalkyl, optionally substituted with 1-3 substituents each independently selected from alkyl and aryl; and
R²² is heteroaryl.

In another embodiment, the compound for use in the compositions and methods provided herein has Formula Vn or Vo:

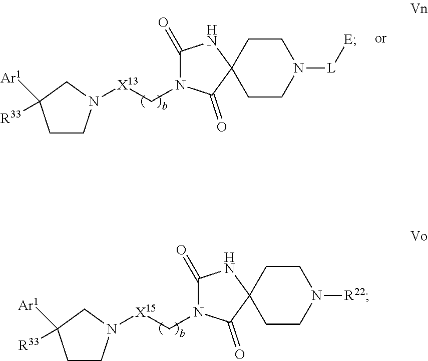

or a pharmaceutically acceptable derivative thereof, wherein:
E is an E3 ligase ligand or a moiety that activates the N-degron pathway;
L is a bond or a divalent chemical linker;
b is 2 or 3;
X¹³ is bicyclic heteroarylene optionally substituted with -L-E;
X¹⁵ is bicyclic heteroarylene substituted with -L-E;
Ar¹ is aryl;
R²² is heteroaryl; and
R³³ is alkyl.

In another embodiment, R³³ is lower alkyl. In another embodiment, R³³ is methyl or ethyl. In another embodiment, R³³ is methyl. In another embodiment, R³³ is ethyl.

In another embodiment, the compound for use in the compositions and methods provided herein has the formula:

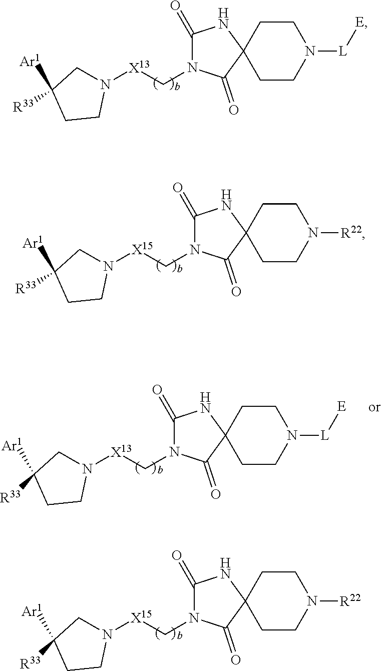

where the variables are as defined herein.

In another embodiment, the compound for use in the compositions and methods provided herein has Formula Vp or Vq:

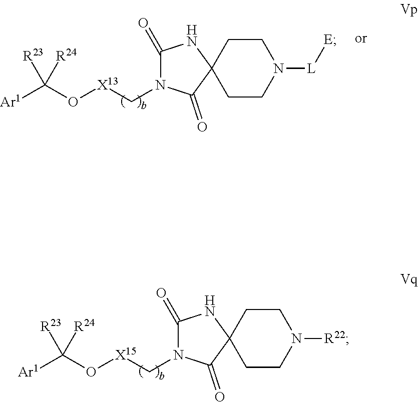

or a pharmaceutically acceptable derivative thereof, wherein:
E is an E3 ligase ligand or a moiety that activates the N-degron pathway;
L is a bond or a divalent chemical linker;
b is 2 or 3;
X¹³ is bicyclic heteroarylene optionally substituted with -L-E;
X¹⁵ is bicyclic heteroarylene substituted with -L-E;
Ar¹ is aryl;
R²² is heteroaryl; and
R²³ and R²⁴ are each independently H or alkyl, or together with the carbon atom to which they are attached form cycloalkyl.

In another embodiment, R²³ is alkyl and R²⁴ is H. In another embodiment, R²³ is methyl and R²⁴ is H. In another embodiment, R²³ and R²⁴ together with the carbon atom to which they are attached form cyclopropyl.

In another embodiment, the compound for use in the compositions and methods provided herein has the formula:

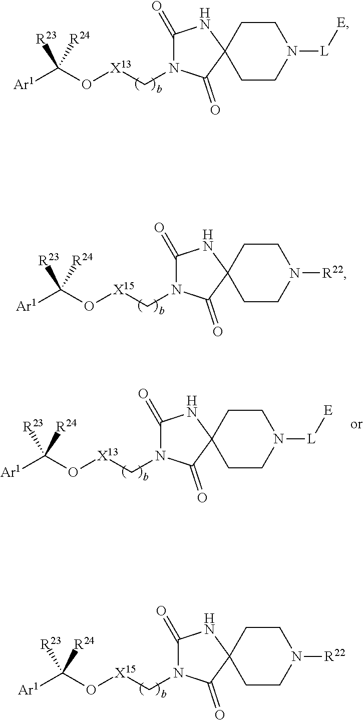

where the variables are as defined herein.

In another embodiment, b is 2. In another embodiment, b is 3.

In another embodiment, X¹³ is imidazopyridinylene or triazolopyridinylene, each optionally substituted with -L-E, alkyl, haloalkyl, hydroxyalkyl or halo. In another embodiment, X¹³ is imidazopyridinylene or triazolopyridinylene, each optionally substituted with -L-E, methyl, halomethyl, hydroxymethyl or halo. In another embodiment, X¹³ is triazolopyridinylene, optionally substituted with -L-E, methyl, halomethyl, hydroxymethyl or halo. In another embodiment, X¹³ is imidazopyridinylene, optionally substituted with -L-E, methyl, halomethyl, hydroxymethyl or halo. In another embodiment, X¹³ is 3-methylimidazo[1,2-a]pyridinyl-2,7-ene or 8-methyl-[1,2,4]triazolo[1,5-a]pyridinyl-2,6-ene, each optionally substituted with -L-E. In another embodiment, X¹³ has the structure:

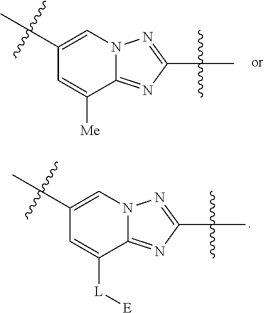

In another embodiment, X¹³ has the structure:

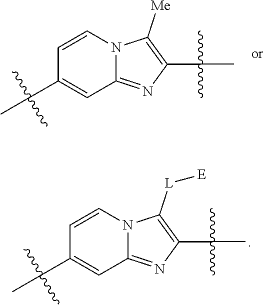

In one embodiment, provided herein is a compound for use in the compositions and methods provided herein having Formula Vr, Vs or Vt:

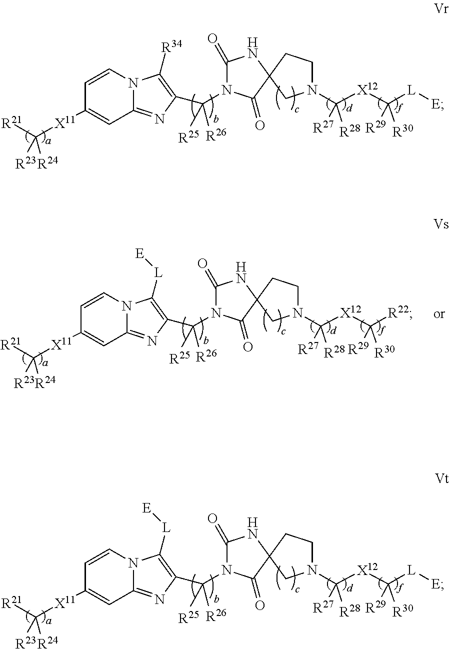

or a pharmaceutically acceptable derivative thereof, wherein
E is an E3 ligase ligand or a moiety that activates the N-degron pathway;
L is a bond or a divalent chemical linker;
b is an integer from 1-4;
c is an integer from 1-3;
a, d and f are each independently an integer from 0-3;
X¹¹ is a bond, O, NR³¹ or CO;
X¹² is a bond, CONR³², SO₂NR³², CO or SO₂;
R²¹ is aryl, heteroaryl or heterocycloalkyl;
R²² is aryl or heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo;
R²⁹ and R³⁰ are each independently H, alkyl or aralkyl, or together with the carbon atom to which they are attached form cycloalkyl; and
R²⁷, R²⁸, R³¹ and R³² are each independently H, alkyl or aralkyl; and
R³⁴ is H, alkyl, haloalkyl, hydroxyalkyl or halo.

In another embodiment, the compounds provided herein for use in the compositions and methods provided herein have Formula Vr, Vs or Vt, or a pharmaceutically acceptable derivative thereof, wherein:
E is an E3 ligase ligand or a moiety that activates the N-degron pathway;
L is a bond or a divalent chemical linker;
b is 2 or 3;
c is 2;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo;
R²⁹ and R³⁰ are each independently H, alkyl or aralkyl, or together with the carbon atom to which they are attached form cycloalkyl; and
R²⁷, R²⁸, R³¹ and R³² are each independently H, alkyl or aralkyl; and
R³⁴ is H, alkyl, haloalkyl, hydroxyalkyl or halo.

In another embodiment, the compounds provided herein for use in the compositions and methods provided herein have Formula Vr, Vs or Vt, or a pharmaceutically acceptable derivative thereof, wherein:
E is an E3 ligase ligand or a moiety that activates the N-degron pathway;
L is a bond or a divalent chemical linker;
b is 2 or 3;
c is 2;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²⁴ to R³² are each independently H; and
R²³ and R³⁴ are each independently alkyl.

In another embodiment, provided herein is a compound for use in the compositions and methods provided herein having Formula Vu, Vv or Vw:

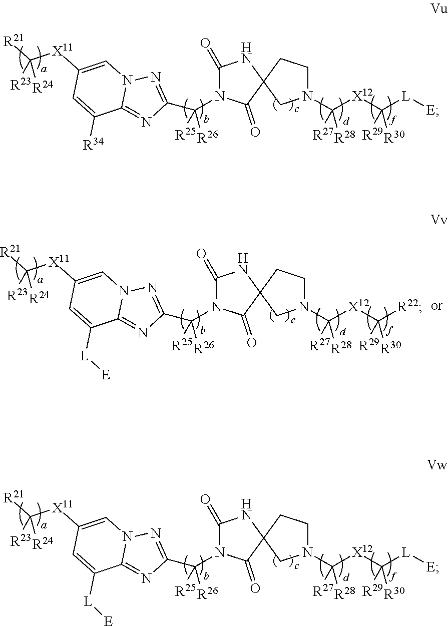

or a pharmaceutically acceptable derivative thereof, wherein
E is an E3 ligase ligand or a moiety that activates the N-degron pathway;
L is a bond or a divalent chemical linker;
b is an integer from 1-4;
c is an integer from 1-3;
a, d and f are each independently an integer from 0-3;
X¹¹ is a bond, O, NR³¹ or CO;
X¹² is a bond, CONR³², SO₂NR³², CO or SO₂;
R²¹ is aryl, heteroaryl or heterocycloalkyl;
R²² is aryl or heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo;
R²⁹ and R³⁰ are each independently H, alkyl or aralkyl, or together with the carbon atom to which they are attached form cycloalkyl; and
R²⁷, R²⁸, R³¹ and R³² are each independently H, alkyl or aralkyl; and
R³⁴ is H, alkyl, haloalkyl, hydroxyalkyl or halo.

In another embodiment, the compounds provided herein for use in the compositions and methods provided herein have Formula Vu, Vv or Vw, or a pharmaceutically acceptable derivative thereof, wherein:
E is an E3 ligase ligand or a moiety that activates the N-degron pathway;
L is a bond or a divalent chemical linker;
b is 2 or 3;
c is 2;
a is 0 or 1;
d and fare each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo;
R²⁹ and R³⁰ are each independently H, alkyl or aralkyl, or together with the carbon atom to which they are attached form cycloalkyl; and
R²⁷ and R²⁸ are each independently H, alkyl or aralkyl; and
R³⁴ is H, alkyl, haloalkyl, hydroxyalkyl or halo.

In another embodiment, the compounds provided herein for use in the compositions and methods provided herein have Formula Vu, Vv or Vw, or a pharmaceutically acceptable derivative thereof, wherein:
E is an E3 ligase ligand or a moiety that activates the N-degron pathway;
L is a bond or a divalent chemical linker;
b is 2 or 3;
c is 2;
a is 0 or 1;
d and f are each 0;
X¹¹ is a bond or 0;
X¹² is a bond;
R²¹ is aryl or heterocycloalkyl, each optionally substituted with 1-3 substituents each independently selected from alkyl and phenyl;
R²² is heteroaryl;
R²⁴ to R³⁰ are each independently H; and
R²³ and R³⁴ are each independently alkyl.

In another embodiment, the compound for use in the compositions and methods provided herein has Formula Vx, Vy or Vz:

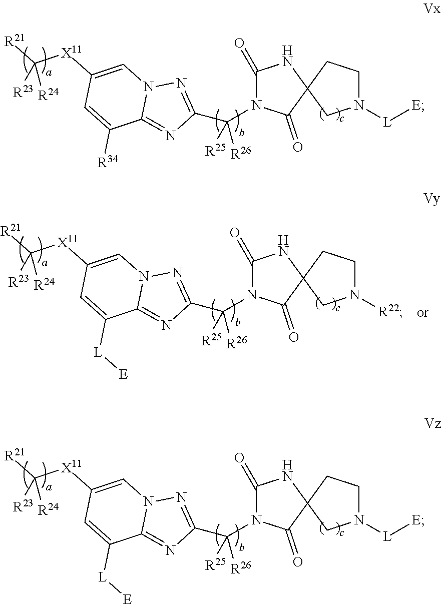

wherein:
E is an E3 ligase ligand or a moiety that activates the N-degron pathway;
L is a bond or a divalent chemical linker;
b is an integer from 1-4;
c is an integer from 1-3;
a is an integer from 0-3;
X¹¹ is a bond, O, NR³¹ or CO;
R²¹ is aryl or heterocycloalkyl;
R²² is heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo; and
R³⁴ is H, alkyl, haloalkyl, hydroxyalkyl or halo.

In another embodiment, the compound for use in the compositions and methods provided herein has Formula Vaa, Vbb or Vcc:

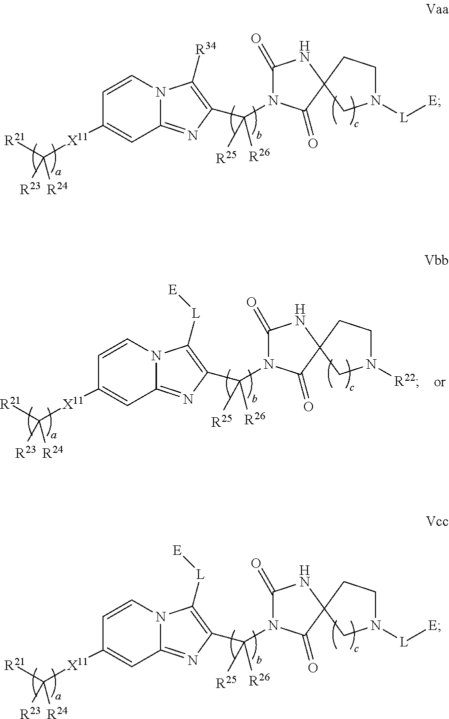

wherein:
E is an E3 ligase ligand or a moiety that activates the N-degron pathway;
L is a bond or a divalent chemical linker;
b is an integer from 1-4;
c is an integer from 1-3;
a is an integer from 0-3;
X¹¹ is a bond, O, NR³¹ or CO;
R²¹ is aryl or heterocycloalkyl;
R²² is heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo; and
R³⁴ is H, alkyl, haloalkyl, hydroxyalkyl or halo.

In another embodiment, he compounds for use in the compositions and methods provided herein have Formula Vx, Vy, Vz, Vaa, Vbb or Vcc, wherein:
E is an E3 ligase ligand or a moiety that activates the N-degron pathway;
L is a bond or a divalent chemical linker;
b is 2 or 3;
c is 2;
a is 0 or 1;
X¹¹ is a bond or 0;
R²¹ is aryl or heterocycloalkyl, each optionally substituted with 1-3 substituents each independently selected from alkyl and aryl;
R²² is heteroaryl;
R²³ and R²⁴ are each independently H, alkyl or aralkyl, or together with the carbon atom to which they are attached form cycloalkyl;
R²⁵ and R²⁶ are each independently H, OH, alkoxy, alkyl or aralkyl, or R²⁵ and R²⁶ together form oxo; and
R³⁴ is H, alkyl, haloalkyl, hydroxyalkyl or halo.

In another embodiment, Ar¹ is unsubstituted aryl. In another embodiment, Ar¹ is optionally substituted phenyl. In another embodiment, Ar¹ is unsubstituted phenyl.

In another embodiment, R²⁵ and R²⁶ are each independently H, alkyl or aralkyl. In another embodiment, R²⁵ and R²⁶ are each independently H, OH, OMe or methyl, or R²⁵ and R²⁶ together form oxo. In another embodiment, R²⁵ and R²⁶ are each independently H, OH, OMe or methyl.

In another embodiment, R²⁹ and R³⁰ are each independently H, alkyl or aralkyl.

In another embodiment, R²⁵ and R²⁶ are each independently H, alkyl or aralkyl, and R³⁰ are each independently H, alkyl or aralkyl.

In another embodiment, the compounds for use in the compositions and methods provided herein has one of the formulae:

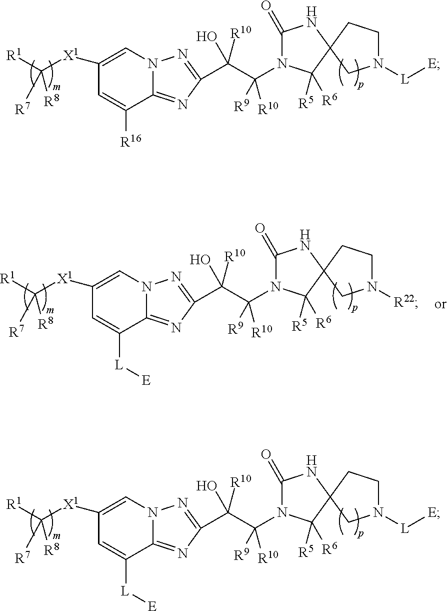

or a pharmaceutically acceptable derivative thereof, where R¹, R², R⁵—R¹⁰, R¹⁶, L, E, m and p are as defined herein.

In another embodiment, the compounds provided herein for use in the compositions and methods provided herein are selected from:

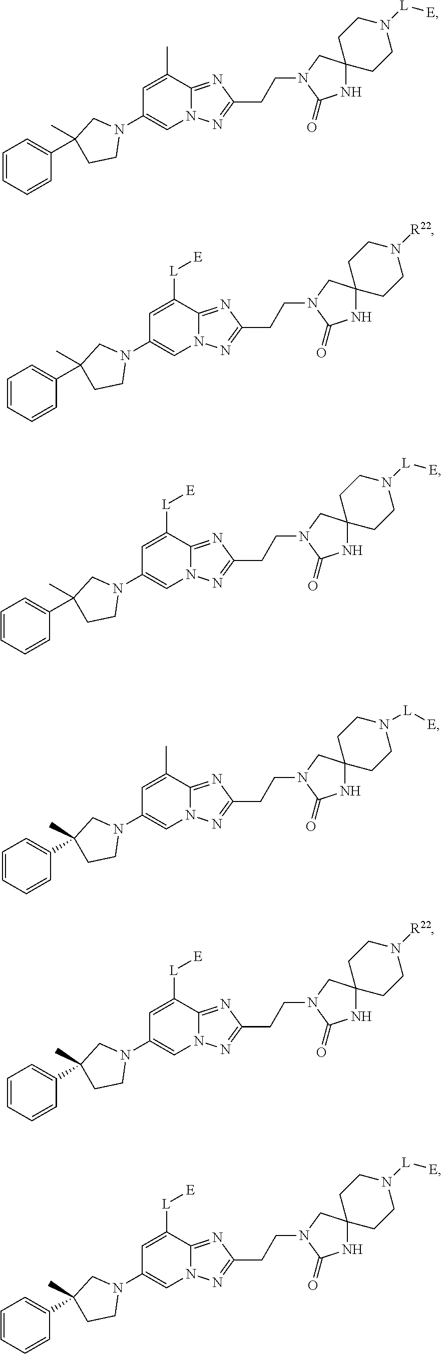

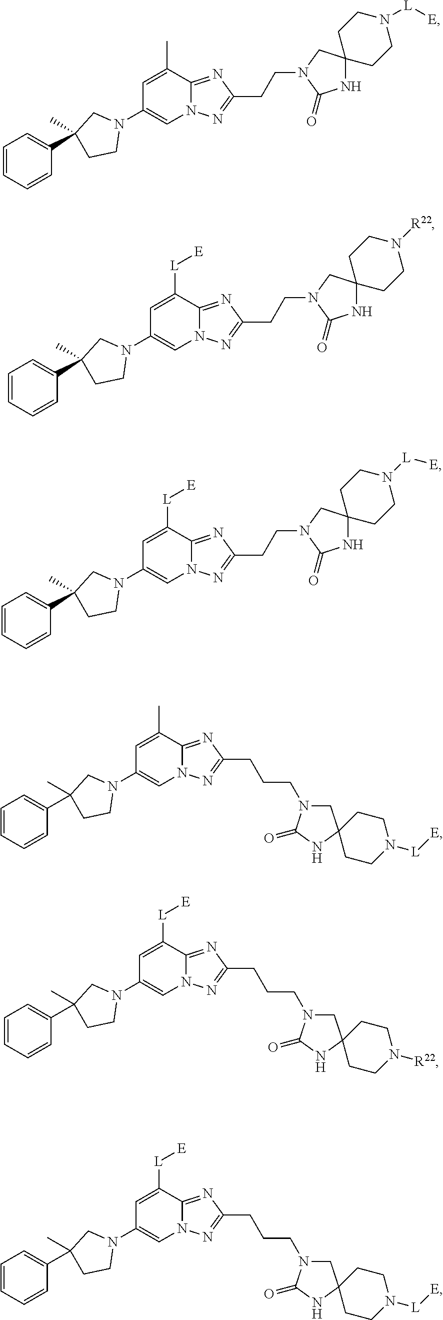

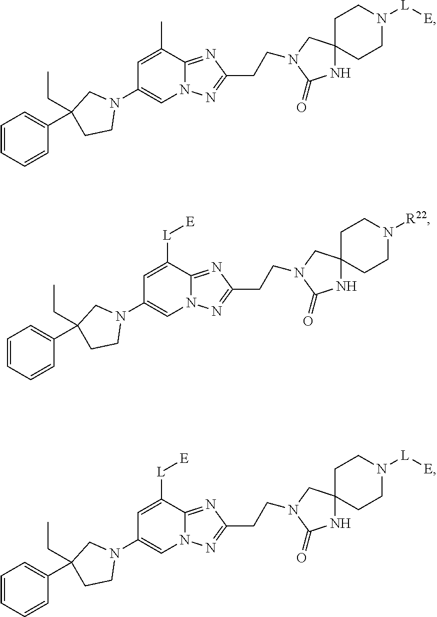

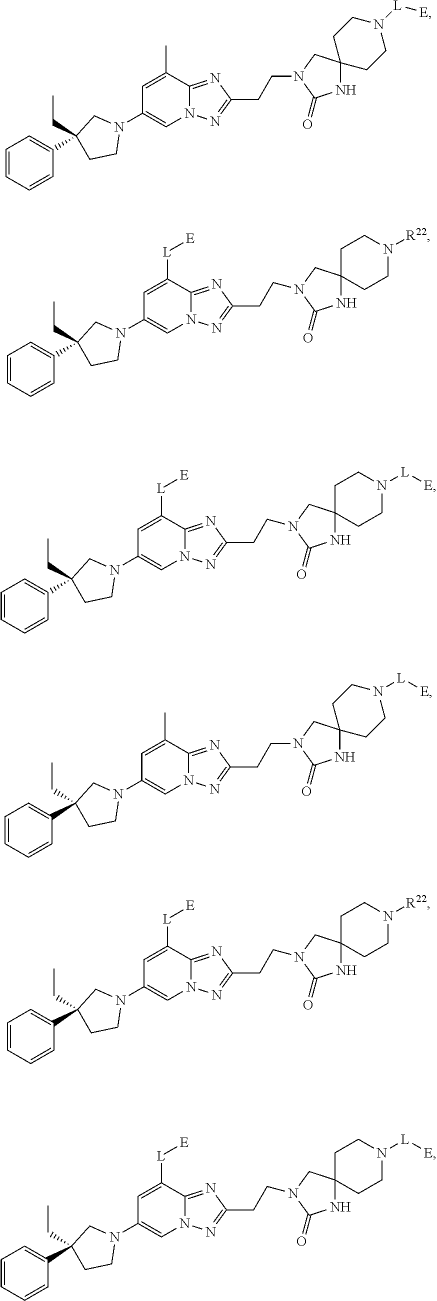

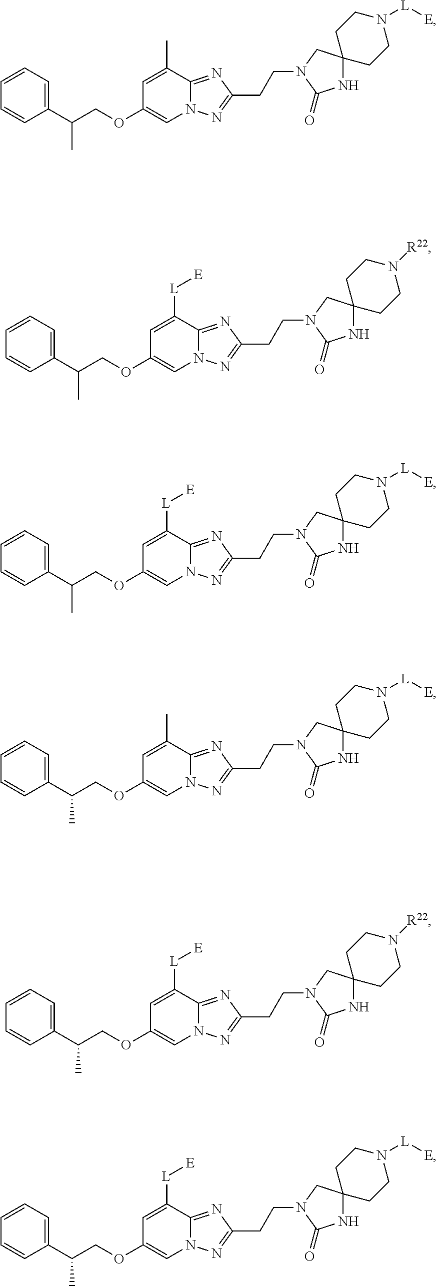

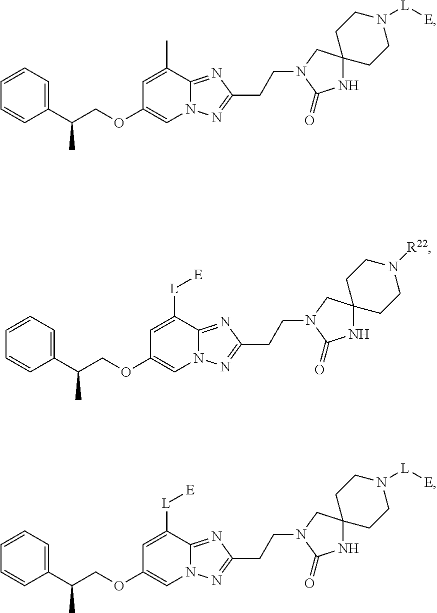

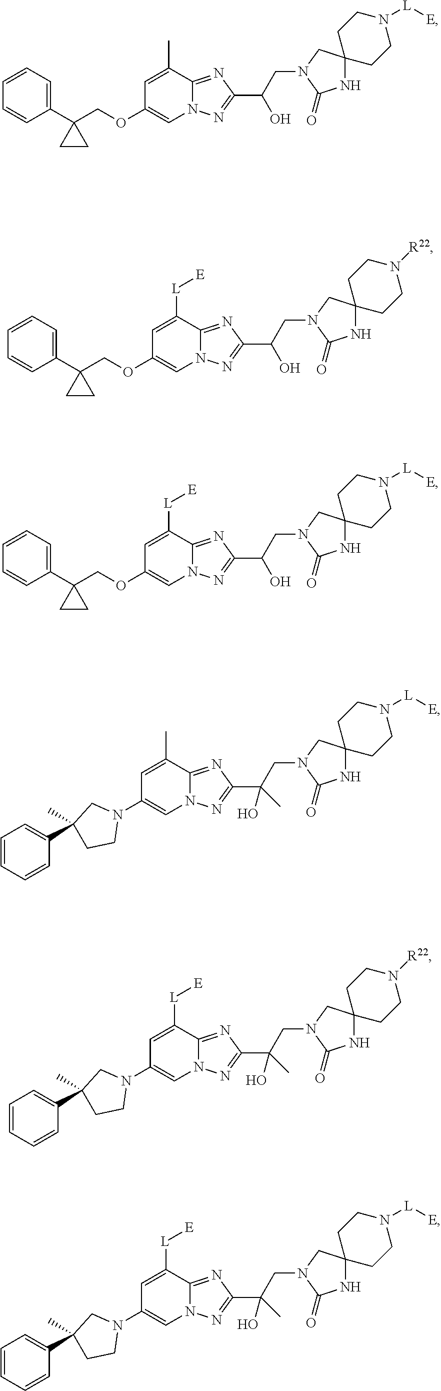

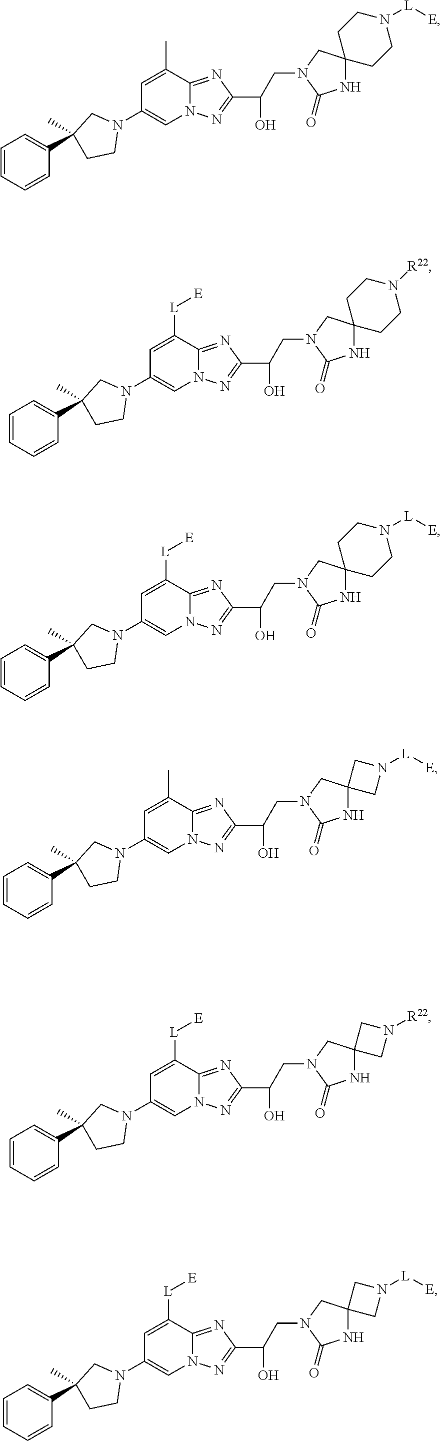

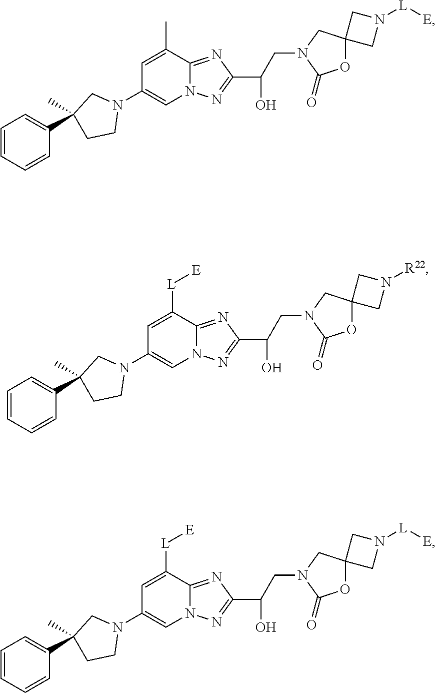

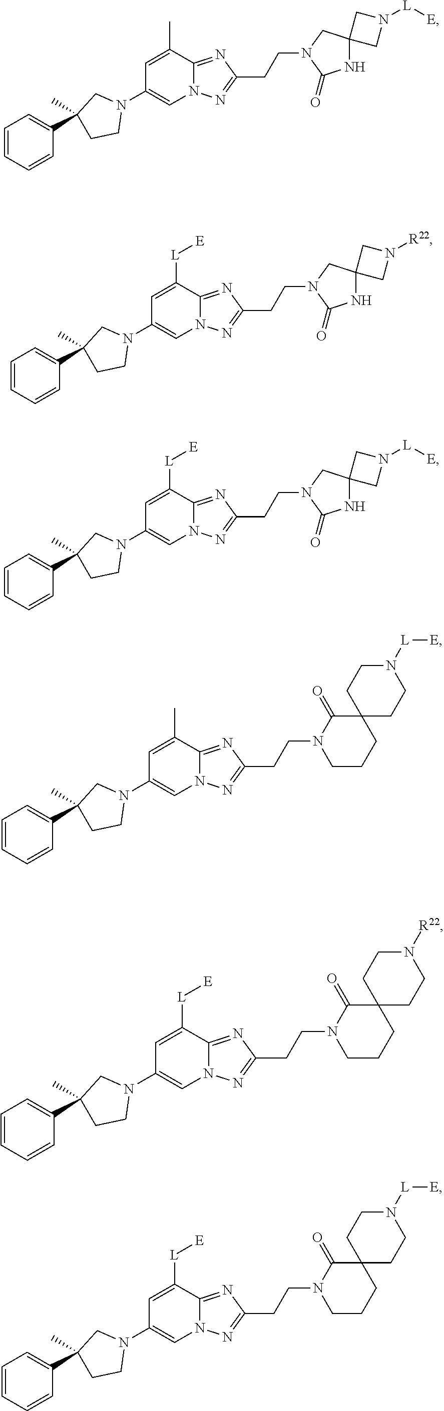

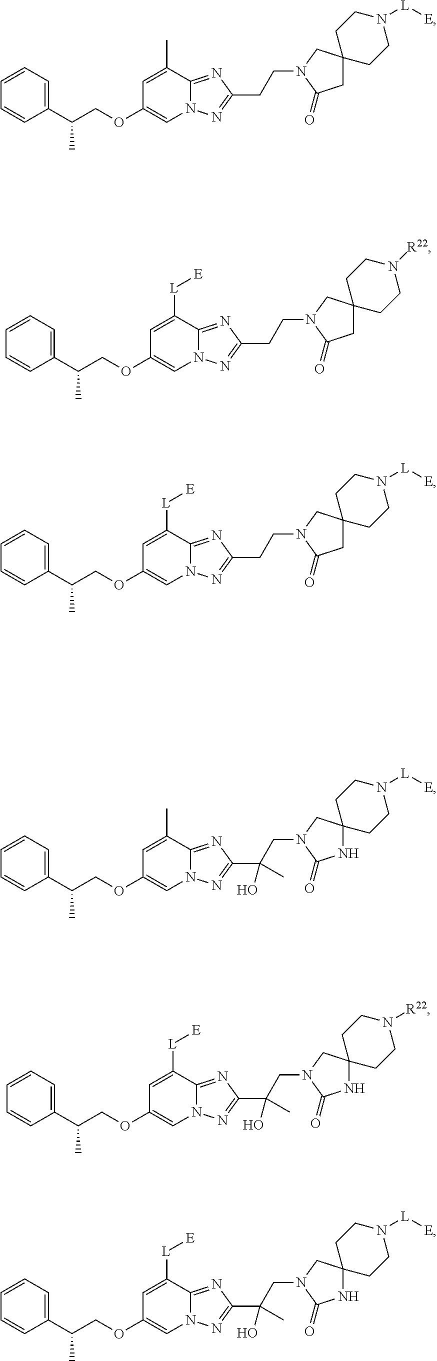

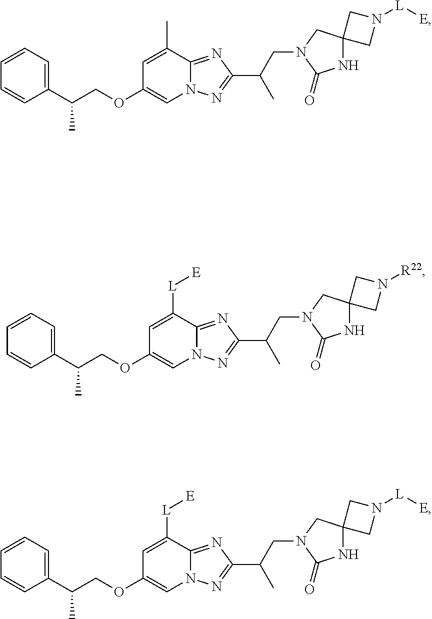

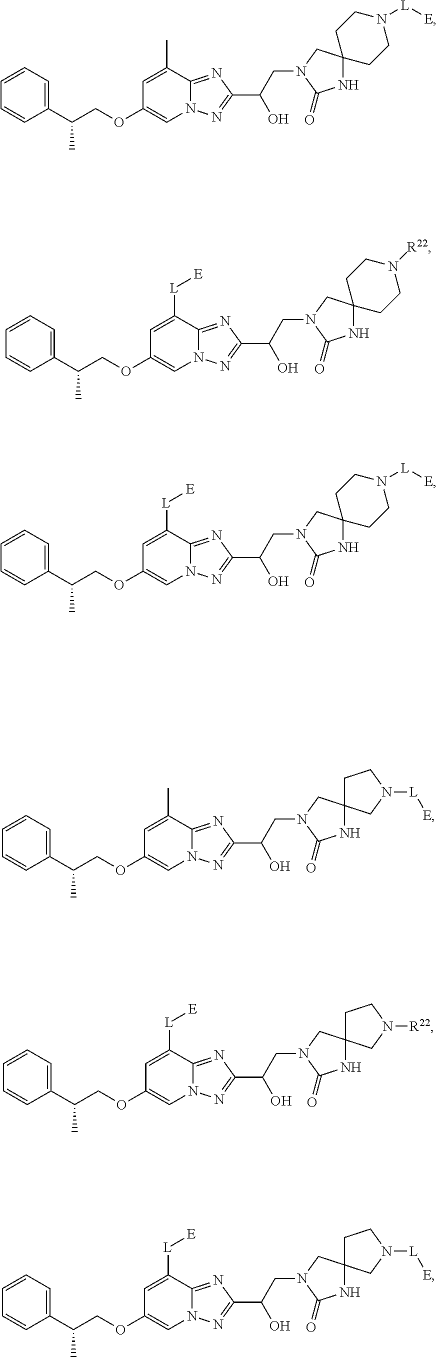

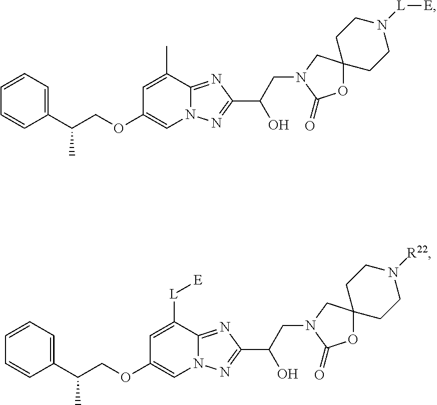

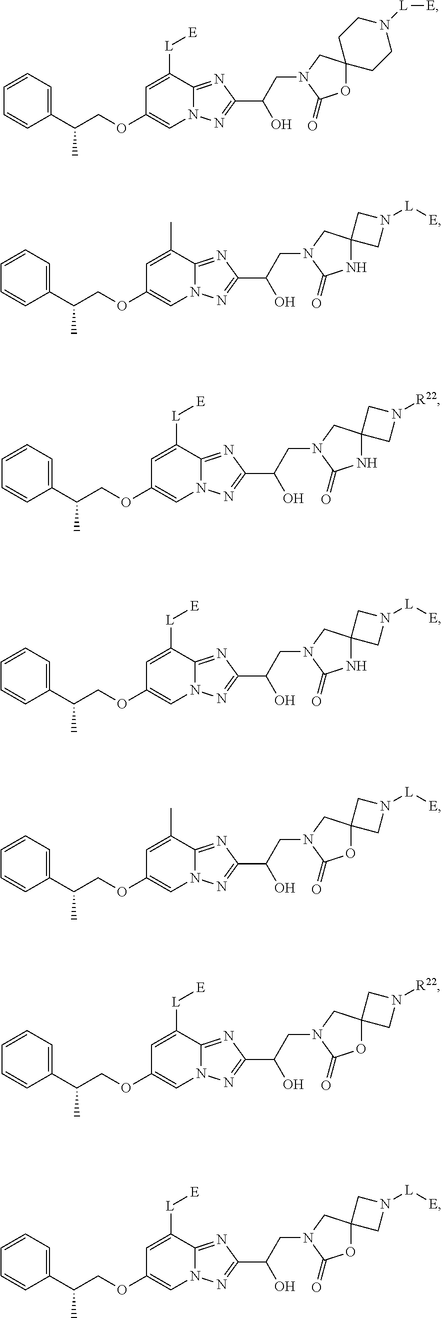

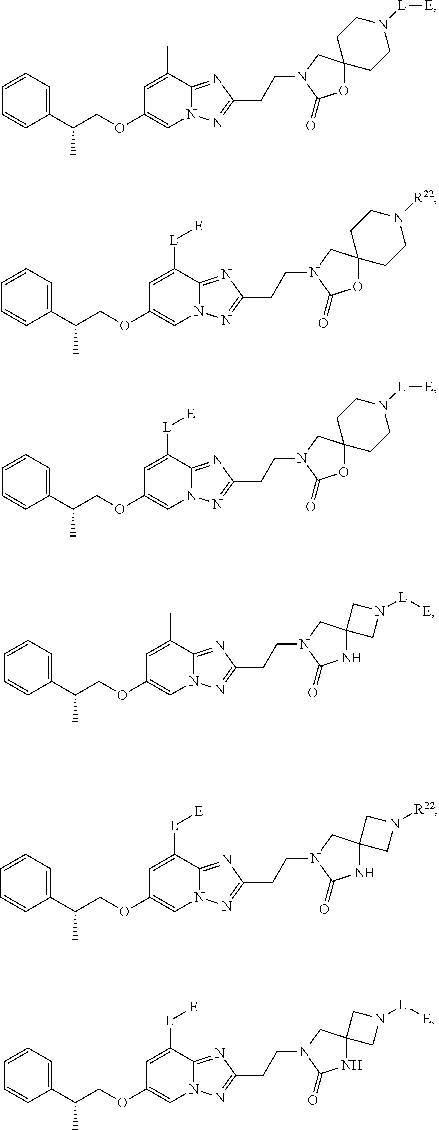

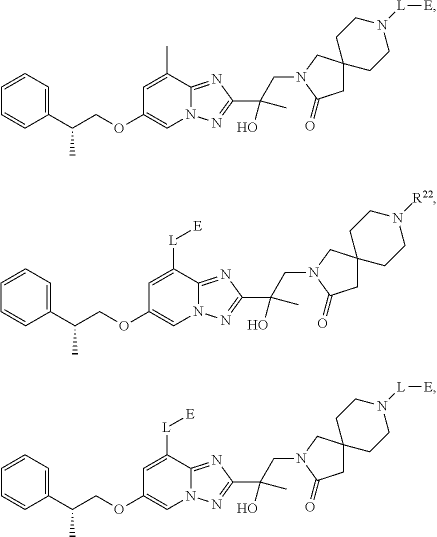

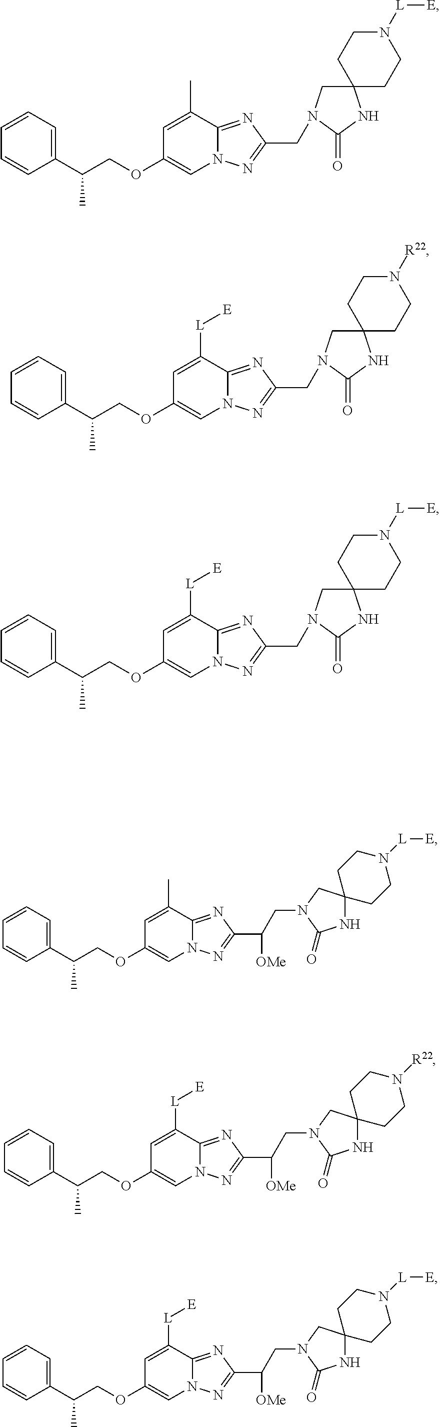

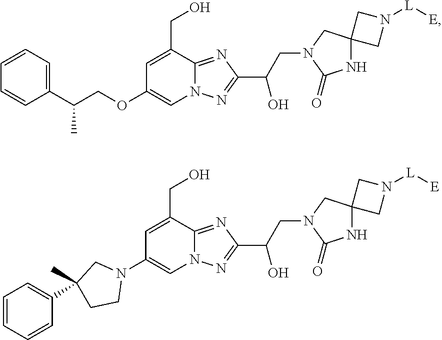

In another embodiment, provided herein is a compound for use in the compositions and methods provided herein having Formula VIa, VIb or VIc.

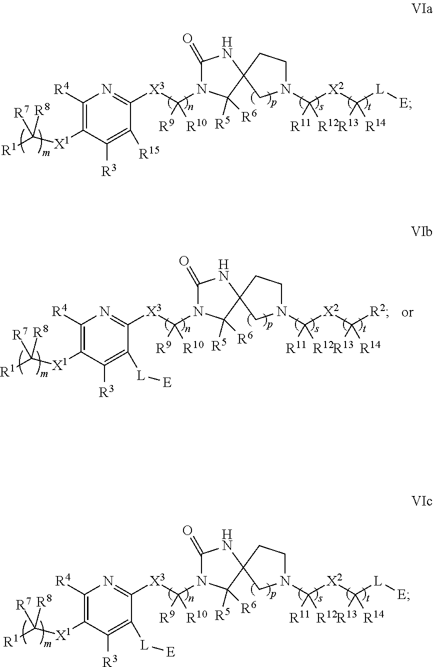

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁷, CO or SO₂;
X³ is a bond, CONR¹⁸, SO₂NR¹⁸, CO or SO₂;
R¹ is aryl, heteroaryl or heterocycloalkyl;
R² is H, COR¹⁹, COOR¹⁹, CONR¹⁹R²⁰, aryl or heteroaryl;
R³ to R⁶, R⁹ to R¹⁴, and R¹⁶ to R²⁰ are each independently H, alkyl or aralkyl;
R⁷ and R⁸ are each independently H, alkyl or aralkyl, or together with the carbon atom to which they are attached form cycloalkyl; and

R¹⁵ is H, alkyl, aralkyl, halo or haloalkyl.

In another embodiment, s and t are each 0, X² is a bond and the compound for use in the compositions and methods provided herein has Formula VId, VIe or VIf:

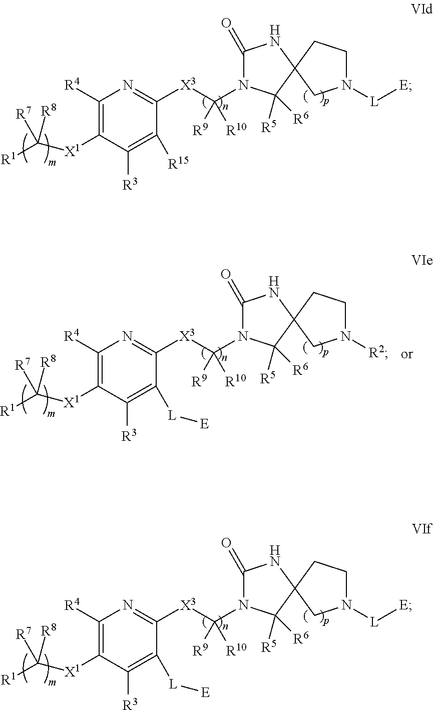

or a pharmaceutically acceptable derivative thereof, wherein
E is an E3 ligase ligand or a moiety that activates the N-degron pathway;
L is a bond or a divalent chemical linker;
n is an integer from 1-4;
p is an integer from 1-3;
m is an integer from 0-3;
X¹ is a bond, O, NR¹⁶ or CO;
X³ is a bond, CONR¹⁸, SO₂NR¹⁸, CO or SO₂;
R¹ is aryl, heteroaryl or heterocycloalkyl;
R² is H, COR¹⁹, COOR¹⁹, CONR¹⁹R²⁰, aryl or heteroaryl;
R³ to R⁶, R⁹, R¹⁰, R¹⁶, R¹⁸, R¹⁹ and R²⁰ are each independently H, alkyl or aralkyl;
R⁷ and R⁸ are each independently H, alkyl or aralkyl, or together with the carbon atom to which they are attached form cycloalkyl; and
R″ is H, alkyl, aralkyl, halo or haloalkyl.

In another embodiment, p is 2, s and t are each 0, X² is a bond and the compound for use in the compositions and methods provided herein has Formula VIg, VIh or VIi:

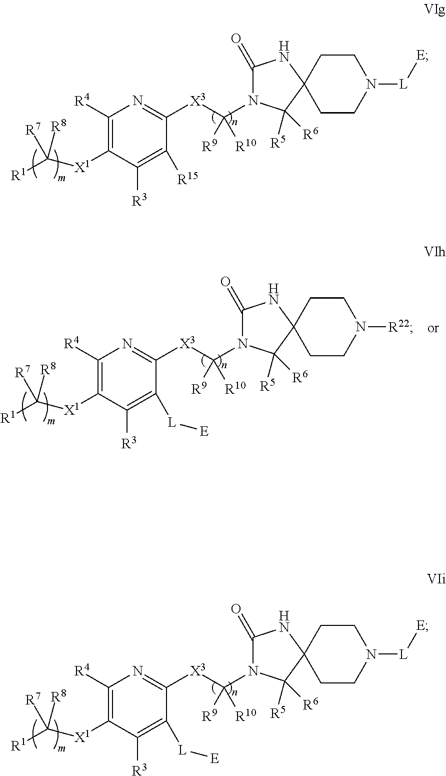

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR¹⁶ or CO;
X³ is a bond, CONR¹⁸, SO₂NR¹⁸, CO or SO₂;
R¹ is aryl, heteroaryl or heterocycloalkyl;
R² is H, COR¹⁹, COOR¹⁹, CONR¹⁹R²⁰, aryl or heteroaryl;
R³ to R⁶, R⁹, R¹⁰, R¹⁶, R¹⁸, R¹⁹ and R²⁰ are each independently H, alkyl or aralkyl;
R⁷ and R⁸ are each independently H, alkyl or aralkyl, or together with the carbon atom to which they are attached form cycloalkyl; and
R″ is H, alkyl, aralkyl, halo or haloalkyl.

In another embodiment, the compound for use in the compositions and methods provided herein has Formula VIj, VIk or VIm:

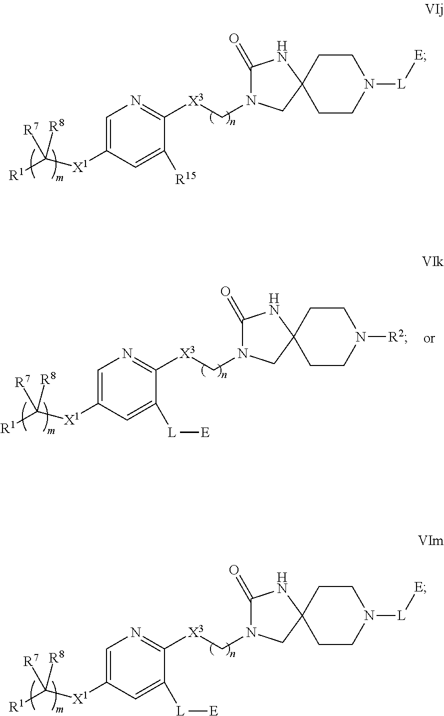

or a pharmaceutically acceptable derivative thereof, wherein
E is an E3 ligase ligand or a moiety that activates the N-degron pathway;
L is a bond or a divalent chemical linker;
n is an integer from 1-4;
m is an integer from 0-3;
X¹ is a bond, O, NR¹⁶ or CO;
X³ is a bond, CONR¹⁸, SO₂NR¹⁸, CO or SO₂;
R¹ is aryl, heteroaryl or heterocycloalkyl;
R² is H, COR¹⁹, COOR¹⁹, CONR¹⁹R²⁰, aryl or heteroaryl;
R¹⁶, R¹⁸, R¹⁹ and R²⁰ are each independently H, alkyl or aralkyl;
R⁷ and R⁸ are each independently H, alkyl or aralkyl, or together with the carbon atom to which they are attached form cycloalkyl; and
R″ is H, alkyl, aralkyl, halo or haloalkyl.

In another embodiment, R¹ in Formulae VIa to VIm is aryl or heterocycloalkyl. In another embodiment, R¹ in Formulae VIa to VIm is aryl. In another embodiment, R¹ in Formulae VIa to VIm is heterocycloalkyl.

In another embodiment, the compound for use in the compositions and methods provided herein has Formula VIn, VIo or VIp:

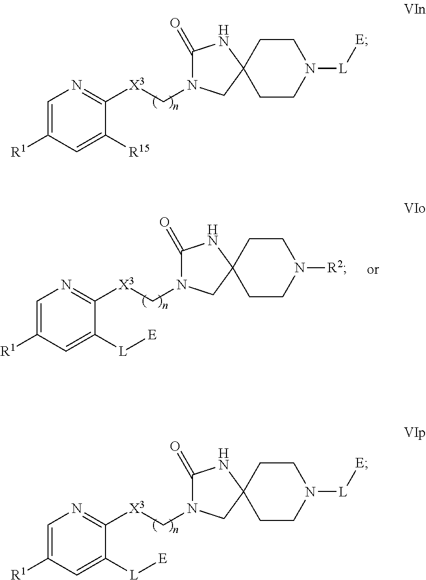

or a pharmaceutically acceptable derivative thereof, wherein
E is an E3 ligase ligand or a moiety that activates the N-degron pathway;
L is a bond or a divalent chemical linker;
n is 2 or 3;
X³ is a bond, CONR¹⁸, SO₂NR¹⁸, CO or SO₂;
R¹ is heterocycloalkyl, optionally substituted with 1-3 substituents each independently selected from alkyl and aryl;
R² is H, COOR¹⁹ or heteroaryl;
R″ is alkyl, halo or haloalkyl;
R¹⁸ is H; and
R¹⁹ is alkyl.

In another embodiment, the compound for use in the compositions and methods provided herein has Formula VIq, VIr or VIs:

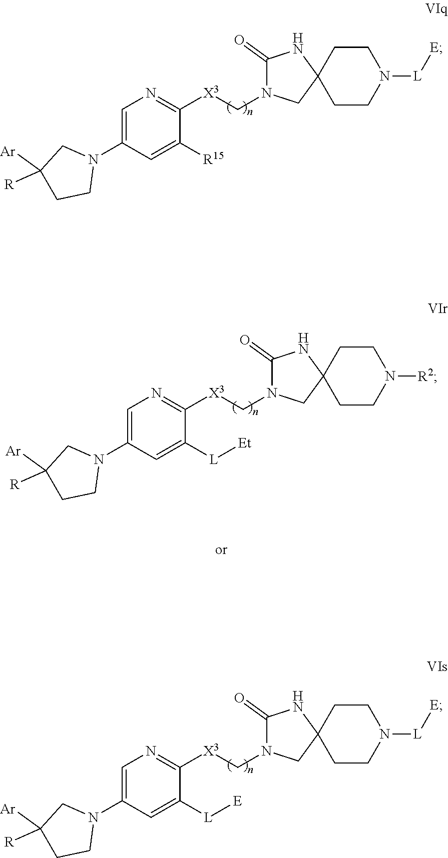

or a pharmaceutically acceptable derivative thereof, wherein
E is an E3 ligase ligand or a moiety that activates the N-degron pathway;
L is a bond or a divalent chemical linker;
n is 2 or 3;
X³ is CONR¹⁸;
Ar is aryl;
R² is H, COOR¹⁹ or heteroaryl;
R¹⁵ is alkyl, halo or haloalkyl;
R¹⁸ is H; and
R¹⁹ and R are each independently alkyl.

In another embodiment, R is lower alkyl. In another embodiment, R is methyl or ethyl. In another embodiment, R is methyl. In another embodiment, R is ethyl.

In another embodiment, the compound for use in the compositions and methods provided herein has the formula:

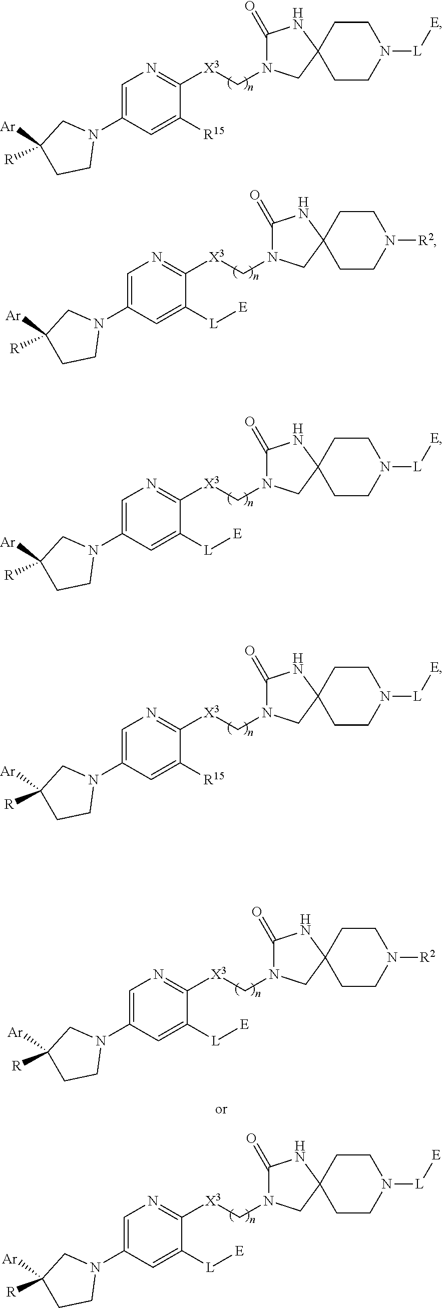

where the variables are as defined herein.

In another embodiment, the compound for use in the compositions and methods provided herein has Formula VIt, VIu or VIv:

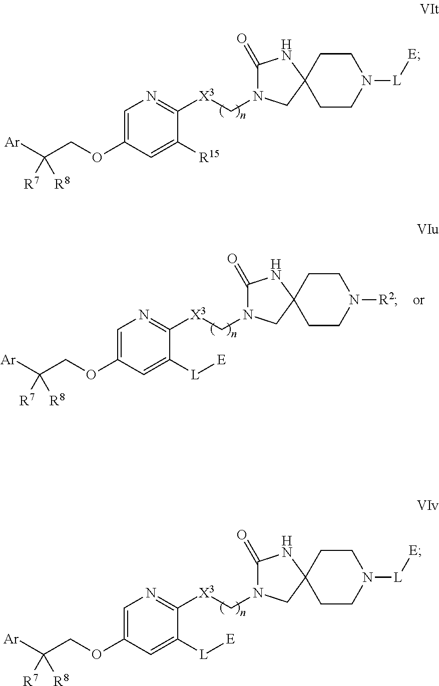

or a pharmaceutically acceptable derivative thereof, wherein
E is an E3 ligase ligand or a moiety that activates the N-degron pathway;
L is a bond or a divalent chemical linker;
n is 2 or 3;
X³ is CONR¹⁸;
Ar is aryl;
R² is H, COOR¹⁹ or heteroaryl;
R⁷ and R⁸ are each independently H or alkyl, or together with the carbon atom to which they are attached form cycloalkyl;
R¹⁵ is alkyl, halo or haloalkyl;
R¹⁸ is H; and
R¹⁹ is alkyl.

In another embodiment, R⁷ is alkyl and R⁸ is H. In another embodiment, R⁷ is methyl and R⁸ is H. In another embodiment, R⁷ and R⁸ together with the carbon atom to which they are attached form cyclopropyl.

In another embodiment, the compound for use in the compositions and methods provided herein has the formula:

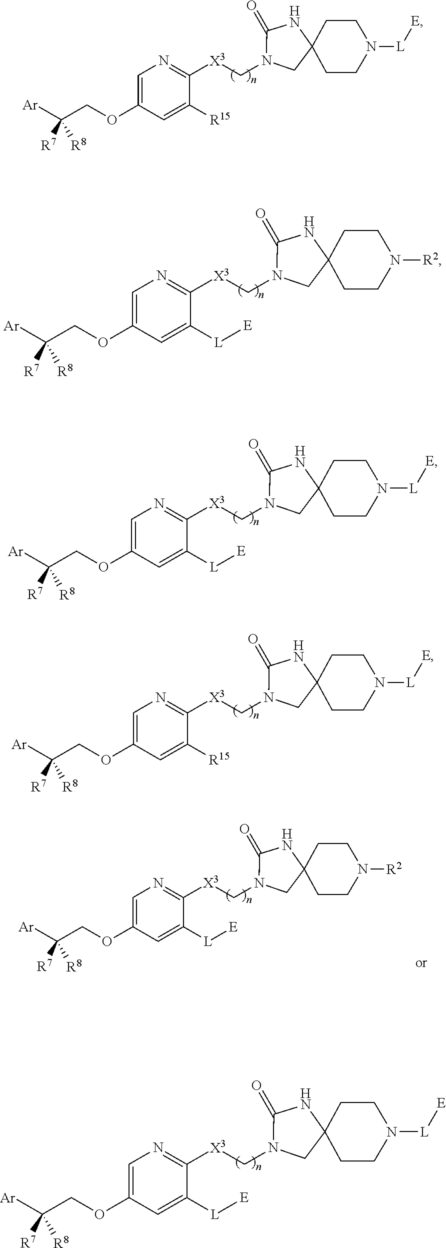

where the variables are as defined herein.

In another embodiment, n is 2. In another embodiment, n is 3.

In another embodiment, R¹⁵ is lower alkyl. In another embodiment, R¹⁵ is methyl or ethyl. In another embodiment, R¹⁵ is methyl. In another embodiment, R¹⁵ is halo. In another embodiment, R¹⁵ is chloro. In another embodiment, R¹⁵ is haloalkyl. In another embodiment, R¹⁵ is trifluoromethyl.

In another embodiment, R¹⁹ is lower alkyl. In another embodiment, R¹⁹ is tert-butyl. In another embodiment, R¹⁹ is methyl.

In another embodiment, R¹⁵ is chloro and R is methyl. In another embodiment, R¹⁵ is chloro and R is ethyl. In another embodiment, n is 2, R¹⁵ is chloro and R is methyl. In another embodiment, n is 2, R¹⁵ is chloro and R is ethyl.

In another embodiment, Ar is unsubstituted aryl. In another embodiment, Ar is optionally substituted phenyl. In another embodiment, Ar is unsubstituted phenyl.

In another embodiment, R² is H. In another embodiment, R² is COO-t-Bu. In another embodiment, R² is COMe. In another embodiment, R² is bicyclic or monocyclic heteroaryl. In another embodiment, R² is pyridyl, pyrimidinyl, pyrizinyl, pyridazinyl, triazolopyrazinyl or imidazopyrazinyl. In another embodiment, R² is triazolopyrazinyl or imidazopyrazinyl. In another embodiment, R² is 2-pyrimidinyl, 2-ethoxycarbonyl-5-pyridyl, 2-(N-methyl)carbamoyl-4-pyridyl, 5-pyrimidinyl, 2-methoxycarbonyl-3-fluoro-5-pyridyl, 6-methoxycarbonyl-3-pyridazinyl, 2-carboxy-3-fluoro-5-pyridyl, 6-carboxy-3-pyridazinyl, 2-carboxy-5-pyridyl, 2-pyrazinyl, [1,2,4]-triazolo[4,3-a]pyrazin-8-yl, imidazo[1,5-a]pyrazin-8-yl or imidazo[1,2-a]pyrazin-8-yl. In another embodiment, R² is [1,2,4]-triazolo[4,3-a]pyrazin-8-yl, imidazo[1,5-a]pyrazin-8-yl or imidazo[1,2-a]pyrazin-8-yl.

In one embodiment, the compound provided herein for use in the compositions and methods provided herein is selected from:

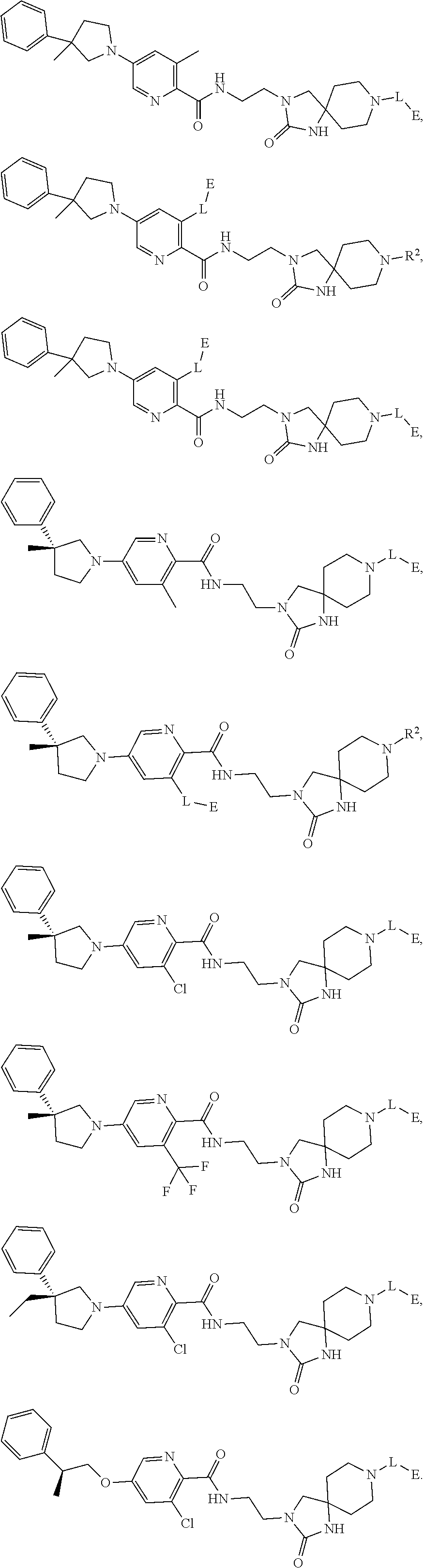

In another embodiment, provided herein is a compound for use in the compositions and methods provided herein having Formula VIIa, VIIb or VIIc:

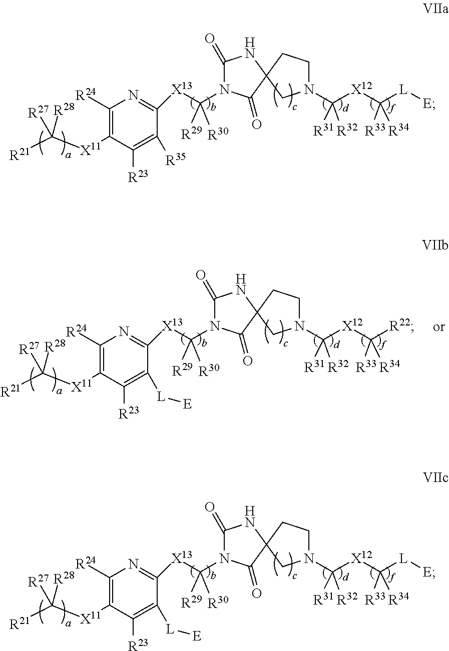

or a pharmaceutically acceptable derivative thereof, wherein:
E is an E3 ligase ligand or a moiety that activates the N-degron pathway;
L is a bond or a divalent chemical linker;
b is an integer from 1-4;
c is an integer from 1-3;
a, d and f are each independently an integer from 0-3;
X¹¹ is a bond, O, NR³⁶ or CO;
X¹² is a bond, CONR³⁷, SO₂NR³⁷, CO or SO₂;
X¹³ is a bond, CONR³⁸, SO₂NR³⁸, CO or SO₂;
R²¹ is aryl, heteroaryl or heterocycloalkyl;
R²² is H, COR³⁹, COOR³⁹, CONR³⁹R⁴⁰, aryl or heteroaryl;
R²³, R²⁴, R²⁹ to R³⁴, and R³⁶ to R⁴⁰ are each independently H, alkyl or aralkyl;
R²⁷ and R²⁸ are each independently H, alkyl or aralkyl, or together with the carbon atom to which they are attached form cycloalkyl; and
R³⁵ is H, alkyl, aralkyl, halo or haloalkyl.

In another embodiment, d and f are each 0, X¹² is a bond and the compound for use in the compositions and methods provided herein has Formula VIId, VIIe or VIIf:

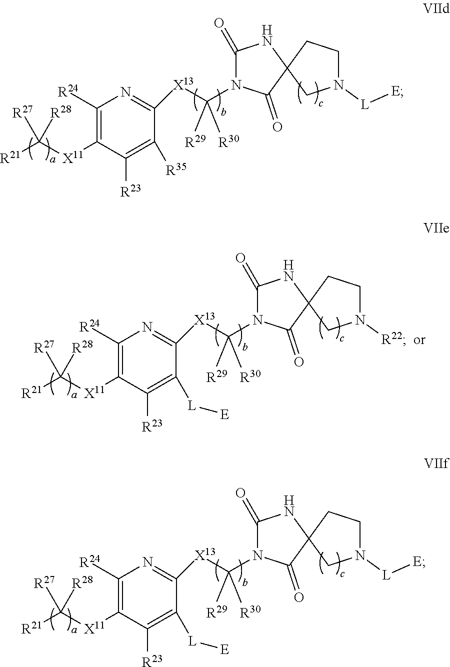

or a pharmaceutically acceptable derivative thereof, wherein:
E is an E3 ligase ligand or a moiety that activates the N-degron pathway;
L is a bond or a divalent chemical linker;
b is an integer from 1-4;
c is an integer from 1-3;
a is an integer from 0-3;
X¹¹ is a bond, O, NRA⁶ or CO;
X¹³ is a bond, CONRW⁸, SO₂NR³⁸, CO or SO₂;
R²¹ is aryl, heteroaryl or heterocycloalkyl;
R²² is H, COR³⁹, COOR³⁹, CONR³⁹R⁴⁰, aryl or heteroaryl;
R²³, R²⁴, R²⁹, R³⁰, R³⁶, R³⁸, R³⁹ and R⁴⁰ are each independently H, alkyl or aralkyl;
R²⁷ and R²⁸ are each independently H, alkyl or aralkyl, or together with the carbon atom to which they are attached form cycloalkyl; and
R³⁵ is H, alkyl, aralkyl, halo or haloalkyl.

In another embodiment, c is 2, d and f are each 0, X¹² is a bond and the compound for use in the compositions and methods provided herein has Formula VIIg, VIIh or VIIi:

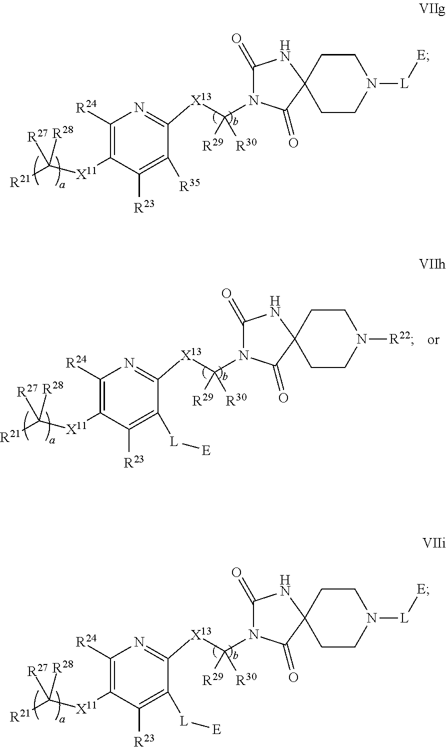

or a pharmaceutically acceptable derivative thereof, wherein:
E is an E3 ligase ligand or a moiety that activates the N-degron pathway;
L is a bond or a divalent chemical linker;
b is an integer from 1-4;
a is an integer from 0-3;
X¹¹ is a bond, O, NR³⁶ or CO;
X¹³ is a bond, CONR³⁸, SO₂NR³⁸, CO or SO₂;
R²¹ is aryl, heteroaryl or heterocycloalkyl;
R²² is H, COR³⁹, COOR³⁹, CONR³⁹R⁴⁰, aryl or heteroaryl;
R²³, R²⁴, R²⁹, R³⁰, R³⁶, R³⁸, R³⁹ and R⁴⁰ are each independently H, alkyl or aralkyl;
R²⁷ and R²⁸ are each independently H, alkyl or aralkyl, or together with the carbon atom to which they are attached form cycloalkyl; and
R³⁵ is H, alkyl, aralkyl, halo or haloalkyl.

In another embodiment, the compound for use in the compositions and methods provided herein has Formula VIIj, VIIk or VIIm:

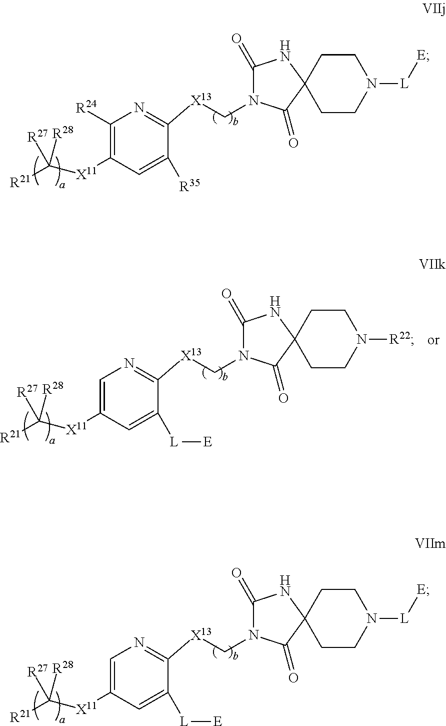

or a pharmaceutically acceptable derivative thereof, wherein:
E is an E3 ligase ligand or a moiety that activates the N-degron pathway;
L is a bond or a divalent chemical linker;
b is an integer from 1-4;
a is an integer from 0-3;
X¹¹ is a bond, O, NR³⁶ or CO;
X¹³ is a bond, CONR³⁸, SO₂NR³⁸, CO or SO₂;
R²¹ is aryl, heteroaryl or heterocycloalkyl;
R²² is H, COR³⁹, COOR³⁹, CONR³⁹R⁴⁰, aryl or heteroaryl;
R³⁶, R³⁸, R³⁹ and R⁴⁰ are each independently H, alkyl or aralkyl;
R²⁷ and R²⁸ are each independently H, alkyl or aralkyl, or together with the carbon atom to which they are attached form cycloalkyl; and
R³⁵ is H, alkyl, aralkyl, halo or haloalkyl.

In another embodiment, R²¹ in Formulae VIIa to VIIm is aryl or heterocycloalkyl. In another embodiment, R²¹ in Formulae VIIa to VIIm is aryl. In another embodiment, R²¹ in Formulae VIIa to VIIm is heterocycloalkyl.

In another embodiment, the compound for use in the compositions and methods provided herein has Formula VIIn, VIIo or VIIp:

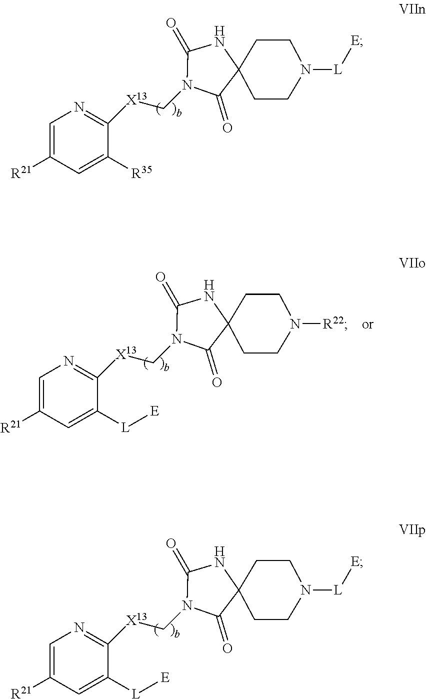

or a pharmaceutically acceptable derivative thereof, wherein:
E is an E3 ligase ligand or a moiety that activates the N-degron pathway;
L is a bond or a divalent chemical linker;
b is 2 or 3;
X¹³ is a bond, CONR³⁸, SO₂NR³⁸, CO or SO₂;
R²¹ is heterocycloalkyl, optionally substituted with 1-3 substituents each independently selected from alkyl and aryl;
R²² is H, COOR³⁹ or heteroaryl;
R³⁵ is alkyl, halo or haloalkyl;
R³⁸ is H; and
R³⁹ is alkyl.

In another embodiment, the compound for use in the compositions and methods provided herein has Formula VIIq, VIIr or VIIs:

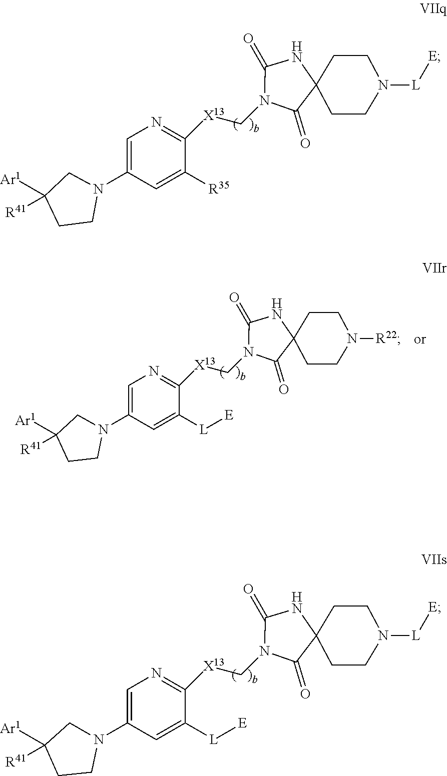

or a pharmaceutically acceptable derivative thereof, wherein:
E is an E3 ligase ligand or a moiety that activates the N-degron pathway;
L is a bond or a divalent chemical linker;
b is 2 or 3;
X¹³ is CONR³⁸;
Ar¹ is aryl;
R²² is H, COOR³⁹ or heteroaryl;
R³⁵ is alkyl, halo or haloalkyl;
R³⁸ is H; and
R³⁹ and R⁴¹ are alkyl.

In another embodiment, R⁴¹ is lower alkyl. In another embodiment, R⁴¹ is methyl or ethyl. In another embodiment, R⁴¹ is methyl. In another embodiment, R⁴¹ is ethyl.

In another embodiment, the compound for use in the compositions and methods provided herein has the formula:

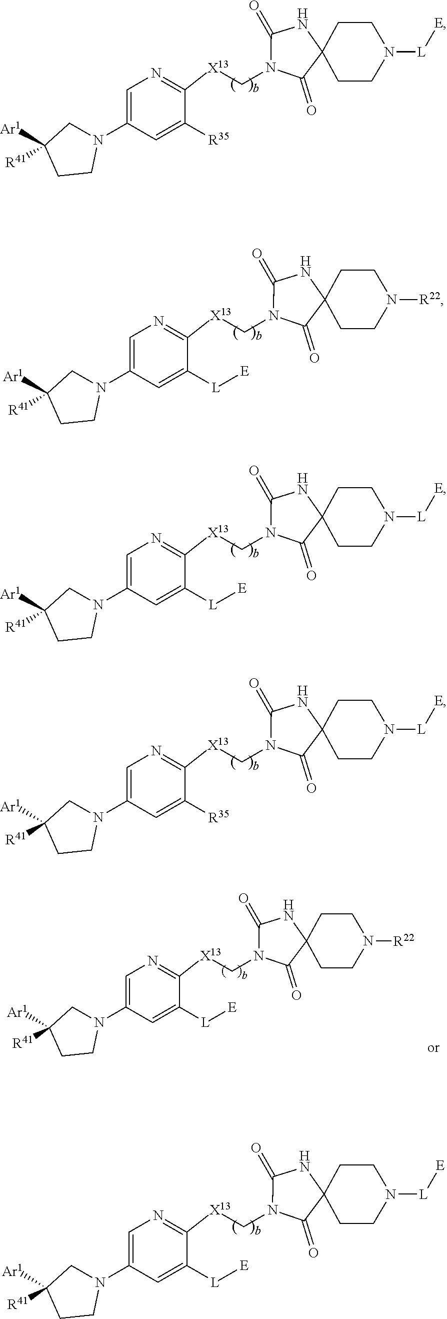

where the variables are as defined herein.

In another embodiment, the compound for use in the compositions and methods provided herein has Formula VIIt, VIIu or VIIv:

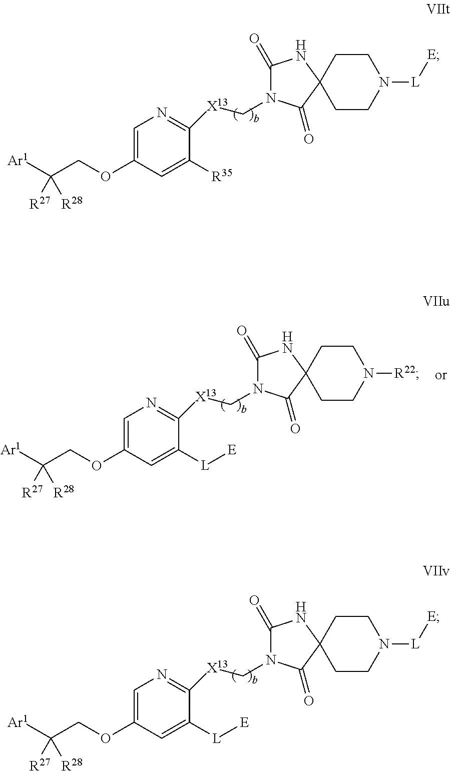

or a pharmaceutically acceptable derivative thereof, wherein:
E is an E3 ligase ligand or a moiety that activates the N-degron pathway;
L is a bond or a divalent chemical linker;
b is 2 or 3;
X¹³ is CONR³⁸;
Ar¹ is aryl;
R²² is H, COOR³⁹ or heteroaryl;
R²⁷ and R²⁸ are each independently H or alkyl, or together with the carbon atom to which they are attached form cycloalkyl;
R³⁵ is alkyl, halo or haloalkyl;
R³⁸ is H; and
R³⁹ is alkyl.

In another embodiment, R²⁷ is alkyl and R²⁸ is H. In another embodiment, R²⁷ is methyl and R²⁸ is H. In another embodiment, R²⁷ and R²⁸ together with the carbon atom to which they are attached form cyclopropyl.

In another embodiment, the compound for use in the compositions and methods provided herein has the formula:

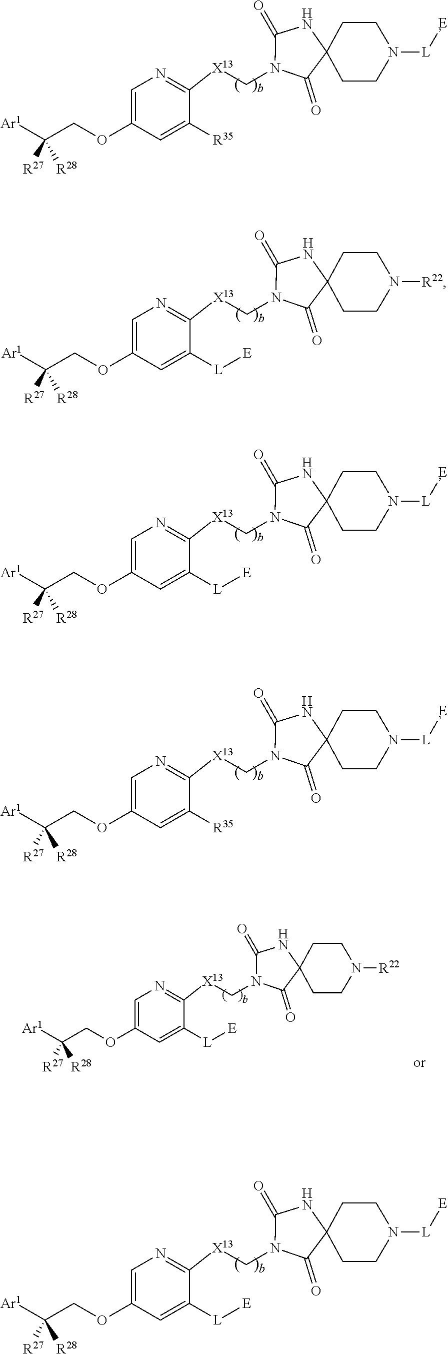

where the variables are as defined herein.

In another embodiment, b is 2. In another embodiment, b is 3.

In another embodiment, R³⁵ is lower alkyl. In another embodiment, R″ is methyl or ethyl. In another embodiment, R³⁵ is methyl. In another embodiment, R″ is halo. In another embodiment, R³⁵ is chloro. In another embodiment, R″ is haloalkyl. In another embodiment, R″ is trifluoromethyl.

In another embodiment, R³⁹ is lower alkyl. In another embodiment, R³⁹ is tert-butyl. In another embodiment R³⁹ is methyl.

In another embodiment, R³⁵ is chloro and R⁴¹ is methyl. In another embodiment, R³⁵ is chloro and R⁴¹ is ethyl. In another embodiment, b is 2, R³⁵ is chloro and R⁴¹ is methyl. In another embodiment, b is 2, R³⁵ is chloro and R⁴¹ is ethyl.

In another embodiment, Ar¹ is unsubstituted aryl. In another embodiment, Ar¹ is optionally substituted phenyl. In another embodiment, Ar¹ is unsubstituted phenyl.

In another embodiment, R²² is H. In another embodiment, R²² is COO-t-Bu. In another embodiment, R²² is COMe. In another embodiment, R²² is bicyclic or monocyclic heteroaryl. In another embodiment, R²² is pyridyl, pyrimidinyl, pyrizinyl, pyridazinyl, triazolopyrazinyl or imidazopyrazinyl. In another embodiment, R²² is triazolopyrazinyl or imidazopyrazinyl. In another embodiment, R²² is 2-pyrimidinyl, 2-ethoxycarbonyl-5-pyridyl, 2-(N-methyl)carbamoyl-4-pyridyl, 5-pyrimidinyl, 2-methoxycarbonyl-3-fluoro-5-pyridyl, 6-methoxycarbonyl-3-pyridazinyl, 2-carboxy-3-fluoro-5-pyridyl, 6-carboxy-3-pyridazinyl, 2-carboxy-5-pyridyl, 2-pyrazinyl, [1,2,4]-triazolo[4,3-a]pyrazin-8-yl, imidazo[1,5-a]pyrazin-8-yl or imidazo[1,2-a]pyrazin-8-yl. In another embodiment, R²² is [1,2,4]-triazolo[4,3-a]pyrazin-8-yl, imidazo[1,5-a]pyrazin-8-yl or imidazo[1,2-a]pyrazin-8-yl.

In one embodiment, the compound provided herein for use in the compositions and methods provided herein is:

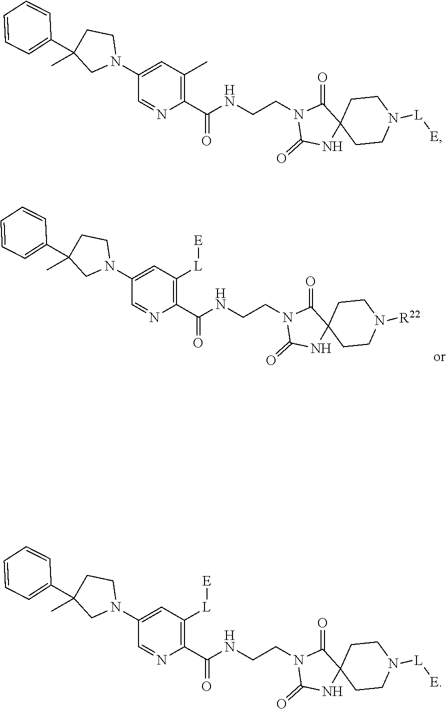

In another embodiment, provided herein is a compound for use in the compositions and methods provided herein having Formula VIIIa, VIIIb or VIIIc:

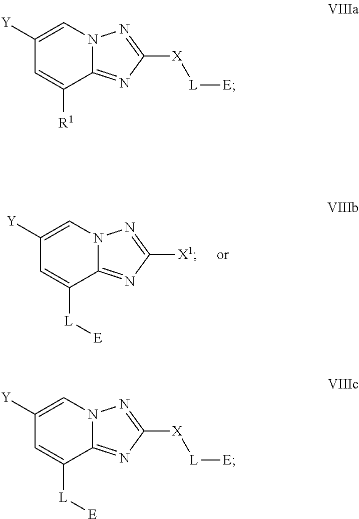

or a pharmaceutically acceptable derivative thereof, where:
E is an E3 ligase ligand or a moiety that activates the N-degron pathway;
L is a bond or a divalent chemical linker;
R¹ is optionally substituted alkyl;
X is bridged bicycloheterocyclylene; and
X¹ is bridged bicycloheterocyclyl;
Y is heterocyclyl or —OR², where R² is aralkyl.

In one embodiment, R¹ is alkyl optionally substituted with OH. In another embodiment, R¹ is —CH₂OH. In another embodiment, R¹ is unsubstituted alkyl. In another embodiment, R¹ is methyl.

In another embodiment, R² is —CH₂CR³R⁴Ph, where R³ and R⁴ are each independently H or methyl, or R³ and R⁴ together with the carbon atom to which they are attached form cyclopropyl. In another embodiment, R² is —CH₂CH(Me)Ph. In another embodiment, R² is

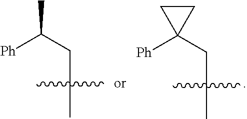

In another embodiment, X is an oxabicyclohexane, an oxabicycloheptane, an oxabicyclooctane or an oxazabicyclooctane. In another embodiment, X is

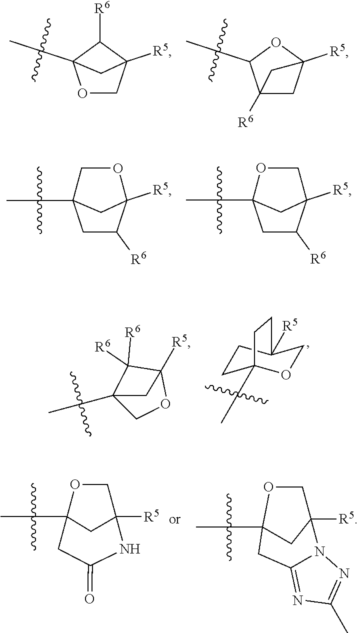

where R⁵ is optionally substituted alkyl, heterocyclyl, cycloalkyl, optionally substituted alkylene, heterocyclylene, cycloalkylene, CN, OR⁷, NR⁸R⁹, C(O)NR¹⁰R¹¹, COOR¹² or —NR¹³C(O)R¹⁴; and each R⁶ is independently optionally substituted alkyl, heterocyclyl, cycloalkyl, heteroaryl, optionally substituted alkylene, heterocyclylene, cycloalkylene, heteroarylene, NR′R⁹ or C(O)NR¹⁰R″;
where R⁷ is H, alkyl or alkylene; R⁸ and R⁹ are each independently H, alkyl, alkylene, —CH₂-heteroaryl, —CH₂-heteroarylene, —CH₂-heterocyclyl or —CH₂-heterocyclylene; R¹⁰ and R″ are each independently H, alkyl or alkylene; R¹² is H, alkyl or alkylene; R¹³ is H, alkyl or alkylene; and R¹⁴ is optionally substituted alkyl or optionally substituted alkylene;
with the proviso that R⁵-R¹⁴ contain at least one divalent group (i.e., alkylene, heterocyclylene cycloalkylene or heteroarylene) to which -L-E is attached.

In another embodiment, R⁵ is H; alkyl optionally substituted with OH, NH₂ or heterocyclyl; alkylene optionally substituted with OH, NH₂ or heterocyclyl; heterocyclyl; heterocyclylene; CN; OR⁷, NR′R⁹; C(O)NR¹⁰R″; COOR¹² or —NR¹³C(O)R¹⁴. In another embodiment, R⁵ is H; alkyl optionally substituted with OH, NH₂ or piperazonyl; alkylene optionally substituted with OH, NH₂ or piperazonyl; pyrrolidinyl; pyrrolidinylene; CN; OH; —O—; NH₂; —NH—; NMe₂; —NMe—; NHCH₂-triazolyl; NHCH₂-triazolylene; C(O)NH₂; —C(O)NH—; C(O)NHMe; —C(O)NMe—; C(O)NHEt; —C(O)NEt-; COOH; —COO—; —NHC(O)CH₂-triazolyl; —NHC(O)CH₂-triazolylene; —NHC(O)CH₂C(Me)₂OH or —NHC(O)Me. In another embodiment, R⁵ is H; CH₂OH, CH₂NH₂, CH₂piperazonyl; pyrrolidinyl; CN; OH; NH₂; NMe₂; NHCH₂-triazolyl; C(O)NH₂; C(O)NHMe; C(O)NHEt; COOH; —NHC(O)CH₂-triazolyl; —NHC(O)CH₂C(Me)₂OH or —NHC(O)Me.

In another embodiment, each R⁶ is independently H; alkyl optionally substituted with OH, NR′R⁹, heteroaryl, C(O)NR¹⁰R¹¹ or —NHC(O)R¹⁴; alkylene optionally substituted with OH, NR⁸R⁹, heteroaryl, C(O)NR¹⁰R¹¹ or —NHC(O)R¹⁴; heterocyclyl; heteroaryl; heterocyclylene; heteroarylene; NR′R⁹ or C(O)NR¹⁰R¹¹. In another embodiment, each R⁶ is independently H; alkyl optionally substituted with OH, NH₂, heteroaryl, C(O)NH₂ or —NHC(O)Et; alkylene optionally substituted with OH, NH₂, heteroaryl, C(O)NH₂ or —NHC(O)Et; heterocyclyl; heteroaryl; heterocyclylene; heteroarylene; NR⁸R⁹ or C(O)NR¹⁰R¹¹. In another embodiment, each R⁶ is independently H; CH₂OH; C(Me)₂OH; CH₂NH₂; CH₂C(O)NH₂; CH₂NHC(O)Et; triazolyl; oxadiazolyl; imidazolidinyl; imidazolyl; tetrazolyl; oxadiazalonyl; dimethylhydantoinyl; C(O)NH₂; C(O)NHMe; C(O)NHCH₂C(Me)₂OH; —NHC(O)Et or NH₂.

In another embodiment, R⁷ is H, alkyl or alkylene. In another embodiment, R⁷ is H, methyl or methylene. In another embodiment, R⁷ is H.

In another embodiment, R⁸ and R⁹ are each independently H, alkyl, alkylene, —CH₂—triazolyl or —CH₂-heterocyclyl. In another embodiment, R⁸ and R⁹ are each independently H, methyl, methylene or —CH₂-triazolyl.

In another embodiment, R¹⁰ and R″ are each independently H, methyl, methylene or ethylene.

In another embodiment, R¹² is H, methyl or methylene. In another embodiment, R¹² is H.

In another embodiment, R¹³ is H, methyl or methylene. In another embodiment, R¹³ is H.

In another embodiment, R¹⁴ is alkyl optionally substituted with OH or heteroaryl; or alkylene optionally substituted with OH or heteroaryl. In another embodiment, R¹⁴ is alkyl optionally substituted with OH or triazolyl; or alkylene optionally substituted with OH or triazolyl. In another embodiment, R¹⁴ is methyl, methylene, —CH₂C(Me)₂OH or CH₂-triazolyl.

In another embodiment, X is an oxabicyclohexane, an oxabicycloheptane, an oxabicyclooctane or an oxazabicyclooctane. In another embodiment, X¹ is

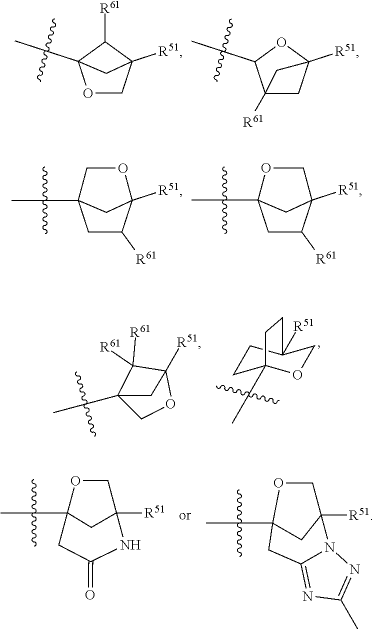

where R⁵¹ is optionally substituted alkyl, heterocyclyl, cycloalkyl, CN, OR⁷¹, NR¹¹R⁹¹, C(O)NR¹⁰¹R¹¹¹, COOR¹²¹ or —NR¹³¹C(O)R¹⁴¹; and each R⁶¹ is independently optionally substituted alkyl, heterocyclyl, cycloalkyl, heteroaryl, NR⁸¹R⁹¹ or C(O)NR¹⁰¹R¹¹¹;
where R⁷¹ is H or alkyl or alkylene; R⁸⁸ and R⁹¹ are each independently H, alkyl, —CH₂—heteroaryl or —CH₂-heterocyclyl; R¹⁰ and R¹¹¹ are each independently H or alkyl; R¹²¹ is H or alkyl; R¹³¹ is H or alkyl; and R¹⁴¹ is optionally substituted alkyl.

In another embodiment, R″ is H; alkyl optionally substituted with OH, N₁₂ or heterocyclyl; heterocyclyl; CN; OR⁷¹, NR⁸¹R⁹¹; C(O)NR¹⁰¹R¹¹¹; COOR¹²¹ or —NR¹³¹C(O)R¹⁴¹. In another embodiment, R″ is H; alkyl optionally substituted with OH, NH₂ or piperazonyl; pyrrolidinyl; CN; OH; NH₂; NMe₂; NHCH₂-triazolyl; C(O)NH₂; C(O)NHMe; C(O)NHEt; COOH; —NHC(O)CH₂-triazolyl; —NHC(O)CH₂C(Me)₂OH or —NHC(O)Me.

In another embodiment, each R⁶¹ is independently H; alkyl optionally substituted with OH, NR⁸¹R⁹¹, heteroaryl, C(O)NR¹⁰¹R¹¹¹ or —NHC(O)R¹⁴¹; heterocyclyl; heteroaryl; NR⁸¹R⁹¹ or C(O)NR¹⁰¹R¹¹. In another embodiment, each R⁶ is independently H; CH₂OH; C(Me)₂OH; CH₂NH₂; CH₂C(O)NH₂; CH₂NHC(O)Et; triazolyl; oxadiazolyl; imidazolidinyl; imidazolyl; tetrazolyl; oxadiazalonyl; dimethylhydantoinyl; C(O)NH₂; C(O)NHMe; C(O)NHCH₂C(Me)₂OH; —NHC(O)Et or NH₂.

In another embodiment, R⁷¹ is H or alkyl. In another embodiment, R⁷¹ is H or methyl. In another embodiment, R⁷¹ is H.

In another embodiment, R⁸⁸ and R⁹¹ are each independently H, alkyl, —CH₂-triazolyl or —CH₂-heterocyclyl. In another embodiment, R⁸⁸ and R⁹¹ are each independently H, methyl or —CH₂-triazolyl.

In another embodiment, R¹⁰¹ and R¹¹¹ are each independently H or methyl.

In another embodiment, R¹²¹ is H or methyl. In another embodiment, R¹²¹ is H.

In another embodiment, R¹³¹ is H or methyl. In another embodiment, R¹³¹ is H.

In another embodiment, R¹⁴¹ is alkyl optionally substituted with OH or heteroaryl. In another embodiment, R¹⁴¹ is alkyl optionally substituted with OH or triazolyl. In another embodiment, R¹⁴¹ is methyl, —CH₂C(Me)₂OH or CH₂-triazolyl.

In another embodiment, Y is heterocyclyl. In another embodiment, Y is pyrrolidinyl. In another embodiment, Y is

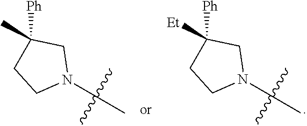 [SMILES: *N1CC[C@](C)(c2ccccc2)C1 ; *N1CC[C@](CC)(c2ccccc2)C1]

In another embodiment, Y is

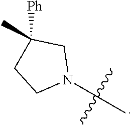 [SMILES: *N1CC[C@](C)(c2ccccc2)C1]

In another embodiment, Y is —OR². In another embodiment, Y is —O—CH₂CR³R⁴Ph, where R³ and R⁴ are each independently H or methyl, or R³ and R⁴ together with the carbon atom to which they are attached form cyclopropyl. In another embodiment, Y is —O—CH₂CH(Me)Ph. In another embodiment, Y is

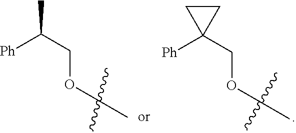 [SMILES: *OC[C@H](C)c1ccccc1 ; *OCC1(c2ccccc2)CC1]

In another embodiment, the compound for use in the compositions and methods provided herein has Formula VIIId or VIIIe:

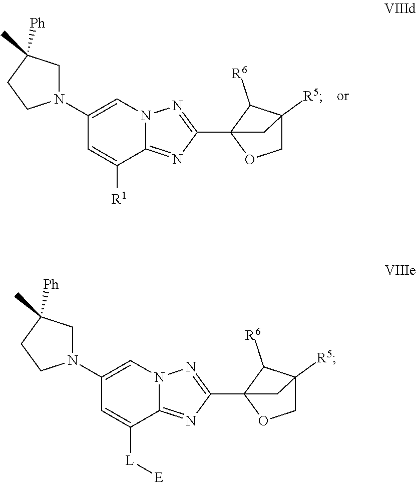

where L, E, R¹, R⁵ and R⁶ are as defined elsewhere herein.

In another embodiment, the compounds for use in the compositions and methods provided herein have Formula IXa, IXb or IXc:

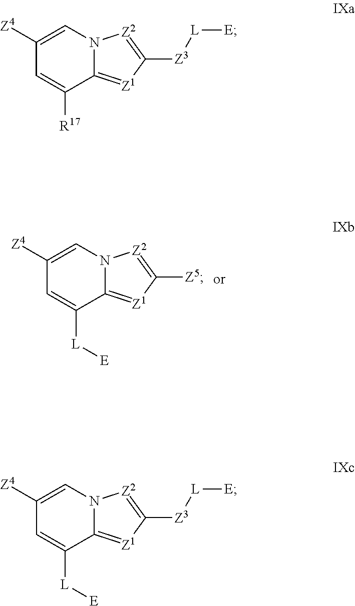

or a pharmaceutically acceptable derivative thereof, where:
E is an E3 ligase ligand or a moiety that activates the N-degron pathway;
L is a bond or a divalent chemical linker;
R¹⁷ is OR¹⁸, NR¹⁹R²⁰, optionally substituted alkyl or CN;
Z¹ is N, CH, C—CH₂OH or C—CH₂OMe;
Z² is N or CH;
Z³ is C(O)NR²¹R²², SO₂NR²¹R²², —C(Me)₂OR²¹ or CO₂R²¹;
Z⁴ is heterocyclyl or OR²³; and
Z⁵ is C(O)NR⁴¹R⁴², SO₂NR⁴¹R⁴², —C(Me)₂OR⁴¹ or CO₂R⁴¹;
where R¹⁸ is H or alkyl;
R¹⁹ and R²⁰ are each independently H or alkyl;
R²¹ and R²² are each independently H, optionally substituted alkyl, optionally substituted cycloalkyl, bicycloalkyl or spirocycloalkyl, optionally substituted heterocyclyl, bicycloheterocyclyl or spirocycloheterocyclyl, aryl or heteroaryl, or together with the nitrogen atom to which they are attached form optionally substituted heterocyclyl, bicycloheterocyclyl or spirocycloheterocyclyl, optionally substituted alkylene, optionally substituted cycloalkylene, bicycloalkylene or spirocycloalkylene, optionally substituted heterocyclylene, bicycloheterocyclylene or spirocycloheterocyclylene, arylene or heteroarylene, or together with the nitrogen atom to which they are attached form optionally substituted heterocyclylene, bicycloheterocyclylene or spirocycloheterocyclylene;
R²³ is aralkyl;
R⁴¹ and R⁴² are each independently H, optionally substituted alkyl, optionally substituted cycloalkyl, bicycloalkyl or spirocycloalkyl, optionally substituted heterocyclyl, bicycloheterocyclyl or spirocycloheterocyclyl, aryl or heteroaryl, or together with the nitrogen atom to which they are attached form optionally substituted heterocyclyl, bicycloheterocyclyl or spirocycloheterocyclyl;
with the proviso that the compound of Formula IXa or IXc contains at least one divalent R²¹ or R²² group.

In one embodiment, R¹⁷ is OR¹⁸, NR¹⁹R²⁰, CN or alkyl optionally substituted with OH, NH₂, NHC(O)Me or NHSO₂Et. In another embodiment, R¹⁷ is OH, OMe, NH₂, CN, methyl, ethyl, isopropyl, CH₂OH, CH₂NH₂, CH₂NHC(O)Me or CH₂NHSO₂Et. In another embodiment, R¹⁷ is methyl.

In another embodiment, R¹⁸ is H or methyl. In another embodiment, R¹⁹ and R²⁰ are H or methyl. In another embodiment, R¹⁹ and R²⁰ are H.

In another embodiment, R²¹ and R²² are each independently H; alkyl optionally substituted with OR²⁴, heterocyclyl, bicycloheterocyclyl, spirocycloheterocyclyl, cycloalkyl or CO₂R²⁵; cycloalkyl, bicycloalkyl or spirocycloalkyl each optionally substituted with halo, haloalkyl, OR²⁴, heterocyclyl, aryl or heteroaryl; heterocyclyl, bicycloheterocyclyl or spirocycloheterocyclyl each optionally substituted with halo, haloalkyl, OR²⁴, NR²⁴R²⁵, COR²⁵, heterocyclyl or heteroaryl; aryl; heteroaryl; alkylene optionally substituted with OR²⁴, heterocyclyl, bicycloheterocyclyl, spirocycloheterocyclyl, cycloalkyl or CO₂R²⁵; cycloalkylene, bicycloalkylene or spirocycloalkylene each optionally substituted with halo, haloalkyl, OR²⁴ heterocyclyl, aryl or heteroaryl; heterocyclylene, bicycloheterocyclylene or spirocycloheterocyclylene each optionally substituted with halo, haloalkyl, OR²⁴, NR²⁴R²⁵ COR²⁵, heterocyclyl or heteroaryl; arylene; heteroarylene; or together with the nitrogen atom to which they are attached form heterocyclylene, bicycloheterocyclylene or spirocycloheterocyclylene each optionally substituted with OR²⁴, CO₂R²⁵, heterocyclyl or heteroaryl; where R²⁴ and R²⁵ are each independently H or alkyl.

In another embodiment, R²¹ and R²² are each independently H; alkyl optionally substituted with OH, methoxy, heterocyclyl, bicycloheterocyclyl, spirocycloheterocyclyl, cycloalkyl or CO₂H; cyclopropyl, cyclobutyl, cyclopentyl, cyclohexyl, bicyclocyclohexyl, bicyclocycloheptyl, spirocycloheptyl or spirocyclooctyl each optionally substituted with fluoro, haloalkyl, OH, methoxy, heterocyclyl, phenyl or heteroaryl; piperidinyl, pyrrolidinyl, piperazinyl, pyranyl, tetrahydrofuranyl, bicycloheterocyclyl, dioxaspirononyl, oxaspiroheptyl or azaspiroheptyl each optionally substituted with fluoro, difluoroethyl, trifluoromethyl, OH, methoxy, NH₂, C(O)Et, heterocyclyl or heteroaryl; or pyridyl; alkylene optionally substituted with OH, methoxy, heterocyclyl, bicycloheterocyclyl, spirocycloheterocyclyl, cycloalkyl or CO₂H; cyclopropylene, cyclobutylene, cyclopentylene, cyclohexylene, bicyclocyclohexylene, bicyclocycloheptylene, spirocycloheptylene or spirocyclooctylene each optionally substituted with fluoro, haloalkyl, OH, methoxy, heterocyclyl, phenyl or heteroaryl; piperidinylene, pyrrolidinylene, piperazinylene, pyranylene, tetrahydrofuranylene, bicycloheterocyclylene, dioxaspirononylene, oxaspiroheptylene or azaspiroheptylene each optionally substituted with fluoro, difluoroethyl, trifluoromethyl, OH, methoxy, NH₂, C(O)Et, heterocyclyl or heteroaryl; or pyridylene; or together with the nitrogen atom to which they are attached form morpholinylene, pyrrolidinylene, piperidinylene, piperazinylene, azetidinylene, bicycloazahexylene, bicyclodiazaoctylene or spirocyclodiazanonylene each optionally substituted with OH, methoxy, CO₂H, heterocyclyl or heteroaryl.

In another embodiment, R²¹ is H, alkyl or alkylene. In another embodiment, R²¹ is H. In another embodiment, R²¹ is methyl or methylene.

In another embodiment, R²² is alkyl optionally substituted with OR²⁴, heterocyclyl, spirocycloheterocyclyl, cycloalkyl or CO₂R²⁵; cycloalkyl, bicycloalkyl or spirocycloalkyl each optionally substituted with halo, haloalkyl, OR²⁴, heterocyclyl, aryl or heteroaryl; heterocyclyl, bicycloheterocyclyl or spirocycloheterocyclyl each optionally substituted with halo, haloalkyl, OR²⁴, NR²⁴R²⁵, heterocyclyl or heteroaryl; aryl; heteroaryl; alkylene optionally substituted with OR²⁴, heterocyclyl, spirocycloheterocyclyl, cycloalkyl or CO₂R²⁵; cycloalkylene, bicycloalkylene or spirocycloalkylene each optionally substituted with halo, haloalkyl, OR²⁴ heterocyclyl, aryl or heteroaryl; heterocyclylene, bicycloheterocyclylene or spirocycloheterocyclylene each optionally substituted with halo, haloalkyl, OR²⁴, NR²⁴R²⁵ heterocyclyl or heteroaryl; arylene; or heteroarylene.

In another embodiment, R²² is alkyl optionally substituted with OH, methoxy, heterocyclyl, bicycloheterocyclyl, spirocycloheterocyclyl, cycloalkyl or CO₂H; cyclopropyl, cyclobutyl, cyclopentyl, cyclohexyl, bicyclocyclohexyl, bicyclocycloheptyl, spirocycloheptyl or spirocyclooctyl each optionally substituted with fluoro, haloalkyl, OH, methoxy, heterocyclyl, phenyl or heteroaryl; piperidinyl, pyrrolidinyl, piperazinyl, pyranyl, tetrahydrofuranyl, bicycloheterocyclyl, dioxaspirononyl, oxaspiroheptyl or azaspiroheptyl each optionally substituted with fluoro, difluoroethyl, trifluoromethyl, OH, methoxy, NH₂, COR²⁵, heterocyclyl or heteroaryl; pyridyl; alkylene optionally substituted with OH, methoxy, heterocyclyl, bicycloheterocyclyl, spirocycloheterocyclyl, cycloalkyl or CO₂H; cyclopropylene, cyclobutylene, cyclopentylene, cyclohexylene, bicyclocyclohexylene, bicyclocycloheptylene, spirocycloheptylene or spirocyclooctylene each optionally substituted with fluoro, haloalkyl, OH, methoxy, heterocyclyl, phenyl or heteroaryl; piperidinylene, pyrrolidinylene, piperazinylene, pyranylene, tetrahydrofuranylene, bicycloheterocyclylene, dioxaspirononylene, oxaspiroheptylene or azaspiroheptylene each optionally substituted with fluoro, difluoroethyl, trifluoromethyl, OH, methoxy, NH₂, COR²⁵, heterocyclyl or heteroaryl; or pyridylene.

In another embodiment, R²¹ and R²² together with the nitrogen atom to which they are attached form heterocyclylene, bicycloheterocyclylene or spirocycloheterocyclylene each optionally substituted with OR²⁴, CO₂R²⁵, heterocyclyl or heteroaryl.

In another embodiment, R²¹ and R²² together with the nitrogen atom to which they are attached form morpholinylene, pyrrolidinylene, piperidinylene, piperazinylene, azetidinylene, bicycloazahexylene, bicyclodiazaoctylene or spirocyclodiazanonylene, each optionally substituted with OH, methoxy, CO₂H, heterocyclyl or heteroaryl.

In another embodiment, R²¹ and R²² together with the nitrogen atom to which they are attached form

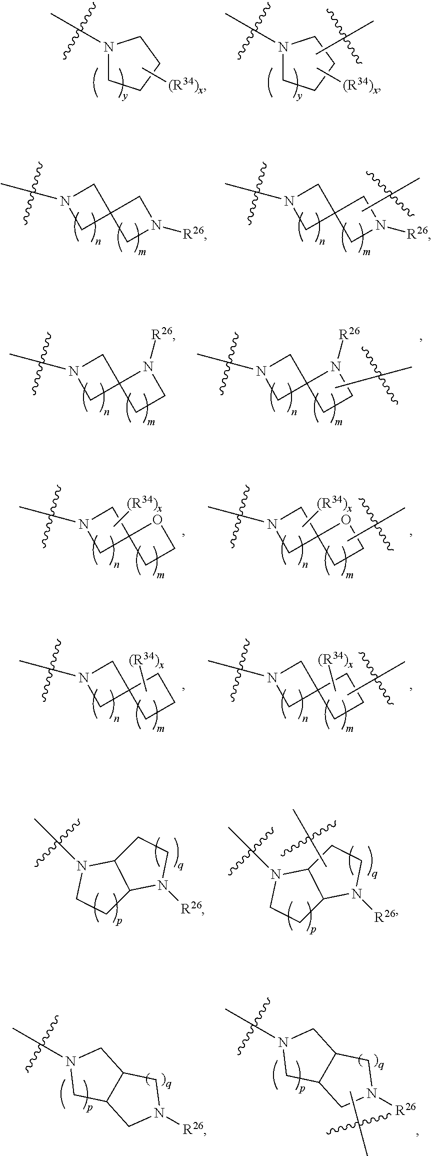

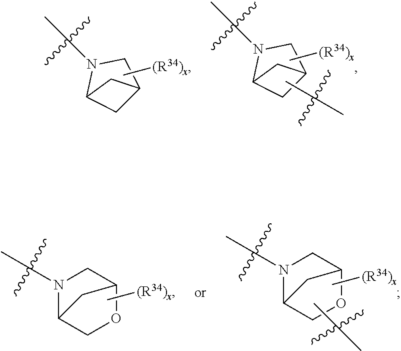

where x is an integer from 0-4; y is 0, 1, or 2; n is 1, 2 or 3; m is 1, 2 or 3; p is 1, 2 or 3;
q is 1, 2 or 3; R²⁶ is heteroaryl optionally substituted with CO₂R²⁷ or heteroarylene optionally substituted with CO₂R²⁷ where R²⁷ is H, alkyl or alkylene; and R³⁴ is OR²⁴, CO₂R²⁵, heterocyclyl, heterocyclylene, heteroaryl or heteroarylene.

In another embodiment, R⁴¹ and R⁴² are each independently H; alkyl optionally substituted with OR⁴⁴, heterocyclyl, bicycloheterocyclyl, spirocycloheterocyclyl, cycloalkyl or CO₂R⁴⁵; cycloalkyl, bicycloalkyl or spirocycloalkyl each optionally substituted with halo, haloalkyl, OR⁴⁴, heterocyclyl, aryl or heteroaryl; heterocyclyl, bicycloheterocyclyl or spirocycloheterocyclyl each optionally substituted with halo, haloalkyl, OR⁴⁴, NR⁴⁴R⁴⁵, COR⁴⁵, heterocyclyl or heteroaryl; aryl; heteroaryl; where R⁴⁴ and R⁴⁵ are each independently H or alkyl.

In another embodiment, R⁴¹ and R⁴² are each independently H; alkyl optionally substituted with OH, methoxy, heterocyclyl, bicycloheterocyclyl, spirocycloheterocyclyl, cycloalkyl or CO₂H; cyclopropyl, cyclobutyl, cyclopentyl, cyclohexyl, bicyclocyclohexyl, bicyclocycloheptyl, spirocycloheptyl or spirocyclooctyl each optionally substituted with fluoro, haloalkyl, OH, methoxy, heterocyclyl, phenyl or heteroaryl; piperidinyl, pyrrolidinyl, piperazinyl, pyranyl, tetrahydrofuranyl, bicycloheterocyclyl, dioxaspirononyl, oxaspiroheptyl or azaspiroheptyl each optionally substituted with fluoro, difluoroethyl, trifluoromethyl, OH, methoxy, NH₂, C(O)Et, heterocyclyl or heteroaryl; or pyridyl.

In another embodiment, R⁴¹ is H or alkyl. In another embodiment, R⁴¹ is H. In another embodiment, R⁴¹ is methyl.

In another embodiment, R⁴² is alkyl optionally substituted with OR⁴⁴, heterocyclyl, spirocycloheterocyclyl, cycloalkyl or CO₂R⁴⁵; cycloalkyl, bicycloalkyl or spirocycloalkyl each optionally substituted with halo, haloalkyl, OR⁴⁴, heterocyclyl, aryl or heteroaryl; heterocyclyl, bicycloheterocyclyl or spirocycloheterocyclyl each optionally substituted with halo, haloalkyl, OR⁴⁴, NR⁴⁴R⁴⁵, heterocyclyl or heteroaryl; aryl; heteroaryl.

In another embodiment, R⁴² is alkyl optionally substituted with OH, methoxy, heterocyclyl, bicycloheterocyclyl, spirocycloheterocyclyl, cycloalkyl or CO₂H; cyclopropyl, cyclobutyl, cyclopentyl, cyclohexyl, bicyclocyclohexyl, bicyclocycloheptyl, spirocycloheptyl or spirocyclooctyl each optionally substituted with fluoro, haloalkyl, OH, methoxy, heterocyclyl, phenyl or heteroaryl; piperidinyl, pyrrolidinyl, piperazinyl, pyranyl, tetrahydrofuranyl, bicycloheterocyclyl, dioxaspirononyl, oxaspiroheptyl or azaspiroheptyl each optionally substituted with fluoro, difluoroethyl, trifluoromethyl, OH, methoxy, NH₂, COR⁴⁵, heterocyclyl or heteroaryl; pyridyl.

In another embodiment, R⁴¹ and R⁴² together with the nitrogen atom to which they are attached form

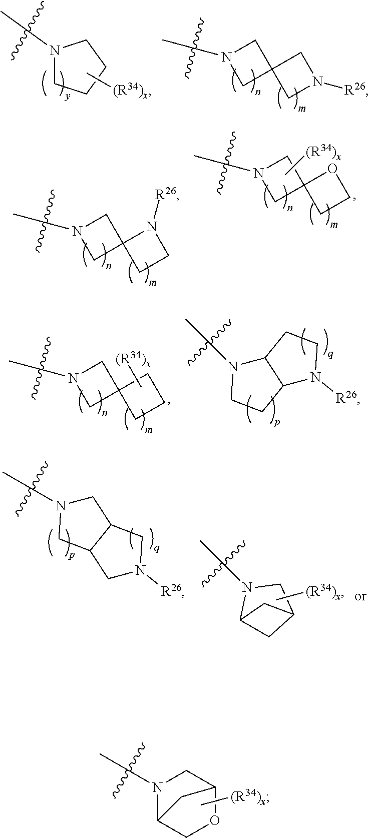

where x is an integer from 0-4; y is 0, 1, or 2; n is 1, 2 or 3; m is 1, 2 or 3; p is 1, 2 or 3; q is 1, 2 or 3; R²⁶ is heteroaryl optionally substituted with CO₂R²⁷ where R²⁷ is H or alkyl; and R³⁴ is OR²⁴, CO₂R²⁵, heterocyclyl or heteroaryl.

In another embodiment, x is 0. In another embodiment, x is 1. In another embodiment, x is 2. In another embodiment, x is 3. In another embodiment, y is 0. In another embodiment, y is 1. In another embodiment, y is 2. In another embodiment, n is 1. In another embodiment, n is 2. In another embodiment, n is 3. In another embodiment, m is 1. In another embodiment, m is 2. In another embodiment, m is 3. In another embodiment, p is 1. In another embodiment, p is 2. In another embodiment, p is 3. In another embodiment, q is 1. In another embodiment, q is 2. In another embodiment, q is 3.

In another embodiment, R²⁶ is pyridyl optionally substituted with CO₂R²⁷, or pyridylene optionally substituted with CO₂R²⁷, where R²⁷ is H or alkyl. In another embodiment, R²⁷ is H. In another embodiment, R³⁴ is OH, methoxy, CO₂H, heterocyclyl or heteroaryl.

In another embodiment, Z⁴ is OR²³. In another embodiment, R²³ is —CH₂CR²⁸R²⁹Ph, where R²⁸ and R²⁹ are each independently H or methyl, or R²⁸ and R²⁹ together with the carbon atom to which they are attached form cyclopropyl. In another embodiment, R²³ is —CH₂CH(Me)Ph. In another embodiment, R²³ is

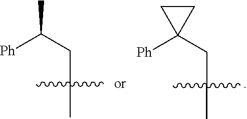

In another embodiment, Z¹ is N or CH. In another embodiment, Z¹ is N. In another embodiment, Z¹ is CH.

In another embodiment, Z² is N. In another embodiment, Z² is CH.

In another embodiment, Z¹ is N and Z² is CH. In another embodiment, Z¹ is CH and Z² is N. In another embodiment, Z¹ and Z² are both N.

In another embodiment, Z³ is C(O)NR²¹R²², SO₂NR²¹R²² or CO₂R²¹. In another embodiment, Z³ is C(O)NR²¹R²² or SO₂NR²¹R²². In another embodiment, Z³ is C(O)NR²¹R²².

In another embodiment, Z⁴ is heterocyclyl. In another embodiment, Z⁴ is pyrrolidinyl, azetidinyl, morpholinyl or azaspiroheptyl. In another embodiment, Z⁴ is pyrrolidinyl, azetidinyl, morpholinyl or azaspiro[3.3.0]heptyl. In another embodiment, Z⁴ is pyrrolidinyl, azetidinyl, morpholinyl or azaspiro[3.3.0]heptyl, each optionally substituted with CN, OH, optionally substituted alkyl, aryl or heteroaryl. In another embodiment, Z⁴ is pyrrolidinyl, azetidinyl, morpholinyl or azaspiro[3.3.0]heptyl, each optionally substituted with CN; OH; oxo; methyl optionally substituted with OH or fluoro; ethyl; phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl. In another embodiment, Z⁴ is azaspiro[3.3.0]heptyl substituted with phenyl. In another embodiment, Z⁴ is morpholinyl substituted with phenyl and methyl. In another embodiment, Z⁴ is azetidinyl substituted with (i) methyl or ethyl, and (ii) phenyl, 3-methylphenyl or 4-methylphenyl.

In another embodiment, Z⁴ is pyrrolidinyl substituted with CN; OH; oxo; methyl optionally substituted with OH or fluoro; ethyl; phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl.

In another embodiment, Z⁴ is

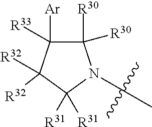

where each R³⁰ is H or the two R³⁰ form oxo;
each R³¹ is H or the two R³¹ form oxo;
each R³² is H or methyl;
R³³ is optionally substituted alkyl, or together with R³¹ forms a bridged bicyclic pyrrolidinyl group; and
Ar is an optionally substituted aryl or heteroaryl group, OH or CN.

In another embodiment, each R³⁰, R³¹ and R³² is H.

In another embodiment, R³³ is optionally substituted alkyl. In another embodiment, R³³ is optionally substituted methyl. In another embodiment, R³³ is methyl.

In another embodiment, Ar is aryl or heteroaryl optionally substituted with methyl, chloro, fluoro, methoxy or hydroxy. In another embodiment, Ar is phenyl, pyridyl, thiazolyl, oxazolyl, pyrazolyl, oxadiazolyl, tetrazolyl, thiadiazolyl or triazolyl, each optionally substituted with methyl, chloro, fluoro, methoxy or hydroxy. In another embodiment, Ar is phenyl optionally substituted with methyl, chloro, fluoro, methoxy or hydroxy; pyridyl; thiazolyl; oxazolyl; pyrazolyl; oxadiazolyl; tetrazolyl; thiadiazolyl; or triazolyl. In another embodiment, Ar is phenyl optionally substituted with methyl, chloro, fluoro, methoxy or hydroxy. In another embodiment, Ar is unsubstituted phenyl.

In another embodiment, Z⁴ is

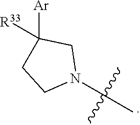

In another embodiment, Z⁴ is

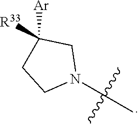

In another embodiment, Z⁴ is

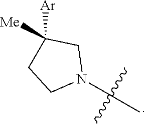

In another embodiment, Z⁴ is

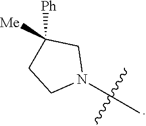 [SMILES: *N1CC[C@](C)(c2ccccc2)C1]

In another embodiment, Z⁵ is C(O)NR⁴¹R⁴², SO₂NR⁴¹R⁴² or CO₂R⁴¹. In another embodiment, Z⁵ is C(O)NR⁴¹R⁴² or SO₂NR⁴¹R⁴². In another embodiment, Z⁵ is C(O)NR⁴¹R⁴²

In another embodiment, the compound for use in the compositions and methods provided herein has one of the following formulae:

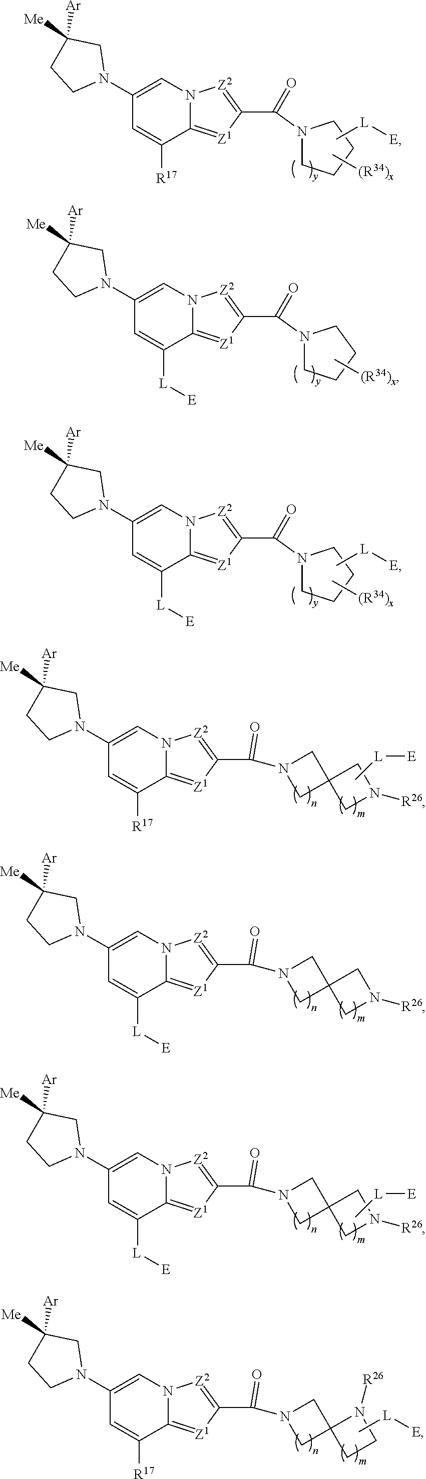

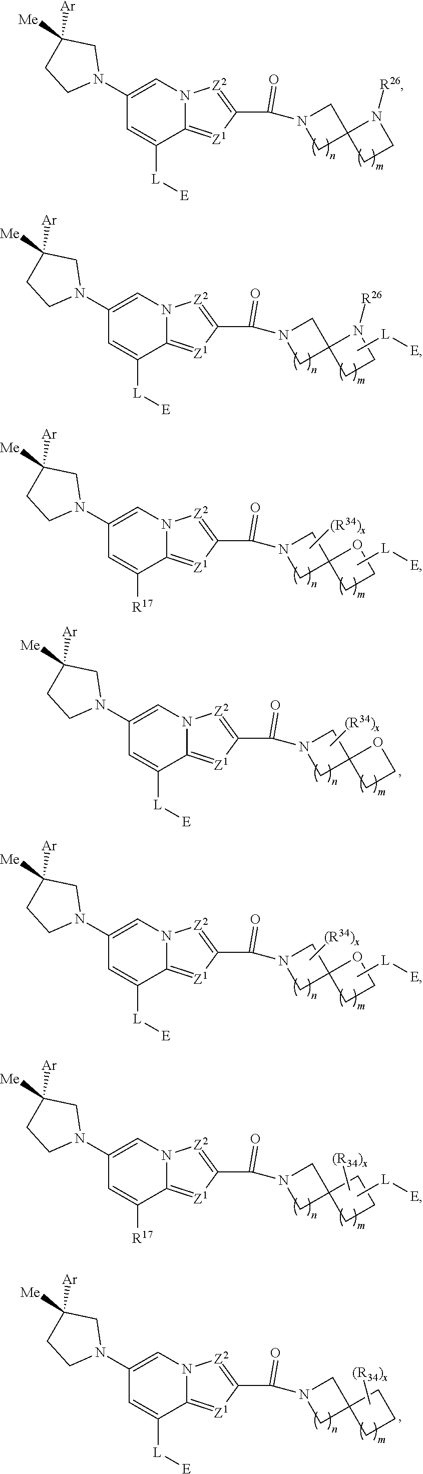

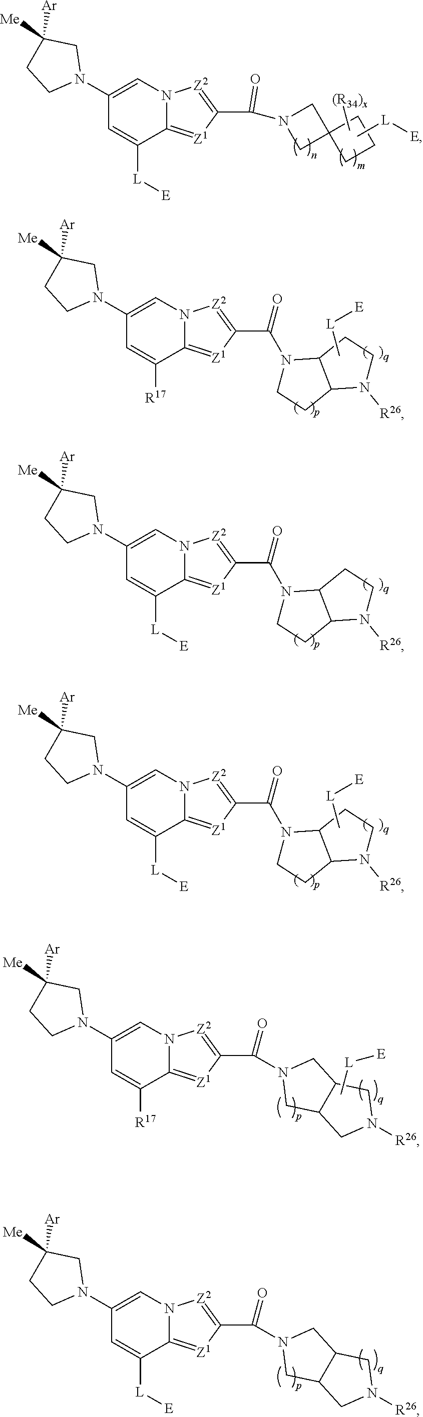

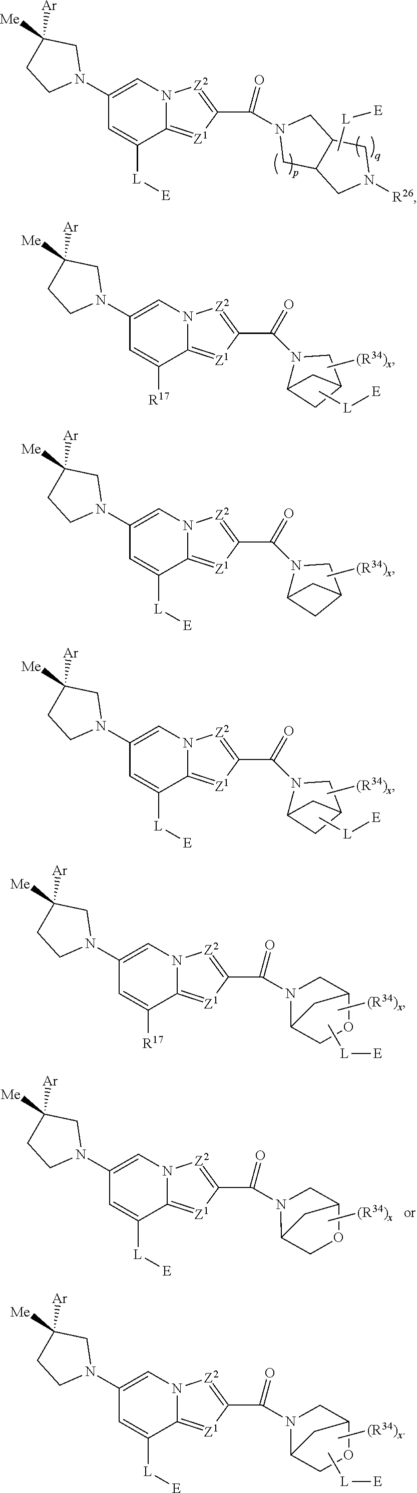

### Divalent Chemical Linkers L

Linkers for use in bifunctional chimeric compounds such as PROTACs and LYTACs are well known in the art. See, e.g., Troup, et al. Explor. Target Antitumor. Ther. 2020, 1, 273-312; WO 2019/060742; US 2021/0220475. In one embodiment, the linker L for use in the compounds provided herein contains glycol including polyethylene glycol (PEG), alkyl, acyl, amino, amido, alkyne, triazole (e.g., from use of click chemistry to connect the two targeting moieties), pyridazines (e.g., CLIPTACs), aryl (e.g., phenyl), piperazine, azetidine and/or piperidine moieties. The linker L is cleavable or non-cleavable. In one embodiment, the linker L is photoswitchable (e.g., diazo linkers).

In another embodiment, the linker L contains a piperazine, a piperidine, an azetidine and/or an alkyl moiety. In another embodiment, L is piperidinylene-alkylene-piperazinylene. In another embodiment, L is piperidinylene-methylene-piperazinylene. In another embodiment, L is

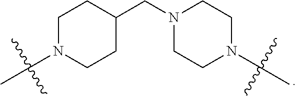 [SMILES: *N1CCC(CN2CCN(*)CC2)CC1]

In another embodiment, L is piperidinylene-alkylene-azetidinylene. In another embodiment, L is piperidinylene-methylene-azetidinylene. In another embodiment, L is

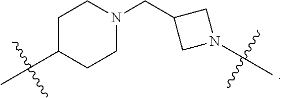 [SMILES: *C1CCN(CC2CN(*)C2)CC1]

In another embodiment, L is an alkylamido linker. In another embodiment, L is C₄₋₁₀-alkylene-C(O)—NH—. In another embodiment, L is

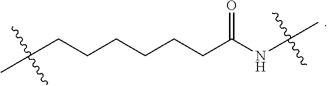 [SMILES: *CCCCCCC(=O)N*]

In another embodiment, L is

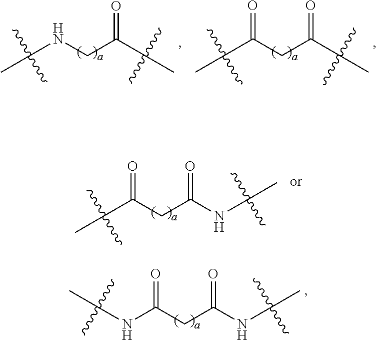

where a in an integer from 2 to 14. In one embodiment, a is an integer from 4 to 10.

In another embodiment, L is a PEG linker. In another embodiment, L is (PEG)₂₋₆-NH—. In another embodiment, L is

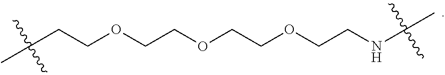 [SMILES: *CCOCCOCCOCCN*]

In one embodiment, L has formula (L¹-L²-L³-L⁴)ₓ, where each of L¹-L⁴ is independently selected from a bond, CR⁴⁶R⁴⁶, O, C(O), NR⁴⁷, SOₙ where n is 0, 1 or 2, cycloalkylene or heterocycloalkylene, where R⁴⁶ is H, halo, alkyl or alkoxy, and R⁴⁷ is H or alkyl; and x is 1-5. In another embodiment, x is 5. In another embodiment, x is 4. In another embodiment, x is 3. In another embodiment, x is 2. In another embodiment, x is 1. In another embodiment, L has formula L¹-L²-L³, where each of L¹-L³ is independently selected from a bond, CR⁴⁶R⁴⁶, O, C(O), NR⁴⁷, SOₙ where n is 0, 1 or 2, cycloalkyl or heterocycloalkyl, where R⁴⁶ is H, halo, alkyl or alkoxy, and R⁴⁷ is H or alkyl. In another embodiment, L has formula L¹-L², where each of L¹ and L² is independently selected from a bond, CR⁴⁶R⁴⁶, O, C(O), NR⁴⁷, SOₙ where n is 0, 1 or 2, cycloalkyl or heterocycloalkyl, where R⁴⁶ is H, halo, alkyl or alkoxy, and R⁴⁷ is H or alkyl. In another embodiment, L is a bond, CR⁴⁶R⁴⁶, O, C(O), NR⁴⁷, SOₙ where n is 0, 1 or 2, cycloalkyl or heterocycloalkyl, where R⁴⁶ is H, halo, alkyl or alkoxy, and R⁴⁷ is H or alkyl.

In another embodiment, L is a bond, CH₂CH₂SO₂, CH₂, CF₂, C(Me)₂, OCF₂, CONH, NHCONH, CH₂O, CH₂CH₂O, O or C(O). In other embodiments, L is CH₂CH₂SO₂, CH₂, CF₂, OCF₂, CONH, NHCONH, CH₂O, CH₂CH₂O, O or C(O). In another embodiment, L is CH₂, CF₂, OCF₂, CH₂O, O or C(O). In another embodiment, L is CH₂, CF₂, O or C(O). In another embodiment, L is CH₂. In another embodiment, L is a bond. In another embodiment, L is CF₂. In another embodiment, L is C(O). In another embodiment, L is C(Me)₂.

### E3 Ligase Binders E

E3 ligase binders E for use in the compounds provided herein are well known in the art. See, e.g., WO 2022/066835, WO 2020/118098, WO 2021/041664, WO 2019/078522, WO 2021/188537, WO 2021/105334, WO 2022/144416, WO 2021/143816, WO 2022/017365, WO 2022/146151, WO 2022/148358, WO 2021/147889, WO 2021/143822, WO 2020/181232, WO 2019/043214, WO 2020/263832, WO 2020/006233, WO 2015/200795, WO 2019/043217, WO 2019/204354, U.S. Patent Publication Nos. US 2022/0062248, US 2019/0017998, 2020/0206201, 2020/0155690, 2021/0009559, 2018/0215731, 2021/0177825, 2019/0076541, 2021/0403454, 2021/0284624, 2021/0032245, 2020/0207764, 2022/0112211, 2019/0233433, 2020/0207733.

In one embodiment, E is a moiety that binds to cereblon, the von-Hippel-Lindau (VHL) protein (e.g., VH032, VH285, VH298, VH101, VHL-e and VHL-g), an inhibitor of apoptosis (IAP) protein (e.g., MV1, ME-BS, GDC-0152, LCL-161, AT-IAP, SNIPER(ER)-110, SNIPER(ER)-126), a mouse double minute 2 homolog (MDM2) protein (e.g., Nutlin-3, RG7112 and idasanutlin), DCAF (e.g., indisulam, E7820, KB02 and chloroquinozaline sulfonamide (CQS)), an RNF protein (e.g., CCW16, nimbolide and EN219), or an aryl hydrocarbon receptor (AhR) protein including FEM1B and KEAP1 (e.g., β-NF, EN106, CDDO-Me, KEAP1-L, PL). See, e.g., Lee, et al. Molecules 2022, 27, 6515.

In another embodiment, E is a moiety that binds to cereblon. In another embodiment, E contains an imide, amide, thioamide or thioimide derived moiety. In another embodiment, E contains a phthalimido group or an analog or derivative thereof. In another embodiment, E contains a phthalimido-glutarimide group or an analog or derivative thereof. In another embodiment, E contains a thalidomide, lenalidomide or pomalidomide moiety, or an analog or derivative thereof.

In another embodiment, E has Formula X:

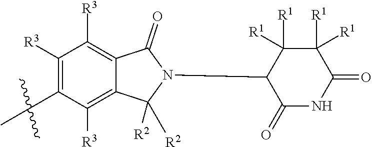

where:
R¹ is selected from (i) or (ii):
(i) each R¹ is independently H or alkyl, or
(ii) two R¹ groups together with the atom(s) to which they are attached form a spiro, bridged or fused cycloalkyl or heterocycloalkyl group and the other R¹ groups are each independently H or alkyl;
R² is selected from (i) or (ii):
(i) each R² is independently H or alkyl, or the two R² groups together form oxo; or
(ii) both R² groups together with the atom to which they are attached form a spiro cycloalkyl or heterocycloalkyl group;
each R³ is independently H, hydroxy, amino, cyano, halo, alkyl, alkoxy, haloalkyl or haloalkoxy.

In another embodiment, E has Formula X, where:
each R¹ is independently H or alkyl;
each R² is independently H or alkyl, or the two R² groups together form oxo;
each R³ is independently H, hydroxy, amino, cyano, halo, alkyl, alkoxy, haloalkyl or haloalkoxy.

In another embodiment, E is one of the following:

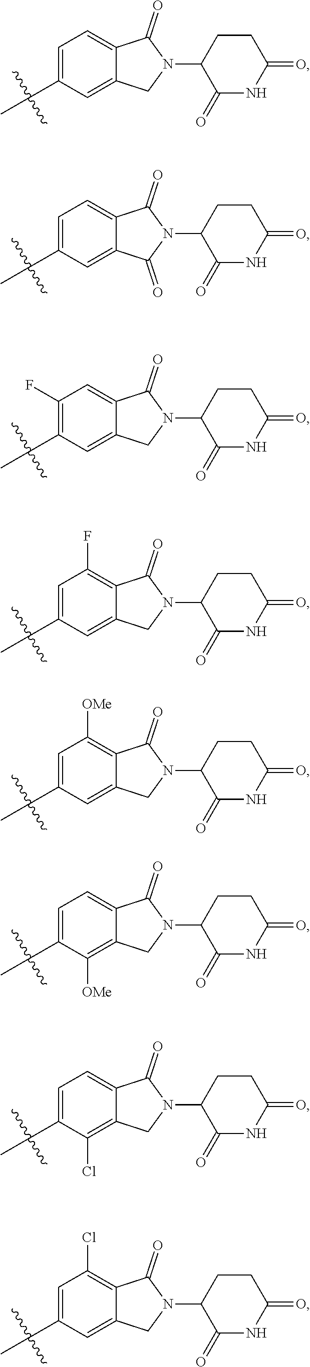

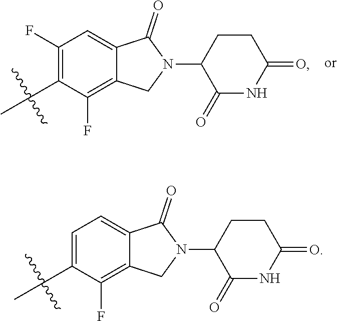 [SMILES: *c1c(F)cc2c(c1F)CN(C1CCC(=O)NC1=O)C2=O ; "or" ; *c1ccc2c(c1F)CN(C1CCC(=O)NC1=O)C2=O]

In another embodiment, E is

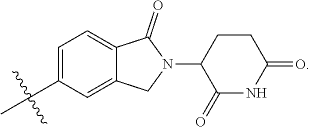 [SMILES: *c1ccc2c(c1)CN(C1CCC(=O)NC1=O)C2=O]

In another embodiment, E is:

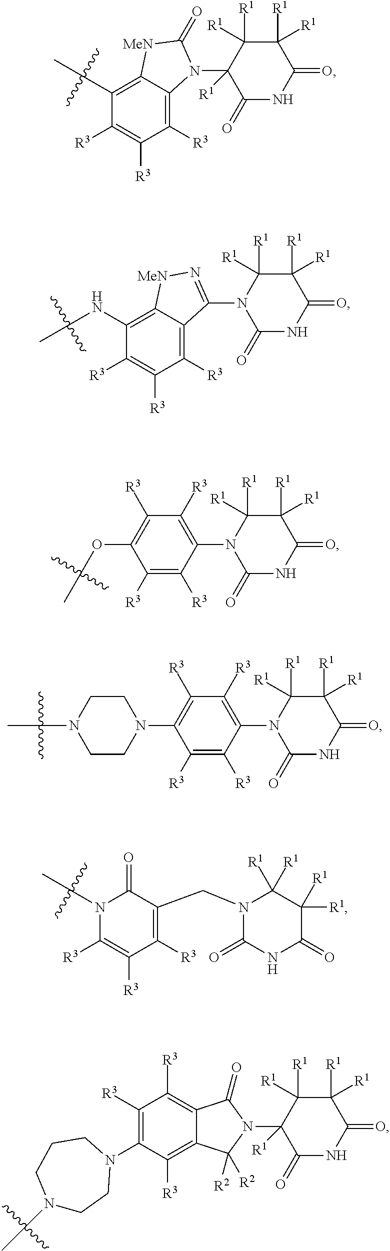

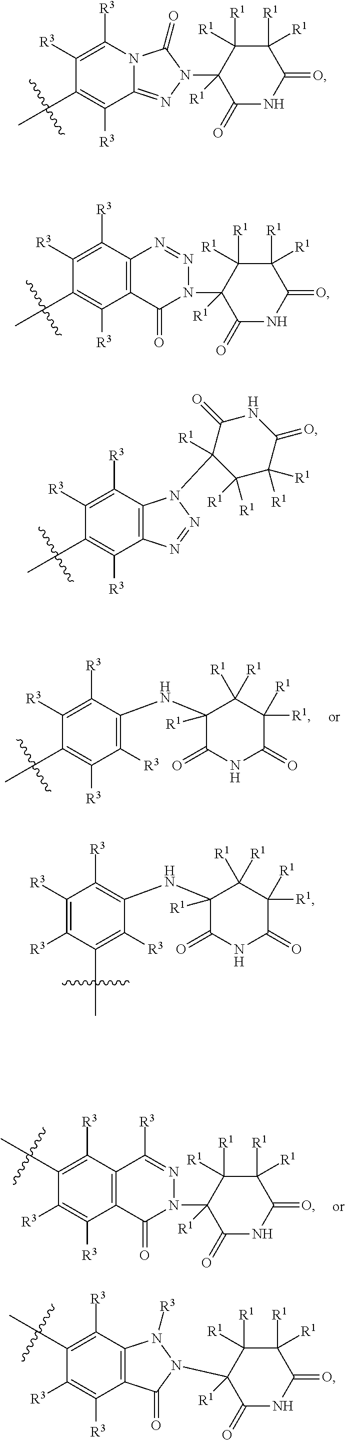

where R¹ and R³ are as defined elsewhere herein.

In another embodiment, E is:

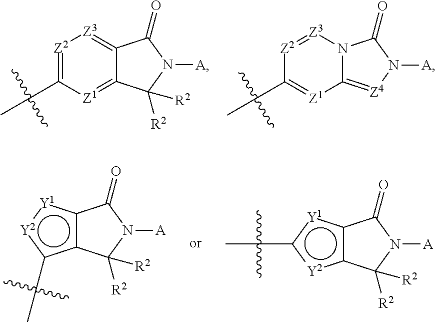

where A is a cyclic amide or cyclic imide or a derivative thereof; R² is as defined elsewhere herein; one of Y¹ and Y² is S and the other is CR³, where R³ is as defined elsewhere herein; and Z¹-Z⁴ are each independently N or CR³, where each R³ is as defined elsewhere herein. In one embodiment, at most two of Z¹-Z⁴ are N. In another embodiment, Z¹ and one of the R² groups, together with the atoms to which they are attached, form a fused phenyl ring; the other R² is absent; and this moiety has the formula:

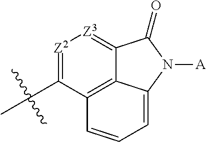

where A is a cyclic amide or cyclic imide or a derivative thereof, and Z² and Z³ are each independently N or CR³, where each R³ is as defined elsewhere herein.

In one embodiment, A is a cyclic imide having the structure:

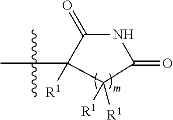

where each R¹ is as defined elsewhere herein; and m is an integer from 1-4.

In another embodiment, m is 1, 2 or 3. In another embodiment, m is 2 or 3. In another embodiment, m is 2. In another embodiment, m is 3.

In another embodiment, A has the structure:

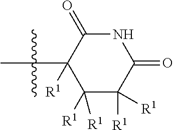

where each R¹ is selected as described elsewhere herein.

In another embodiment, A has the structure:

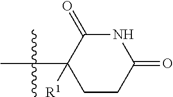

where R¹ is selected as described elsewhere herein.

In another embodiment, A has one of the following structures with the absolute stereochemistry shown:

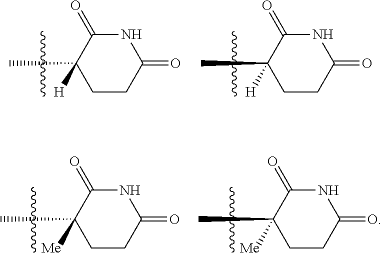

In another embodiment, Y¹ is S and Y² is CR³. In another embodiment, Y¹ is S and Y² is CH. In another embodiment, Y¹ is CR³ and Y² is S. In another embodiment, Y¹ is CH and Y² is S.

In another embodiment, Z¹ is N and Z²—Z⁴ are CR³. In another embodiment, Z¹ is N and Z²—Z⁴ are CH.

In another embodiment, Z² is N and Z¹, Z³ and Z⁴ are CR³. In another embodiment, Z² is N and Z¹, Z³ and Z⁴ are CH.

In another embodiment, Z³ is N and Z¹, Z² and Z⁴ are CR³. In another embodiment, Z³ is N and Z¹, Z² and Z⁴ are CH.

In another embodiment, Z⁴ is N and Z¹-Z³ are CR³. In another embodiment, Z⁴ is N and Z¹-Z³ are CH.

In another embodiment, E is an IMiD®.

In another embodiment, E is:

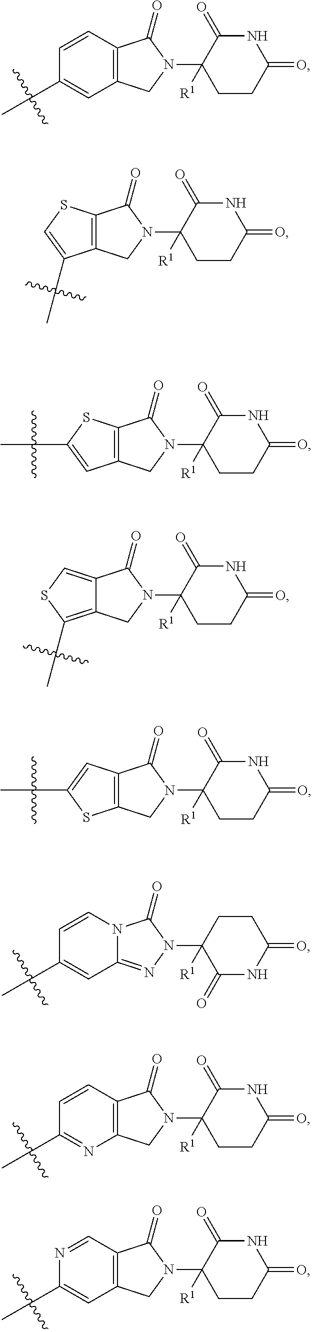

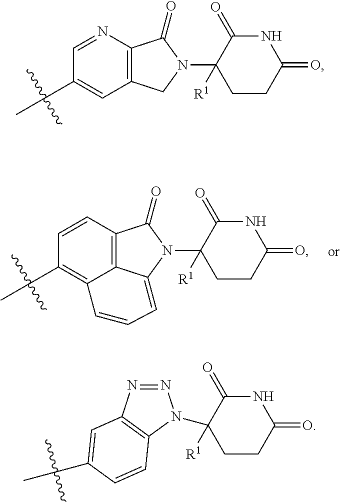

where R¹ is as defined elsewhere herein.

In another embodiment, E is selected from:

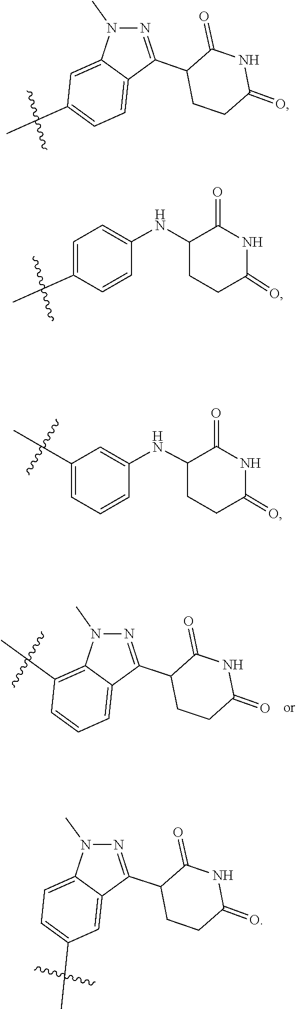 [SMILES: *c1ccc2c(C3CCC(=O)NC3=O)nn(C)c2c1 ; *c1ccc(NC2CCC(=O)NC2=O)cc1 ; *c1cccc(NC2CCC(=O)NC2=O)c1 ; *c1cccc2c(C3CCC(=O)NC3=O)nn(C)c12 ; *c1ccc2c(c1)c(C1CCC(=O)NC1=O)nn2C]

In another embodiment, E is an E3 ligase VHL ligand. See, e.g., Diehl et al. Chem. Soc. Rev. 2022, 51(19), 8216-8257; Buckley et al. J. Am. Chem. Soc. 2012, 134(10), 4465-4468; Bricelj et al. Front. Chem. 2021, 9; Crew et al. J. Med. Chem. 2018, 61, 583-598; Buckley et al. Angew. Chem. Int. Ed. 2012, 51, 11463-11467; Galdeano et al. J. Med. Chem. 2014, 57, 8657-8663; Steinebach et al. Chem. Sci. 2020, 11, 3474-3486; Raina et al. Proc. Natl. Acad. Sci. 2016, 113, 7124-7129; Han et al. J. Med. Chem. 2019, 62, 941-964; Hu et al J. Med. Chem. 2019, 62, 1420-1442.

In another embodiment, the E3 ligase VHL ligand is selected from:

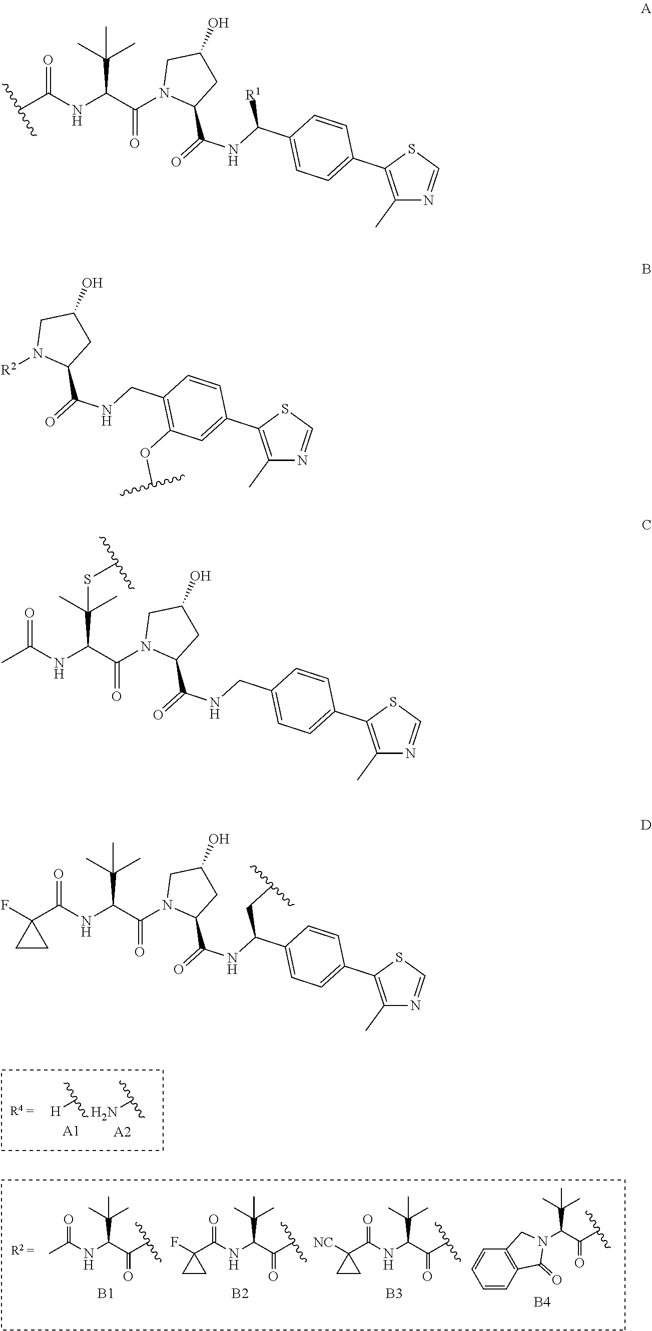

In another embodiment, the E3 ligase VHL ligand is A1 or B2.

In another embodiment, the moiety E that targets an E3 ubiquitin ligase is an N-terminal degradation signal sequence (N-degron). In another embodiment, E is a type 1 N-degron. In one embodiment, the type 1 N-degron is a positively charged amino acid. In another embodiment, the positively charged amino acid is Arg, Lys or His. In another embodiment, E is a type 2 N-degron. In one embodiment, the type 2 N-degron is a hydrophobic amino acid. In another embodiment, the hydrophobic amino acid is Phe, Trp, Tyr, Leu or Ile. In another embodiment, E is Arg, Lys, His, Phe, Trp, Tyr, Leu or Ile. In one embodiment, E is an arginine residue.

In one embodiment, the compound for use in the compositions and methods provided herein is selected from Table 1 below:

| Ex. No. | Structure |
|---|---|
| 1 | 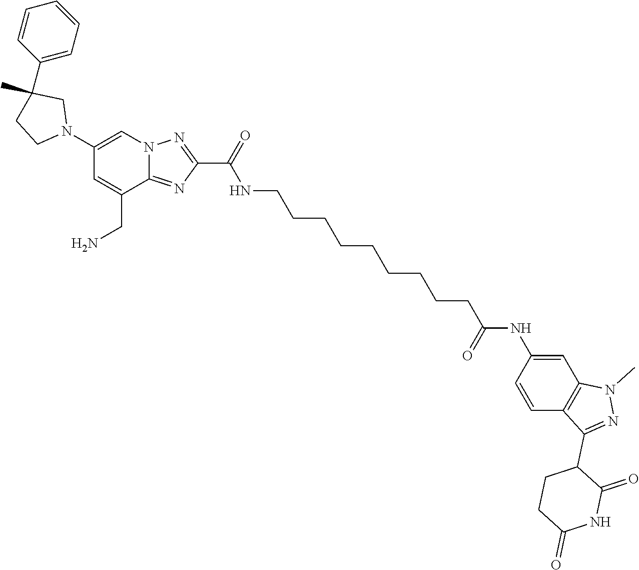 |
| 2 | 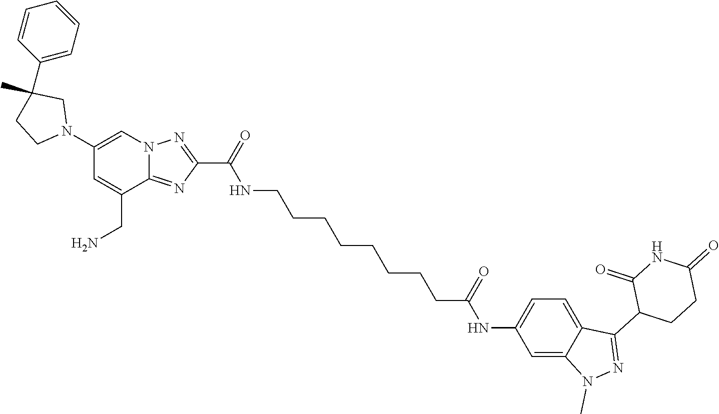 [SMILES: Cn1nc(C2CCC(=O)NC2=O)c2ccc(NC(=O)CCCCCCCCNC(=O)c3nc4c(CN)cc(N5CC[C@](C)(c6ccccc6)C5)cn4n3)cc21] |
| 3 | 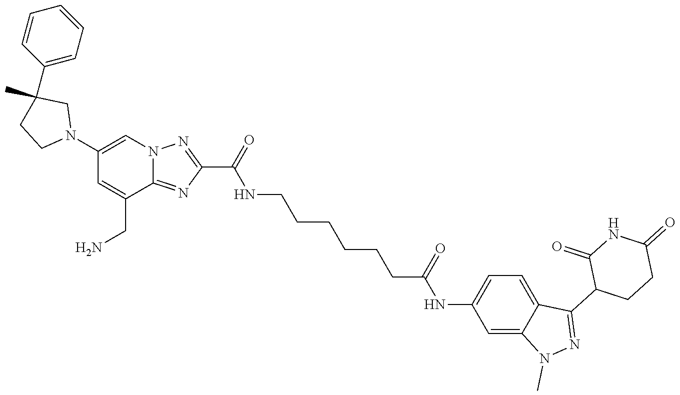 [SMILES: Cn1nc(C2CCC(=O)NC2=O)c2ccc(NC(=O)CCCCCCNC(=O)c3nc4c(CN)cc(N5CC[C@](C)(c6ccccc6)C5)cn4n3)cc21] |
| 4 | 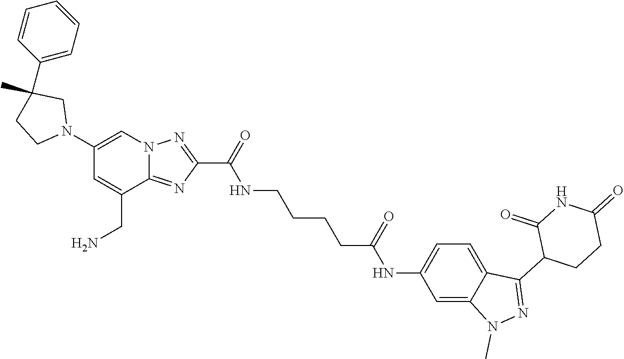 [SMILES: Cn1nc(C2CCC(=O)NC2=O)c2ccc(NC(=O)CCCCNC(=O)c3nc4c(CN)cc(N5CC[C@](C)(c6ccccc6)C5)cn4n3)cc21] |
| 5 | 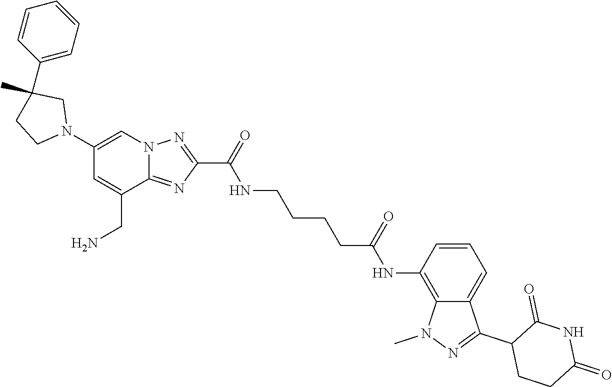 [SMILES: Cn1nc(C2CCC(=O)NC2=O)c2cccc(NC(=O)CCCCNC(=O)c3nc4c(CN)cc(N5CC[C@](C)(c6ccccc6)C5)cn4n3)c21] |
| 6 | 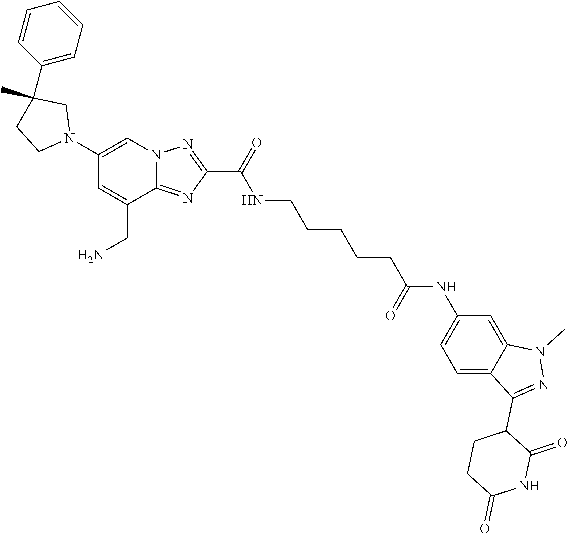 |
| 7 | 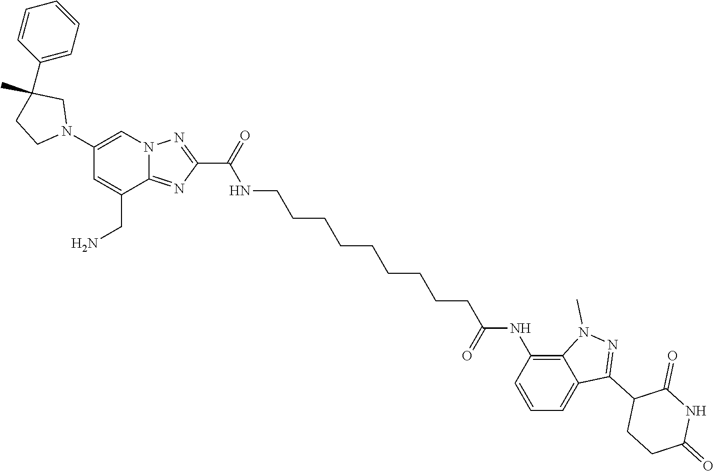 [SMILES: Cn1nc(C2CCC(=O)NC2=O)c2cccc(NC(=O)CCCCCCCCCNC(=O)c3nc4c(CN)cc(N5CC[C@](C)(c6ccccc6)C5)cn4n3)c21] |
| 8 | 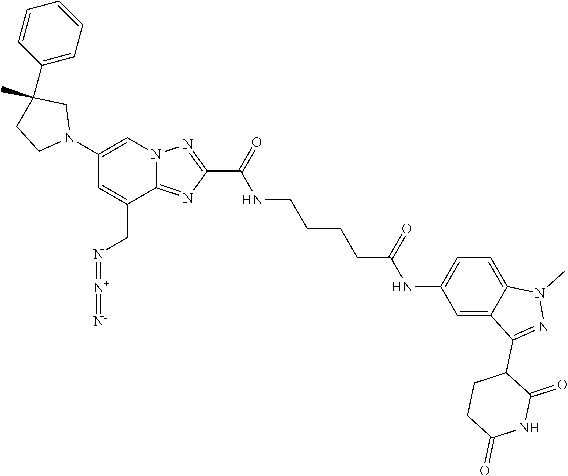 |
| 9 | 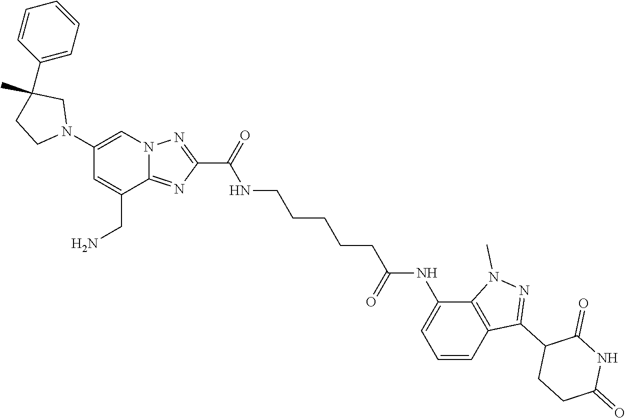 [SMILES: Cn1nc(C2CCC(=O)NC2=O)c2cccc(NC(=O)CCCCCNC(=O)c3nc4c(CN)cc(N5CC[C@](C)(c6ccccc6)C5)cn4n3)c21] |
| 10 | 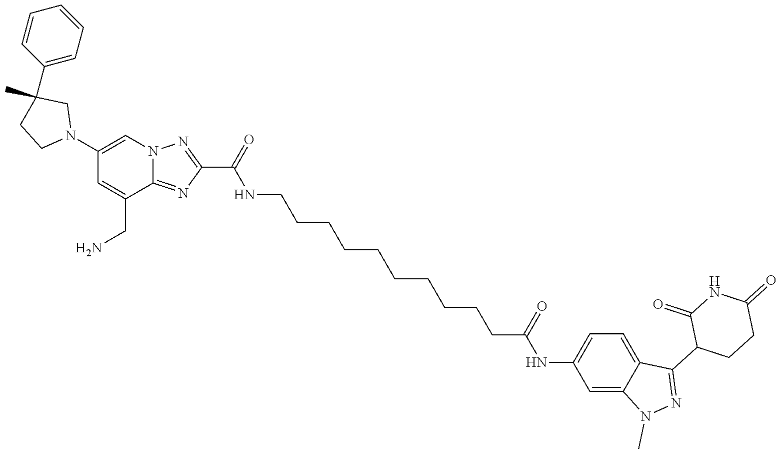 [SMILES: Cn1nc(C2CCC(=O)NC2=O)c2ccc(NC(=O)CCCCCCCCCCNC(=O)c3nc4c(CN)cc(N5CC[C@](C)(c6ccccc6)C5)cn4n3)cc21] |
| 11 | 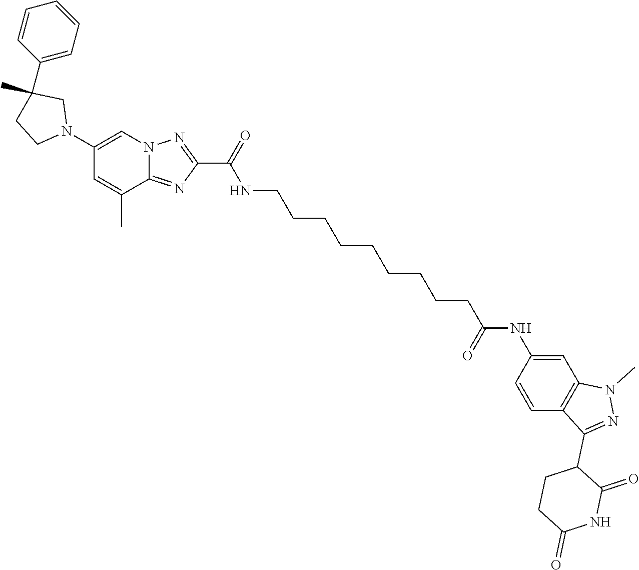 |
| 12 | 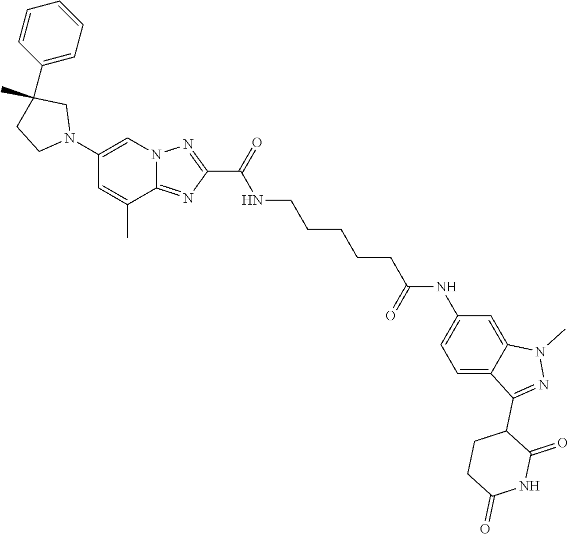 |
| 13 | 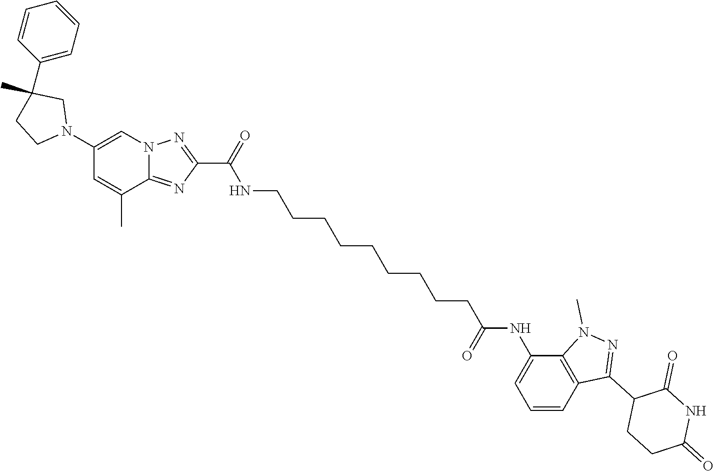 [SMILES: Cc1cc(N2CC[C@](C)(c3ccccc3)C2)cn2nc(C(=O)NCCCCCCCCCC(=O)Nc3cccc4c(C5CCC(=O)NC5=O)nn(C)c34)nc12] |
| 14 | 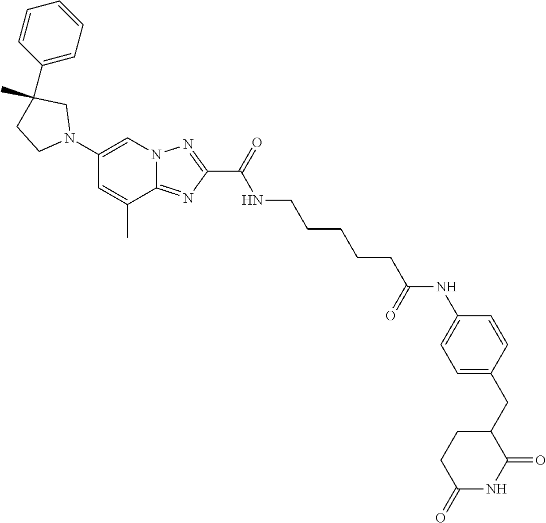 [SMILES: Cc1cc(N2CC[C@](C)(c3ccccc3)C2)cn2nc(C(=O)NCCCCCC(=O)Nc3ccc(CC4CCC(=O)NC4=O)cc3)nc12] |
| 15 | 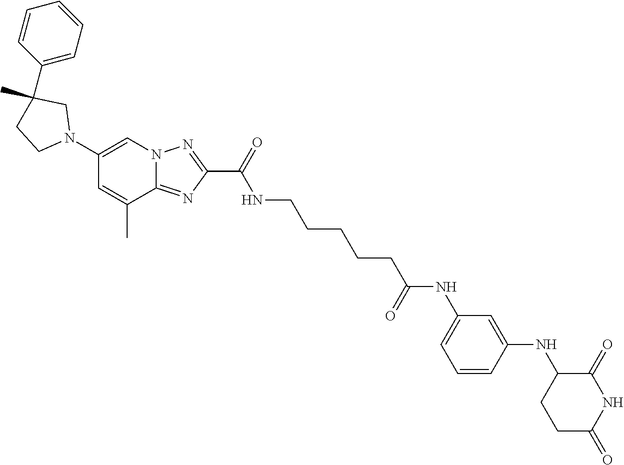 [SMILES: Cc1cc(N2CC[C@](C)(c3ccccc3)C2)cn2nc(C(=O)NCCCCCC(=O)Nc3cccc(NC4CCC(=O)NC4=O)c3)nc12] |
| 16 | 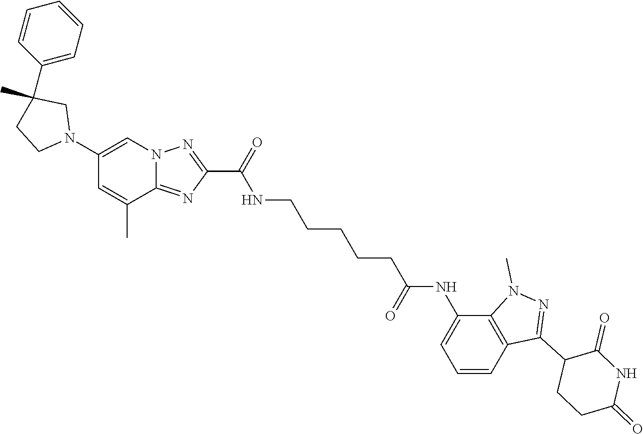 [SMILES: Cc1cc(N2CC[C@](C)(c3ccccc3)C2)cn2nc(C(=O)NCCCCCC(=O)Nc3cccc4c(C5CCC(=O)NC5=O)nn(C)c34)nc12] |
| 17 | 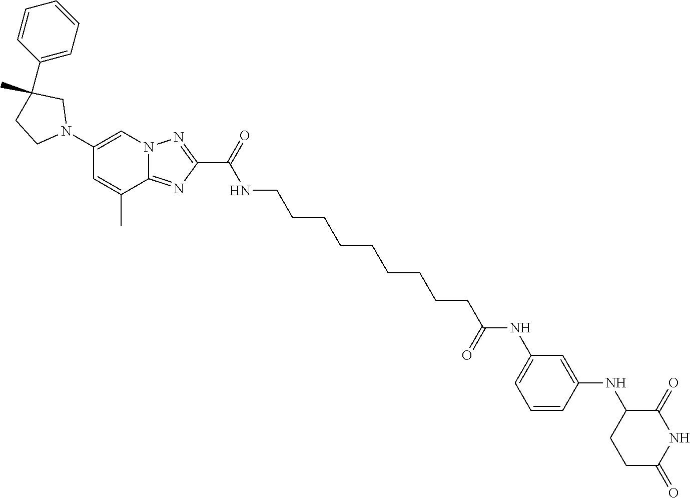 [SMILES: Cc1cc(N2CC[C@](C)(c3ccccc3)C2)cn2nc(C(=O)NCCCCCCCCCC(=O)Nc3cccc(NC4CCC(=O)NC4=O)c3)nc12] |
| 18 | 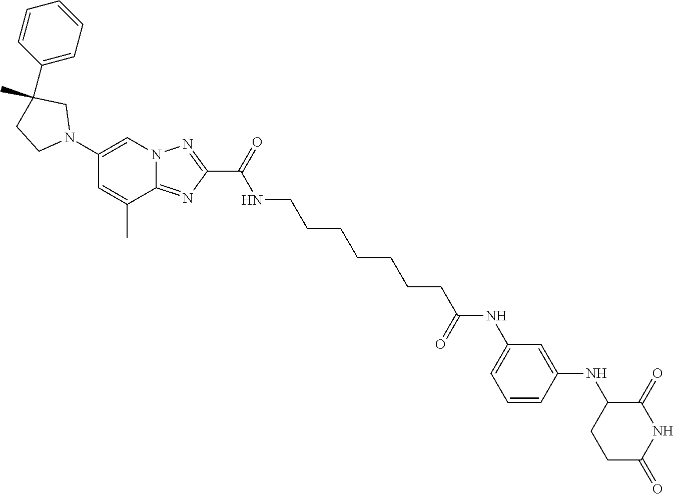 [SMILES: Cc1cc(N2CC[C@](C)(c3ccccc3)C2)cn2nc(C(=O)NCCCCCCCC(=O)Nc3cccc(NC4CCC(=O)NC4=O)c3)nc12] |
| 19 | 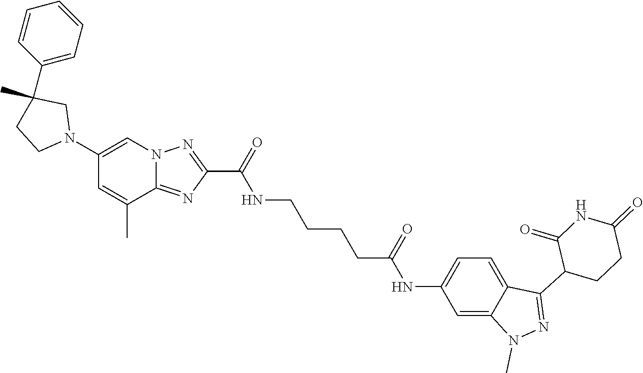 [SMILES: Cc1cc(N2CC[C@](C)(c3ccccc3)C2)cn2nc(C(=O)NCCCCC(=O)Nc3ccc4c(C5CCC(=O)NC5=O)nn(C)c4c3)nc12] |
| 20 | 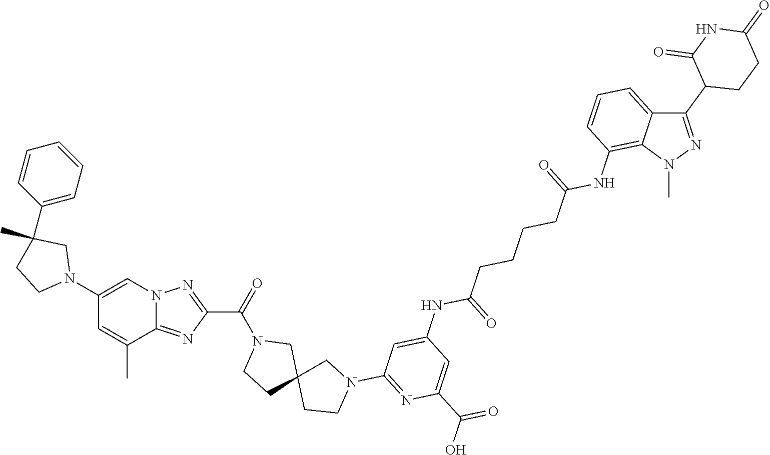 [SMILES: Cc1cc(N2CC[C@](C)(c3ccccc3)C2)cn2nc(C(=O)N3CC[C@@]4(CCN(c5cc(NC(=O)CCCCC(=O)Nc6cccc7c(C8CCC(=O)NC8=O)nn(C)c67)cc(C(=O)O)n5)C4)C3)nc12] |
| 21 | 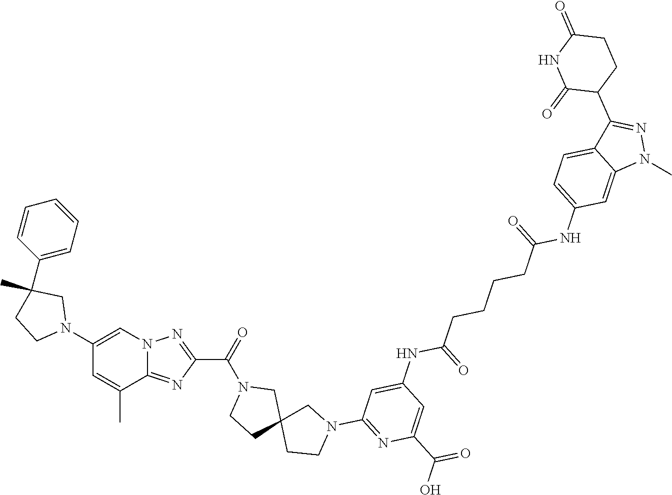 |
| 22 | 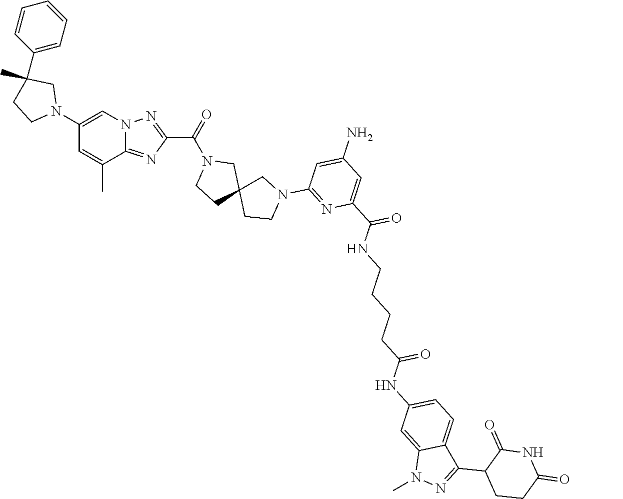 [SMILES: Cc1cc(N2CC[C@](C)(c3ccccc3)C2)cn2nc(C(=O)N3CC[C@@]4(CCN(c5cc(N)cc(C(=O)NCCCCC(=O)Nc6ccc7c(C8CCC(=O)NC8=O)nn(C)c7c6)n5)C4)C3)nc12] |
| 23 | 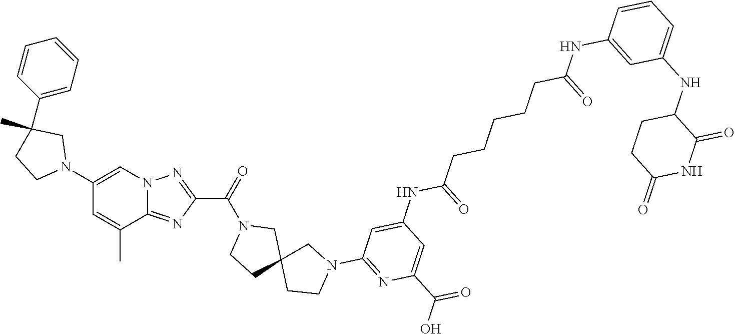 [SMILES: Cc1cc(N2CC[C@](C)(c3ccccc3)C2)cn2nc(C(=O)N3CC[C@@]4(CCN(c5cc(NC(=O)CCCCCC(=O)Nc6cccc(NC7CCC(=O)NC7=O)c6)cc(C(=O)O)n5)C4)C3)nc12] |
| 24 | 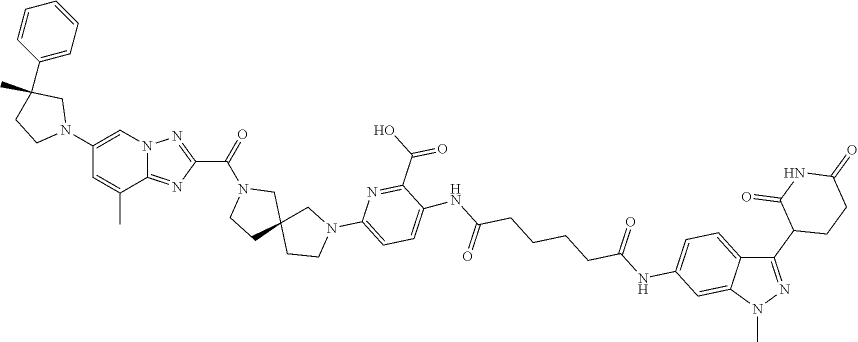 [SMILES: Cc1cc(N2CC[C@](C)(c3ccccc3)C2)cn2nc(C(=O)N3CC[C@@]4(CCN(c5ccc(NC(=O)CCCCC(=O)Nc6ccc7c(C8CCC(=O)NC8=O)nn(C)c7c6)c(C(=O)O)n5)C4)C3)nc12] |
| 24A | 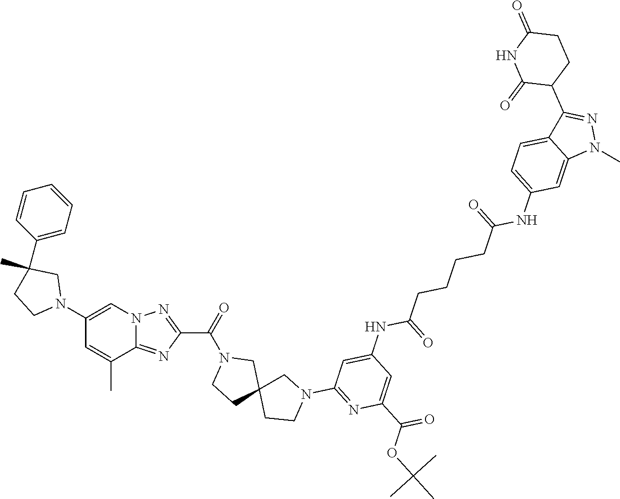 |
| 25 | 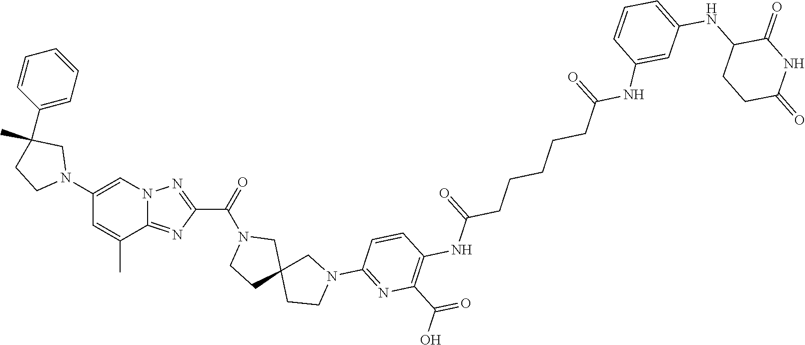 [SMILES: Cc1cc(N2CC[C@](C)(c3ccccc3)C2)cn2nc(C(=O)N3CC[C@@]4(CCN(c5ccc(NC(=O)CCCCCC(=O)Nc6cccc(NC7CCC(=O)NC7=O)c6)c(C(=O)O)n5)C4)C3)nc12] |
| 26 | 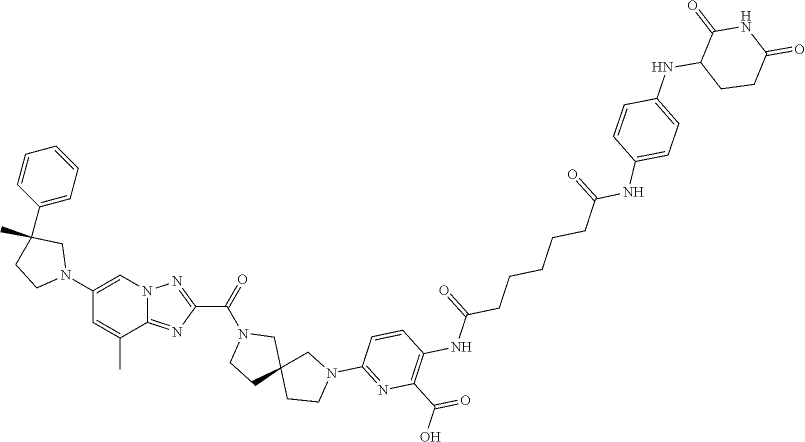 [SMILES: Cc1cc(N2CC[C@](C)(c3ccccc3)C2)cn2nc(C(=O)N3CC[C@@]4(CCN(c5ccc(NC(=O)CCCCCC(=O)Nc6ccc(NC7CCC(=O)NC7=O)cc6)c(C(=O)O)n5)C4)C3)nc12] |
| 27 | 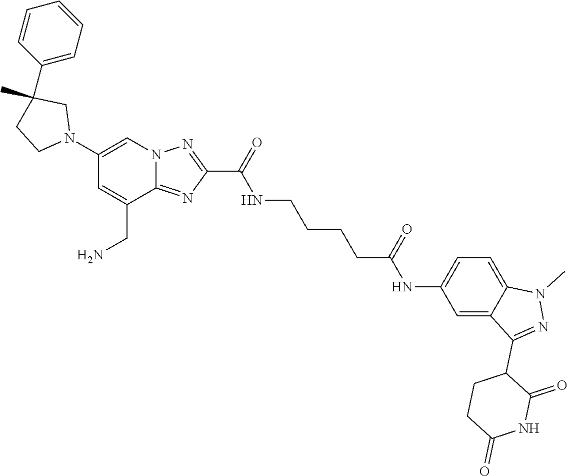 |
| 28 | 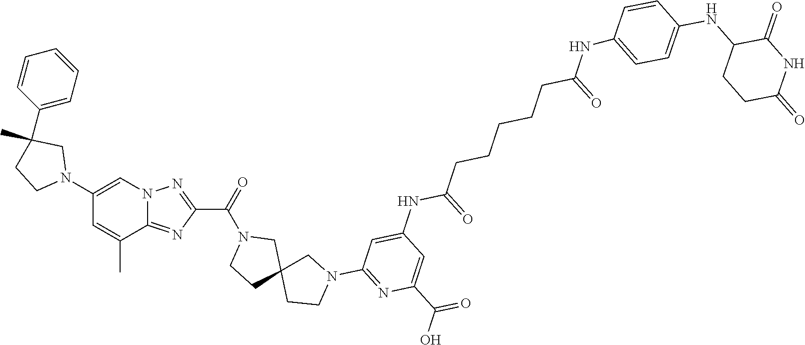 [SMILES: Cc1cc(N2CC[C@](C)(c3ccccc3)C2)cn2nc(C(=O)N3CC[C@@]4(CCN(c5cc(NC(=O)CCCCCC(=O)Nc6ccc(NC7CCC(=O)NC7=O)cc6)cc(C(=O)O)n5)C4)C3)nc12] |

### III. Synthesis of the Compounds

The compounds provided herein may be prepared according to methods well known to those of skill in the art, such as the methods provided in the Examples and routine modification thereof. See, e.g., WO 2024/092037, WO 2024/092040, WO 2024/092043, WO 2017/184995; WO 2019/144117; WO 2020/163823; WO 2021/078301; WO 2021/146536; WO 2021/007307; WO 2021/222114; WO 2021/078301; WO 2022/169780; WO 2023/044046; Chamberlain and Hamann, Nature Chemical Biology 15.10 (2019): 937-944; Li and Song, Journal of Hematology & Oncology 13 (2020): 1-14; Wu, et al. Nature Structural & Molecular Biology 27.7 (2020): 605-614; Dong, et al., Journal of Medicinal Chemistry 64.15 (2021): 10606-10620; Yang, et al., Targeted Oncology 16.1 (2021): 1-12; Lv, et al., Nature Communications 12.1 (2021): 6896.

### IV. Pharmaceutical Compositions

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically, the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MHLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and then extrapolated therefrom for dosages for humans. In some embodiments, the active compound is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC. 2014.00105) is used to determine the therapeutic concentration and safety profile of the active compound in specific subjects or subject populations.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to oral, parenteral, subcutaneous, intravenous, intramuscular, intraperitoneal, intrathecal, mucosal, dermal, transdermal, buccal, rectal, topical, local, nasal or inhalation. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001%-100% active ingredient, in certain embodiments, about 0.1-85% active ingredient, or, in other embodiments, about 75-95% active ingredient.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmacopeia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

a. Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, crospovidone, alginic acid, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene lauryl ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil in-water or water in oil. In some embodiments, the suspension is a suspension of microparticles or nanoparticles. In some embodiments, the emulsion is an emulsion of microparticles or nanoparticles.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### B. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN®80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### C. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

### D. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### E. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

### F. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

### G. Targeted Formulations

The compounds provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### H. Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In certain embodiments, provided herein also are kits which, when used by the medical practitioner, can simplify the administration of appropriate amounts of active ingredients to a subject. In certain embodiments, the kit provided herein includes a container and a dosage form of a compound provided herein, including a single enantiomer or a mixture of diastereomers thereof, or a pharmaceutically acceptable salt, solvate, or prodrug thereof.

In certain embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### V. Dosing

The compounds and pharmaceutical compositions provided herein may be dosed in certain therapeutically or prophylactically effective amounts, certain time intervals, certain dosage forms, and certain dosage administration methods as described below.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the compound provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 50 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In one embodiment, the compound provided herein, or a derivative thereof, is administered orally. In another embodiment, the compound provided herein, or a derivative thereof, is administered parenterally. In yet another embodiment, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the compound provided herein, or a derivative thereof, is administered twice a day. In yet another embodiment, the compound provided herein, or a derivative thereof, is administered three times a day. In still another embodiment, the compound provided herein, or a derivative thereof, is administered four times a day.

In certain embodiments, the compound provided herein, or a derivative thereof, is administered once per day from one day to six months, from one week to three months, from one week to four weeks, from one week to three weeks, or from one week to two weeks. In certain embodiments, the compound provided herein, or a derivative thereof, is administered once per day for one week, two weeks, three weeks, or four weeks. In one embodiment, the compound provided herein, or a derivative thereof, is administered once per day for 4 days. In one embodiment, the compound provided herein, or a derivative thereof, is administered once per day for 5 days. In one embodiment, the compound provided herein, or a derivative thereof, is administered once per day for 6 days. In one embodiment, the compound provided herein, or a derivative thereof, is administered once per day for one week. In another embodiment, the compound provided herein, or a derivative thereof, is administered once per day for two weeks. In yet another embodiment, the compound provided herein, or a derivative thereof, is administered once per day for three weeks. In still another embodiment, the compound provided herein, or a derivative thereof, is administered once per day for four weeks.

### VI. Methods of Treatment

Free light chains (FLCs) adopt a well-defined homodimeric structure, wherein the monomers may be covalently linked by an interchain disulfide bond. LC monomers comprise an N-terminal variable (V) domain attached to a C-terminal constant (C) domain. Amyloidogenic FLCs involved in AL patients, are less stable than non-amyloidogenic FLCs, can misfold and misassemble into nonnative species including cross-p-sheet amyloid fibrils, a hallmark of AL amyloidosis. The structure-proteotoxicity relationship is not fully understood but several processes have been described, including destabilization-dependent endoproteolysis that releases amyloidogenic LC fragments. LC fragments including V domains are observed in patient deposits alongside full length LCs. Without being bound by any theory, it is believed that the compounds and compositions provided herein stop light chain conformational excursions at the beginning of the aggregation cascade via kinetic stabilization of FLCs. It is also believed that the compounds provided herein bind to conserved regions of LCs, thereby allowing the compounds to stabilize many immunoglobulin light chains and to be effective in many AL subjects.

Kinetic stabilization of LCs is unlikely to contribute to plasma cell death but could reduce organ proteotoxicity and the progression of AL. Subjects with prominent cardiac involvement currently have few available options for treatment and represent an urgent unmet medical need, as they are often too sick to tolerate chemotherapy. Reduction of organ proteotoxicity could allow the subject to tolerate chemotherapy. Thus, in one embodiment, provided herein is a method of pretreatment of a subject having AL with prominent cardiac involvement with a compound provided herein, followed by chemotherapy.

In another embodiment, provided herein is a method of treating light chain amyloidosis by administering to a subject a compound or composition provided herein. In one embodiment, the subject has light chain amyloidosis and is treatment naive. In another embodiment, the subject has relapsed or refractory light chain amyloidosis.

In another embodiment, provided herein is a method of stabilizing immunoglobulin light chains by contacting the immunoglobulin light chains with a compound provided herein. In one embodiment, the immunoglobulin light chains are stabilized in a native conformation thereof. In certain embodiments, the immunoglobulin light chains are dimers. Thus, in certain embodiments, provided herein is a method of stabilizing immunoglobulin light chain dimers in a native conformation. As used herein, “native conformation” refers to a conformation of immunoglobulin light chains present in subjects not having light chain amyloidosis.

In another embodiment, provided herein is a method of preventing or lessening immunoglobulin light chain misfolding and/or endoproteolysis by contacting the immunoglobulin light chains with a compound provided herein.

In another embodiment, provided is a method of maintenance therapy upon recurrence of light chain amyloidosis following primary treatment by administering to a subject a compound or composition provided herein. In such embodiments, reemergence of the clonal plasma cells is generally slow, and thus organ toxicity caused by conformationally unstable circulating LC can be minimized by kinetic stabilizer treatment.

In another embodiment, provided is a method of degrading immunoglobulin light chains by contacting the immunoglobulin light chain or a composition containing an immunoglobulin light chain with a compound or composition provided herein.

### VII. Combination Therapy with a Second Active Agent

The compound provided herein, or a derivative thereof, can also be combined or used in combination with other therapeutic agents useful in the treatment and/or prevention of light chain amyloidosis.

In one embodiment, provided herein is a method of treating, preventing, or managing light chain amyloidosis, comprising administering to a subject a compound provided herein, or a derivative thereof, in combination with one or more second active agents. In one embodiment, the second active agent is a proteasome inhibitor (e.g., bortezomib, ixazomib, carfilzomib). In another embodiment, the second active agent is a chemotherapeutic agent, including but not limited to alkylating agents (e.g., bendamustine, melphalan, cyclophosphamide), steroids (e.g., dexamethasone), immunomodulatory agents (e.g., thalidomide, lenalidomide, pomalidomide), an anti-CD38 antibody (e.g., daratumumab, isatuximab), an anti-CD20 antibody (e.g., rituximab), an anti-IL-6 antibody (e.g., siltuximab), a UPR activator (e.g., an ATF-6 activator), an antibody-drug-conjugate (e.g., belantamab mafodotin, STI-6129 (sorrentotherapeutics.com)) and/or an anti-amyloid antibody. In another embodiment, the compounds provided herein, or a derivative thereof, are used in combination with stem cell transplant therapy. In further embodiments, the second active agent is selected from those disclosed in PCT Publication Nos. WO 2020/205683, WO 2019/191558, WO 2017/117430 or WO 2021/007594. In another embodiment, the second active agent is a therapeutic agent that promotes clearance of amyloid deposits, such as CAEL-101 (caelumbio.com) or NEOD001 (birtamimab).

In one embodiment, the second active agent is a plasma cell directed therapy, including high-dose cyclophosphamide combined with an anti-thymocyte antibody (e.g., Thymoglobulin®, Atgam®)(with subsequent autologous stem cell transplantation), an immunomodulatory agent (e.g., lenalidomide), a steroid (e.g., dexamethasone), a proteasome inhibitor (e.g., bortezomib), atacicept, or an anti-CD38 antibody (e.g., daratumumab, isatuximab). In another embodiment, the second active agent is a combination of daratumumab, bortezomib, cyclophosphamide and dexamethasone.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, a compound provided herein, e.g., the compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the compound provided herein, or a derivative thereof, is independent of the route of administration of a second therapy. In one embodiment, the compound provided herein, or a derivative thereof, is administered orally. In another embodiment, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein, or a derivative thereof, and a second therapy are administered by the same mode of administration, orally or by IV. In another embodiment, the compound provided herein, or a derivative thereof, is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more second active ingredients or agents can be used together with the compound provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins.

In one embodiment, the compound provided herein, or a derivative thereof, can be administered in an amount ranging from about 0.1 to about 150 mg, from about 1 to about 25 mg, or from about 2 to about 10 mg orally and daily alone, or in combination with a second active agent, prior to, during, or after the use of conventional therapy.

### VIII. Examples

The examples below are meant to illustrate certain embodiments provided herein, and not to limit the scope of this disclosure.

### Example 1

### (R)-8-(azidomethyl)-6-(3-methyl-3-phenylpyrrolidin-1-yl)-[1,2,4]triazolo[1,5-a]pyridine-2-carboxylic acid

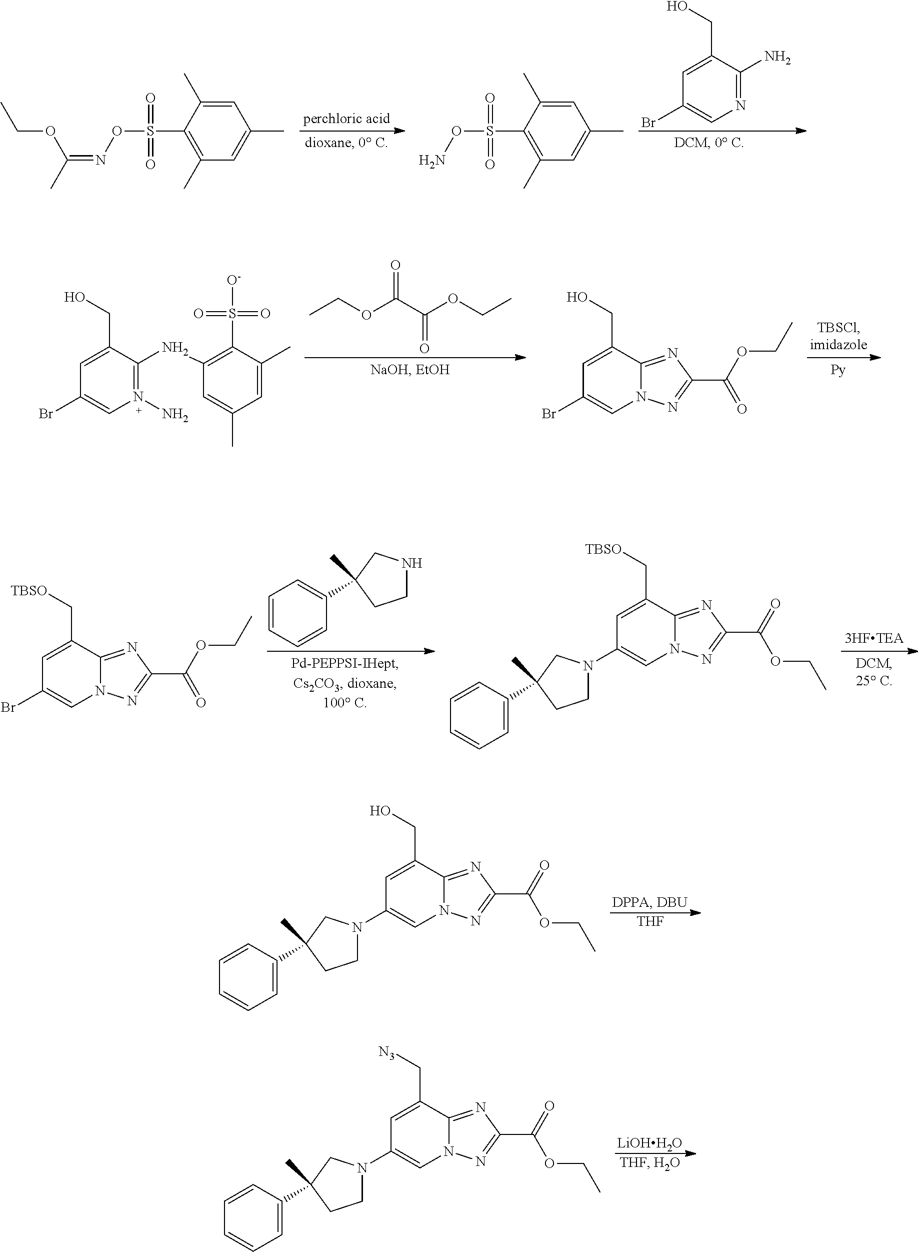

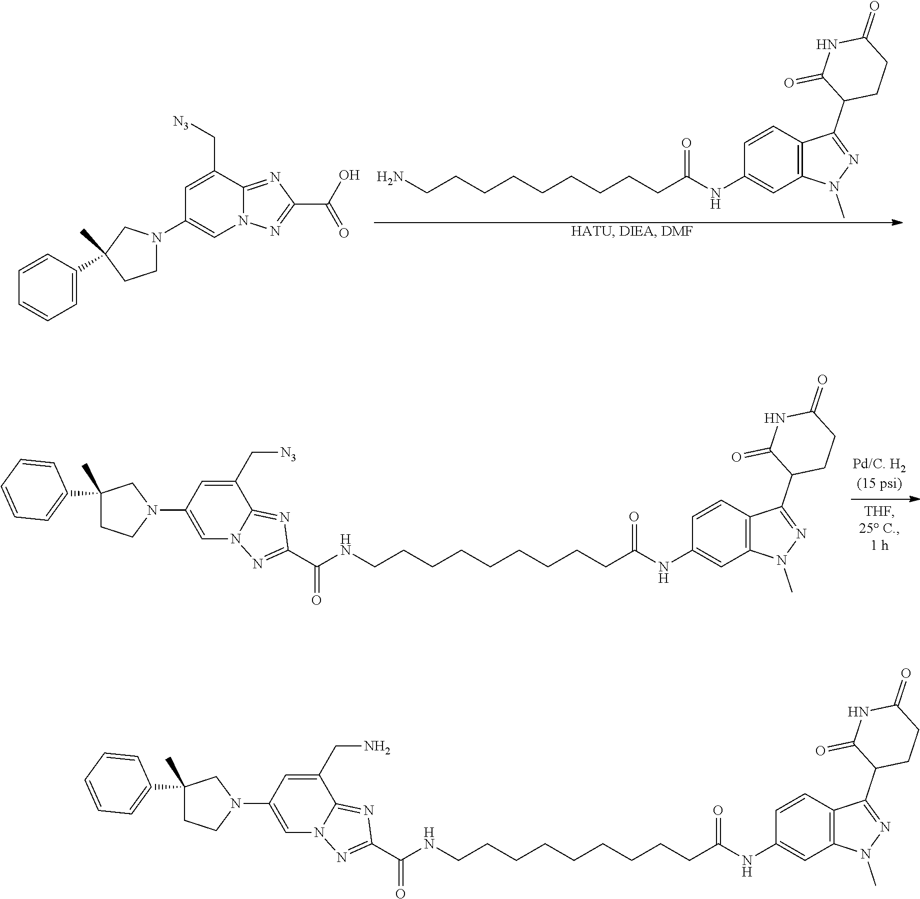

### Step 1: amino 2,4,6-trimethylbenzenesulfonate

To a solution of ethyl (1E)-N-(2,4,6-trimethylphenyl) sulfonyl oxyeth animidate (60 g, 210.26 mmol, 1 eq) in dioxane (240 mL) was added dropwise perchloric acid (150.88 g, 1.05 mol, 90.89 mL, 70% purity, 5 eq) while keeping inner temperature between −5-0° C. under N₂, then the mixture was stirred at 0° C. for 0.5 h under N₂. The reaction was monitored by TLC (PE: EA=3:1, Rf=0.4) and one new spot formed. After completed, ice-water (500 mL) was added in portions, the result precipitate was collected by filtration and washed with water to give the amino 2,4,6-trimethylbenzenesulfonate (45 g, crude) as a white solid.

### Step 2: 1,2-diamino-5-bromo-3-(hydroxymethyl)pyridin-1-ium 2,4,6-trimethylbenzenesulfonate

To a mixture of (2-amino-5-bromo-3-pyridyl) methanol (34 g, 167.46 mmol, 1 eq) in DCM (300 mL) was added dropwise a solution of amino 2,4,6-trimethylbenzenesulfonate (43.26 g, 200.95 mmol, 1.2 eq) in DCM (100 mL) at 0° C., then the mixture was stirred at 0° C. for 0.5 h. LCMS showed starting material was consumed completely. The mixture was filtered and the filter cake was concentrated under the reduced pressure to give the product to give the 1,2-diamino-5-bromo-3-(hydroxymethyl) pyridin-1-ium 2,4,6-trimethylbenzenesulfonate (68 g, 97% yield) as a brown solid.

LC-MS [ESI, M+1]: 418.2

¹H NMR (400 MHz, DMSO-d₆) δ=8.32 (br d, J=8.4 Hz, 3H), 7.88 (s, 1H), 6.84 (s, 2H), 6.74 (s, 3H), 4.44 (br s, 2H), 2.48-2.48 (m, 6H), 2.18 (s, 3H)

### Step 3: ethyl 6-bromo-8-(hydroxymethyl)-[1,2,4]triazolo[1,5-a]pyridine-2-carboxylate

To a solution of (1,2-diamino-5-bromo-pyridin-1-ium-3-yl) methanol; 2,4,6-trimethylbenzenesulfonate (20 g, 47.81 mmol, 1 eq) in EtOH (200 mL) was added NaOH (2.87 g, 71.72 mmol, 1.5 eq) and diethyl oxalate (13.97 g, 95.62 mmol, 13.06 mL, 2 eq). The mixture was stirred at 70° C. for 0.5 h. LCMS showed starting material was consumed completely and one main peak with desired mass was detected. The reaction mixture was concentrated under the reduced pressure to give the crude product. The residue was purified by flash silica gel chromatography (ISCO®; 80 g SepaFlash® Silica Flash Column, Eluent of 0-60% EA/PE gradient @100 mL/min) to give the ethyl 6-bromo-8-(hydroxymethyl)-[1,2,4]triazolo[1,5-a]pyridine-2-carboxylate (6 g, 42% yield).

LC-MS [ESI, M+1]: 301.11.

¹H NMR (400 MHz, DMSO-d₆) δ=9.38 (br s, 1H), 7.78 (s, 1H), 5.76 (t, J=5.6 Hz, 1H), 4.88 (br d, J=5.6 Hz, 2H), 4.40 (q, J=7.2 Hz, 2H), 1.32 (t, J=7.2 Hz, 3H)

### Step 4: ethyl 6-bromo-8-(((tert-butyldimethylsilyl)oxy)methyl)-[1,2,4]triazolo[1,5-a]pyridine-2-carboxylate

To a solution of ethyl 6-bromo-8-(hydroxymethyl)-[1,2,4]triazolo[1,5-a]pyridine-2-carboxylate (6 g, 19.99 mmol, 1 eq) in pyridine (60 mL) was added imidazole (3.40 g, 49.98 mmol, 2.5 eq) and then was added TBSCI (7.53 g, 49.98 mmol, 6.15 mL, 2.5 eq) at 0° C. The mixture was stirred at 25° C. for 2 h. LCMS showed starting material was consumed completely and one main peak with desired mass was detected. The reaction mixture was concentrated under the reduced pressure to give the crude product. The residue was purified by flash silica gel chromatography (ISCO®; 20 g SepaFlash® Silica Flash Column, Eluent of 0-X % Ethylacetate/Petroleum ethergradient @80 mL/min) to give the ethyl 6-bromo-8-(((tert-butyldimethylsilyl)oxy)methyl)-[1,2,4]triazolo[1,5-a]pyridine-2-carboxylate (3.8 g, 46% yield)/LC-MS [ESI, M+1]: 415.37.

¹H NMR (400 MHz, CHLOROFORM-d)δ=8.68 (d, J=0.8 Hz, 1H), 7.80 (d, J=1.6 Hz, 1H), 5.18 (s, 2H), 4.58 (q, J=7.2 Hz, 2H), 1.48 (t, J=7.2 Hz, 3H), 0.98 (s, 9H), 0.16 (s, 6H)

### Step 5: ethyl (R)-8-(((tert-butyldimethylsilyl)oxy)methyl)-6-(3-methyl-3-phenylpyrrolidin-1-yl)-[1,2,4]triazolo[1,5-a]pyridine-2-carboxylate

To a solution of ethyl 6-bromo-8-[[tert-butyl(dimethyl)silyl]oxymethyl]-[1,2,4]triazolo[1,5-a]pyridine-2-carboxylate (3.5 g, 8.45 mmol, 1 eq) and (3R)-3-methyl-3-phenyl-pyrrolidine (1.36 g, 8.45 mmol, 1 eq) in dioxane (40 mL) was added Pd-PEPPSI-IHeptCl (410.83 mg, 422.33 mol, 0.05 eq) and Cs₂CO₃ (8.26 g, 25.34 mmol, 3 eq). The mixture was stirred at 100° C. for 12 h. LCMS showed starting material was consumed completely and one main peak with desired mass was detected. The reaction mixture was concentrated under the reduced pressure to give the crude product. The residue was purified by prep-HPLC (FA) and lyophilized to give the ethyl (R)-8-(((tert-butyldimethylsilyl)oxy)methyl)-6-(3-methyl-3-phenylpyrrolidin-1-yl)-[1,2,4]triazolo[1,5-a]pyridine-2-carboxylate (3.6 g, 85% yield)

LC-MS [ESI, M+1]: 495.3

¹H NMR (400 MHz, CHLOROFORM-d) 6=7.70 (s, 1H), 7.44 (br s, 1H), 7.41-7.36 (m, 2H), 7.31 (br d, J=7.6 Hz, 3H), 5.18 (s, 2H), 4.55 (q, J=7.2 Hz, 2H), 3.63 (br d, J=8.8 Hz, 1H), 3.59-3.52 (m, 1H), 3.51-3.42 (m, 2H), 2.45-2.34 (m, 1H), 2.33-2.24 (m, 1H), 1.50-1.46 (m, 6H), 1.01 (s, 9H), 0.16 (s, 6H)

### Step 6: ethyl (R)-8-(hydroxymethyl)-6-(3-methyl-3-phenylpyrrolidin-1-yl)-[1,2,4]triazolo[1,5-a]pyridine-2-carboxylate

To a solution of ethyl 8-[[tert-butyl(dimethyl)silyl]oxymethyl]-6-[(3R)-3-methyl-3-phenyl-pyrrolidin-1-yl]-[1,2,4]triazolo[1,5-a]pyridine-2-carboxylate (3.2 g, 6.47 mmol, 1 eq) in DCM (35 mL) was added 3HF·TEA (7.91 g, 49.08 mmol, 8.00 mL, 7.59 eq). The mixture was stirred at 25° C. for 1 h. LCMS showed starting material was consumed and the desired mass was detected. The reaction mixture was extracted with EA (60 mL×3), the combined organic layers were dried over anhydrous Na₂SO₄, filtered and dried to give a residue. The residue was purified by flash silica gel chromatography (ISCO®; 20 g SepaFlash® Silica Flash Column, Eluent of 0˜80% Ethylacetate/Petroleum ethergradient @40 mL/min) TLC (PE:EA)=0 1 Rf=0.5) to give the ethyl (R)-8-(hydroxymethyl)-6-(3-methyl-3-phenylpyrrolidin-1-yl)-[1,2,4]triazolo[1,5-a]pyridine-2-carboxylate (2.4 g, 98% yield)

LC-MS [ESI, M+1]: 381.4

¹H NMR (CHLOROFORM-d, 400 MHz): δ=7.63 (d, J=2.0 Hz, 1.0H), 7.16-7.33 (m, 6.1H), 5.06 (br d, J=4.0 Hz, 2.0H), 4.44 (q, J=7.2 Hz, 2.0H), 3.52-3.66 (m, 2.1H), 3.38-3.50 (m, 2.0H), 3.35 (td, J=8.8, 3.6 Hz, 1.0H), 2.14-2.36 (m, 2.0H), 1.23-1.50 ppm (m, 6.0H)

### Step 7: ethyl (R)-8-(azidomethyl)-6-(3-methyl-3-phenylpyrrolidin-1-yl)-[1,2,4]triazolo[1,5-a]pyridine-2-carboxylate

To a solution of ethyl (R)-8-(hydroxymethyl)-6-(3-methyl-3-phenylpyrrolidin-1-yl)-[1,2,4]triazolo[1,5-a]pyridine-2-carboxylate (2.3 g, 6.05 mmol, 1 eq) in THE (20 mL) was added DPPA (2.50 g, 9.07 mmol, 1.96 mL, 1.5 eq) and DBU (2.30 g, 15.11 mmol, 2.28 mL, 2.5 eq). The mixture was stirred at 25° C. for 2 h. LCMS showed starting material was consumed completely and one main peak with desired m/z or desired mass was detected. The reaction mixture was diluted with water (30 mL), then was extracted with ethyl acetate (30 mL×3). The combined organic phase was washed with brine (30 mL), dried with anhydrous Na₂SO₄, filtered and concentrated in vacuum. The residue was purified by flash silica gel chromatography (ISCO®; 20 g SepaFlash® Silica Flash Column, Eluent of 0˜60% Ethyl acetate/Petroleum ethergradient @80 mL/min) to give the ethyl (R)-8-(azidomethyl)-6-(3-methyl-3-phenylpyrrolidin-1-yl)-[1,2,4]triazolo[1,5-a]pyridine-2-carboxylate (2.13 g, 86% yield)

LC-MS [ESI, M+1]: 406.1

¹H NMR (400 MHz, CHLOROFORM-d) 6=7.76 (s, 1H), 7.42-7.36 (m, 2H), 7.36-7.27 (m, 3H), 7.26-7.24 (m, 1H), 4.92 (s, 2H), 4.55 (q, J=7.2 Hz, 2H), 3.65 (d, J=8.8 Hz, 1H), 3.61-3.50 (m, 2H), 3.46 (dt, J=3.2, 8.8 Hz, 1H), 2.42 (td, J=8.4, 12.0 Hz, 1H), 2.34-2.25 (m, 1H), 1.53-1.42 (m, 6H)

### Step 8: (R)-8-(azidomethyl)-6-(3-methyl-3-phenylpyrrolidin-1-yl)-[1,2,4]triazolo[1,5-a]pyridine-2-carboxylic acid

To a solution of ethyl 8-(azidomethyl)-6-[(3R)-3-methyl-3-phenyl-pyrrolidin-1-yl]-[1,2,4]triazolo[1,5-a]pyridine-2-carboxylate (2 g, 4.93 mmol, 1 eq) in THF (20 mL) was added LiOH·H₂O (827.99 mg, 19.73 mmol, 4 eq) and H₂O (2 mL). The mixture was stirred at 25° C. for 1 h. LCMS showed starting material was consumed completely and one main peak with desired mass was detected. The reaction mixture was adjusted pH to around 4 by 1 M HCl, and then extracted with ethyl acetate (30 mL×3). The combined organic layers were washed with brine (30 mL), dried over Na₂SO₄, filtered and concentrated under reduced pressure to give the (R)-8-(azidomethyl)-6-(3-methyl-3-phenylpyrrolidin-1-yl)-[1,2,4]triazolo[1,5-a]pyridine-2-carboxylic acid (1.8 g, crude).

LC-MS [ESI, M+1]: 378.2

### Step 9: 8-(azidomethyl)-N-(10-((3-(2,6-dioxopiperidin-3-yl)-1-methyl-1H-indazol-6-yl)amino)-10-oxodecyl)-6-((R)-3-methyl-3-phenylpyrrolidin-1-yl)-[1,2,4]triazolo[1,5-a]pyridine-2-carboxamide

To a solution of 8-(azidomethyl)-6-[(3R)-3-methyl-3-phenyl-pyrrolidin-1-yl]-[1,2,4]triazolo[1,5-a]pyridine-2-carboxylic acid (80 mg, 211.98 mol, 1 eq) and 10-amino-N-[3-(2,6-dioxo-3-piperidyl)-1-methyl-indazol-6-yl]decanamide (88.52 mg, 190.78 mol, 0.9 eq, HCl) in DMF (1 mL) was added HATU (161.20 mg, 423.95 mol, 2 eq) and then was added DIEA (82.19 mg, 635.93 mol, 110.77 μL, 3 eq) after 0.1 h. The mixture was stirred at 25° C. for 1 h. LCMS showed starting material was consumed completely and the desired mass was detected. The reaction mixture was added water, and then was extracted with ethyl acetate (30 mL×3). The combined organic phase was washed with brine (30 mL), dried with anhydrous Na₂SO₄, filtered and concentrated in vacuum. The crude product was used into the next step without further purification to give the 8-(azidomethyl)-N-(10-((3-(2,6-dioxopiperidin-3-yl)-1-methyl-1H-indazol-6-yl)amino)-10-oxodecyl)-6-((R)-3-methyl-3-phenylpyrrolidin-1-yl)-[1,2,4]triazolo[1,5-a]pyridine-2-carboxamide (140 mg, 84% yield) as a yellow solid.

LC-MS [ESI, M+1]: 787.92

### Step 10: 8-(aminomethyl)-N-(10-((3-(2,6-dioxopiperidin-3-yl)-1-methyl-1H-indazol-6-yl)amino)-10-oxodecyl)-6-((R)-3-methyl-3-phenylpyrrolidin-1-yl)-[1,2,4]triazolo[1,5-a]pyridine-2-carboxamide

To a solution of 8-(azidomethyl)-N-[10-[[3-(2,6-dioxo-3-piperidyl)-1-methyl-indazol-6-yl]amino]-10-oxo-decyl]-6-[(3R)-3-methyl-3-phenyl-pyrrolidin-1-yl]-[1,2,4]triazolo[1,5-a]pyridine-2-carboxamide (140 mg, 177.91 mol, 1 eq) in THE (2 mL) was added Pd/C (18.93 mg, 17.79 mol, 10% purity, 0.1 eq). The mixture was stirred at 25° C. for 2 h under H₂(15 Psi). LCMS showed starting material was consumed completely and one main peak with desired mass was detected. The reaction mixture was filtered and the filtrate was concentrated under the reduced pressure to give the product. Washed the cake with THE several times and then added water to seal. The residue was purified by prep-HPLC (column: CD01-Phenomenex luna C18 150*25*10 um; mobile phase: [water(FA)-ACN]; gradient:20%-50% B over 11 min) and lyophilized to give a 8-(aminomethyl)-N-(10-((3-(2,6-dioxopiperidin-3-yl)-1-methyl-1H-indazol-6-yl)amino)-10-oxodecyl)-6-((R)-3-methyl-3-phenylpyrrolidin-1-yl)-[1,2,4]triazolo [1,5-a]pyridine-2-carboxamide (4.69 mg, 3% yield, FA) was obtained as a white solid

LC-MS [ESI, M+1]:807.95

¹H NMR (400 MHz, CCDCl₃) δ 8.16 (d, 1H, J=8.0 Hz), 8.10 (br s, 1H), 7.96 (d, 1H, J=4.0 Hz), 7.60 (d, 1H, J=4.0 Hz), 7.3-7.4 (m, 2H), 7.3-7.3 (m, 3H), 7.25 (br d, 1H, J=4.0 Hz), 7.09 (s, 1H), 6.67 (dd, 1H, J=8.0, 5.6 Hz), 4.0-4.1 (m, 1H), 3.9-4.0 (m, 2H), 3.63 (d, 1H, J=16.0 Hz), 3.40 (br s, 1H), 3.3-3.4 (m, 3H), 3.2-3.3 (m, 3H), 3.01 (d, 3H, J=4.0 Hz), 2.57 (s, 3H), 2.30 (d, 2H, J=4.0 Hz), 1.67 (s, 5H), 1.5-1.6 (m, 2H), 1.44 (d, 3H, J=4.0 Hz).

### Example 2

N-[6-[[3-(2,6-dioxo-3-piperidyl)-1-methyl-indazol-6-yl]amino]-6-oxo-hexyl]-8-methyl-6-[(3R)-3-methyl3-phenyl-pyrrolidin-1-yl]-[1,2,4]triazolo[1,5-a]pyridine-2-carboxamide

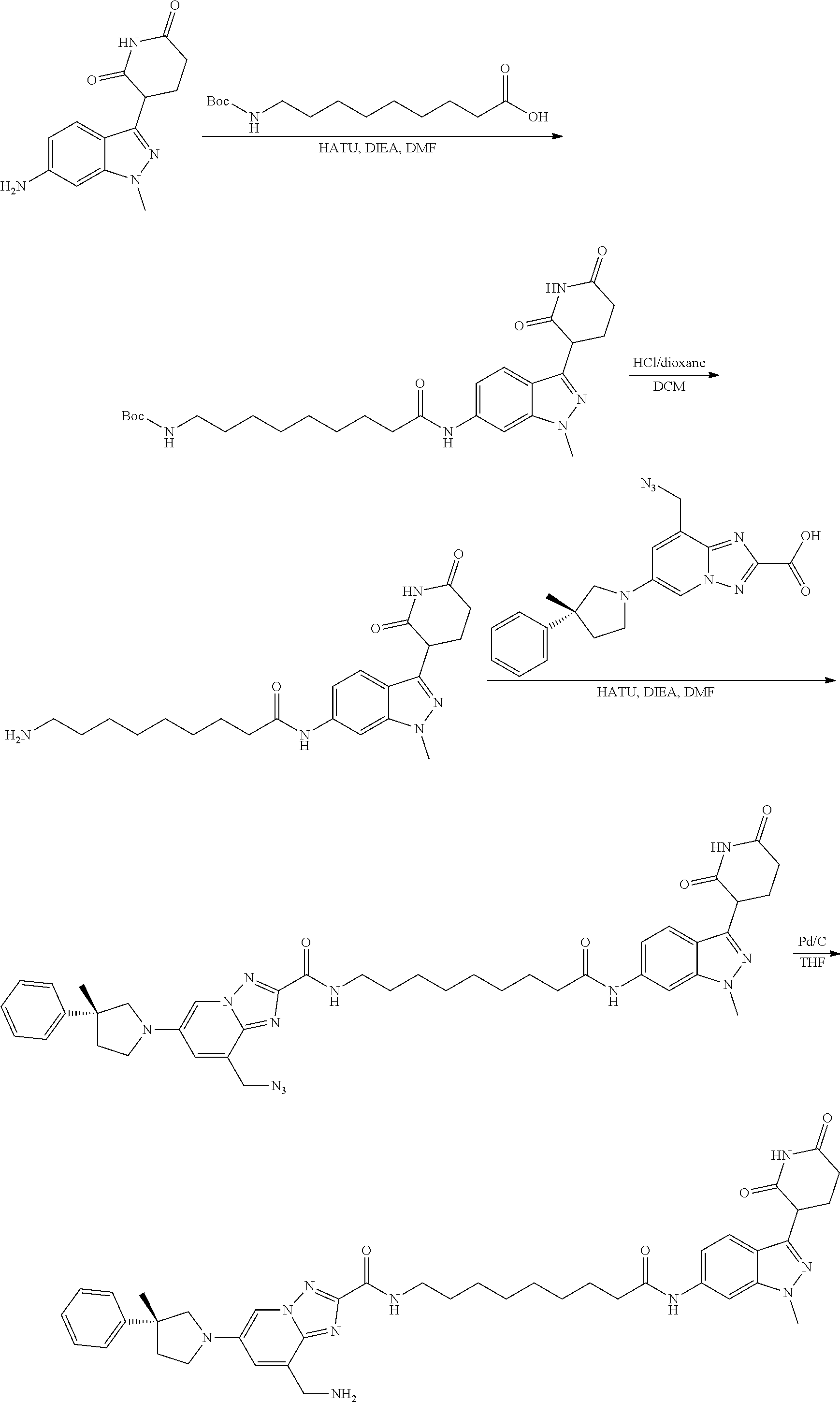

### Step 1: tert-butyl N-[9-[[3-(2,6-dioxo-3-piperidyl)-1-methyl-indazol-6-yl]amino]-9-oxo-nonyl]carbamate

To a solution of 3-(6-amino-1-methyl-indazol-3-yl)piperidine-2,6-dione (200 mg, 774.37 mol, 1 eq) and 9-(tertbutoxycarbonylamino)nonanoic acid (211.69 mg, 774.37 mol, 1 eq) in DMF (1.5 mL) was added HATU (441.66 mg, 1.16 mmol, 1.5 eq) and DIEA (300.24 mg, 2.32 mmol, 404.64 μL, 3 eq). The mixture was stirred at 25° C. for 0.5 h. LCMS showed starting material was consumed and one main peak with desired mass was detected. The mixture was quenched with H₂O (10 mL) and extracted with EtOAc (10 mL×3), the organic phase was dried, filtered, and concentrated under pressure to give the product. The residue was purified by flash silica gel chromatography (ISCO®; 20 g SepaFlash® Silica Flash Column, Eluent of 0˜80% EtOAc/PE gradient) to give compound tert-butyl N-[9-[[3-(2,6-dioxo-3-piperidyl)-1-methyl-indazol-6-yl]amino]-9-oxo-nonyl]carbamate (0.13 g, 33% yield) as a yellow solid.

¹H NMR (400 MHz, CHLOROFORM-d) 6=8.23 (s, 1H), 7.94 (s, 1H), 7.58 (br d, J=8.4 Hz, 2H), 6.82 (dd, J=1.6, 8.8 Hz, 1H), 4.52 (br s, 1H), 4.28 (dd, J=5.2, 7.2 Hz, 1H), 3.99 (s, 3H), 3.12 (br d, J=6.4 Hz, 2H), 3.01 (ddd, J=4.4, 8.8, 13.2 Hz, 1H), 2.74-2.64 (m, 1H), 2.57-2.48 (m, 1H), 2.42-2.38 (m, 2H), 1.81-1.73 (m, 2H), 1.50-1.29 (m, 20H).

### Step 2: 9-amino-N-[3-(2,6-dioxo-3-piperidyl)-1-methyl-indazol-6-yl]nonanamide

To a solution of tert-butyl N-[9-[[3-(2,6-dioxo-3-piperidyl)-1-methyl-indazol-6-yl]amino]-9-oxo-nonyl]carbamate (130 mg, 253.10 mol, 1 eq) in DCM (1 mL) was added HCl/dioxane (2 M, 1.27 mL, 10 eq). The mixture was stirred at 25° C. for 1 h. LCMS showed starting material was consumed and one main peak with desired mass was detected. The mixture was filtered, and concentrated under pressure to give compound 9-amino-N-[3-(2,6-dioxo-3-piperidyl)-1-methyl-indazol-6-yl]nonanamide (113 mg, 99% yield, HCl) as a white solid.

LC-MS [ESI, M+1]: 414.2.

### Step 3: 8-(azidomethyl)-N-[9-[[3-(2,6-dioxo-3-piperidyl)-1-methyl-indazol-6-yl]amino]-9-oxo-nonyl]-6-[(3R)-3-methyl-3-phenyl-pyrrolidin-1-yl]-[1,2,4]triazolo[1,5-a]pyridine-2-carboxamide

To a solution of 9-amino-N-[3-(2,6-dioxo-3-piperidyl)-1-methyl-indazol-6-yl]nonanamide (70 mg, 155.56 mol, 1 eq, HCl) and 8-(azidomethyl)-6-[(3R)-3-methyl-3-phenyl-pyrrolidin-1-yl]-[1,2,4]triazolo[1,5-a]pyridine-2-carboxylic acid (58.71 mg, 155.56 mol, 1 eq) in DMF (1 mL) was added HATU (88.73 mg, 233.35 mol, 1.5 eq) and DIEA (60.32 mg, 466.69 mol, 81.29 μL, 3 eq). The mixture was stirred at 25° C. for 0.5 h. LCMS showed starting material was consumed and one main peak with desired mass was detected. The mixture was quenched with H₂O (10 mL) and extracted with EtOAc (10 mL×3), the organic phase was dried, filtered, and concentrated under pressure to give the product. The residue was purified by flash silica gel chromatography (ISCO®; 12 g SepaFlash® Silica Flash Column, Eluent of 0˜80% EtOAc/PE gradient) to give compound 8-(azidomethyl)-N-[9-[[3-(2,6-dioxo-3-piperidyl)-1-methyl-indazol-6-yl]amino]-9-oxo-nonyl]-6-[(3R)-3-methyl-3-phenyl-pyrrolidin-1-yl]-[1,2,4]triazolo[1,5-a]pyridine-2-carboxamide (0.13 g, 33% yield) as a brown solid.

¹H NMR (400 MHz, DMSO-d₆) δ=10.88 (s, 1H), 10.06 (s, 1H), 8.47 (s, 1H), 8.29 (br d, J=5.2 Hz, 1H), 8.10-7.95 (m, 2H), 7.61 (d, J=8.8 Hz, 1H), 7.54 (s, 1H), 7.42-7.36 (m, 3H), 7.26 (br d, J=7.2 Hz, 1H), 7.08 (br d, J=8.4 Hz, 1H), 4.81 (s, 2H), 4.31 (dd, J=5.2, 9.6 Hz, 1H), 3.91 (s, 3H), 3.64-3.58 (m, 3H), 3.55 (br s, 1H), 3.31-3.25 (m, 2H), 3.16-3.13 (m, 2H), 2.65 (br d, J=19.2 Hz, 2H), 2.37-2.29 (m, 4H), 1.62 (br s, 4H), 1.38 (s, 3H), 1.32-1.28 (m, 8H).

### Step 4: 8-(aminomethyl)-N-[9-[[3-(2,6-dioxo-3-piperidyl)-1-methyl-indazol-6-yl]amino]-9-oxo-nonyl]-6-[(3R)-3-methyl-3-phenyl-pyrrolidin-1-yl]-[1,2,4]triazolo[1,5-a]pyridine-2-carboxamide

To a solution of 8-(azidomethyl)-N-[9-[[3-(2,6-dioxo-3-piperidyl)-1-methyl-indazol-6-yl]amino]-9-oxo-nonyl]-6-[(3R)-3-methyl-3-phenyl-pyrrolidin-1-yl]-[1,2,4]triazolo[1,5-a]pyridine-2-carboxamide (78 mg, 100.92 mol, 1 eq) in THE (2 mL) was added Pd/C (10.74 mg, 10.09 mol, 10% purity, 0.1 eq). The suspension was degassed and purged with H₂ for 3 times. The mixture was stirred under H₂ (15 Psi) at 25° C. for 1 h. LCMS showed starting material was consumed and one main peak with desired mass was detected. The mixture was filtered, and concentrated under pressure to give the product. The residue was purified by prep-HPLC (column: CD07-Daisogel SP-100-8-ODS-PK 150*25*10 um; mobile phase: [water(NH₄HCO₃)-ACN]; gradient:32%-62% B over 10 min) to give compound 8-(aminomethyl)-N-[9-[[3-(2,6-dioxo-3-piperidyl)-1-methyl-indazol-6-yl]amino]-9-oxo-nonyl]-6-[(3R)-3-methyl-3-phenyl-pyrrolidin-1-yl]-[1,2,4]triazolo[1,5-a]pyridine-2-carboxamide (11.16 mg, 15% yield) as yellow solid.

LC-MS [ESI, M+1]: 747.4.

¹H NMR (400 MHz, DMSO-d₆) δ=11.17-10.62 (m, 1H), 10.08 (s, 1H), 8.51 (t, J=5.6 Hz, 1H), 8.09 (s, 1H), 7.90 (d, J=1.6 Hz, 1H), 7.61 (d, J=8.8 Hz, 1H), 7.51-7.33 (m, 5H), 7.30-7.19 (m, 1H), 7.09 (dd, J=1.6, 8.8 Hz, 1H), 4.31 (dd, J=5.2, 9.6 Hz, 1H), 4.03 (s, 2H), 3.91 (s, 3H), 3.67-3.50 (m, 3H), 3.39 (br d, J=15.6 Hz, 3H), 3.27 (br d, J=6.8 Hz, 2H), 2.63 (br dd, J=5.6, 8.8 Hz, 2H), 2.34 (br d, J=7.2 Hz, 3H), 2.28 (br t, J=7.6 Hz, 2H), 2.19 (br d, J=5.6 Hz, 1H), 1.62 (br s, 2H), 1.54 (br s, 2H), 1.41-1.28 (m, 11H).

### Example 3

### 8-(aminomethyl)-N-[7-[[3-(2,6-dioxo-3-piperidyl)-1-methyl-indazol-6-yl]amino]-7-oxo-heptyl]-6-[(3R)-3-methyl-3-phenyl-pyrrolidin-1-yl]-[1,2,4]triazolo[1,5-a]pyridine-2-carboxamide

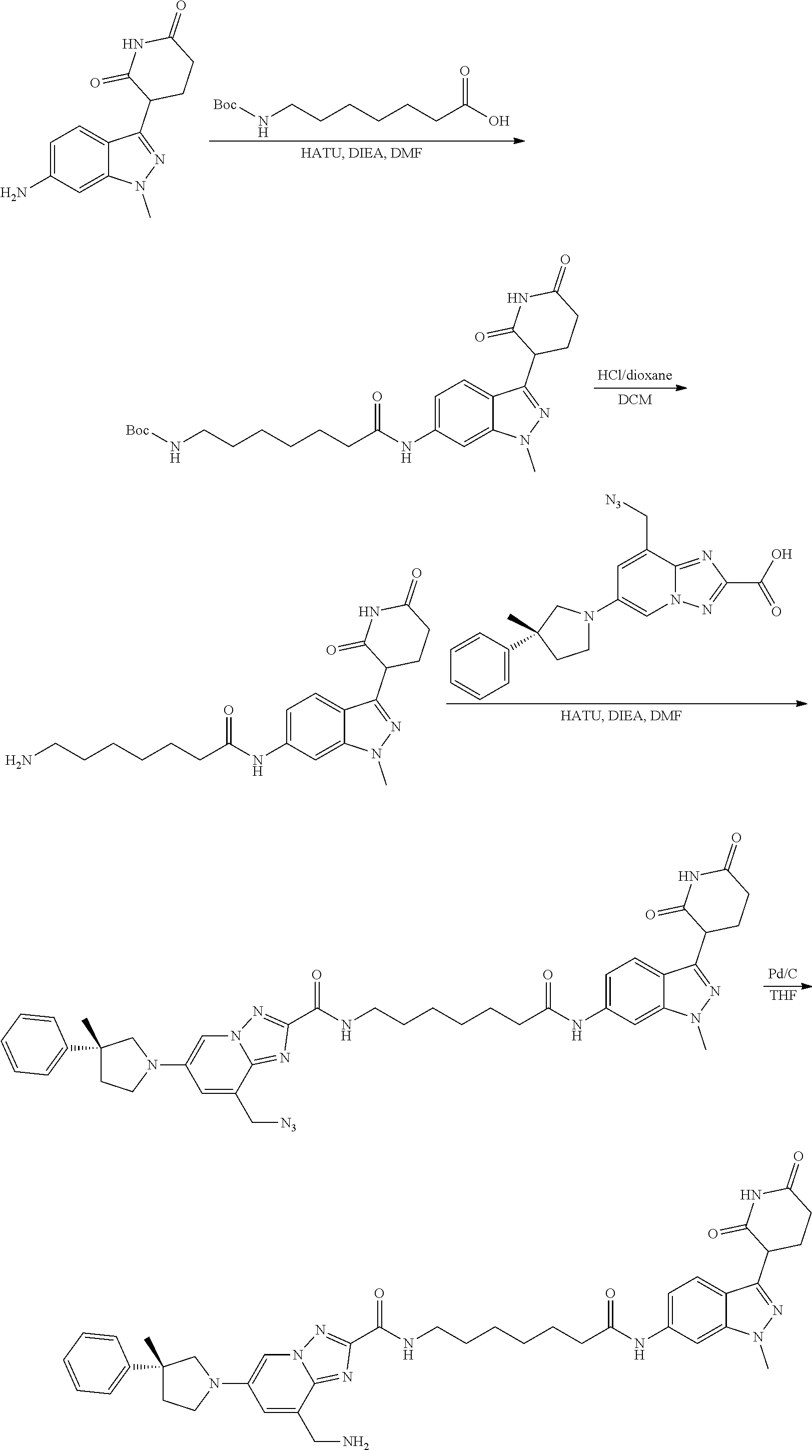

### Step 1: tert-butyl N-[7-[[3-(2,6-dioxo-3-piperidyl)-1-methyl-indazol-6-yl]amino]-7-oxo-heptyl]carbamate

To a solution of 3-(6-amino-1-methyl-indazol-3-yl)piperidine-2,6-dione (200 mg, 774.37 mol, 1 eq) and 7-(tertbutoxycarbonylamino)heptanoic acid (189.96 mg, 774.37 mol, 1 eq) in DMF (1.5 mL) was added HATU (441.66 mg, 1.16 mmol, 1.5 eq) and DIEA (300.24 mg, 2.32 mmol, 404.64 μL, 3 eq). The mixture was stirred at 25° C. for 0.5 h. LCMS showed starting material was consumed and one main peak with desired mass was detected. The mixture was quenched with H₂O (10 mL) and extracted with EtOAc (15 mL×3), the organic phase was dried, filtered, and concentrated under pressure to give the product. The residue was purified by flash silica gel chromatography (ISCO®; 20 g SepaFlash® Silica Flash Column, Eluent of 0˜70% EtOAc/PE gradient) to give compound tert-butyl N-[7-[[3-(2,6-dioxo-3-piperidyl)-1-methyl-indazol-6-yl]amino]-7-oxo-heptyl]carbamate (0.14 g, 37% yield) as a brown solid.

¹H NMR (400 MHz, CHLOROFORM-d) 6=8.23 (s, 1H), 7.94 (br s, 1H), 7.79 (br s, 1H), 7.58 (d, J=8.4 Hz, 1H), 6.86 (br d, J=8.4 Hz, 1H), 4.55 (br d, J=1.6 Hz, 1H), 4.28 (dd, J=5.2, 7.2 Hz, 1H), 3.99 (s, 3H), 3.14 (br d, J=6.0 Hz, 2H), 3.02 (ddd, J=5.2, 8.4, 17.6 Hz, 1H), 2.74-2.64 (m, 1H), 2.57-2.47 (m, 1H), 2.42-2.38 (m, 2H), 1.78 (br t, J=7.2 Hz, 2H), 1.52-1.36 (m, 16H).

### Step 2: 7-amino-N-[3-(2,6-dioxo-3-piperidyl)-1-methyl-indazol-6-yl]heptanamide

To a solution of tert-butyl N-[7-[[3-(2,6-dioxo-3-piperidyl)-1-methyl-indazol-6-yl]amino]-7-oxo-heptyl]carbamate (140 mg, 288.32 mol, 1 eq) in DCM (1 mL) was added HCl/dioxane (2 M, 1.44 mL, 10 eq). The mixture was stirred at 25° C. for 1 h. LCMS showed starting material was consumed and one main peak with desired mass was detected. The mixture was filtered, and concentrated under pressure to give compound 7-amino-N-[3-(2,6-dioxo-3-piperidyl)-1-methyl-indazol-6-yl]heptanamide (120 mg, 99% yield, HCl) as a brown solid.

LC-MS [ESI, M+1]: 386.2.

### Step 3: 8-(azidomethyl)-N-[7-[[3-(2,6-dioxo-3-piperidyl)-1-methyl-indazol-6-yl]amino]-7-oxo-heptyl]-6-[(3R)-3-methyl-3-phenyl-pyrrolidin-1-yl]-[1,2,4]triazolo[1,5-a]pyridine-2-carboxamide

To a solution of 7-amino-N-[3-(2,6-dioxo-3-piperidyl)-1-methyl-indazol-6-yl]heptanamide (70 mg, 165.91 mol, 1 eq, HCl) and 8-(azidomethyl)-6-[(3R)-3-methyl-3-phenyl-pyrrolidin-1-yl]-[1,2,4]triazolo[1,5-a]pyridine-2-carboxylic acid (62.61 mg, 165.91 mol, 1 eq) in DMF (1 mL) was added HATU (94.62 mg, 248.86 mol, 1.5 eq) and DIEA (64.33 mg, 497.72 mol, 86.69 μL, 3 eq). The mixture was stirred at 25° C. for 0.5 h. LCMS showed starting material was consumed and one main peak with desired mass was detected. The mixture was quenched with H₂O (10 mL) and extracted with EtOAc (10 mL×3), the organic phase was dried, filtered, and concentrated under pressure to give the product. The residue was purified by flash silica gel chromatography (ISCO®; 12 g SepaFlash® Silica Flash Column, Eluent of 0˜75% EtOAc/PE gradient) to give compound 8-(azidomethyl)-N-[7-[[3-(2,6-dioxo-3-piperidyl)-1-methyl-indazol-6-yl]amino]-7-oxo-heptyl]-6-[(3R)-3-methyl-3-phenyl-pyrrolidin-1-yl]-[1,2,4]triazolo[1,5-a]pyridine-2-carboxamide (68 mg, 55% yield) as a brown solid.

¹H NMR (400 MHz, DMSO-d₆) δ=10.87 (s, 1H), 10.06 (s, 1H), 8.47 (s, 1H), 8.13-8.01 (m, 2H), 7.60 (d, J=8.4 Hz, 1H), 7.54 (d, J=1.6 Hz, 1H), 7.42-7.34 (m, 4H), 7.25 (br d, J=7.2 Hz, 1H), 7.08 (dd, J=1.2, 8.8 Hz, 1H), 4.80 (s, 2H), 4.30 (br d, J=4.4 Hz, 1H), 3.90 (s, 3H), 3.64-3.52 (m, 3H), 3.42-3.37 (m, 1H), 3.29 (br d, J=6.4 Hz, 2H), 2.62 (br d, J=2.4 Hz, 2H), 2.38-2.33 (m, 2H), 2.28 (br s, 2H), 1.62 (br d, J=7.2 Hz, 2H), 1.55 (br s, 2H), 1.41-1.32 (m, 7H), 1.26 (br t, J=6.4 Hz, 2H).

### Step 4: 8-(aminomethyl)-N-[7-[[3-(2,6-dioxo-3-piperidyl)-1-methyl-indazol-6-yl]amino]-7-oxo-heptyl]-6-[(3R)-3-methyl-3-phenyl-pyrrolidin-1-yl]-[1,2,4]triazolo[1,5-a]pyridine-2-carboxamide

To a solution of 8-(azidomethyl)-N-[7-[[3-(2,6-dioxo-3-piperidyl)-1-methyl-indazol-6-yl]amino]-7-oxo-heptyl]-6-[(3R)-3-methyl-3-phenyl-pyrrolidin-1-yl]-[1,2,4]triazolo[1,5-a]pyridine-2-carboxamide (50 mg, 67.13 mol, 1 eq) in THE (2 mL) was added Pd/C (7.14 mg, 6.71 mol, 10% purity, 0.1 eq). The suspension was degassed and purged with H₂ for 3 times. The mixture was stirred under H₂ (15 Psi) at 25° C. for 1 h. LCMS showed starting material was consumed and one main peak with desired mass was detected. The mixture was filtered, and concentrated under pressure to give the product. The residue was purified by prep-HPLC(column: CD07-Daisogel SP-100-8-ODS-PK 150*25*10 um; mobile phase: [water(NH₄HCO₃)-ACN]; gradient:26%-56% B over 10 min) to give compound 8-(aminomethyl)-N-[7-[[3-(2,6-dioxo-3-piperidyl)-1-methyl-indazol-6-yl]amino]-7-oxo-heptyl]-6-[(3R)-3-methyl-3-phenyl-pyrrolidin-1-yl]-[1,2,4]triazolo[1,5-a]pyridine-2-carboxamide (12.74 mg, 27% yield) as yellow solid.

LC-MS [ESI, M+1]: 719.4.

¹H NMR (400 MHz, DMSO-d₆) δ=10.87 (br s, 1H), 10.07 (s, 1H), 8.52 (br t, J=6.0 Hz, 1H), 8.08 (s, 1H), 7.89 (d, J=1.6 Hz, 1H), 7.60 (d, J=8.8 Hz, 1H), 7.47-7.32 (m, 5H), 7.24 (br d, J=7.2 Hz, 1H), 7.08 (dd, J=1.6, 8.8 Hz, 1H), 4.30 (dd, J=5.2, 9.6 Hz, 1H), 4.03 (s, 2H), 3.90 (s, 3H), 3.65-3.46 (m, 4H), 3.39 (br d, J=8.0 Hz, 2H), 3.28-3.24 (m, 2H), 2.62 (br dd, J=5.6, 8.4 Hz, 2H), 2.35 (br t, J=7.2 Hz, 3H), 2.30-2.24 (m, 2H), 2.21-2.13 (m, 1H), 1.66-1.59 (m, 2H), 1.55 (br s, 2H), 1.41-1.30 (m, 7H).

### Example 4

### 8-(aminomethyl)-N-[5-[[3-(2,6-dioxo-3-piperidyl)-1-methyl-indazol-6-yl]amino]-5-oxo-pentyl]-6-[(3R)-3-methyl-3-phenyl-pyrrolidin-1-yl]-[1,2,4]triazolo[1,5-a]pyridine-2-carboxamide

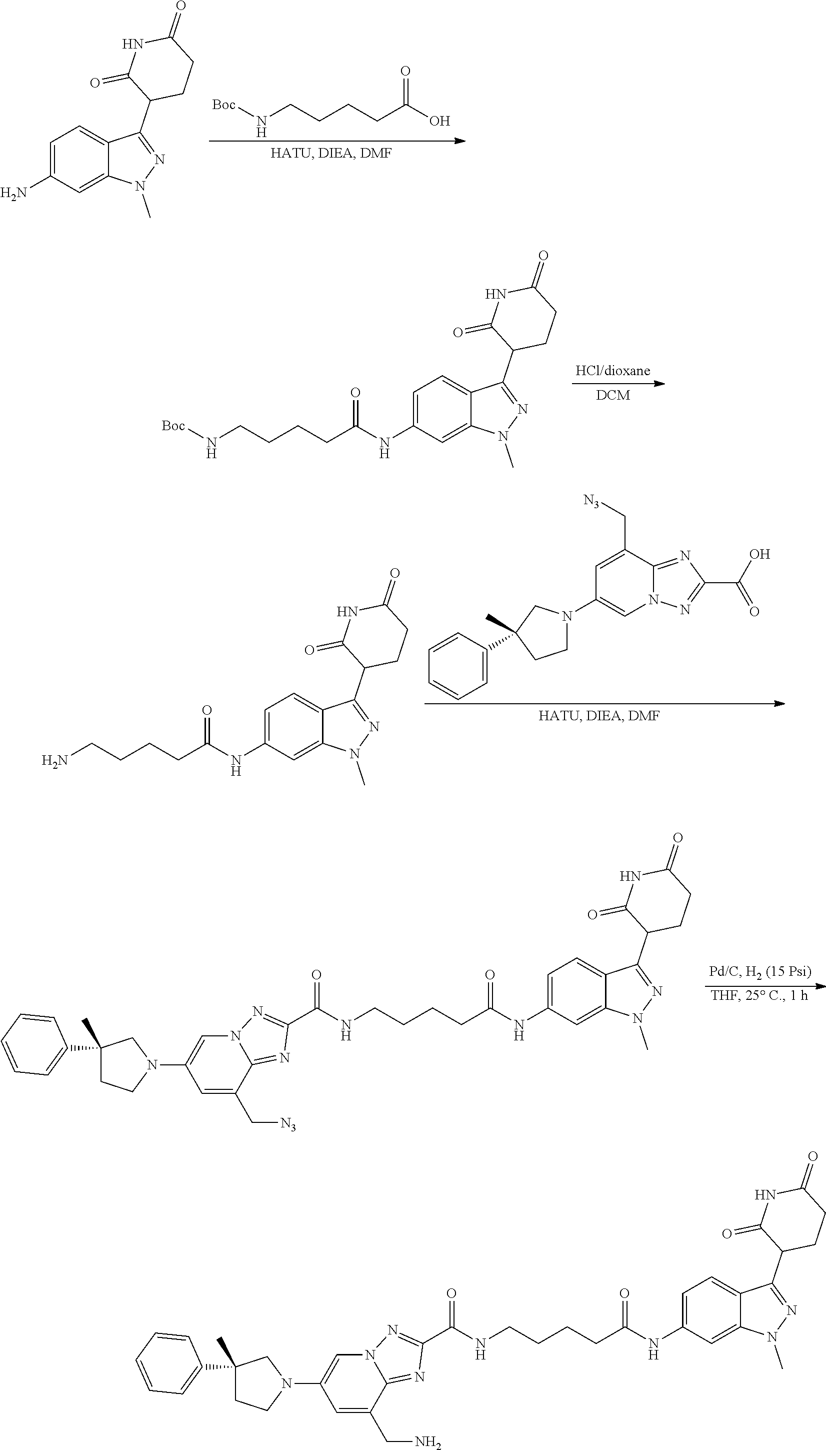

### Step 1: tert-butyl N-[5-[[3-(2,6-dioxo-3-piperidyl)-1-methyl-indazol-6-yl]amino]-5-oxo-pentyl]carbamate

To a solution of 3-(6-amino-1-methyl-indazol-3-yl)piperidine-2,6-dione (200 mg, 774.37 mol, 1 eq) and 5-(tertbutoxycarbonylamino) pentanoic acid (168.24 mg, 774.37 mol, 1 eq) in DMF (2 mL) was added HATU (441.66 mg, 1.16 mmol, 1.5 eq) and DIEA (300.24 mg, 2.32 mmol, 404.64 μL, 3 eq). The mixture was stirred at 25° C. for 2 h. LCMS showed starting material was consumed and the desired mass was detected. The reaction mixture was filtered and the filter was concentrated under reduced pressure to give a residue. The crude product was purified by reversed-phase HPLC (0.1% FA condition) to give compound tert-butyl N-[5-[[3-(2,6-dioxo-3-piperidyl)-1-methyl-indazol-6-yl]amino]-5-oxo-pentyl]carbamate (120 mg, 34% yield) as a white solid.

LC-MS [ESI, M+1]: 458.1

¹H NMR (400 MHz, DMSO-d₆) δ=10.88 (s, 1H), 10.06 (s, 1H), 8.08 (s, 1H), 7.61 (d, J=8.8 Hz, 1H), 7.08 (dd, J=1.6, 8.8 Hz, 1H), 6.81 (br t, J=5.6 Hz, 1H), 4.31 (dd, J=5.2, 9.6 Hz, 1H), 3.91 (s, 3H), 2.94 (q, J=6.8 Hz, 2H), 2.71-2.58 (m, 2H), 2.54 (s, 1H), 2.39-2.27 (m, 3H), 1.99 (s, 1H), 1.63-1.52 (m, 2H), 1.37 (s, 9H)

### Step 2: 5-amino-N-[3-(2,6-dioxo-3-piperidyl)-1-methyl-indazol-6-yl]pentanamide

To a solution of tert-butyl N-[5-[[3-(2,6-dioxo-3-piperidyl)-1-methyl-indazol-6-yl]amino]-5-oxo-pentyl]carbamate (120 mg, 262.28 mol, 1 eq) in DCM (1 mL) was added HCl/dioxane (2 M, 1.31 mL, 10 eq). The mixture was stirred at 25° C. for 16 h. LCMS showed starting material was consumed and the desired mass was detected. The reaction was filtered and the filter was concentrated under reduced pressure to give compound 5-amino-N-[3-(2,6-dioxo-3-piperidyl)-1-methyl-indazol-6-yl]pentanamide (100 mg, 97% yield, HCl salt) as a white solid.

LC-MS [ESI, M+1]: 358.0

### Step 3: 8-(azidomethyl)-N-[5-[[3-(2,6-dioxo-3-piperidyl)-1-methyl-indazol-6-yl]amino]-5-oxo-pentyl]-6-[(3R)-3-methyl-3-phenyl-pyrrolidin-1-yl]-[1,2,4]triazolo[1,5-a]pyridine-2-carboxamide

To a solution of 5-amino-N-[3-(2,6-dioxo-3-piperidyl)-1-methyl-indazol-6-yl]pentanamide (87.67 mg, 222.58 mol, 1.2 eq, HCl) and 8-(azidomethyl)-6-[(3R)-3-methyl-3-phenyl-pyrrolidin-1-yl]-[1,2,4]triazolo[1,5-a]pyridine-2-carboxylic acid (70 mg, 185.48 mol, 1 eq) in DMF (1 mL) was added DIEA (71.92 mg, 556.44 mol, 96.92 μL, 3 eq) and HATU (105.79 mg, 278.22 mol, 1.5 eq). The mixture was stirred at 25° C. for 2 h. LCMS showed starting material was consumed and the desired mass was detected. The reaction was filtered and the filter was concentrated under reduced pressure to give a residue. The crude product was purified by reversed-phase HPLC(0.1% FA condition) to give compound 8-(azidomethyl)-N-[5-[[3-(2,6-dioxo-3-piperidyl)-1-methyl-indazol-6-yl]amino]-5-oxo-pentyl]-6-[(3R)-3-methyl-3-phenyl-pyrrolidin-1-yl]-[1,2,4]triazolo[1,5-a]pyridine-2-carboxamide (90 mg, 68% yield) was obtained as a white solid.

LC-MS [ESI, M+1]: 717.4

1H NMR (400 MHz, DMSO-d₆) δ=10.87 (s, 1H), 10.09 (s, 1H), 8.54 (t, J=6.0 Hz, 1H), 8.13-8.00 (m, 2H), 7.65-7.49 (m, 2H), 7.44-7.31 (m, 4H), 7.28-7.18 (m, 1H), 7.08 (dd, J=1.6, 8.8 Hz, 1H), 4.80 (s, 2H), 4.30 (dd, J=5.2, 9.6 Hz, 1H), 3.90 (s, 3H), 3.66-3.46 (m, 3H), 2.72-2.57 (m, 2H), 2.54 (s, 2H), 2.40 (br t, J=6.8 Hz, 2H), 2.35-2.24 (m, 3H), 2.22-2.11 (m, 1H), 1.71-1.53 (m, 4H), 1.37 (s, 3H)

### Step 4: 8-(aminomethyl)-N-[5-[[3-(2,6-dioxo-3-piperidyl)-1-methyl-indazol-6-yl]amino]-5-oxo-pentyl]-6-[(3R)-3-methyl-3-phenyl-pyrrolidin-1-yl]-[1,2,4]triazolo[1,5-a]pyridine-2-carboxamide

A mixture of 8-(azidomethyl)-N-[5-[[3-(2,6-dioxo-3-piperidyl)-1-methyl-indazol-6-yl]amino]-5-oxo-pentyl]-6-[(3R)-3-methyl-3-phenyl-pyrrolidin-1-yl]-[1,2,4]triazolo[1,5-a]pyridine-2-carboxamide (50 mg, 69.76 mol, 1 eq) and Pd/C (7.42 mg, 6.98 mol, 10% purity, 0.1 eq) in THE (2 mL) was degassed and purged with H₂ for 3 times, and then the mixture was stirred at 25° C. for 2 h under H₂ atmosphere. LCMS showed starting material was consumed and the desired mass was detected. The reaction was filtered and the filtered concentrated under reduced pressure to give a residue. The residue was purified by prep-HPLC (column: CD02-Waters Xbidge BEH C18 150*25*10 um; mobile phase: [water(NH₄HCO₃)-ACN]; gradient:25%-55% B over 10 min). The fraction was lyophilized to give a residue. The residue was purified by prep-HPLC (column: CD02-Waters Xbidge BEH C18 150*25*10 um; mobile phase: [water(NH₄HCO₃)-ACN]; gradient:22%-52% B over 10 min). The fraction was lyophilized to give compound 8-(aminomethyl)-N-[5-[[3-(2,6-dioxo-3-piperidyl)-1-methyl-indazol-6-yl]amino]-5-oxo-pentyl]-6-[(3R)-3-methyl-3-phenyl-pyrrolidin-1-yl]-[1,2,4]triazolo[1,5-a]pyridine-2-carboxamide (25 mg, 52% yield) was obtained as a white solid.

LC-MS [ESI, M+]: 691.2

¹H NMR (400 MHz, DMSO-d₆) δ=11.37-10.44 (m, 1H), 10.09 (s, 1H), 8.58 (t, J=6.0 Hz, 1H), 8.09 (s, 1H), 7.90 (d, J=1.6 Hz, 1H), 7.60 (d, J=8.8 Hz, 1H), 7.47-7.32 (m, 5H), 7.27-7.20 (m, 1H), 7.08 (dd, J=1.6, 8.8 Hz, 1H), 4.30 (dd, J=5.2, 9.6 Hz, 1H), 4.03 (s, 2H), 3.90 (s, 3H), 3.66-3.59 (m, 1H), 3.58-3.48 (m, 2H), 3.43-3.33 (m, 3H), 2.67-2.58 (m, 2H), 2.40 (br t, J=6.8 Hz, 2H), 2.36-2.23 (m, 3H), 2.22-2.10 (m, 1H), 1.74-1.54 (m, 4H), 1.37 (s, 3H)

### Example 5

### N-[10-[[3-(2,6-dioxo-3-piperidyl)-1-methyl-indazol-7-yl]amino]-10-oxo-decyl]-8-methyl-6-[(3R)-3-methyl-3-phenyl-pyrrolidin-1-yl]-[1,2,4]triazolo[1,5-a]pyridine-2-carboxamide

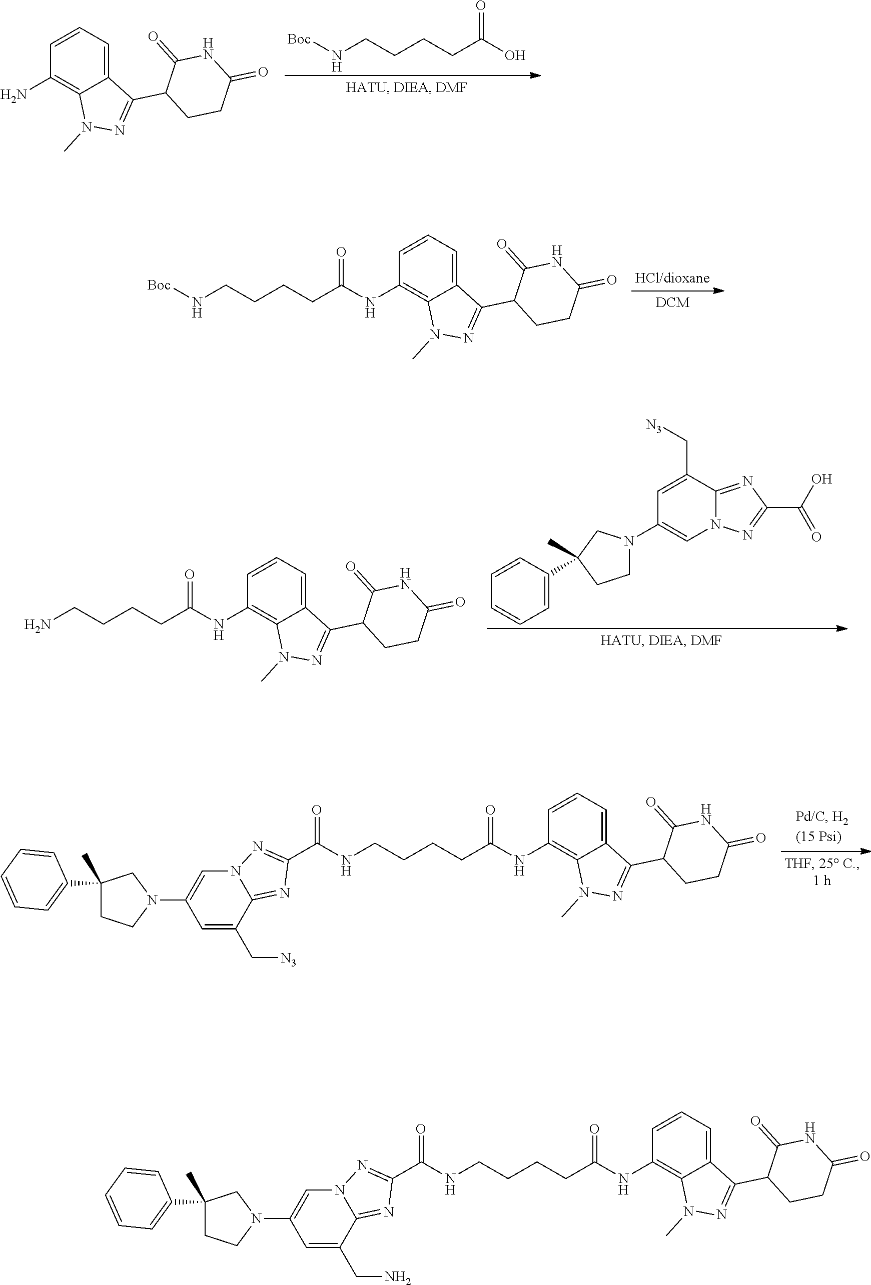

### Step 1: tert-butyl N-[5-[[3-(2,6-dioxo-3-piperidyl)-1-methyl-indazol-7-yl]amino]-5-oxo-pentyl]carbamate

To a solution of 3-(7-amino-1-methyl-indazol-3-yl)piperidine-2,6-dione (350 mg, 1.36 mmol, 1 eq), 5-(tertbutoxycarbonylamino)pentanoic acid (353.30 mg, 1.63 mmol, 1.2 eq) in DMF (4 mL) was added HATU (1.03 g, 2.71 mmol, 2 eq) and DIEA (525.43 mg, 4.07 mmol, 708.12 μL, 3 eq). The mixture was stirred at 25° C. for 0.5 h. LCMS showed starting material was consumed completely and the desired compound was detected. The reaction mixture was extracted with EtOAc (10 mL×2). The combined organic phase was washed with brine (5 mL), dried with anhydrous Na₂SO₄, filtered and concentrated in vacuum. The residue was purified by prep-HPLC (column: CD01-Phenomenex luna C18 150*25*10 um; mobile phase: [water(FA)-ACN]; gradient:15%-45% B over 10 min) and lyophilizated to give tert-butyl N-[5-[[3-(2,6-dioxo-3-piperidyl)-1-methyl-indazol-7-yl]amino]-5-oxo-pentyl]carbamate (420 mg, 68% yield) was obtained as a white solid.

LC-MS [ESI, M+1]:358.2

### Step 2: 5-amino-N-[3-(2,6-dioxo-3-piperidyl)-1-methyl-indazol-7-yl]pentanamide

A solution of tert-butyl N-[5-[[3-(2,6-dioxo-3-piperidyl)-1-methyl-indazol-7-yl]amino]-5-oxo-pentyl]carbamate (420 mg, 917.99 mol, 1 eq) in HCl/dioxane (4M, 10 mL) was stirred at 25° C. for 0.1 h. LCMS showed starting material was consumed completely and the desired compound was detected. The reaction was concentrated under reduced pressure to give compound 5-amino-N-[3-(2,6-dioxo-3-piperidyl)-1-methyl-indazol-7-yl]pentanamide (345 mg, 96% yield) was obtained as a white solid.

LC-MS [ESI, M+1]:358.1

### Step 3: 8-(azidomethyl)-N-[5-[[3-(2,6-dioxo-3-piperidyl)-1-methyl-indazol-7-yl]amino]-5-oxo-pentyl]-6-[(3R)-3-methyl-3-phenyl-pyrrolidin-1-yl]-[1,2,4]triazolo[1,5-a]pyridine-2-carboxamide

To a solution of 5-amino-N-[3-(2,6-dioxo-3-piperidyl)-1-methyl-indazol-7-yl]pentanamide (80 mg, 223.84 mol, 1 eq), 8-(azidomethyl)-6-[(3R)-3-methyl-3-phenyl-pyrrolidin-1-yl]-[1,2,4]triazolo[1,5-a]pyridine-2-carboxylic acid (84.48 mg, 223.84 mol, 1 eq) in DMF (2 mL) was added HATU (212.77 mg, 559.59 mol, 2.5 eq) and DIEA (86.79 mg, 671.51 mol, 116.96 μL, 3 eq). The mixture was stirred at 25° C. for 1 h. LCMS showed starting material was consumed completely and the desired compound was detected. The reaction was concentrated under reduced pressure to give a residue. The residue was purified by prep-HPLC (column: CD20-Waters Xbidge BEH C18 250*25*10 um; mobile phase: [water(NH₄HCO₃)-ACN]; gradient:34%-64% B over 15 min) and lyophilizated to give compound 8-(azidomethyl)-N-[5-[[3-(2,6-dioxo-3-piperidyl)-1-methyl-indazol-7-yl]amino]-5-oxo-pentyl]-6-[(3R)-3-methyl-3-phenyl-pyrrolidin-1-yl]-[1,2,4]triazolo[1,5-a]pyridine-2-carboxamide (20 mg, 12% yield) was obtained as a white solid.

LC-MS [ESI, M+1]:717.3

### Step 4: 8-(aminomethyl)-N-[5-[[3-(2,6-dioxo-3-piperidyl)-1-methyl-indazol-7-yl]amino]-5-oxo-pentyl]-6-[(3R)-3-methyl-3-phenyl-pyrrolidin-1-yl]-[1,2,4]triazolo[1,5-a]pyridine-2-carboxamide

To a solution of 8-(azidomethyl)-N-[5-[[3-(2,6-dioxo-3-piperidyl)-1-methyl-indazol-7-yl]amino]-5-oxo-pentyl]-6-[(3R)-3-methyl-3-phenyl-pyrrolidin-1-yl]-[1,2,4]triazolo[1,5-a]pyridine-2-carboxamide (20 mg, 27.90 mol, 1 eq) in THE (2 mL) was added Pd/C (2.97 mg, 2.79 mol, 10% purity, 0.1 eq) under N₂ atmosphere. The suspension was degassed and purged with H₂ for 3 times. The mixture was stirred under H₂ (15 Psi) at 25° C. for 1 h. LCMS showed starting material was consumed completely and the desired compound was detected. The reaction mixture was filtered. The reaction was concentrated under reduced pressure to give a residue. The residue was purified by prep-HPLC (column: CD02-Waters Xbidge BEH C18 150*25*10 um; mobile phase: [water(NH₃H₂O)-ACN]; gradient:25%-55% B over 10 min) and lyophilizated to give compound 8-(aminomethyl)-N-[5-[[3-(2,6-dioxo-3-piperidyl)-1-methyl-indazol-7-yl]amino]-5-oxo-pentyl]-6-[(3R)-3-methyl-3-phenyl-pyrrolidin-1-yl]-[1,2,4]triazolo[1,5-a]pyridine-2-carboxamide (1.78 mg, 9% yield).

LC-MS [ESI, M+1]:691.4

¹H NMR (400 MHz, DMSO-d₆) δ=11.07-10.75 (m, 1H), 9.85 (s, 1H), 8.60 (t, J=6 Hz, 1H), 7.91 (s, 1H), 7.59 (d, J=8.0 Hz, 1H), 7.48-7.32 (m, 5H), 7.29-7.20 (m, 1H), 7.15-6.98 (m, 2H), 4.37 (dd, J=5.2, 10.0 Hz, 1H), 4.03 (s, 5H), 3.68-3.45 (m, 6H), 2.69-2.62 (m, 2H), 2.45 (br t, J=7.2 Hz, 2H), 2.34 (d, J=4.8 Hz, 1H), 2.31-2.24 (m, 2H), 2.20-2.12 (m, 1H), 1.77-1.58 (m, 4H), 1.37 (s, 3H)

### Example 6

### 8-(aminomethyl)-N-(6-((3-(2,6-dioxopiperidin-3-yl)-1-methyl-1H-indazol-6-yl)amino)-6-oxohexyl)-6-((R)-3-methyl-3-phenylpyrrolidin-1-yl)-[1,2,4]triazolo[1,5-a]pyridine-2-carboxamide

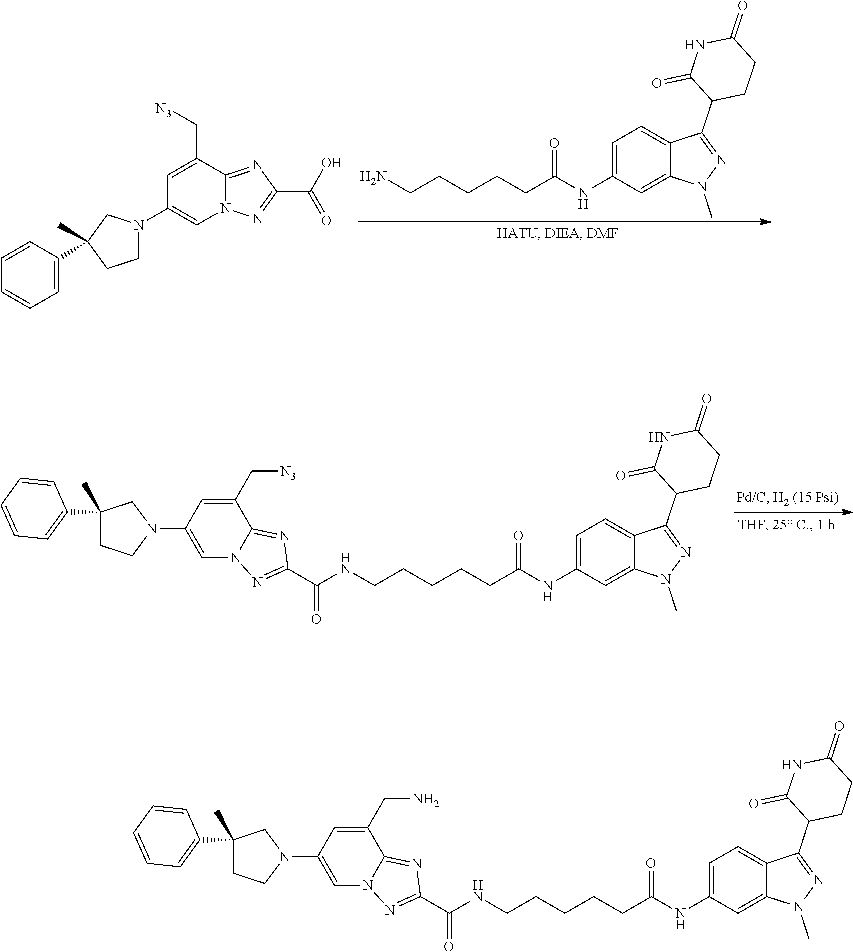 [SMILES: C[C@]1(c2ccccc2)CCN(c2cc(CN=[N+]=[N-])c3nc(C(=O)O)nn3c2)C1 ; Cn1nc(C2CCC(=O)NC2=O)c2ccc(NC(=O)CCCCCN)cc21 ; Cn1nc(C2CCC(=O)NC2=O)c2ccc(NC(=O)CCCCCNC(=O)c3nc4c(CN=[N+]=[N-])cc(N5CC[C@](C)(c6ccccc6)C5)cn4n3)cc21 ; "THF, 25° C., 1 h" ; Cn1nc(C2CCC(=O)NC2=O)c2ccc(NC(=O)CCCCCNC(=O)c3nc4c(CN)cc(N5CC[C@](C)(c6ccccc6)C5)cn4n3)cc21]

### Step 1: 8-(azidomethyl)-N-(6-((3-(2,6-dioxopiperidin-3-yl)-1-methyl-1H-indazol-6-yl)amino)-6-oxohexyl)-6-((R)-3-methyl-3-phenylpyrrolidin-1-yl)-[1,2,4]triazolo[1,5-a]pyridine-2-carboxamide

To a solution of 8-(azidomethyl)-6-[(3R)-3-methyl-3-phenyl-pyrrolidin-1-yl]-[1,2,4]triazolo[1,5-a]pyridine-2-carboxylic acid (80 mg, 211.98 mol, 1 eq) and 6-amino-N-[3-(2,6-dioxo-3-piperidyl)-1-methyl-indazol-6-yl]hexanamide (78.74 mg, 211.98 mol, 1 eq) in DMF (1 mL) was added HATU (161.20 mg, 423.95 mol, 2 eq) and then was added DIEA (82.19 mg, 635.93 mol, 110.77 μL, 3 eq) after 0.1 h. The mixture was stirred at 25° C. for 1 h. LCMS showed starting material was consumed completely. The reaction mixture was added water, then was extracted with ethyl acetate (30 mL×3). The combined organic phase was washed with brine (30 mL), dried with anhydrous Na₂SO₄, filtered and concentrated in vacuum to give the crude 8-(azidomethyl)-N-(6-((3-(2,6-dioxopiperidin-3-yl)-1-methyl-1H-indazol-6-yl)amino)-6-oxohexyl)-6-((R)-3-methyl-3-phenylpyrrolidin-1-yl)-[1,2,4]triazolo[1,5-a]pyridine-2-carboxamide (140 mg, 90% yield) as a yellow solid.

LC-MS [ESI, M+1]: 787.92

### Step 2: 8-(aminomethyl)-N-(6-((3-(2,6-dioxopiperidin-3-yl)-1-methyl-1H-indazol-6-yl)amino)-6-oxohexyl)-6-((R)-3-methyl-3-phenylpyrrolidin-1-yl)-[1,2,4]triazolo[1,5-a]pyridine-2-carboxamide

To a solution of 8-(azidomethyl)-N-[6-[[3-(2,6-dioxo-3-piperidyl)-1-methyl-indazol-6-yl]amino]-6-oxo-hexyl]-6-[(3R)-3-methyl-3-phenyl-pyrrolidin-1-yl]-[1,2,4]triazolo[1,5-a]pyridine-2-carboxamide (140 mg, 191.57 mol, 1 eq) in THE (2 mL) was added Pd/C (20.39 mg, 19.16 mol, 10% purity, 0.1 eq). The mixture was stirred at 25° C. for 2 h under H₂ (15 Psi). LCMS showed starting material was consumed completely and one main peak with desired mass was detected. The reaction mixture was filtered and the filtrate was concentrated under the reduced pressure to give the product. The residue was purified by prep-HPLC (column: CD02-Waters Xbidge BEH C18 150*25*10 um; mobile phase: [water(HCl)-ACN]; gradient:28%-58% B over 10 min) and lyophilized to give the 8-(aminomethyl)-N-(6-((3-(2,6-dioxopiperidin-3-yl)-1-methyl-1H-indazol-6-yl)amino)-6-oxohexyl)-6-((R)-3-methyl-3-phenylpyrrolidin-1-yl)-[1,2,4]triazolo[1,5-a]pyridine-2-carboxamide (24.5 mg, 16% yield, FA) as a brown solid

LC-MS [ESI, M+1]:784.4

¹H NMR (400 MHz, DMSO-d₆) δ=10.87 (s, 1H), 10.25 (s, 1H), 8.89 (br s, 3H), 8.78 (br t, J=6.0 Hz, 1H), 8.07 (d, J=11.6 Hz, 2H), 7.82 (s, 1H), 7.59 (d, J=8.8 Hz, 1H), 7.42-7.31 (m, 4H), 7.28-7.20 (m, 1H), 7.13 (dd, J=1.2, 8.8 Hz, 1H), 4.38 (br d, J=5.6 Hz, 2H), 4.30 (dd, J=5.2, 9.6 Hz, 1H), 3.89 (s, 3H), 3.65-3.59 (m, 1H), 3.58-3.47 (m, 2H), 3.44-3.36 (m, 1H), 3.30 (q, J=6.8 Hz, 2H), 2.72-2.53 (m, 2H), 2.38 (br t, J=7.2 Hz, 2H), 2.35-2.23 (m, 3H), 2.16 (br dd, J=5.6, 13.6 Hz, 1H), 1.70-1.53 (m, 4H), 1.42-1.30 (m, 5H)

### Example 7

### 8-(aminomethyl)-N-(10-((3-(2,6-dioxopiperidin-3-yl)-1-methyl-1H-indazol-7-yl)amino)-10-oxodecyl)-6-((R)-3-methyl-3-phenylpyrrolidin-1-yl)-[1,2,4]triazolo[1,5-a]pyridine-2-carboxamide

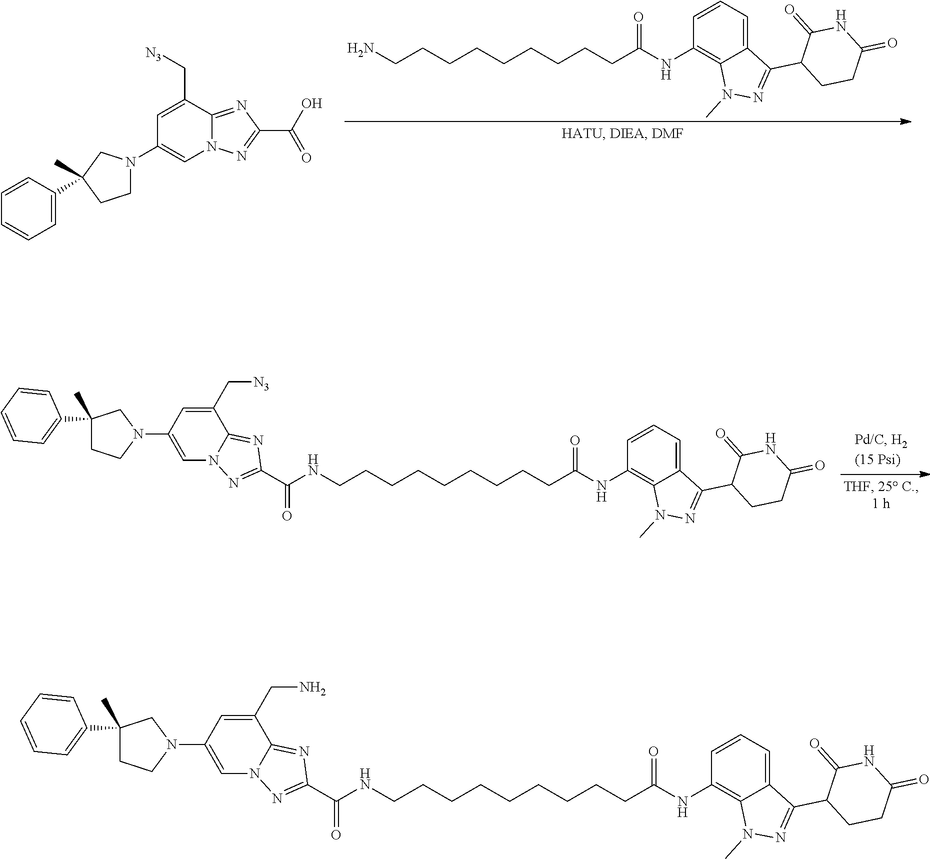 [SMILES: C[C@]1(c2ccccc2)CCN(c2cc(CN=[N+]=[N-])c3nc(C(=O)O)nn3c2)C1 ; Cn1nc(C2CCC(=O)NC2=O)c2cccc(NC(=O)CCCCCCCCCN)c21 ; Cn1nc(C2CCC(=O)NC2=O)c2cccc(NC(=O)CCCCCCCCCNC(=O)c3nc4c(CN=[N+]=[N-])cc(N5CC[C@](C)(c6ccccc6)C5)cn4n3)c21 ; Cn1nc(C2CCC(=O)NC2=O)c2cccc(NC(=O)CCCCCCCCCNC(=O)c3nc4c(CN)cc(N5CC[C@](C)(c6ccccc6)C5)cn4n3)c21]

### Step 1: 8-(azidomethyl)-N-(10-((3-(2,6-dioxopiperidin-3-yl)-1-methyl-1H-indazol-7-yl)amino)-10-oxodecyl)-6-((R)-3-methyl-3-phenylpyrrolidin-1-yl)-[1,2,4]triazolo[1,5-a]pyridine-2-carboxamide

To a solution of 8-(azidomethyl)-6-[(3R)-3-methyl-3-phenyl-pyrrolidin-1-yl]-[1,2,4]triazolo[1,5-a]pyridine-2-carboxylic acid (60 mg, 158.98 mol, 1 eq) in DMF (2 mL) was added 10-amino-N-[3-(2,6-dioxo-3-piperidyl)-1-methyl-indazol-7-yl]decanamide (88.52 mg, 190.78 mol, 1.2 eq, HCl), HATU (72.54 mg, 190.78 mol, 1.2 eq) and DIEA (61.64 mg, 476.95 mol, 83.08 μL, 3 eq). The mixture was stirred at 25° C. for 12 h. LCMS showed starting material was consumed and one main peak with desired mass was detected. The reaction mixture was concentrated under the reduced pressure to give the crude product. The residue was purified by prep-HPLC (FA condition) to give compound 8-(azidomethyl)-N-[10-[[3-(2,6-dioxo-3-piperidyl)-1-methyl-indazol-7-yl]amino]-10-oxo-decyl]-6-[(3R)-3-methyl-3-phenyl-pyrrolidin-1-yl]-[1,2,4]triazolo[1,5-a]pyridine-2-carboxamide (95 mg, 75.93% yield) was obtained as a yellow solid.

LC-MS [ESI, M+1]: 787.2.

¹H NMR (400 MHz, CHLOROFORM-d) 6=8.36-8.19 (m, 1H), 8.02-7.86 (m, 1H), 7.53 (br d, J=7.6 Hz, 1H), 7.44-7.36 (m, 3H), 7.35-7.31 (m, 2H), 7.29 (br d, J=7.2 Hz, 2H), 7.18-7.06 (m, 2H), 4.69 (s, 2H), 4.25-4.11 (m, 4H), 3.63-3.34 (m, 7H), 3.05-2.88 (m, 1H), 2.72-2.59 (m, 1H), 2.54 (br t, J=7.6 Hz, 2H), 2.49-2.20 (m, 5H), 1.92-1.75 (m, 2H), 1.67 (br d, J=6.4 Hz, 3H), 1.47 (s, 3H), 1.39 (br d, J=10.4 Hz, 8H)

### Step 2: 8-(aminomethyl)-N-(10-((3-(2,6-dioxopiperidin-3-yl)-1-methyl-1H-indazol-7-yl)amino)-10-oxodecyl)-6-((R)-3-methyl-3-phenylpyrrolidin-1-yl)-[1,2,4]triazolo[1,5-a]pyridine-2-carboxamide

To a solution of 8-(azidomethyl)-N-[10-[[3-(2,6-dioxo-3-piperidyl)-1-methyl-indazol-7-yl]amino]-10-oxo-decyl]-6-[(3R)-3-methyl-3-phenyl-pyrrolidin-1-yl]-[1,2,4]triazolo[1,5-a]pyridine-2-carboxamide (50 mg, 63.54 mol, 1 eq) in THE (3 mL) was added Pd/C (6.76 mg, 6.35 mol, 10% purity, 0.1 eq) under N₂ atmosphere. The suspension was degassed and purged with H₂ for 3 times. The mixture was stirred under H₂ (15 Psi) at 25° C. for 1 h. LCMS showed starting material was consumed and one main peak with desired mass was detected. The reaction mixture was concentrated under the reduced pressure to give the crude product. The residue was purified by prep-HPLC (neutral condition column: CD02-Waters Xbidge BEH C18 150*25*10 um; mobile phase: [water(NH₄HCO₃)-ACN]; gradient:34%-64% B over 10 min) to give compound 8-(aminomethyl)-N-[10-[[3-(2,6-dioxo-3-piperidyl)-1-methyl-indazol-7-yl]amino]-10-oxo-decyl]-6-[(3R)-3-methyl-3-phenyl-pyrrolidin-1-yl]-[1,2,4]triazolo[1,5-a]pyridine-2-carboxamide (15 mg, 31% yield) was obtained as a white solid.

LC-MS [ESI, M+1]: 761.3.

¹H NMR (400 MHz, DMSO-d₆) δ=9.83 (s, 1H), 8.51 (t, J=6.0 Hz, 1H), 7.89 (d, J=1.6 Hz, 1H), 7.58 (d, J=7.6 Hz, 1H), 7.47-7.31 (m, 5H), 7.28-7.20 (m, 1H), 7.13-7.01 (m, 2H), 4.37 (dd, J=5.2, 10.0 Hz, 1H), 4.03 (s, 5H), 3.66-3.58 (m, 1H), 3.56-3.48 (m, 2H), 3.43-3.37 (m, 1H), 3.29-3.24 (m, 2H), 2.74-2.65 (m, 1H), 2.64-2.56 (m, 1H), 2.43-2.32 (m, 3H), 2.30-2.24 (m, 2H), 2.20-2.12 (m, 1H), 1.69-1.59 (m, 2H), 1.53 (br d, J=5.6 Hz, 2H), 1.43-1.24 (m, 13H)

### Example 8

### 8-(aminomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide

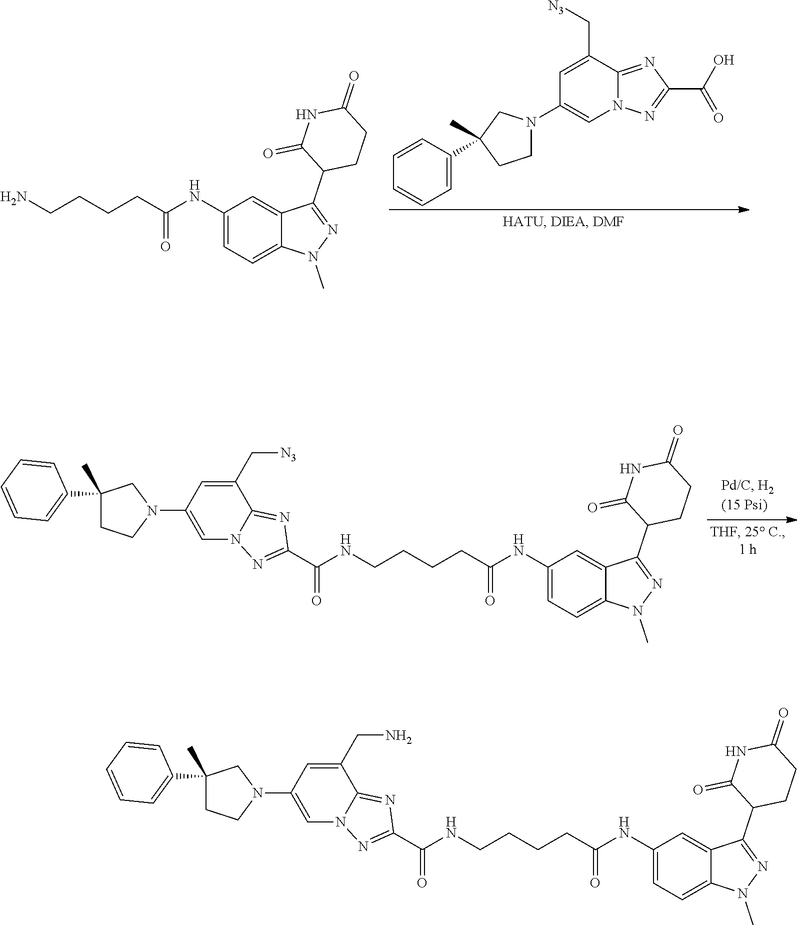

### Step 1: 8-(azidomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide

To a solution of 5-amino-N-[3-(2,6-dioxo-3-piperidyl)-1-methyl-indazol-5-yl]pentanamide (93.76 mg, 262.32 mol, 0.9 eq) and 8-(azidomethyl)-6-[(3R)-3-methyl-3-phenyl-pyrrolidin-1-yl]-[1,2,4]triazolo[1,5-a]pyridine-2-carboxylic acid (110 mg, 291.47 mol, 1 eq) in DMF (2 mL) was added HATU (221.65 mg, 582.94 mol, 2 eq) and DIEA (113.01 mg, 874.41 mol, 152.31 μL, 3 eq). The mixture was stirred at 25° C. for 10 min. LCMS showed starting material was consumed completely and the desired compound was detected. The residue was purified by prep—HPLC(column: CD02-Waters Xbidge BEH C18 150*25*10 um; mobile phase: [water(NH₄HCO₃)-ACN]; gradient:36%-66% B over 10 min) to give 8-(azidomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide (54.38 mg, 26% yield) as green solid.

LC-MS [ESI, M+1]: 717.3.

¹H NMR (400 MHz, CHLOROFORM-d) 6=8.43 (br s, 1H), 8.20 (br s, 1H), 8.05 (s, 1H), 7.74 (s, 1H), 7.56 (br d, J=7.2 Hz, 2H), 7.40-7.35 (m, 2H), 7.34-7.30 (m, 2H), 7.28 (br s, 1H), 7.27-7.24 (m, 1H), 7.20 (s, 1H), 4.78 (s, 2H), 4.30 (dd, J=5.2, 7.2 Hz, 1H), 3.97 (s, 3H), 3.64-3.48 (m, 5H), 3.43 (dt, J=3.6, 8.8 Hz, 1H), 2.88 (ddd, J=4.8, 8.0, 17.6 Hz, 1H), 2.64 (ddd, J=4.8, 8.0, 17.6 Hz, 1H), 2.54-2.24 (m, 6H), 1.82 (br dd, J=6.8, 14.0 Hz, 2H), 1.78-1.72 (m, 2H), 1.47 (s, 3H).

### Step 2: 8-(aminomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide

To a solution of 8-(azidomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide (40 mg, 55.80 mol, 1 eq) in THE (2 mL) was added Pd/C (11.88 mg, 11.16 mol, 10% purity, 0.2 eq) under H₂ (112.49 g, 55.80 mol, 1 eq). The mixture was stirred at 25° C. for 2 h. LCMS showed starting material was consumed completely and the desired compound was detected. The mixture was filtered and concentrated to give a residue. The crude product was purified by prep—HPLC(column: CD06-Waters Xbidge C18 150*40*10 um; mobile phase: [water(HCl)-ACN]; gradient:16%-46% B over 11 min) to give 8-(aminomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide (4.21 mg, 11% yield) as yellow solid.

LC-MS [ESI, M+23]: 691.4.

¹H NMR (400 MHz, DMSO-d₆) δ=10.89 (s, 1H), 9.98 (s, 1H), 8.81-8.72 (m, 1H), 8.65 (br s, 3H), 8.06 (br d, J=16.4 Hz, 2H), 7.72 (s, 1H), 7.57-7.44 (m, 2H), 7.42-7.30 (m, 4H), 7.25 (br d, J=7.2 Hz, 1H), 4.40 (br d, J=5.6 Hz, 2H), 4.34-4.26 (m, 1H), 3.96 (s, 3H), 3.63-3.51 (m, 6H), 2.74-2.58 (m, 2H), 2.40-2.27 (m, 5H), 2.19 (br d, J=5.6 Hz, 1H), 1.62 (br s, 4H), 1.37 (s, 3H).

### Example 9

### 8-(aminomethyl)-N-[6-[[3-(2,6-dioxo-3-piperidyl)-1-methyl-indazol-7-yl]amino]-6-oxo-hexyl]-6-[(3R)-3-methyl-3-phenyl-pyrrolidin-1-yl]-[1,2,4]triazolo[1,5-a]pyridine-2-carboxamide

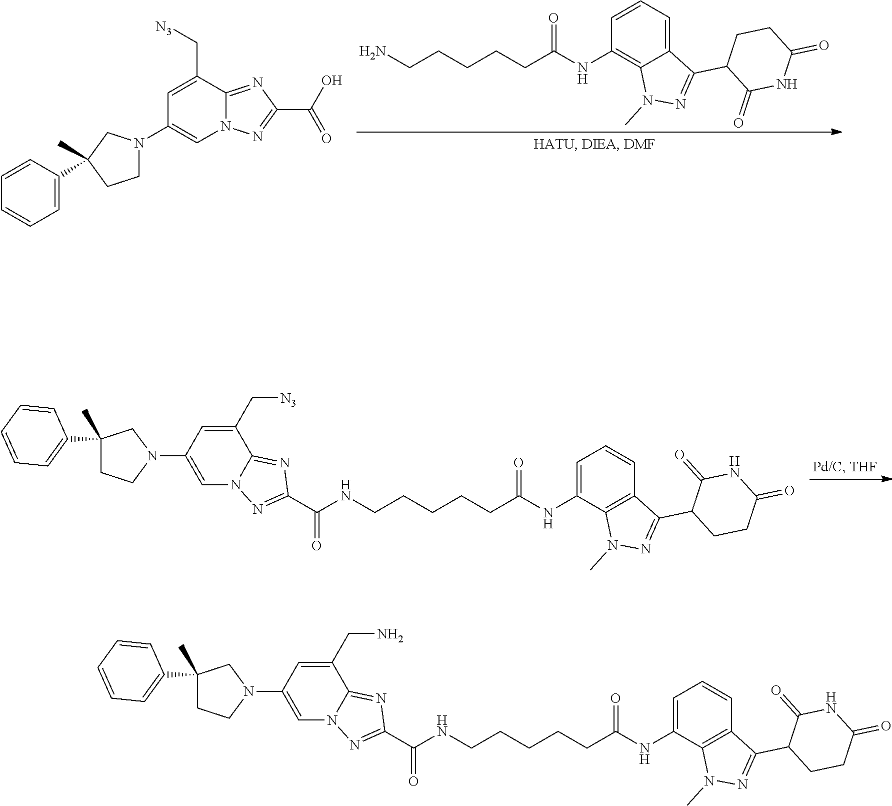 [SMILES: C[C@]1(c2ccccc2)CCN(c2cc(CN=[N+]=[N-])c3nc(C(=O)O)nn3c2)C1 ; Cn1nc(C2CCC(=O)NC2=O)c2cccc(NC(=O)CCCCCN)c21 ; Cn1nc(C2CCC(=O)NC2=O)c2cccc(NC(=O)CCCCCNC(=O)c3nc4c(CN=[N+]=[N-])cc(N5CC[C@](C)(c6ccccc6)C5)cn4n3)c21 ; Cn1nc(C2CCC(=O)NC2=O)c2cccc(NC(=O)CCCCCNC(=O)c3nc4c(CN)cc(N5CC[C@](C)(c6ccccc6)C5)cn4n3)c21]

### Step 1:8-(azidomethyl)-N-[6-[[3-(2,6-dioxo-3-piperidyl)-1-methyl-indazol-7-yl]amino]-6-oxo-hexyl]-6-[(3R)-3-methyl-3-phenyl-pyrrolidin-1-yl]-[1,2,4]triazolo[1,5-a]pyridine-2-carboxamide

To a solution of 6-amino-N-[3-(2,6-dioxo-3-piperidyl)-1-methyl-indazol-7-yl]hexanamide (93.21 mg, 228.50 mol, 1.1 eq, HCl), 8-(azidomethyl)-6-[(3R)-3-methyl-3-phenyl-pyrrolidin-1-yl]-[1,2,4]triazolo[1,5-a]pyridine-2-carboxylic acid (78.40 mg, 207.73 mol, 1 eq) in DMF (1 mL) was added HATU (157.97 mg, 415.46 mol, 2 eq) and DIEA (80.54 mg, 623.19 mol, 108.55 μL, 3 eq). The mixture was stirred at 25° C. for 0.5 h. LCMS showed starting material was consumed completely and the desired compound was detected. The reaction was concentrated under reduced pressure to give a residue. The residue was purified by flash silica gel chromatography (ISCO®; 4 g Sepa Flash® Silica Flash Column, Eluent of 0˜50% ACN/H2O @60 mL/min). Compound 8-(azidomethyl)-N-[6-[[3-(2,6-dioxo-3-piperidyl)-1-methyl-indazol-7-yl]amino]-6-oxo-hexyl]-6-[(3R)-3-methyl-3-phenyl-pyrrolidin-1-yl]-[1,2,4]triazolo[1,5-a]pyridine-2-carboxamide (100 mg, 66% yield) was obtained as a white solid.

LC-MS [ESI, M+1]:731.5

### Step 2: 8-(aminomethyl)-N-[6-[[3-(2,6-dioxo-3-piperidyl)-1-methyl-indazol-7-yl]amino]-6-oxo-hexyl]-6-[(3R)-3-methyl-3-phenyl-pyrrolidin-1-yl]-[1,2,4]triazolo[1,5-a]pyridine-2-carboxamide

To a solution of 8-(azidomethyl)-N-[6-[[3-(2,6-dioxo-3-piperidyl)-1-methyl-indazol-7-yl]amino]-6-oxo-hexyl]-6-[(3R)-3-methyl-3-phenyl-pyrrolidin-1-yl]-[1,2,4]triazolo[1,5-a]pyridine-2-carboxamide (100 mg, 136.83 mol, 1 eq) in THE (5 mL) was added Pd/C (14.56 mg, 13.68 mol, 10% purity, 0.1 eq) under N₂ atmosphere. The suspension was degassed and purged with H₂ for 3 times. The mixture was stirred under H₂ (15 Psi or atm.) at 25° C. for 1 h. LCMS showed starting material was consumed completely and the desired compound was detected. The reaction mixture was filtered. The residue was purified by prep-IPLC (column: CD07-Daisogel SP-100-8-ODS-PK 150*25*10 um; mobile phase: [water(NH₄HCO₃)-ACN]; gradient:22%-52% B over 11 min) and lyophilizated to give 8-(aminomethyl)-N-[6-[[3-(2,6-dioxo-3-piperidyl)-1-methyl-indazol-7-yl]amino]-6-oxo-hexyl]-6-[(3R)-3-methyl-3-phenyl-pyrrolidin-1-yl]-[1,2,4]triazolo[1,5-a]pyridine-2-carboxamide (25.08 mg, 26% yield).

LC-MS [ESI, M+1]: 705.4.

¹H NMR (400 MHz, DMSO-d₆) δ=11.06-10.68 (m, 1H), 9.83 (s, 1H), 8.54 (t, J=5.6 Hz, 1H), 7.91 (d, J=1.6 Hz, 1H), 7.58 (d, J=7.6 Hz, 1H), 7.46-7.34 (m, 5H), 7.28-7.21 (m, 1H), 7.12-7.02 (m, 2H), 4.37 (dd, J=5.2, 10.4 Hz, 1H), 4.03 (s, 5H), 3.67-3.59 (m, 1H), 3.58-3.49 (m, 2H), 3.45-3.39 (m, 1H), 3.23 (s, 2H), 2.71-2.60 (m, 2H), 2.45-2.38 (m, 3H), 2.31-2.26 (m, 2H), 2.20-2.15 (m, 1H), 1.74-1.66 (m, 2H), 1.64-1.56 (m, 2H), 1.46-1.39 (m, 2H), 1.38 (s, 3H)

### Example 10

### 8-(aminomethyl)-N-[11-[[3-(2,6-dioxo-3-piperidyl)-1-methyl-indazol-6-yl]amino]-11-oxo-undecyl]-6-[(3R)-3-methyl-3-phenyl-pyrrolidin-1-yl]-[1,2,4]triazolo[1,5-a]pyridine-2-carboxamide

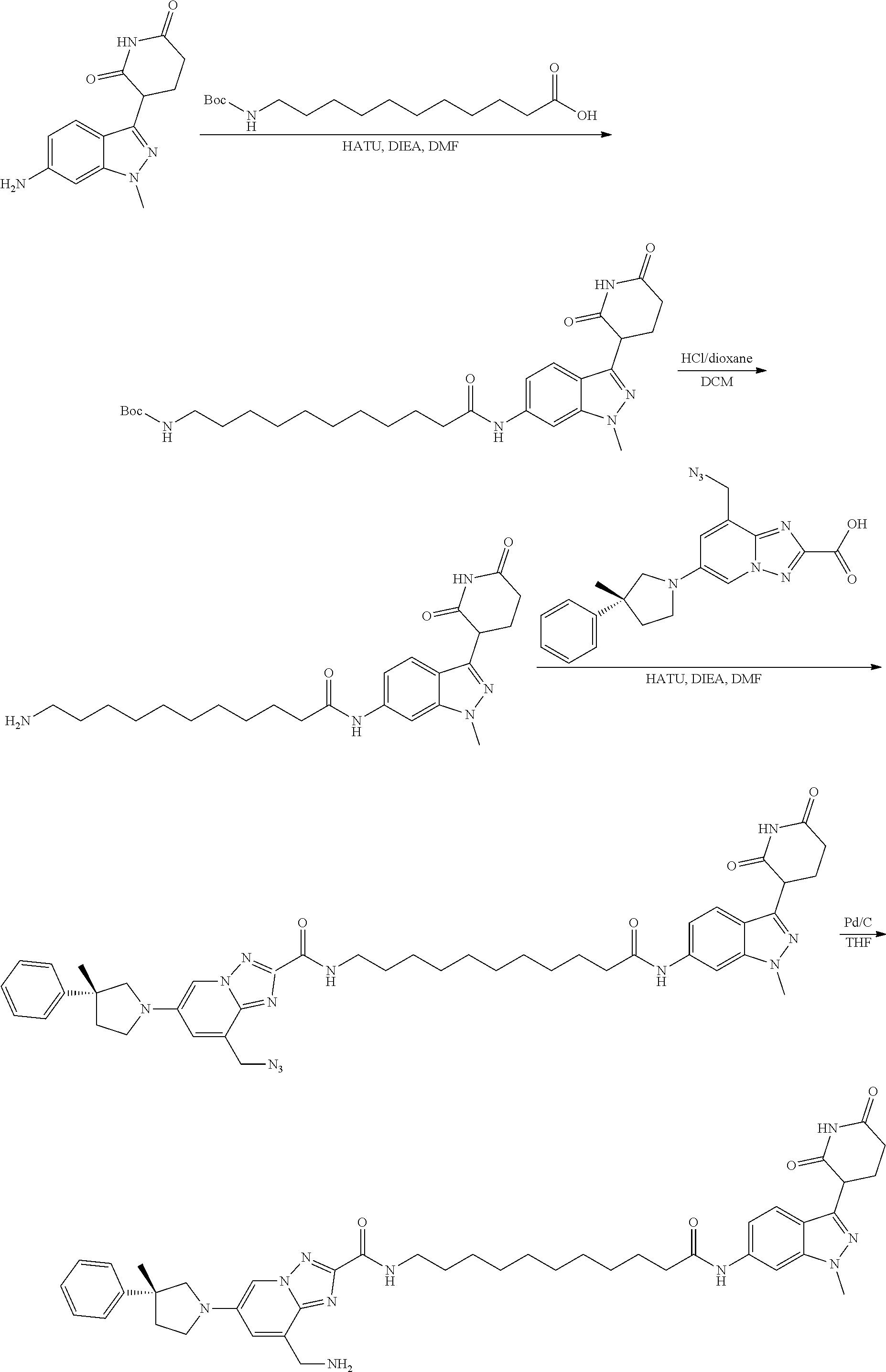

### Step 1: tert-butyl N-[11-[[3-(2,6-dioxo-3-piperidyl)-1-methyl-indazol-6-yl]amino]-11-oxo-undecyl]carbamate

To a solution of 11-(tert-butoxycarbonylamino)undecanoic acid (455.15 mg, 1.51 mmol, 1.5 eq) in DMF (6 mL) was added HATU (765.54 mg, 2.01 mmol, 2 eq) at 0° C. After addition, the mixture was stirred at this temperature for 0.5 h, and then 3-(6-amino-1-methyl-indazol-3-yl)piperidine-2,6-dione (260 mg, 1.01 mmol, 1 eq) and DIEA (650.52 mg, 5.03 mmol, 876.71 μL, 5 eq) was added at 0° C. The resulting mixture was stirred at 25° C. for 1 h. LCMS showed starting material was consumed completely and the desired compound was detected. The reaction mixture was quenched by H₂O (5 mL) and extracted with EtOAc (10 mL×3), The combined organic layers were washed with brine (10 mL×2), dried over anhydrous Na₂SO₄, filtered and dried to give a residue. The residue was purified by flash silica gel chromatography (ISCO®; 20 g SepaFlash® Silica Flash Column, Eluent of 0˜70% EtOAc: PE @60 mL/min) to give compound tert-butyl N-[11-[[3-(2,6-dioxo-3-piperidyl)-1-methyl-indazol-6-yl]amino]-11-oxo-undecyl]carbamate (380 mg, 68.79%) as a yellow solid.

LC-MS [ESI, M+1]: 542.4.

¹H NMR (400 MHz, DMSO-d₆) δ=10.88 (s, 1H), 10.05 (s, 1H), 8.08 (s, 1H), 7.60 (d, J=8.8 Hz, 1H), 7.07 (dd, J=1.2, 8.8 Hz, 1H), 6.75 (br t, J=5.2 Hz, 1H), 4.31 (dd, J=5.2, 9.6 Hz, 1H), 3.90 (s, 3H), 2.89-2.84 (m, 2H), 2.69-2.57 (m, 2H), 2.39-2.27 (m, 3H), 2.22-2.12 (m, 1H), 1.66-1.56 (m, 2H), 1.36 (s, 9H), 1.33-1.20 (m, 14H).

### Step 2:11-amino-N-[3-(2,6-dioxo-3-piperidyl)-1-methyl-indazol-6-yl]undecanamide

To a solution of tert-butyl N-[11-[[3-(2,6-dioxo-3-piperidyl)-1-methyl-indazol-6-yl]amino]-11-oxo-undecyl]carbamate (380 mg, 701.52 mol, 1 eq) in DCM (3 mL) was added HCl/dioxane (2 M, 2.17 mL, 6.19 eq). The mixture was stirred at 25° C. for 0.5 h. LCMS showed starting material was consumed completely and the desired compound was detected. The mixture was concentrated to give 11-amino-N-[3-(2,6-dioxo-3-piperidyl)-1-methyl-indazol-6-yl]undecanamide (335 mg, crude, HCl) was obtained as a yellow gum.

LC-MS [ESI, M+23]: 442.4.

### Step 3: 8-(azidomethyl)-N-[11-[[3-(2,6-dioxo-3-piperidyl)-1-methyl-indazol-6-yl]amino]-11-oxo-undecyl]-6-[(3R)-3-methyl-3-phenyl-pyrrolidin-1-yl]-[1,2,4]triazolo[1,5-a]pyridine-2-carboxamide

To a solution of 8-(azidomethyl)-6-[(3R)-3-methyl-3-phenyl-pyrrolidin-1-yl]-[1,2,4]triazolo[1,5-a]pyridine-2-carboxylic acid (170 mg, 450.45 mol, 1.3 eq) in DCM (2 mL) was added HATU (263.50 mg, 693.00 mol, 2 eq) at 0° C. After addition, the mixture was stirred at this temperature for 0.5 h, and then 11-amino-N-[3-(2,6-dioxo-3-piperidyl)-1-methyl-indazol-6-yl]undecanamide (165.64 mg, 346.50 mol, 1 eq, HCl) and DIEA (223.91 mg, 1.73 mmol, 301.76 μL, 5 eq) in DMF (0.5 mL) was added at 0° C. The resulting mixture was stirred at 25° C. for 1 h. LCMS showed starting material was consumed completely and the desired compound was detected. The reaction solution was concentrated under the reduced pressure to give the residue. The residue was purified by flash silica gel chromatography (ISCO®; 12 g SepaFlash® Silica Flash Column, Eluent of 0-100% EtOAc: PE @40 mL/min) to give compound 8-(azidomethyl)-N-[11-[[3-(2,6-dioxo-3-piperidyl)-1-methyl-indazol-6-yl]amino]-11-oxo-undecyl]-6-[(3R)-3-methyl-3-phenyl-pyrrolidin-1-yl]-[1,2,4]triazolo[1,5-a]pyridine-2-carboxamide (145 mg, 52%) as a yellow gum.

LC-MS [ESI, M+1]: 801.5

### Step 4: 8-(aminomethyl)-N-[11-[[3-(2,6-dioxo-3-piperidyl)-1-methyl-indazol-6-yl]amino]-11-oxo-undecyl]-6-[(3R)-3-methyl-3-phenyl-pyrrolidin-1-yl]-[1,2,4]triazolo[1,5-a]pyridine-2-carboxamide

A solution of 8-(azidomethyl)-N-[11-[[3-(2,6-dioxo-3-piperidyl)-1-methyl-indazol-6-yl]amino]-11-oxo-undecyl]-6-[(3R)-3-methyl-3-phenyl-pyrrolidin-1-yl]-[1,2,4]triazolo[1,5-a]pyridine-2-carboxamide (70 mg, 87.40 mol, 1 eq) in THE (0.5 mL) was added Pd/C (9.30 mg, 8.74 mol, 10% purity, 0.1 eq). The mixture was stirred under H₂ (15 Psi) at 20° C. for 1 h. LCMS showed starting material was consumed completely and the desired compound was detected. The reaction solution was filtered and the filtrate was concentrated under the reduced pressure to give the residue. The residue was purified by prep-HPLC (column: CD01-Phenomenex luna C18 150*25*10 um; mobile phase: [water(FA)-ACN]; gradient:25%-55% B over 11 min.) and lyophilized to give 8-(aminomethyl)-N-[11-[[3-(2,6-dioxo-3-piperidyl)-1-methyl-indazol-6-yl]amino]-11-oxo-undecyl]-6-[(3R)-3-methyl-3-phenyl-pyrrolidin-1-yl]-[1,2,4]triazolo[1,5-a]pyridine-2-carboxamide (20 mg, 25.10 mol, 28.72% yield, 97.262% purity) as a yellow solid

LC-MS [ESI, M+1]:775.5.

¹H NMR (400 MHz, DMSO-d₆) δ=11.11-10.67 (m, 1H), 10.06 (s, 1H), 8.54 (br t, J=5.6 Hz, 1H), 8.26-8.17 (m, 1H), 8.08 (s, 1H), 7.95 (s, 1H), 7.60 (d, J=8.8 Hz, 1H), 7.49 (br s, 1H), 7.42-7.32 (m, 4H), 7.27-7.21 (m, 1H), 7.07 (d, J=8.8 Hz, 1H), 4.30 (dd, J=5.2, 9.6 Hz, 1H), 4.14 (br d, J=5.2 Hz, 2H), 3.90 (s, 3H), 3.64-3.59 (m, 1H), 3.58-3.48 (m, 3H), 3.27-3.23 (m, 2H), 2.72-2.54 (m, 2H), 2.38-2.23 (m, 5H), 2.21-2.11 (m, 1H), 1.65-1.45 (m, 4H), 1.37 (s, 3H), 1.28 (br s, 12H)

### Example 11

### N-[10-[[3-(2,6-dioxo-3-piperidyl)-1-methyl-indazol-6-yl]amino]-10-oxo-decyl]-8-methyl-6-[(3R)-3-methyl-3-phenyl-pyrrolidin-1-yl]-[1,2,4]triazolo[1,5-a]pyridine-2-carboxamide

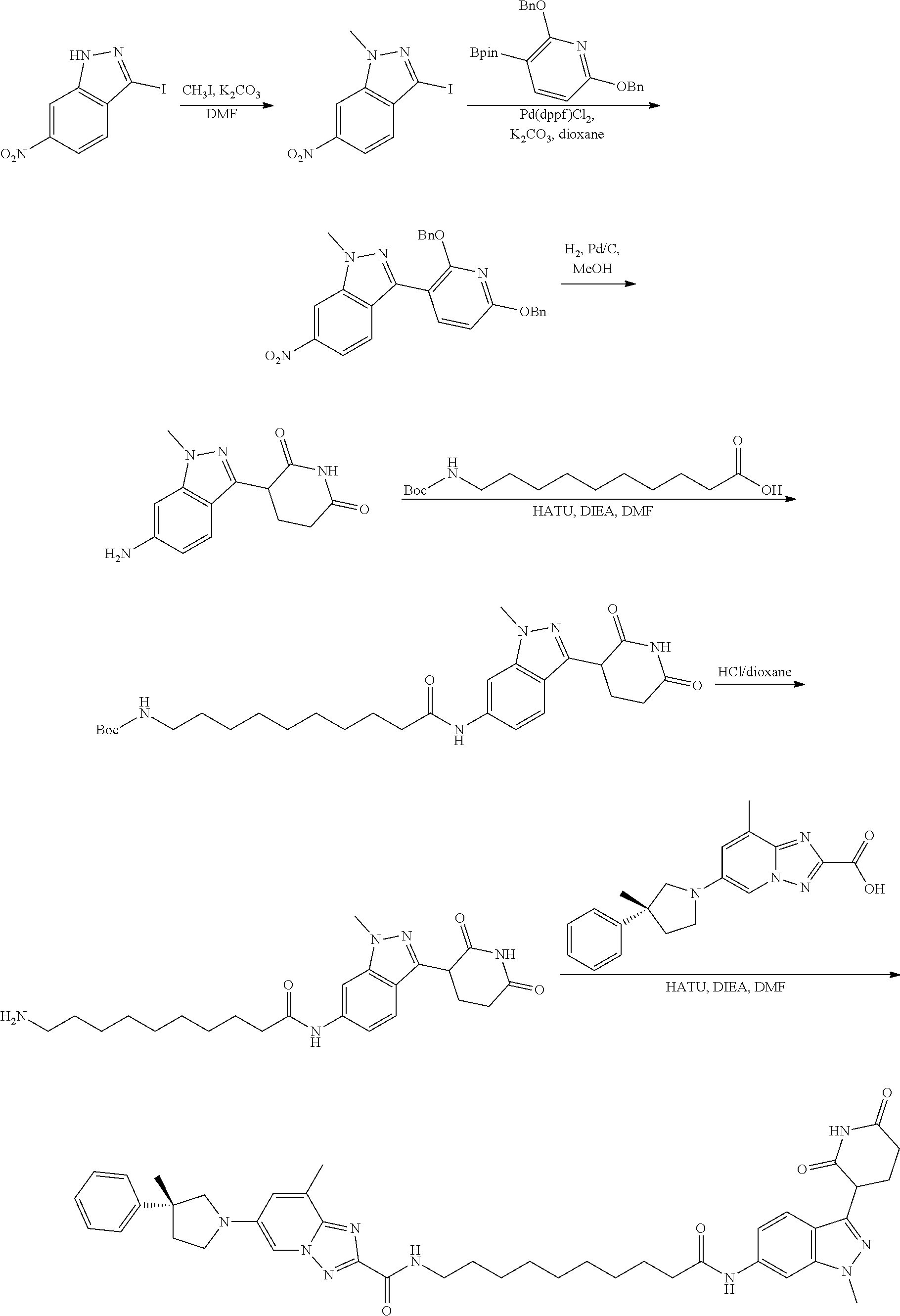

### Step 1: 3-iodo-1-methyl-6-nitro-indazole

To a solution of 3-iodo-6-nitro-1H-indazole (10 g, 34.60 mmol, 1 eq) in DMF (40 mL) was added Mel (5.89 g, 41.52 mmol, 2.58 mL, 1.2 eq) and K₂CO₃ (4.78 g, 34.60 mmol, 1 eq). The mixture was stirred at 25° C. for 6 h. TLC (PE: EtOAc=3:1, Rf=0.43) indicated reactant was consumed completely and one new spot was formed. The reaction mixture was quenched by H₂O (100 mL) at 0° C., then extracted with EtOAc (40 mL×3), the combined organic layers dried over anhydrous sodium sulfate, filtered and the filtrate was concentrated under reduced pressure to give a residue. The residue was purified by flash silica gel chromatography (ISCO®; 80 g SepaFlash® Silica Flash Column, Eluent of 0˜25% EtOAc/PE gradient) to give compound 3-iodo-1-methyl-6-nitro-indazole (7.2 g, 68% yield) as yellow solid.

LC-MS [ESI, M]: 303.8.

¹H NMR (400 MHz, CHLOROFORM-d) 6=8.39 (d, J=1.6 Hz, 1H), 8.08 (dd, J=1.6, 8.8 Hz, 1H), 7.63 (d, J=8.8 Hz, 1H), 4.22 (s, 3H).

### Step 2: 3-(2,6-bis(benzyloxy)pyridin-3-yl)-1-methyl-6-nitro-1H-indazole

A mixture of 3-iodo-1-methyl-6-nitro-indazole (4.1 g, 13.53 mmol, 1 eq), 2,6-dibenzyloxy-3-(4,4,5,5-tetramethyl-1,3,2-dioxaborolan-2-yl)pyridine (5.65 g, 13.53 mmol, 1 eq), cyclopentyl(diphenyl)phosphane; dichloromethane; dichloropalladium; iron (662.89 mg, 811.73 mol, 0.06 eq) and Cs₂CO₃ (8.82 g, 27.06 mmol, 2 eq) in dioxane (30 mL) and H₂O (6 mL) was degassed and purged with N₂ for 3 times, and then the mixture was stirred at 100° C. for 12 h under N₂ atmosphere. LCMS showed starting material was consumed and the desired mass was detected. The mixture was quenched with H₂O (20 mL) and extracted with EtOAc (20 mL×3), the organic phase was dried, filtered, and concentrated under pressure to give the product. The residue was purified by flash silica gel chromatography (ISCO®; 40 g SepaFlash® Silica Flash Column, Eluent of 0˜25% EtOAc/PE gradient to give compound 3-(2,6-bis(benzyloxy)pyridin-3-yl)-1-methyl-6-nitro-1H-indazole (3 g, 48% yield) as a pink solid.

LC-MS [ESI, M+1]: 467.2.

¹H NMR (400 MHz, CHLOROFORM-d) 6=8.34 (s, 1H), 7.94 (d, J=8.4 Hz, 1H), 7.86-7.80 (m, 2H), 7.51-7.44 (m, 2H), 7.43-7.27 (m, 8H), 6.57 (d, J=8.0 Hz, 1H), 5.46 (d, J=13.2 Hz, 4H), 4.21 (s, 3H).

### Step 3: 3-(6-amino-1-methyl-indazol-3-yl)piperidine-2,6-dione

To a solution of 3-(2,6-dibenzyloxy-3-pyridyl)-1-methyl-6-nitro-indazole (3 g, 6.43 mmol, 1 eq) in THE (60 mL) and EtOH (60 mL) was added Pd/C (1.57 g, 1.48 mmol, 10% purity, 0.23 eq) and AcOH (386.20 mg, 6.43 mmol, 368.16 μL, 1 eq). The suspension was degassed and purged with H₂ for 3 times. The mixture was stirred under H₂ (50 Psi) at 45° C. for 48 h. LCMS showed starting material was consumed and the desired mass was detected. The reaction was concentrated under reduced pressure to give compound 3-(6-amino-1-methyl-indazol-3-yl)piperidine-2,6-dione (1.6 g, 98% yield) as a pink oil.

LC-MS [ESI, M+1]: 259.1.

¹H NMR (400 MHz, CHLOROFORM-d) 6=8.32 (br s, 1H), 7.43 (d, J=8.8 Hz, 1H), 6.57 (dd, J=1.6, 8.8 Hz, 1H), 6.49 (d, J=1.6 Hz, 1H), 4.84-4.68 (m, 2H), 4.24 (dd, J=5.2, 8.0 Hz, 1H), 3.88 (s, 3H), 3.06-2.89 (m, 1H), 2.73-2.60 (m, 1H), 2.54-2.43 (m, 1H), 2.40-2.31 (m, 1H).

### Step 4: tert-butyl N-[10-[[3-(2,6-dioxo-3-piperidyl)-1-methyl-indazol-6-yl]amino]-10-oxo-decyl]carbamate

To a solution of 3-(6-amino-1-methyl-indazol-3-yl)piperidine-2,6-dione (920 mg, 3.56 mmol, 1 eq) and 10-(tertbutoxycarbonylamino)decanoic acid (1.02 g, 3.56 mmol, 1 eq) in DMF (10 mL) was added HATU (2.03 g, 5.34 mmol, 1.5 eq) and DIEA (1.38 g, 10.69 mmol, 1.86 mL, 3 eq). The mixture was stirred at 25° C. for 0.5 h. LCMS showed starting material was consumed and one main peak with desired mass was detected. The mixture was quenched with H₂O (20 mL) and extracted with EtOAc (20 mL×3), the organic phase was dried, filtered, and concentrated under pressure to give the product. The residue was purified by flash silica gel chromatography (ISCO®; 20 g SepaFlash® Silica Flash Column, Eluent of 0˜80% EtOAc/PE gradient) to give compound tert-butyl N-[10-[[3-(2,6-dioxo-3-piperidyl)-1-methyl-indazol-6-yl]amino]-10-oxo-decyl]carbamate (1.35 g, 72% yield) as a white solid.

LC-MS [ESI, M+1]: 528.6.

¹H NMR (400 MHz, CHLOROFORM-d) 6=8.21 (s, 1H), 8.09 (br s, 1H), 7.64-7.50 (m, 2H), 6.81 (dd, J=1.6, 8.8 Hz, 1H), 4.54 (br s, 1H), 4.27 (dd, J=5.2, 7.2 Hz, 1H), 3.98 (s, 3H), 3.10 (br d, J=6.4 Hz, 2H), 2.99 (br d, J=3.6 Hz, 1H), 2.75-2.64 (m, 1H), 2.42-2.38 (m, 2H), 1.79-1.71 (m, 2H), 1.45 (s, 23H).

### Step 5: 10-amino-N-[3-(2,6-dioxo-3-piperidyl)-1-methyl-indazol-6-yl]decanamide

To a solution of tert-butyl N-[10-[[3-(2,6-dioxo-3-piperidyl)-1-methyl-indazol-6-yl]amino]-10-oxo-decyl]carbamate (50 mg, 94.76 mol, 1 eq) in DCM (1 mL) was added HCl/dioxane (2 M, 473.80 μL, 10 eq). The mixture was stirred at 25° C. for 0.5 h. LCMS showed starting material was consumed and one main peak with desired mass was detected. The mixture was filtered, and concentrated under pressure to give compound 10-amino-N-[3-(2,6-dioxo-3-piperidyl)-1-methyl-indazol-6-yl]decanamide (43 mg, 98% yield, HCl) as a white solid.

LC-MS [ESI, M+1]: 428.3.

### Step 6: N-[10-[[3-(2,6-dioxo-3-piperidyl)-1-methyl-indazol-6-yl]amino]-10-oxo-decyl]-8-methyl-6-[(3R)-3-methyl-3-phenyl-pyrrolidin-1-yl]-[1,2,4]triazolo[1,5-a]pyridine-2-carboxamide

To a solution of 8-methyl-6-[(3R)-3-methyl-3-phenyl-pyrrolidin-1-yl]-[1,2,4]triazolo[1,5-a]pyridine-2-carboxylic acid (25 mg, 74.32 mol, 1 eq) and 10-amino-N-[3-(2,6-dioxo-3-piperidyl)-1-methyl-indazol-6-yl]decanamide (31.77 mg, 74.32 mol, 1 eq) in DMF (1 mL) was added DIEA (28.82 mg, 222.96 mol, 38.83 μL, 3 eq) and HATU (42.39 mg, 111.48 mol, 1.5 eq). The mixture was stirred at 25° C. for 0.5 h. LCMS showed starting material was consumed and one main peak with desired mass was detected. The mixture was filtered. The residue was purified by prep-HPLC (column: CD01-Phenomenex luna C18 150*25*10 um; mobile phase: [water(FA)-ACN]; gradient:48%-78% B over 11 min) to give compound N-[10-[[3-(2,6-dioxo-3-piperidyl)-1-methyl-indazol-6-yl]amino]-10-oxo-decyl]-8-methyl-6-[(3R)-3-methyl-3-phenyl-pyrrolidin-1-yl]-[1,2,4]triazolo[1,5-a]pyridine-2-carboxamide (31.84 mg, 55% yield) as a yellow solid.

LC-MS [ESI, M]: 746.5.

¹H NMR (400 MHz, CHLOROFORM-d) 6=8.21 (s, 1H), 8.14 (br d, J=17.6 Hz, 2H), 7.63 (s, 1H), 7.53 (d, J=8.8 Hz, 1H), 7.45 (br s, 1H), 7.40-7.33 (m, 2H), 7.33-7.28 (m, 2H), 7.26 (br s, 1H), 7.01 (s, 1H), 6.95 (d, J=8.8 Hz, 1H), 4.33-4.19 (m, 1H), 3.95 (s, 3H), 3.57 (d, J=9.2 Hz, 1H), 3.54-3.44 (m, 4H), 3.41-3.33 (m, 1H), 2.99 (ddd, J=4.8, 8.4, 17.6 Hz, 1H), 2.72-2.63 (m, 1H), 2.60 (s, 3H), 2.54-2.46 (m, 1H), 2.43-2.32 (m, 4H), 2.28-2.22 (m, 1H), 1.80-1.69 (m, 2H), 1.68-1.56 (m, 2H), 1.47-1.42 (m, 3H), 1.40-1.26 (m, 10H).

### Example 12

### N-[6-[[3-(2,6-dioxo-3-piperidyl)-1-methyl-indazol-6-yl]amino]-6-oxo-hexyl]-8-methyl-6-[(3R)-3-methyl3-phenyl-pyrrolidin-1-yl]-[1,2,4]triazolo[1,5-a]pyridine-2-carboxamide

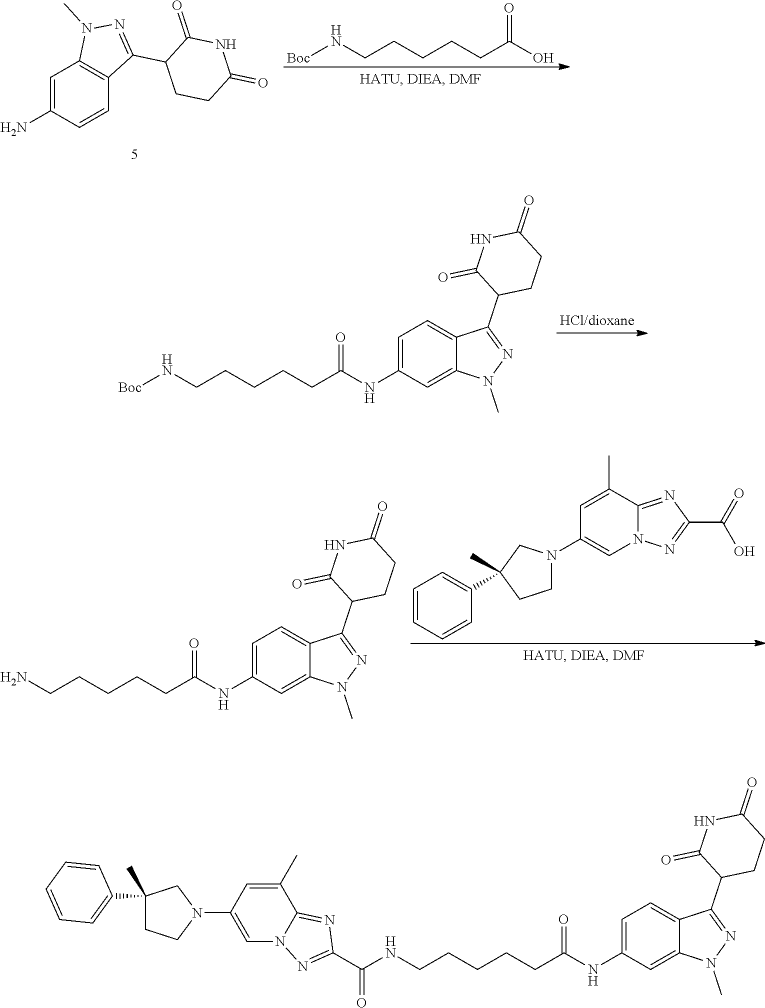

### Step 1: tert-butyl N-[6-[[3-(2,6-dioxo-3-piperidyl)-1-methyl-indazol-6-yl]amino]-6-oxo-hexyl]carbamate

To a solution of 3-(6-amino-1-methyl-indazol-3-yl)piperidine-2,6-dione (280 mg, 1.08 mmol, 1 eq) and 6-(tertbutoxycarbonylamino)hexanoic acid (275.82 mg, 1.19 mmol, 1.1 eq) in DMF (5 mL) was added HATU (618.32 mg, 1.63 mmol, 1.5 eq) and DIEA (420.34 mg, 3.25 mmol, 566.50 μL, 3 eq). The mixture was stirred at 25° C. for 0.5 h. LCMS showed starting material was consumed and one main peak with desired mass was detected. The mixture was quenched with H₂O (10 mL) and extracted with EtOAc (10 mL×3), the organic phase was dried, filtered, and concentrated under pressure to give the product. The residue was purified by flash silica gel chromatography (ISCO®; 20 g SepaFlash® Silica Flash Column, Eluent of 0˜80% EtOAc/PE gradient) to give compound tert-butyl N-[6-[[3-(2,6-dioxo-3-piperidyl)-1-methyl-indazol-6-yl]amino]-6-oxo-hexyl]carbamate (0.45 g, 88% yield) as a white solid.

¹H NMR (400 MHz, CHLOROFORM-d) 6=8.24-8.06 (m, 2H), 7.70 (br s, 1H), 7.55 (d, J=8.8 Hz, 1H), 6.83 (br d, J=8.4 Hz, 1H), 4.61 (br s, 1H), 4.27 (dd, J=5.2, 7.2 Hz, 1H), 3.97 (s, 3H), 3.22-3.09 (m, 3H), 3.01 (ddd, J=4.8, 8.4, 17.6 Hz, 1H), 2.69 (ddd, J=5.2, 7.6, 17.6 Hz, 1H), 2.57-2.47 (m, 1H), 2.43-2.39 (m, 2H), 1.82-1.76 (m, 2H), 1.54 (br d, J=7.2 Hz, 2H), 1.45 (s, 11H).

### Step 2: 6-amino-N-[3-(2,6-dioxo-3-piperidyl)-1-methyl-indazol-6-yl]hexanamide

To a solution of tert-butyl N-[6-[[3-(2,6-dioxo-3-piperidyl)-1-methyl-indazol-6-yl]amino]-6-oxo-hexyl]carbamate (45 mg, 95.43 mol, 1 eq) in DCM (2 mL) was added HCl/dioxane (2 M, 477.15 μL, 10 eq). The mixture was stirred at 25° C. for 0.5 h. LCMS showed starting material was consumed and one main peak with desired mass was detected. The mixture was filtered, and concentrated under pressure to give compound 6-amino-N-[3-(2,6-dioxo-3-piperidyl)-1-methyl-indazol-6-yl]hexanamide (38 mg, 97% yield, HCl) as a white solid.

LC-MS [ESI, M+1]: 372.1.

### Step 3: N-[6-[[3-(2,6-dioxo-3-piperidyl)-1-methyl-indazol-6-yl]amino]-6-oxo-hexyl]-8-methyl-6-[(3R)-3-methyl3-phenyl-pyrrolidin-1-yl]-[1,2,4]triazolo[1,5-a]pyridine-2-carboxamide

To a solution of 6-amino-N-[3-(2,6-dioxo-3-piperidyl)-1-methyl-indazol-6-yl]hexanamide (38 mg, 102.31 mol, 1 eq) and 8-methyl-6-[(3R)-3-methyl-3-phenyl-pyrrolidin-1-yl]-[1,2,4]triazolo[1,5-a]pyridine-2-carboxylic acid (34.41 mg, 102.31 mol, 1 eq) in DMF (1 mL) was added DIEA (39.67 mg, 306.92 mol, 53.46 μL, 3 eq) and HATU (58.35 mg, 153.46 mol, 1.5 eq). The mixture was stirred at 25° C. for 0.5 h. LCMS showed starting material was consumed and one main peak with desired mass was detected. The mixture was quenched with H₂O (5 mL) and extracted with EtOAc (10 mL×3), the organic phase was dried, filtered, and concentrated under pressure to give the product. The residue was purified by prep-HPLC(column: CD01-Phenomenex luna C18 150*25*10 um; mobile phase: [water(FA)-ACN]; gradient:34%-64% B over 11 min) to give compound N-[6-[[3-(2,6-dioxo-3-piperidyl)-1-methyl-indazol-6-yl]amino]-6-oxo-hexyl]-8-methyl-6-[(3R)-3-methyl3-phenyl-pyrrolidin-1-yl]-[1,2,4]triazolo[1,5-a]pyridine-2-carboxamide (29.29 mg, 41% yield) as yellow solid.

LC-MS [ESI, M+1]: 690.4.

¹H NMR (400 MHz, CHLOROFORM-d) 6=8.16 (br s, 2H), 8.06 (br s, 1H), 7.66 (s, 2H), 7.53 (br d, J=8.4 Hz, 1H), 7.42-7.29 (m, 5H), 7.05 (s, 1H), 6.95 (br d, J=8.8 Hz, 1H), 4.27 (t, J=6.4 Hz, 1H), 3.95 (s, 3H), 3.61 (br d, J=8.8 Hz, 1H), 3.57-3.47 (m, 4H), 3.41 (br d, J=3.2 Hz, 1H), 3.06-2.94 (m, 1H), 2.73-2.65 (m, 1H), 2.61 (s, 3H), 2.52-2.45 (m, 2H), 2.39 (br d, J=4.4 Hz, 2H), 2.28 (br s, 2H), 1.87-1.79 (m, 2H), 1.76-1.69 (m, 2H), 1.52 (br d, J=3.2 Hz, 2H), 1.48 (s, 3H).

### Example 13

### N-[10-[[3-(2,6-dioxo-3-piperidyl)-1-methyl-indazol-7-yl]amino]-10-oxo-decyl]-8-methyl-6-[(3R)-3-methyl-3-phenyl-pyrrolidin-1-yl]-[1,2,4]triazolo[1,5-a]pyridine-2-carboxamide

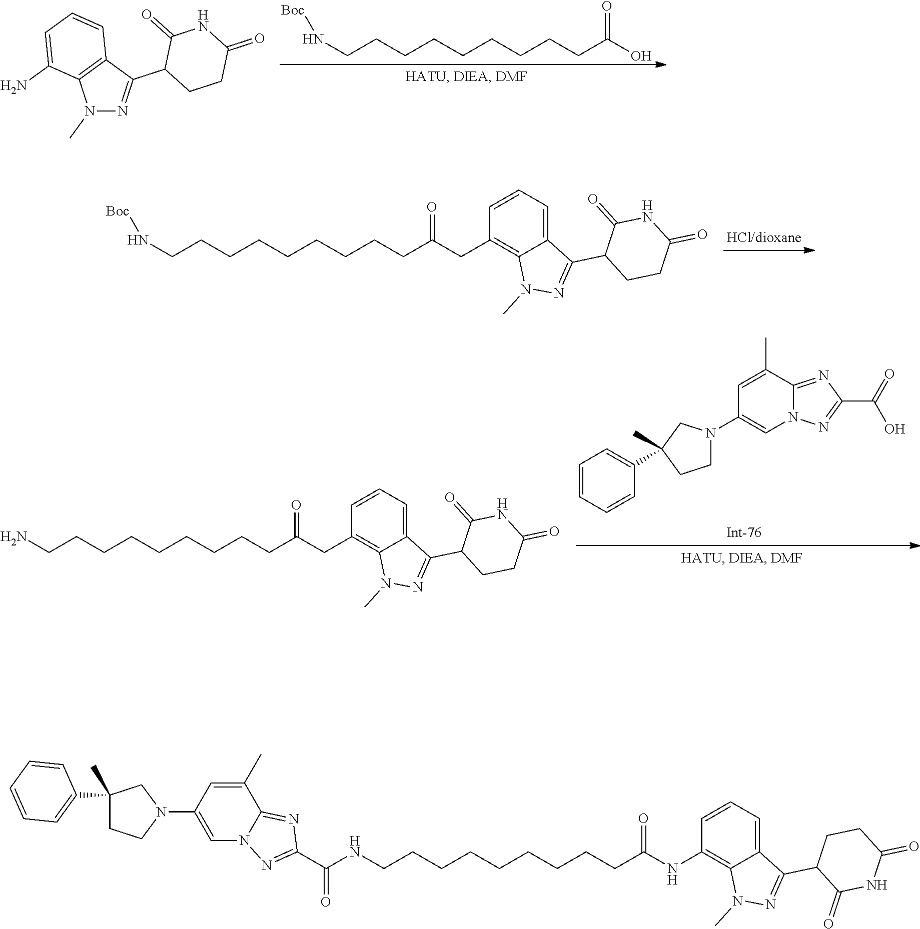

### Step 1: tert-butyl N-[10-[[3-(2,6-dioxo-3-piperidyl)-1-methyl-indazol-7-yl]amino]-10-oxo-decyl]carbamate

To a solution of 3-(7-amino-1-methyl-indazol-3-yl)piperidine-2,6-dione (150 mg, 580.78 mol, 1 eq), 10-(tertbutoxycarbonylamino)decanoic acid (166.91 mg, 580.78 mol, 1 eq) in DMF (3 mL) was added HATU (552.07 mg, 1.45 mmol, 2.5 eq) and DIEA (225.18 mg, 1.74 mmol, 303.48 μL, 3 eq). The mixture was stirred at 25° C. for 1 h. LCMS showed starting material was consumed completely and the desired compound was detected. The reaction was concentrated under reduced pressure to give a residue. The residue was purified by prep-HPLC (column: CD03-Welch Xtimate C18 150*25*5 um; mobile phase: [water(FA)-ACN]; gradient:54%-84% B over 10 min) and lyophilizated to give compound tert-butyl N-[10-[[3-(2,6-dioxo-3-piperidyl)-1-methyl-indazol-7-yl]amino]-10-oxo-decyl]carbamate (150 mg, 49% yield) was obtained as a white solid.

LC-MS [ESI, M-100]:428.2

### Step 2: 10-amino-N-[3-(2,6-dioxo-3-piperidyl)-1-methyl-indazol-7-yl]decanamide

A solution of tert-butyl N-[10-[[3-(2,6-dioxo-3-piperidyl)-1-methyl-indazol-7-yl]amino]-10-oxo-decyl]carbamate (140 mg, 265.33 mol, 1 eq) in HCl/dioxane (4M, 10 mL). was stirred at 25° C. for 0.5 h. LCMS showed starting material was consumed completely and the desired compound was detected. The reaction was concentrated under reduced pressure to give compound 10-amino-N-[3-(2,6-dioxo-3-piperidyl)-1-methyl-indazol-7-yl]decanamide (140 mg, crude) was obtained as a white solid.

LC-MS [ESI, M+1]:428.2

### Step 3: N-[10-[[3-(2,6-dioxo-3-piperidyl)-1-methyl-indazol-7-yl]amino]-10-oxo-decyl]-8-methyl-6-[(3R)-3-methyl-3-phenyl-pyrrolidin-1-yl]-[1,2,4]triazolo[1,5-a]pyridine-2-carboxamide

To a solution of 10-amino-N-[3-(2,6-dioxo-3-piperidyl)-1-methyl-indazol-7-yl]decanamide (30 mg, 70.17 mol, 1 eq), 8-methyl-6-[(3R)-3-methyl-3-phenyl-pyrrolidin-1-yl]-[1,2,4]triazolo[1,5-a]pyridine-2-carboxylic acid (23.60 mg, 70.17 mol, 1 eq) in DMF (0.5 mL) was added HATU (53.36 mg, 140.34 mol, 2 eq) and DIEA (27.21 mg, 210.51 mol, 36.67 μL, 3 eq). The mixture was stirred at 25° C. for 0.5 h. LCMS showed starting material was consumed completely and the desired compound was detected. The reaction was concentrated under reduced pressure to give a residue. The residue was purified by prep-HPLC (column: CD01-Phenomenex luna C18 150*25*10 um; mobile phase: [water(FA)-ACN]; gradient:50%-80% B over 8 min) and lyophilizated to give compound N-[10-[[3-(2,6-dioxo-3-piperidyl)-1-methyl-indazol-7-yl]amino]-10-oxo-decyl]-8-methyl-6-[(3R)-3-methyl-3-phenyl-pyrrolidin-1-yl]-[1,2,4]triazolo[1,5-a]pyridine-2-carboxamide (30 mg, 57% yield) was obtained as a white solid.

LC-MS [ESI, M+1]:746.3

¹H NMR (400 MHz, DMSO-d₆) δ=10.8 (s, 1H), 9.82 (s, 1H), 8.48 (s, 1H), 7.87 (s, 1H), 7.58 (d, J=7.2 Hz, 1H), 7.31 (s, 1H), 7.42-7.26 (m, 4H), 7.24 (d, J=6.4 Hz, 1H), 7.13-7.00 (m, 2H), 4.37 (dd, J=4.8, 9.6 Hz, 1H), 4.03 (s, 3H), 3.63-3.55 (m, 1H), 3.54-3.38 (m, 5H), 3.27 (d, J=6.0 Hz, 2H), 2.73-2.59 (m, 2H), 2.52 (d, J=18.8 Hz, 4H), 2.39 (t, J=7.2 Hz, 3H), 2.29-2.12 (m, 3H), 1.69-1.48 (m, 4H), 1.31 (s, 10H)

### Example 14

### N-[6-[4-[(2,6-dioxo-3-piperidyl)amino]anilino]-6-oxo-hexyl]-8-methyl-6-[(3R)-3-methyl-3-phenyl-pyrrolidin-1-yl]-[1,2,4]triazolo[1,5-a]pyridine-2-carboxamide

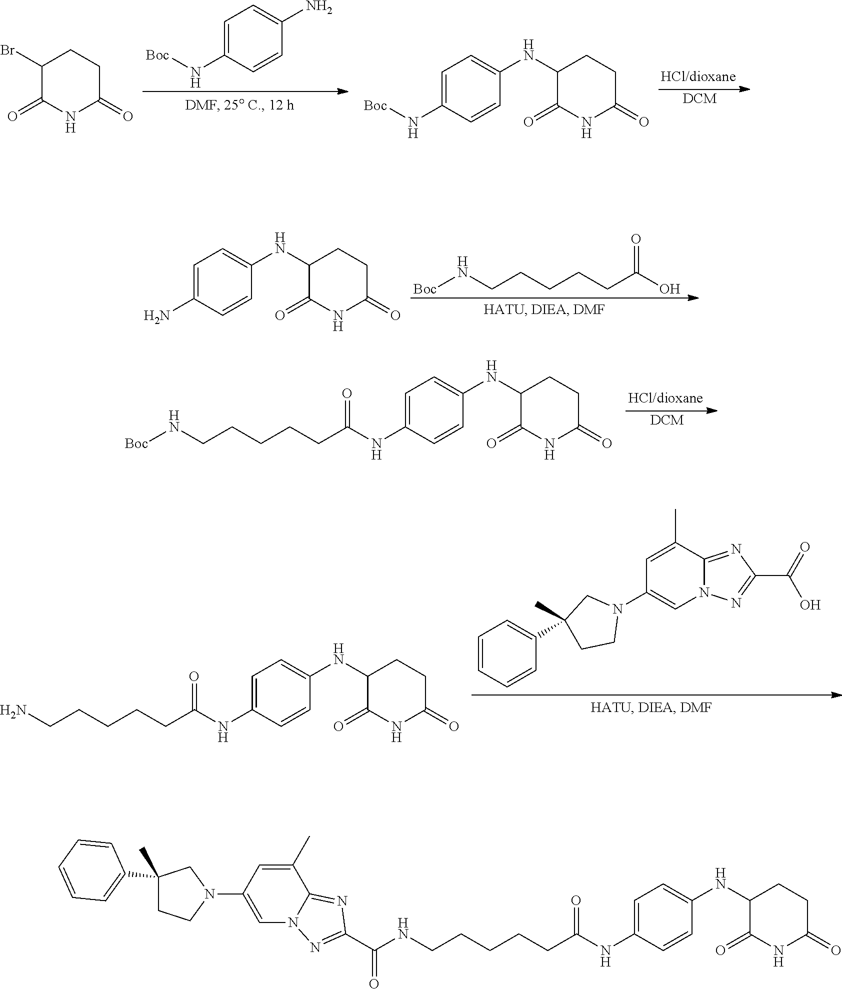

### Step 1: tert-butyl N-[4-[(2,6-dioxo-3-piperidyl)amino]phenyl]carbamate

A mixture of 3-bromopiperidine-2,6-dione (1 g, 5.21 mmol, 1 eq), tert-butyl N-(4-aminophenyl)carbamate (1.08 g, 5.21 mmol, 1 eq) in DMF (10 mL) was degassed and purged with N₂ for 3 times, and then the mixture was stirred at 25° C. for 2 h under N₂ atmosphere. LCMS showed starting material was consumed completely and the desired compound was detected. The reaction solution was concentrated under the reduced pressure to give the residue. The residue was purified by prep-HPLC (column:120 g Flash Column Welch Ultimate XB_C18 20-40 m; 120 A; mobile phase: [water(FA)-ACN]; B %: 5%-42%, 25 min.) and lyophilized to give tert-butyl N-[4-[(2,6-dioxo-3-piperidyl)amino]phenyl]carbamate (680 mg, 41% yield) was obtained as a gray solid

LC-MS [ESI, M]: 320.2.

### Step 2: 3-(4-aminoanilino)piperidine-2,6-dione

To a solution of tert-butyl N-[4-[(2,6-dioxo-3-piperidyl)amino]phenyl]carbamate (680 mg, 2.13 mmol, 1 eq) in DCM (4 mL) was added HCl/dioxane (2 M, 6 mL, 5.64 eq). The mixture was stirred at 20° C. for 0.5 h. LCMS showed starting material was consumed and the desired mass was detected. The reaction solution was concentrated under the reduced pressure to give compound 3-(4-aminoanilino)piperidine-2,6-dione (544 mg, crude, HCl) as a violet solid.

LC-MS [ESI, M+1]: 220.1.

¹H NMR (400 MHz, DMSO-d₆) δ=10.80 (s, 1H), 9.99 (br s, 3H), 7.11 (d, J=8.8 Hz, 2H), 6.74 (d, J=8.8 Hz, 2H), 4.35 (dd, J=4.8, 11.6 Hz, 1H), 2.80-2.67 (m, 1H), 2.63-2.55 (m, 1H), 2.13-2.02 (m, 1H), 1.96-1.83 (m, 1H)

### Step 3: tert-butyl N-[6-[4-[(2,6-dioxo-3-piperidyl)amino]anilino]-6-oxo-hexyl]carbamate

To a solution of 3-(4-aminoanilino)piperidine-2,6-dione (244 mg, 954.24 mol, 1 eq, HCl) in DCM (10 mL) and DMF (2 mL) was added HATU (1.09 g, 2.86 mmol, 3 eq) at 0° C. After addition, the mixture was stirred at this temperature for 0.5 h, and then 6-(tert-butoxycarbonylamino)hexanoic acid (441.41 mg, 1.91 mmol, 2 eq) and DIEA (616.63 mg, 4.77 mmol, 831.04 μL, 5 eq) was added at 0° C. The resulting mixture was stirred at 25° C. for 1 h. LCMS showed starting material was consumed and the desired mass was detected. The reaction solution was filtered and the filtrate was concentrated under the reduced pressure to give the residue. The residue was purified by flash silica gel chromatography (ISCO®; 12 g SepaFlash® Silica Flash Column, Eluent of 0˜50% EtOAc: PE @50 mL/min) to give tert-butyl N-[6-[4-[(2,6-dioxo-3-piperidyl)amino]anilino]-6-oxo-hexyl]carbamate (200 mg, 462.41 mol, 48.46% yield) was obtained as a blue solid

LC-MS [ESI, M+1]:432.2.

¹H NMR (400 MHz, DMSO-d₆) δ=10.77 (s, 1H), 9.47 (s, 1H), 7.27 (d, J=8.8 Hz, 2H), 6.77 (br s, 1H), 6.60 (br d, J=8.8 Hz, 2H), 5.63 (d, J=7.6 Hz, 1H), 4.31-4.18 (m, 1H), 2.89 (q, J=6.8 Hz, 2H), 2.79-2.67 (m, 1H), 2.62-2.54 (m, 1H), 2.20 (br t, J=7.2 Hz, 2H), 2.11 (td, J=4.4, 8.8 Hz, 1H), 1.91-1.78 (m, 1H), 1.54 (q, J=7.2 Hz, 2H), 1.36 (s, 11H), 1.30-1.21 (m, 2H).

### Step 4: 6-amino-N-[4-[(2,6-dioxo-3-piperidyl)amino]phenyl]hexanamide

To a solution of tert-butyl N-[6-[4-[(2,6-dioxo-3-piperidyl)amino]anilino]-6-oxo-hexyl]carbamate (100 mg, 231.21 mol, 1 eq) in DCM (1 mL) was added HCl/dioxane (2 M, 1.00 mL, 8.65 eq). The mixture was stirred at 25° C. for 0.2 h. LCMS showed starting material was consumed and one main peak with desired mass was detected. The reaction solution was concentrated under the reduced pressure to give compound 6-amino-N-[4-[(2,6-dioxo-3-piperidyl)amino]phenyl]hexanamide (85 mg, 99%, HCl) as a green solid.

LC-MS [ESI, M+1]: 333.2.

### Step 5: N-[6-[4-[(2,6-dioxo-3-piperidyl)amino]anilino]-6-oxo-hexyl]-8-methyl-6-[(3R)-3-methyl-3-phenyl-pyrrolidin-1-yl]-[1,2,4]triazolo[1,5-a]pyridine-2-carboxamide

To a solution of 8-methyl-6-[(3R)-3-methyl-3-phenyl-pyrrolidin-1-yl]-[1,2,4]triazolo[1,5-a]pyridine-2-carboxylic acid (30 mg, 89.18 mol, 1 eq) in DMF (0.5 mL) was added HATU (67.82 mg, 178.37 mol, 2 eq) at 0° C. After addition, the mixture was stirred at this temperature for 0.5 h. and then 6-amino-N-[4-[(2,6-dioxo-3-piperidyl)amino]phenyl]hexanamide (39.47 mg, 107.02 mol, 1.2 eq, HCl) and DIEA (57.63 mg, 445.92 mol, 77.67 μL, 5 eq) was added dropwise at 0° C. The resulting mixture was stirred at 25° C. for 0.5 h. LCMS showed starting material was consumed and one main peak with desired mass was detected. The mixture was filtered. The residue was purified by prep-HPLC (column: CD04-Welch Utimate C18 150*25*7 um; mobile phase: [Water(FA)-MeCN]; gradient:37%-67% B over 15 min) to give compound N-[6-[4-[(2,6-dioxo-3-piperidyl)amino]anilino]-6-oxo-hexyl]-8-methyl-6-[(3R)-3-methyl-3-phenyl-pyrrolidin-1-yl]-[1,2,4]triazolo[1,5-a]pyridine-2-carboxamide (11.28 mg, 19%) as a white solid.

LC-MS [ESI, M]: 651.4.

¹H NMR (400 MHz, DMSO-d₆) δ=10.76 (br s, 1H), 9.48 (s, 1H), 8.49 (br t, J=6.0 Hz, 1H), 7.89 (d, J=1.2 Hz, 1H), 7.44-7.33 (m, 4H), 7.32-7.21 (m, 4H), 6.60 (d, J=8.8 Hz, 2H), 5.62 (d, J=7.6 Hz, 1H), 4.31-4.17 (m, 1H), 3.63-3.57 (m, 1H), 3.55-3.46 (m, 2H), 3.41-3.36 (m, 1H), 3.27 (br d, J=6.8 Hz, 2H), 2.79-2.66 (m, 1H), 2.61-2.53 (m, 4H), 2.30-2.19 (m, 4H), 2.15-2.05 (m, 1H), 1.90-1.77 (m, 1H), 1.65-1.51 (m, 4H), 1.38-1.29 (m, 5H).

### Example 15

### N-[6-[3-[(2,6-dioxo-3-piperidyl)amino]anilino]-6-oxo-hexyl]-8-methyl-6-[(3R)-3-methyl-3-phenylpyrrolidin-1-yl]-[1,2,4]triazolo[1,5-a]pyridine-2-carboxamide

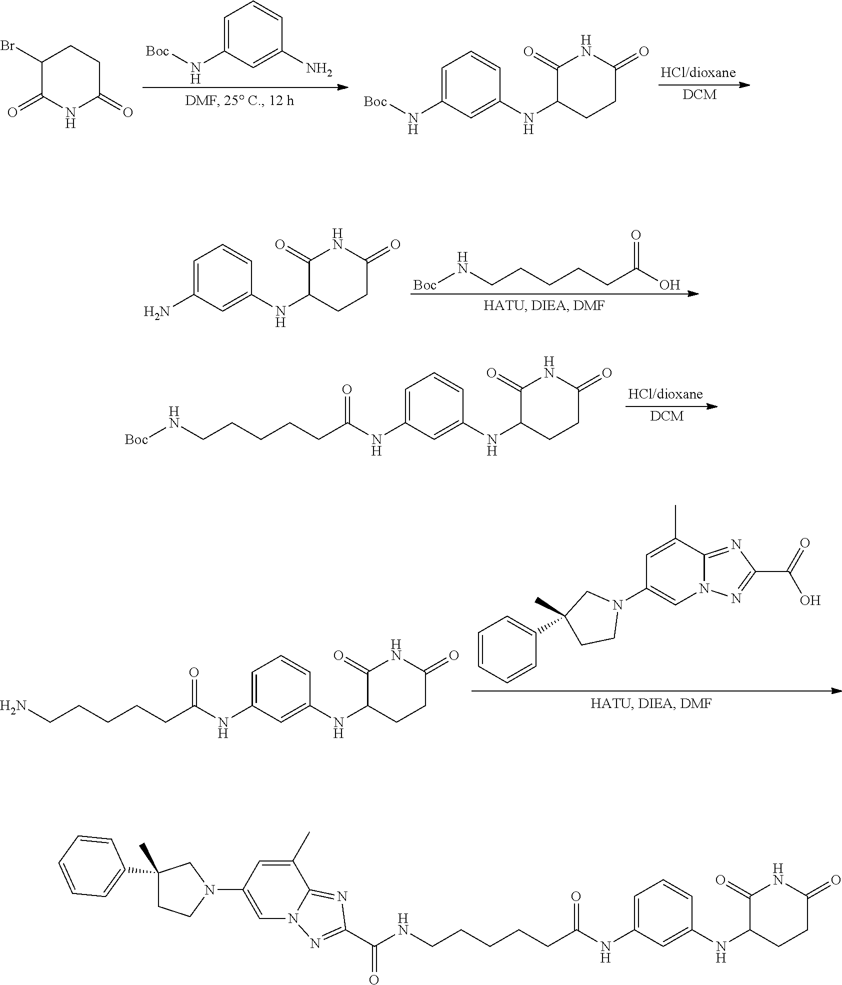

### Step 1: tert-butyl N-[3-[(2,6-dioxo-3-piperidyl)amino]phenyl]carbamate

To a solution of 3-bromopiperidine-2,6-dione (10 g, 52.08 mmol, 1 eq) in DMF (100 mL) was added tert-butyl N-(3-aminophenyl)carbamate (10.85 g, 52.08 mmol, 1 eq). The mixture was stirred at 30° C. for 12 h. LCMS showed starting material was consumed completely and the desired compound was detected. The reaction mixture was diluted with water(200 mL), then was extracted with ethyl acetate (200 mL×3). The combined organic phase was washed with brine (30 mL), dried with anhydrous Na₂SO₄, filtered and concentrated in vacuo to give a residue, The residue was purified by prep-HPLC (FA) and lyophilized to give tert-butyl N-[3-[(2,6-dioxo-3-piperidyl)amino]phenyl]carbamate (5.1 g, 30.66% yield) as green solid.

LC-MS [ESI, M-55]: 264.1.

¹H NMR (400 MHz, DMSO-d₆) δ=10.77 (s, 1H), 9.03 (s, 1H), 6.92 (t, J=8.0 Hz, 1H), 6.85 (s, 1H), 6.65 (br d, J=8.0 Hz, 1H), 6.29 (dd, J=1.2, 8.0 Hz, 1H), 5.79 (d, J=7.6 Hz, 1H), 4.25-4.16 (m, 1H), 2.78-2.65 (m, 1H), 2.63-2.54 (m, 1H), 2.10 (br dd, J=4.4, 8.8 Hz, 1H), 1.87 (br dd, J=4.4, 12.4 Hz, 1H).

### Step 2: 3-(3-aminoanilino)piperidine-2,6-dione

To a solution of tert-butyl N-[3-[(2,6-dioxo-3-piperidyl)amino]phenyl]carbamate (700 mg, 2.19 mmol, 1 eq) in DCM (5 mL) was added HCl/dioxane (2 M, 7 mL, 6.39 eq). The mixture was stirred at 25° C. for 1 h. LCMS showed starting material was consumed completely and the desired compound was detected. The reaction mixture was filtered and the filtrate was concentrated under the reduced pressure to give 3-(3-aminoanilino)piperidine-2,6-dione (700 mg, crude, HCl) as white solid.

### Step 3: tert-butyl N-[6-[3-[(2,6-dioxo-3-piperidyl)amino]anilino]-6-oxo-hexyl]carbamate

To a solution of 6-(tert-butoxycarbonylamino)hexanoic acid (90.45 mg, 391.08 mol, 1 eq) in DMF (2 mL) was added HATU (297.40 mg, 782.17 mol, 2 eq) and then was added 3-(3-aminoanilino)piperidine-2,6-dione (100 mg, 391.08 mol, 1 eq, HCl) and DIEA (151.63 mg, 1.17 mmol, 204.36 μL, 3 eq) after 0.1 h. The mixture was stirred at 25° C. for 1 h. LCMS showed starting material was consumed completely and the desired mass was detected. The reaction mixture was diluted with water (30 mL), then was extracted with ethyl acetate (30 mL×3). The combined organic phase was washed with brine (30 mL), dried with anhydrous Na₂SO₄, filtered and concentrated in vacuum to give a residue. The residue was purified by prep-HPLC (FA) and lyophilized to give tert-butyl N-[6-[3-[(2,6-dioxo-3-piperidyl)amino]anilino]-6-oxo-hexyl]carbamate (90 mg, 53% yield) as yellow solid.

LC-MS [ESI, M-99]: 333.2.

¹H NMR (400 MHz, CHLOROFORM-d) 6=7.39 (br s, 1H), 7.17-7.10 (m, 1H), 6.71-6.58 (m, 1H), 6.48 (br d, J=1.6 Hz, 1H), 4.59 (br d, J=1.2 Hz, 1H), 3.13 (br d, J=6.0 Hz, 2H), 2.88-2.77 (m, 2H), 2.66-2.56 (m, 1H), 2.36 (br t, J=7.2 Hz, 2H), 1.80-1.71 (m, 4H), 1.57-1.50 (m, 4H), 1.45 (s, 9H), 0.91-0.83 (m, 2H).

### Step 4: 6-amino-N-[3-[(2,6-dioxo-3-piperidyl)amino]phenyl]hexanamide

To a solution of tert-butyl N-[6-[3-[(2,6-dioxo-3-piperidyl)amino]anilino]-6-oxo-hexyl]carbamate (90 mg, 208.09 mol, 1 eq) in DCM (1 mL) was added HCl/dioxane (2 M, 1 mL, 9.61 eq). The mixture was stirred at 25° C. for 0.5 h. LCMS showed starting material was consumed completely and the desired mass was detected. The reaction mixture was concentrated under the reduced pressure to give methyl 6-amino-N-[3-[(2,6-dioxo-3-piperidyl)amino]phenyl]hexanamide (90 mg, crude, HCl) as white solid.

LC-MS [ESI, M+1]: 333.2.

### Step 5: N-[6-[3-[(2,6-dioxo-3-piperidyl)amino]anilino]-6-oxo-hexyl]-8-methyl-6-[(3R)-3-methyl-3-phenylpyrrolidin-1-yl]-[1,2,4]triazolo[1,5-a]pyridine-2-carboxamide

To a solution of 8-methyl-6-[(3R)-3-methyl-3-phenyl-pyrrolidin-1-yl]-[1,2,4]triazolo[1,5-a]pyridine-2-carboxylic acid (68.40 mg, 203.33 mol, 1 eq) in DMF (1 mL) was added HATU (154.62 mg, 406.66 mol, 2 eq) and then was added 6-amino-N-[3-[(2,6-dioxo-3-piperidyl)amino]phenyl]hexanamide (75 mg, 203.33 mol, 1 eq, HCl) and DIEA (78.84 mg, 609.99 mol, 106.25 μL, 3 eq) after 0.1 h. The mixture was stirred at 25° C. for 1 h. LCMS showed starting material was consumed completely and the desired compound was detected. The reaction mixture was concentrated under the reduced pressure to give a residue. The residue was purified by prep-HPLC (column: CD01-Phenomenex luna C18 150*25*10 um; mobile phase: [water(FA)-ACN]; gradient:40%-70% B over 10 min) and lyophilized to give N-[6-[3-[(2,6-dioxo-3-piperidyl)amino]anilino]-6-oxo-hexyl]-8-methyl-6-[(3R)-3-methyl-3-phenylpyrrolidin-1-yl]-[1,2,4]triazolo[1,5-a]pyridine-2-carboxamide (43.93 mg, 33% yield) as white solid.

LC-MS [ESI, M+1]: 651.5.

¹H NMR (400 MHz, DMSO-d₆) δ=10.77 (s, 1H), 9.59 (s, 1H), 8.62-8.36 (m, 1H), 7.88 (s, 1H), 7.42-7.30 (m, 5H), 7.27-7.20 (m, 1H), 7.03-6.89 (m, 2H), 6.77 (br d, J=7.6 Hz, 1H), 6.35 (br d, J=8.0 Hz, 1H), 5.82 (d, J=7.6 Hz, 1H), 4.23 (ddd, J=4.8, 7.2, 11.6 Hz, 1H), 3.63-3.56 (m, 1H), 3.55-3.45 (m, 2H), 3.37 (br dd, J=2.8, 5.6 Hz, 1H), 3.30-3.24 (m, 2H), 2.79-2.68 (m, 1H), 2.63-2.57 (m, 1H), 2.55 (s, 3H), 2.31-2.22 (m, 4H), 2.15-2.04 (m, 1H), 1.88 (br dd, J=4.4, 12.0 Hz, 1H), 1.58 (td, J=7.6, 15.2 Hz, 4H), 1.39-1.28 (m, 5H).

### Example 16

### N-[6-[[3-(2,6-dioxo-3-piperidyl)-1-methyl-indazol-7-yl]amino]-6-oxo-hexyl]-8-methyl-6-[(3R)-3-methyl3-phenyl-pyrrolidin-1-yl]-[1,2,4]triazolo[1,5-a]pyridine-2-carboxamide

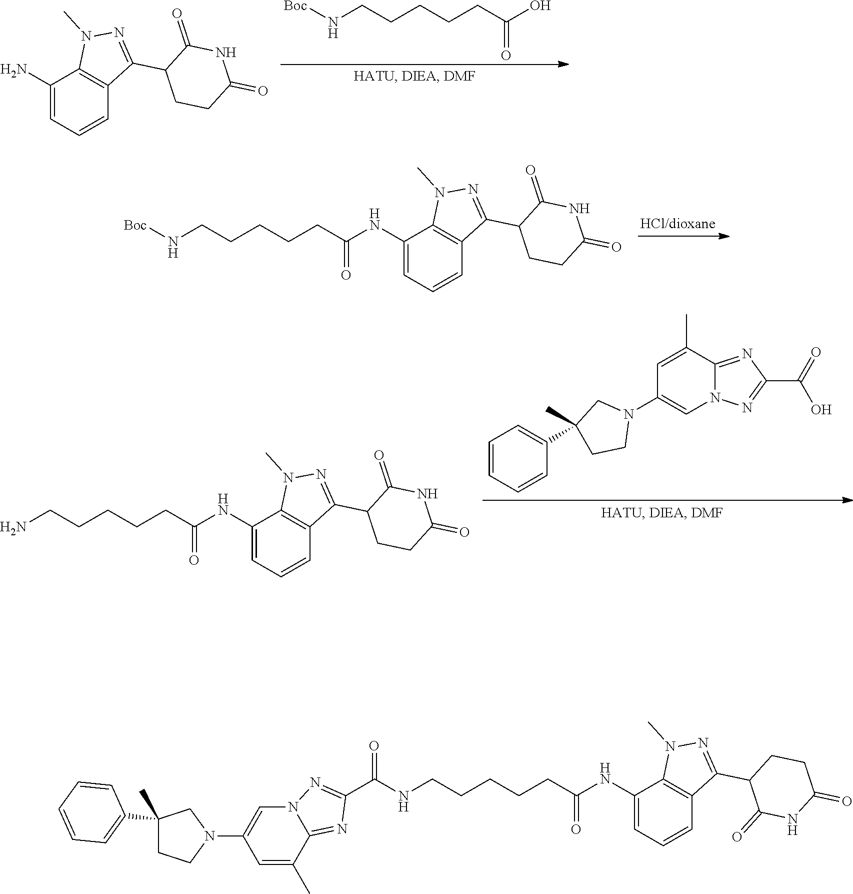 [SMILES: Cn1nc(C2CCC(=O)NC2=O)c2cccc(N)c21 ; CC(C)(C)OC(=O)NCCCCCC(=O)O ; Cn1nc(C2CCC(=O)NC2=O)c2cccc(NC(=O)CCCCCNC(=O)OC(C)(C)C)c21 ; Cn1nc(C2CCC(=O)NC2=O)c2cccc(NC(=O)CCCCCN)c21 ; Cc1cc(N2CC[C@](C)(c3ccccc3)C2)cn2nc(C(=O)O)nc12 ; Cc1cc(N2CC[C@](C)(c3ccccc3)C2)cn2nc(C(=O)NCCCCCC(=O)Nc3cccc4c(C5CCC(=O)NC5=O)nn(C)c34)nc12]

### Step 1: tert-butyl N-[6-[[3-(2,6-dioxo-3-piperidyl)-1-methyl-indazol-7-yl]amino]-6-oxo-hexyl]carbamate

To a solution of 3-(7-amino-1-methyl-indazol-3-yl)piperidine-2,6-dione (120 mg, 464.62 mol, 1 eq), 6-(tertbutoxycarbonylamino)hexanoic acid (107.46 mg, 464.62 mol, 1 eq) in DMF (2.5 mL) was added HATU (353.33 mg, 929.24 mol, 2 eq) and DIEA (180.15 mg, 1.39 mmol, 242.78 μL, 3 eq). The mixture was stirred at 25° C. for 2 h. LCMS showed starting material was consumed and the desired mass was detected. The reaction was concentrated under reduced pressure to give a residue. The residue was purified by prep-HPLC (column: CD03-Welch Xtimate C18 150*25*5 um; mobile phase: [water(FA)—ACN]; gradient:21%-51% B over 10 min) and lyophilizated to give compound tert-butyl N-[6-[[3-(2,6-dioxo-3-piperidyl)-1-methyl-indazol-7-yl]amino]-6-oxo-hexyl]carbamate (150 mg, 68% yield) was obtained as a white solid.

¹H NMR (400 MHz, DMSO-d₆) δ=10.88 (s, 1H), 10.06 (s, 1H), 8.08 (s, 1H), 7.61 (d, J=8.8 Hz, 1H), 7.08 (dd, J=1.2, 8.8 Hz, 1H), 6.81 (br t, J=5.6 Hz, 1H), 4.31 (dd, J=5.2, 9.6 Hz, 1H), 3.91 (s, 3H), 2.94 (q, J=6.8 Hz, 2H), 2.71-2.58 (m, 2H), 2.54 (s, 1H), 2.39-2.27 (m, 3H), 1.99 (s, 1H), 1.63-1.52 (m, 2H), 1.37 (s, 9H)

LC-MS [ESI, M-100]:372.2

### Step 2: 6-amino-N-[3-(2,6-dioxo-3-piperidyl)-1-methyl-indazol-7-yl]hexanamide

A solution of tert-butyl N-[6-[[3-(2,6-dioxo-3-piperidyl)-1-methyl-indazol-7-yl]amino]-6-oxo-hexyl]carbamate (140 mg, 296.89 mol, 1 eq) in HCl/dioxane (4M, 10 mL) was stirred at 25° C. for 0.5 h. TLC (DCM: MeOH=10:1, Rf=0.3) indicated reactant was consumed and one new spot formed. The reaction was filtered and the filtered concentrated under reduced pressure to compound 6-amino-N-[3-(2,6-dioxo-3-piperidyl)-1-methyl-indazol-7-yl]hexanamide (140 mg, crude, HCl) was obtained as a white solid.

### Step 3: N-[6-[[3-(2,6-dioxo-3-piperidyl)-1-methyl-indazol-7-yl]amino]-6-oxo-hexyl]-8-methyl-6-[(3R)-3-methyl3-phenyl-pyrrolidin-1-yl]-[1,2,4]triazolo[1,5-a]pyridine-2-carboxamide

To a solution of 6-amino-N-[3-(2,6-dioxo-3-piperidyl)-1-methyl-indazol-7-yl]hexanamide (30 mg, 80.77 mol, 1 eq), 8-methyl-6-[(3R)-3-methyl-3-phenyl-pyrrolidin-1-yl]-[1,2,4]triazolo[1,5-a]pyridine-2-carboxylic acid (27.17 mg, 80.77 mol, 1 eq) in DMF (0.5 mL) was added HATU (61.42 mg, 161.54 mol, 2 eq) and DIEA (31.32 mg, 242.31 mol, 42.20 μL, 3 eq). The mixture was stirred at 25° C. for 1 h LCMS showed starting material was consumed and the desired mass was detected. The reaction was filtered and the filtered concentrated under reduced pressure to give a residue. The reaction was concentrated under reduced pressure to give a residue. The residue was purified by prep-HPLC (column: CD01-Phenomenex luna C18 150*25*10 um; mobile phase: [water(FA)-ACN]; gradient:36%-66% B over 8 min) andlyophilizated to give compound N-[6-[[3-(2,6-dioxo-3-piperidyl)-1-methyl-indazol-7-yl]amino]-6-oxo-hexyl]-8-methyl-6-[(3R)-3-methyl3-phenyl-pyrrolidin-1-yl]-[1,2,4]triazolo[1,5-a]pyridine-2-carboxamide (13.56 mg, 24% yield) was obtained as a white solid.

LC-MS [ESI, M+1]: 690.4

¹H NMR (400 MHz, DMSO-d₆) δ ppm 1.34-1.44 (m, 5H) 1.55-1.73 (m, 4H) 2.16 (dq, J=13.2, 5.2 Hz, 1H) 2.22-2.30 (m, 2H) 2.31-2.38 (m, 1H) 2.41 (brt, J=7.6 Hz, 2H) 2.55 (s, 3H) 2.59-2.74 (m, 2H) 3.27-3.32 (m, 2H) 3.35-3.41 (m, 1H) 3.46-3.55 (m, 2H) 3.58-3.62 (m, 1H) 4.03 (s, 3H) 4.36 (dd, J=10.0, 5.2 Hz, 1H) 7.01-7.11 (m, 2H) 7.21-7.26 (m, 1H) 7.31-7.42 (m, 5H) 7.58 (d, J=7.6 Hz, 1H) 7.89 (s, 1H) 8.51 (t, J=6.0 Hz, 1H) 9.83 (s, 1H) 10.90 (s, 1H)

### Example 17

### N-(10-((3-((2,6-dioxopiperidin-3-yl)amino)phenyl)amino)-10-oxodecyl)-8-methyl-6-((R)-3-methyl-3-phenylpyrrolidin-1-yl)-[1,2,4]triazolo[1,5-a]pyridine-2-carboxamide

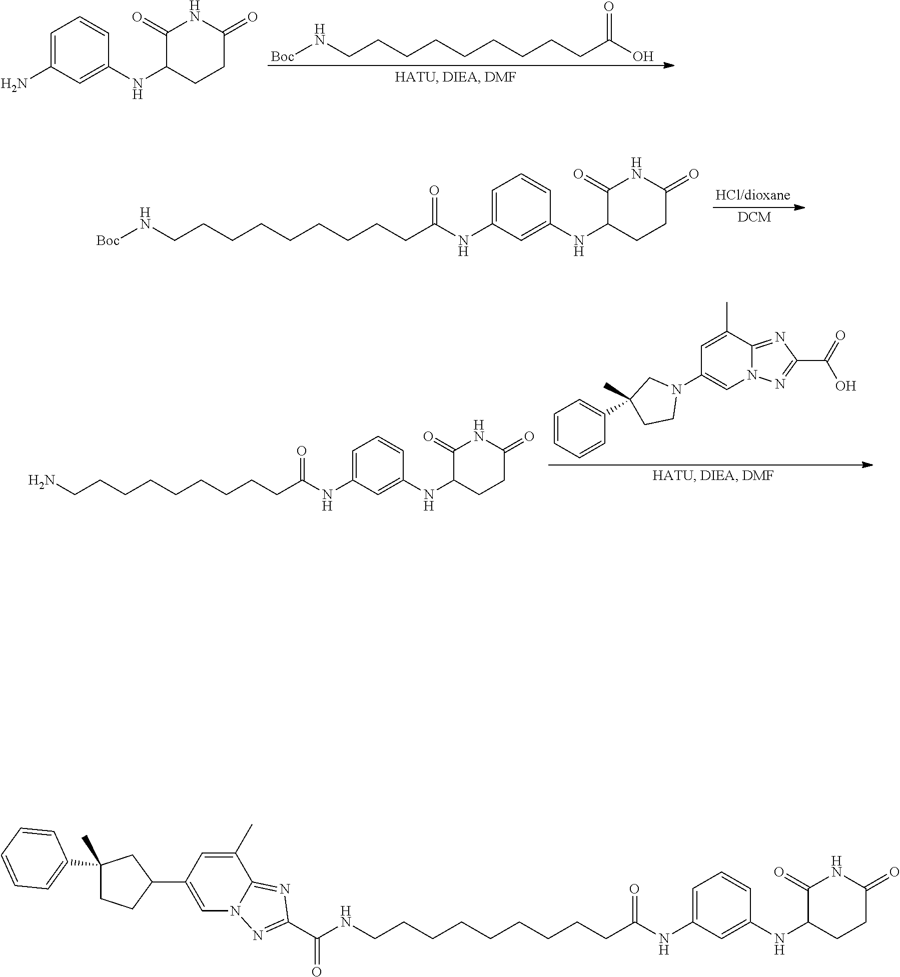

### Step 1: tert-butyl (10-((3-((2,6-dioxopiperidin-3-yl)amino)phenyl)amino)-10-oxodecyl)carbamate

To a solution of 3-(3-aminoanilino)piperidine-2,6-dione (100 mg, 391.08 mol, 1 eq, HCl) and 10-(tertbutoxycarbonylamino)decanoic acid (89.92 mg, 312.87 mol, 0.8 eq) in DMF (3 mL) was added HATU (297.40 mg, 782.17 mol, 2 eq) and DIEA (151.63 mg, 1.17 mmol, 204.36 μL, 3 eq). The mixture was stirred at 25° C. for 15 min, LCMS showed starting material was consumed completely and the desired compound was detected. The residue was concentrated to give the product tert-butyl N-[10-[3-[(2,6-dioxo-3-piperidyl)amino]anilino]-10-oxodecyl]carbamate (70 mg, 37% yield) was obtained as a white solid

LC-MS [ESI, M+1]: 489.3.

¹H NMR (CHLOROFORM-d, 400 MHz): δ=7.99 (br s, 1.0H), 7.39 (br s, 0.9H), 7.21 (br s, 1.0H), 7.13 (t, J=8.0 Hz, 1.2H), 6.61 (br d, J=8.0 Hz, 1.2H), 6.44 (br d, J=8.4 Hz, 1.2H), 4.52 (br s, 1.2H), 4.13 (dd, J=12.4, 4.8 Hz, 1.0H), 3.10 (br d, J=6.4 Hz, 2.0H), 2.74-2.91 (m, 2.1H), 2.56-2.65 (m, 1.1H), 2.34 (t, J=7.6 Hz, 2.1H), 1.89 (br dd, J=13.2, 5.2 Hz, 1.4H), 1.71 (br d, J=7.2 Hz, 3.2H), 1.45 (s, 9.0H), 1.33 (br s, 4.1H), 1.30 (br s, 7.2H), 1.26 ppm (br s, 2.0H)

### Step 2: 10-amino-N-(3-((2,6-dioxopiperidin-3-yl)amino)phenyl)decanamide

To a solution of tert-butyl N-[10-[3-[(2,6-dioxo-3-piperidyl)amino]anilino]-10-oxo-decyl]carbamate (70 mg, 143.26 mol, 1 eq) in DCM (2 mL) was added HCl/dioxane (2 M, 71.63 μL, 1 eq). The mixture was stirred at 25° C. for 15 min. LCMS showed starting material was consumed completely and the desired compound was detected. The reaction mixture was concentrated under the reduced pressure to give the product. Compound10-amino-N-[3-[(2,6-dioxo-3-piperidyl)amino]phenyl]decanamide (60 mg, 141.19 mol, 99% yield, HCl) was obtained as a yellow gum.

LC-MS [ESI, M+23]: 389.1.

### Step 3: N-[10-[3-[(2,6-dioxo-3-piperidyl)amino]anilino]-10-oxo-decyl]-8-methyl-6-[(3R)-3-methyl-3-phenyl-pyrrolidin-1-yl]-[1,2,4]triazolo[1,5-a]pyridine2-carboxamide

To a solution of 10-amino-N-[3-[(2,6-dioxo-3-piperidyl)amino]phenyl]decanamide (50 mg, 117.66 mol, 0.9 eq, HCl) and 8-methyl-6-[(3R)-3-methyl-3-phenyl-pyrrolidin-1-yl]-[1,2,4]triazolo[1,5-a]pyridine-2-carboxylic acid (43.98 mg, 130.73 mol, 1 eq) in DMF (2 mL) was added HATU (99.42 mg, 261.46 mol, 2 eq) and DIEA (50.69 mg, 392.19 mol, 68.31 μL, 3 eq). The mixture was stirred at 25° C. for 15 min. LCMS showed starting material was consumed completely and the desired mass was detected. The reaction mixture was concentrated under the reduced pressure to give the product. The residue was purified by prep-HPLC (column: CD01-Phenomenex luna C18 150*25*10 um; mobile phase: [water(FA)-ACN]; gradient:50%-80% B over 8 min and lyophilized to give compound N-[10-[3-[(2,6-dioxo-3-piperidyl)amino]anilino]-10-oxo-decyl]-8-methyl-6-[(3R)-3-methyl-3-phenyl-pyrrolidin-1-yl]-[1,2,4]triazolo[1,5-a]pyridine2-carboxamide (32.94 mg, 100% purity) was obtained as a yellow solid.

LC-MS [ESI, M+1]: 707.5.

¹H NMR (CHLOROFORM-d, 400 MHz): δ=8.33 (br s, 1.0H), 7.89 (br s, 1.0H), 7.63 (s, 1.0H), 7.42 (br t, J=5.6 Hz, 1.0H), 7.36 (br d, J=7.2 Hz, 2.9H), 7.29-7.33 (m, 2.0H), 7.26 (br s, 1.0H), 7.09 (br t, J=8.0 Hz, 1.0H), 7.00 (s, 1.0H), 6.78 (br d, J=7.6 Hz, 1.0H), 6.41 (br d, J=8.0 Hz, 1.2H), 4.10 (br dd, J=12.4, 4.4 Hz, 1.0H), 3.58 (d, J=9.2 Hz, 1.0H), 3.44-3.55 (m, 4.1H), 3.35-3.43 (m, 1.1H), 2.68-2.83 (m, 2.3H), 2.61 (s, 3.2H), 2.55 (br dd, J=9.6, 3.6 Hz, 1.2H), 2.31-2.38 (m, 3.3H), 2.26 (br dd, J=7.2, 3.6 Hz, 1.3H), 1.84 (br dd, J=12.4, 4.8 Hz, 1.1H), 1.59-1.73 (m, 4.2H), 1.45 (s, 3.1H), 1.30 ppm (br s, 10.1H)

### Example 18

### N-(8-((3-((2,6-dioxopiperidin-3-yl)amino)phenyl)amino)-8-oxooctyl)-8-methyl-6-((R)-3-methyl-3-phenylpyrrolidin-1-yl)-[1,2,4]triazolo[1,5-a]pyridine-2-carboxamide

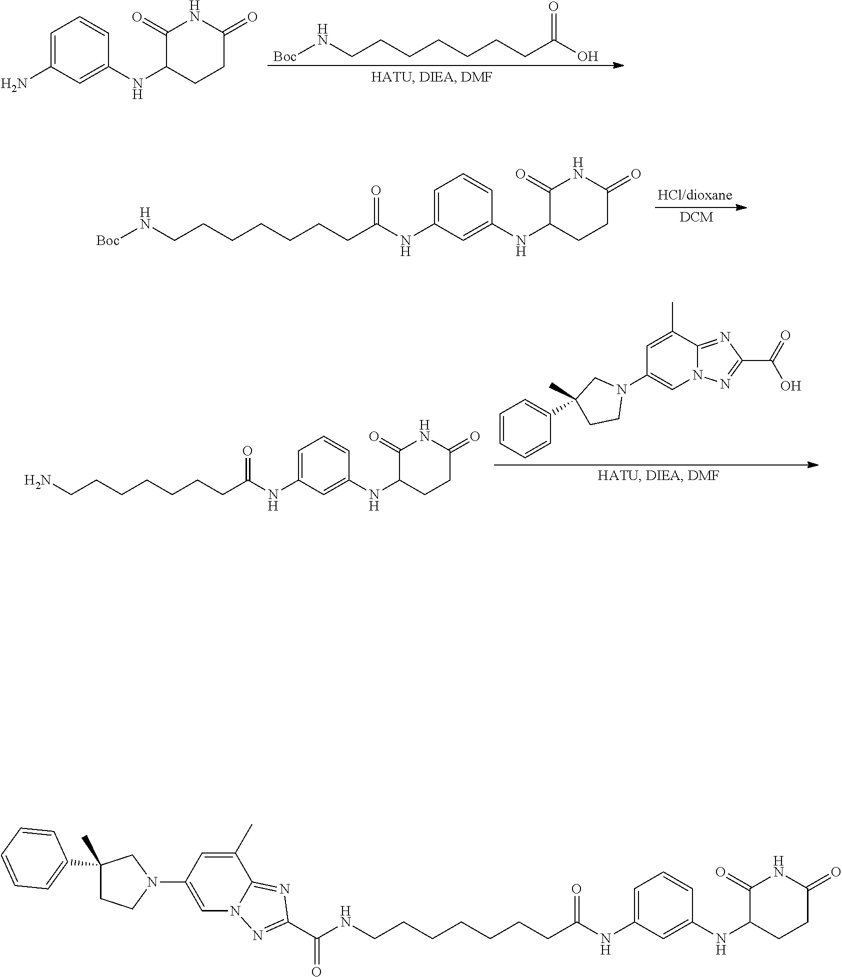

### Step 1: tert-butyl (8-((3-((2,6-dioxopiperidin-3-yl)amino)phenyl)amino)-8-oxooctyl)carbamate

To a solution of 3-(3-aminoanilino)piperidine-2,6-dione (100 mg, 391.08 mol, 1 eq, HCl) and 8-(tertbutoxycarbonylamino)octanoic acid (81.14 mg, 312.87 mol, 0.8 eq) in DMF (4 mL) was added HATU (297.40 mg, 782.17 mol, 2 eq) and DIEA (151.63 mg, 1.17 mmol, 204.36 μL, 3 eq). The mixture was stirred at 25° C. for 15 min, LCMS showed starting material was consumed completely and the desired compound was detected. The residue was concentrated to give the compound tert-butyl N-[8-[3-[(2,6-dioxo-3-piperidyl)amino]anilino]-8-oxooctyl]carbamate (100 mg, 56% yield) was obtained as a white solid

LC-MS [ESI, M+1]: 461.3.

¹H NMR (CHLOROFORM-d, 400 MHz): δ=7.99 (br s, 1.0H), 7.39 (br s, 1.0H), 7.13 (t, J=8.0 Hz, 1.2H), 6.62 (br d, J=7.2 Hz, 1.2H), 6.44 (br d, J=8.0 Hz, 1.2H), 4.53 (br d, J=2.0 Hz, 1.2H), 4.13 (dd, J=12.4, 4.4 Hz, 1.1H), 3.12 (br d, J=5.6 Hz, 2.0H), 2.73-2.91 (m, 2.2H), 2.57-2.65 (m, 1.0H), 2.34 (t, J=7.2 Hz, 2.0H), 1.83-1.96 (m, 1.3H), 1.72 (br d, J=7.2 Hz, 2.0H), 1.61 (br s, 3.4H), 1.45 (s, 9.3H), 1.35 (br s, 6.0H), 1.26 ppm (s, 1.1H)

### Step 2: 8-amino-N-(3-((2,6-dioxopiperidin-3-yl)amino)phenyl)octanamide

To a solution of tert-butyl N-[8-[3-[(2,6-dioxo-3-piperidyl)amino]anilino]-8-oxo-octyl]carbamate (90 mg, 195.41 mol, 1 eq) in DCM (2 mL) was added HCl/dioxane (2 M, 97.71 μL, 1 eq). The mixture was stirred at 25° C. for 15 min. LCMS showed starting material was consumed completely and the desired compound was detected. The reaction mixture was concentrated under the reduced pressure to give the compound8-amino-N-[3-[(2,6-dioxo-3-piperidyl)amino]phenyl]octanamide (77 mg, 99% yield, HCl) was obtained as a yellow gum.

LC-MS [ESI, M+23]: 361.1.

### Step 3: N-(8-((3-((2,6-dioxopiperidin-3-yl)amino)phenyl)amino)-8-oxooctyl)-8-methyl-6-((R)-3-methyl-3-phenylpyrrolidin-1-yl)-[1,2,4]triazolo[1,5-a]pyridine-2-carboxamide

To a solution of 8-amino-N-[3-[(2,6-dioxo-3-piperidyl)amino]phenyl]octanamide (55 mg, 138.57 mol, 0.8 eq, HCl) and 8-methyl-6-[(3R)-3-methyl-3-phenyl-pyrrolidin-1-yl]-[1,2,4]triazolo[1,5-a]pyridine-2-carboxylic acid (58.27 mg, 173.21 mol, 1 eq) in DMF (4 mL) was added HATU (131.72 mg, 346.43 mol, 2 eq) and DIEA (67.16 mg, 519.64 mol, 90.51 μL, 3 eq). The mixture was stirred at 25° C. for 15 min. LCMS showed starting material was consumed completely and the desired mass was detected. The reaction mixture was concentrated under the reduced pressure to give the product. The residue was purified by prep-HPLC ((column: CD01-Phenomenex luna C18 150*25*10 um; mobile phase: [water(FA)-ACN]; gradient:42%-72% B over 8 min) and lyophilized to give compound N-[8-[3-[(2,6-dioxo-3-piperidyl)amino]anilino]-8-oxo-octyl]-8-methyl6-[(3R)-3-methyl-3-phenyl-pyrrolidin-1-yl]-[1,2,4]triazolo[1,5-a]pyridine-2-carboxamide (69.08 mg, 57% yield) was obtained as a gray solid

LC-MS [ESI, M+1]: 679.4

¹H NMR (CHLOROFORM-d, 400 MHz): δ=8.27 (br s, 0.9H), 7.95 (br s, 0.9H), 7.62 (s, 1.0H), 7.41-7.52 (m, 1.0H), 7.29-7.40 (m, 5.1H), 7.26 (br s, 1.1H), 7.10 (br t, J=8.0 Hz, 1.2H), 7.00 (s, 1.0H), 6.83 (br d, J=7.2 Hz, 1.2H), 6.42 (br d, J=8.0 Hz, 1.0H), 4.04-4.17 (m, 1.1H), 3.58 (d, J=8.8 Hz, 1.2H), 3.44-3.55 (m, 4.1H), 3.36-3.43 (m, 1.0H), 2.77 (br s, 2.0H), 2.61 (s, 3.1H), 2.55 (br d, J=12.8 Hz, 1.2H), 2.30-2.41 (m, 4.1H), 2.22-2.28 (m, 2.0H), 1.85 (br dd, J=12.4, 5.6 Hz, 1.2H), 1.59-1.75 (m, 4.2H), 1.43-1.50 (m, 3.3H), 1.37 ppm (br s, 5.1H)

### Example 19

### N-[5-[[3-(2,6-dioxo-3-piperidyl)-1-methyl-indazol-6-yl]amino]-5-oxo-pentyl]-8-methyl-6-[(3R)-3-methyl3-phenyl-pyrrolidin-1-yl]-[1,2,4]triazolo[1,5-a]pyridine-2-carboxamide acid

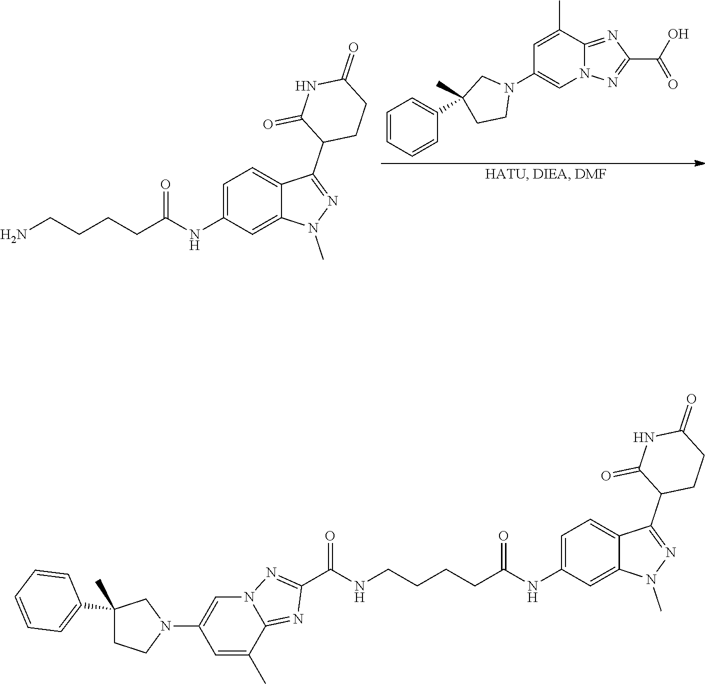

### Step 1: N-[5-[[3-(2,6-dioxo-3-piperidyl)-1-methyl-indazol-6-yl]amino]-5-oxo-pentyl]-8-methyl-6-[(3R)-3-methyl3-phenyl-pyrrolidin-1-yl]-[1,2,4]triazolo[1,5-a]pyridine-2-carboxamide acid

To a solution of 5-amino-N-[3-(2,6-dioxo-3-piperidyl)-1-methyl-indazol-6-yl]pentanamide (35.13 mg, 89.18 mol, 1 eq, HCl) and 8-methyl-6-[(3R)-3-methyl-3-phenyl-pyrrolidin-1-yl]-[1,2,4]triazolo[1,5-a]pyridine-2-carboxylic acid (30 mg, 89.18 mol, 1 eq) in DMF (2 mL) was added HATU (50.87 mg, 133.77 mol, 1.5 eq) and DIEA (34.58 mg, 267.55 mol, 46.60 μL, 3 eq). The mixture was stirred at 25° C. for 2 h. LCMS showed starting material was consumed and the desired mass was detected. The reaction was filtered and the filter was concentrated under reduced pressure to give a residue. The residue was purified by prep-HPLC (column: CD01-Phenomenex luna C18 150*25*10 um; mobile phase: [Water(FA)-MeCN]; gradient:56%-86% B over 8 min). The fraction was lyophilized to give compound N-[5-[[3-(2,6-dioxo-3-piperidyl)-1-methyl-indazol-6-yl]amino]-5-oxo-pentyl]-8-methyl-6-[(3R)-3-methyl3-phenyl-pyrrolidin-1-yl]-[1,2,4]triazolo[1,5-a]pyridine-2-carboxamide (28.19 mg, 46% yield) was obtained as a white solid

LC-MS [ESI, M+1]: 676.4

¹H NMR (400 MHz, DMSO-d₆) δ=10.89 (s, 1H), 10.10 (s, 1H), 8.55 (t, J=6.0 Hz, 1H), 8.09 (s, 1H), 7.86 (s, 1H), 7.60 (d, J=8.8 Hz, 1H), 7.40-7.28 (m, 5H), 7.26-7.19 (m, 1H), 7.09 (dd, J=1.2, 8.8 Hz, 1H), 4.31 (dd, J=5.2, 9.6 Hz, 1H), 3.90 (s, 3H), 3.62-3.55 (m, 1H), 3.53-3.43 (m, 2H), 3.33 (br d, J=6.4 Hz, 3H), 2.73-2.57 (m, 2H), 2.54 (s, 3H), 2.40 (br t, J=6.8 Hz, 2H), 2.33 (dt, J=4.4, 9.2 Hz, 1H), 2.27-2.21 (m, 2H), 2.20-2.11 (m, 1H), 1.73-1.55 (m, 4H), 1.34 (s, 3H)

### Example 20

### 4-[[6-[[3-(2,6-dioxo-3-piperidyl)-1-methyl-indazol-7-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

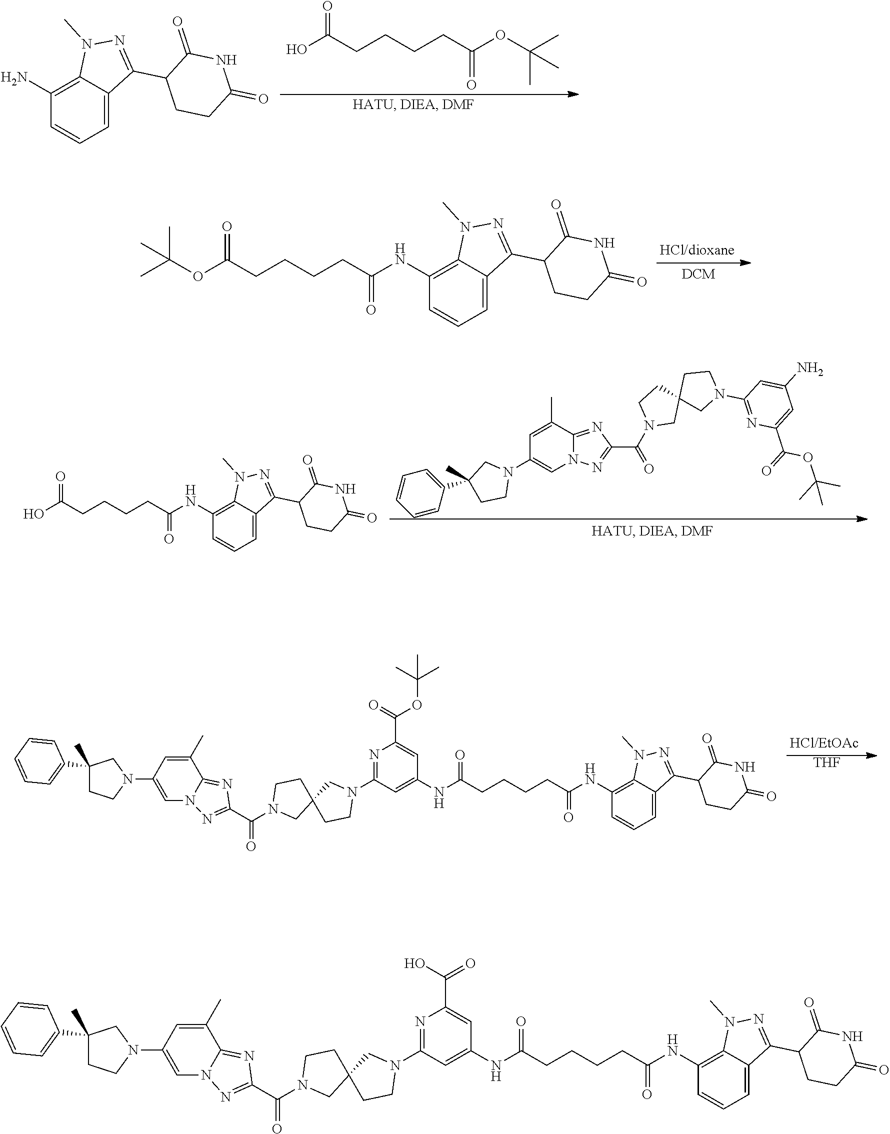

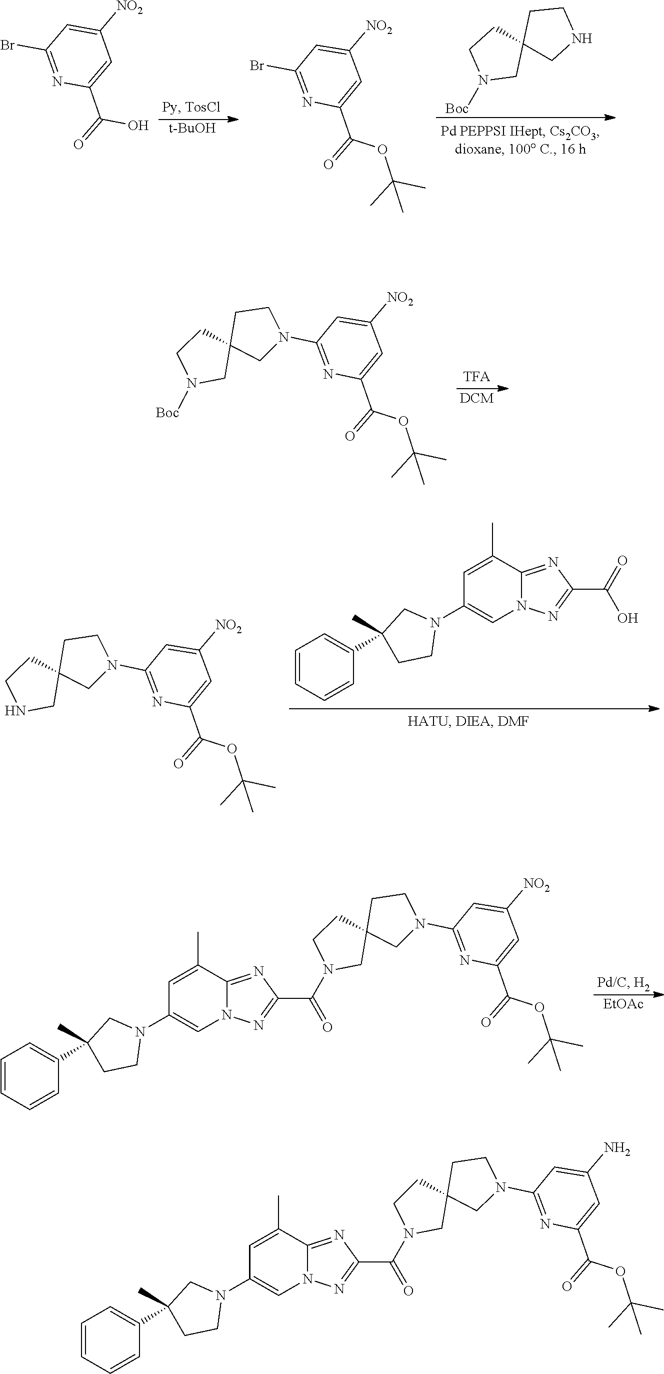

### Step 1: tert-butyl 6-[[3-(2,6-dioxo-3-piperidyl)-1-methyl-indazol-7-yl]amino]-6-oxo-hexanoate

To a solution of 6-tert-butoxy-6-oxo-hexanoic acid (234.92 mg, 1.16 mmol, 1.5 eq) in DMF (3 mL) was added HATU (441.66 mg, 1.16 mmol, 1.5 eq) at 25° C. After addition, the mixture was stirred at this temperature for 0.5 h, and then 3-(7-amino-1-methyl-indazol-3-yl)piperidine-2,6-dione (200 mg, 774.37 mol, 1 eq) and DIEA (400.32 mg, 3.10 mmol, 539.51 L, 4 eq) was added. The resulting mixture was stirred at 25° C. for 11.5 h. LCMS showed starting material consumed. One main peak with desired MS was detected. The reaction mixture was diluted with H₂O (10 mL) and extracted with ethyl acetate (15 mL×3). The combined organic layers were washed with brine 30 mL (10 mL×3), dried over anhydrous sodium sulfate, filtered and concentrated under reduced pressure to give a residue. The residue was purified by flash silica gel chromatography (ISCO®; 12 g SepaFlash® Silica Flash Column, Eluent of 0˜90% Ethyl acetate/Petroleum ethergradient @60 mL/min, PE: EtOAc=0:1, Rf=0.4) to give compound tert-butyl 6-[[3-(2,6-dioxo-3-piperidyl)-1-methyl-indazol-7-yl]amino]-6-oxo-hexanoate (335 mg, 96% yield) as a red solid.

LC-MS [ESI, M+1]: 443.1.

1H NMR (400 MHz, CHLOROFORM-d) 6=8.26 (s, 1H), 7.77 (s, 1H), 7.54 (d, J=8.0 Hz, 1H), 7.14 (br d, J=7.2 Hz, 1H), 7.09-7.02 (m, 1H), 4.32-4.24 (m, 1H), 4.07 (s, 3H), 2.96 (s, 1H), 2.89 (s, 1H), 2.73-2.62 (m, 1H), 2.52-2.41 (m, 3H), 2.31 (br t, J=6.8 Hz, 2H), 1.83-1.75 (m, 2H), 1.74-1.66 (m, 2H), 1.46 (s, 9H)

### Step 2: 6-[[3-(2,6-dioxo-3-piperidyl)-1-methyl-indazol-7-yl]amino]-6-oxo-hexanoic acid

To a solution of tert-butyl 6-[[3-(2,6-dioxo-3-piperidyl)-1-methyl-indazol-7-yl]amino]-6-oxo-hexanoate (80 mg, 180.79 mol, 1 eq) in DCM (0.5 mL) was added HCl/dioxane (8 mL). The mixture was stirred at 35° C. for 12 hr. LCMS showed starting material consumed. The reaction mixture was concentrated under reduced pressure to give a residue 6-[[3-(2,6-dioxo-3-piperidyl)-1-methyl-indazol-7-yl]amino]-6-oxo-hexanoic acid as a white solid.

LC-MS [ESI, M+1]: 386.9.

### Step 3: tert-butyl 4-[[6-[[3-(2,6-dioxo-3-piperidyl)-1-methyl-indazol-7-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of 6-[[3-(2,6-dioxo-3-piperidyl)-1-methyl-indazol-7-yl]amino]-6-oxo-hexanoic acid (39.84 mg, 94.22 mol, 1.2 eq, HCl) in pyridine (1 mL) was added tert-butyl 4-amino-6-[(5R)-2-[8-methyl-6-[(3R)-3-methyl-3-phenylpyrrolidin-1-yl]-[1,2,4]triazolo[1,5-a]pyridine-2-carbonyl]-2,7-diazaspiro[4.4]nonan-7-yl]pyridine-2-carboxylate (50 mg, 78.52 mol, 1 eq). Then POCl3 (12.40 mg, 80.88 mol, 7.54 μL, 1.03 eq) was added at 0° C. The mixture was stirred at 0° C. for 1 hr. LCMS showed starting material consumed. The reaction mixture was quenched with H₂O (0.1 mL) and then diluted with H₂O (2 mL) and extracted with DCM (10 mL×3). The combined organic layers were washed with brine (3 mL×3), dried over anhydrous sodium sulfate, filtered and concentrated under reduced pressure to give a residue. The residue was purified by prep-HPLC (FA condition; column: CD24-XPT C18 150×25×7 um; mobile phase: [water(FA)-ACN]; gradient: 34%-64% B over 11 min) to give compound tert-butyl 4-[[6-[[3-(2,6-dioxo-3-piperidyl)-1-methyl-indazol-7-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (22.55 mg, 27% yield) as a gray solid.

LC-MS [ESI, M+1]: 1005.5.

1H NMR (400 MHz, DMSO-d₆) δ=10.90 (br s, 1H), 10.19 (br d, J=10.0 Hz, 1H), 9.85 (br d, J=2.4 Hz, 1H), 7.91 (br d, J=16.0 Hz, 1H), 7.59 (br d, J=8.4 Hz, 1H), 7.42-7.19 (m, 7H), 7.16-7.01 (m, 3H), 4.37 (br dd, J=4.8, 9.8 Hz, 1H), 4.03 (d, J=2.4 Hz, 3H), 3.98-3.88 (m, 1H), 3.84-3.75 (m, 1H), 3.67 (br t, J=6.8 Hz, 1H), 3.63-3.44 (m, 6H), 3.42-3.36 (m, 3H), 2.70-2.60 (m, 2H), 2.54 (s, 3H), 2.44-2.32 (m, 5H), 2.29-2.20 (m, 2H), 2.16 (br dd, J=4.4, 13.2 Hz, 1H), 2.06-1.94 (m, 4H), 1.67 (br d, J=5.2 Hz, 4H), 1.52 (d, J=5.6 Hz, 9H), 1.34 (br d, J=10.4 Hz, 3H)

### Step 4: 4-[[6-[[3-(2,6-dioxo-3-piperidyl)-1-methyl-indazol-7-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of tert-butyl 4-[[6-[[3-(2,6-dioxo-3-piperidyl)-1-methyl-indazol-7-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (20 mg, 19.90 mol, 1 eq) in THE (0.5 mL) was added HCl/EtOAc (1.5 mL). The mixture was stirred at 25° C. for 3 h. LCMS showed starting material consumed. The residue was purified by prep-HPLC (FA condition, column: CD01-Phenomenex luna C18 150×25×10 um; mobile phase: [water(FA)-ACN]; gradient: 28%-58% B over 10 min) to give compound 4-[[6-[[3-(2,6-dioxo-3-piperidyl)-1-methyl-indazol-7-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (4.09 mg, 20% yield) as a yellow solid.

LC-MS [ESI, M+1]: 949.5.

¹H NMR (400 MHz, DMSO-d₆) δ=10.90 (s, 1H), 10.29 (br d, J=12.0 Hz, 1H), 9.86 (br d, J=2.8 Hz, 1H), 7.91 (br d, J=14.8 Hz, 1H), 7.59 (br d, J=7.6 Hz, 1H), 7.49-7.19 (m, 7H), 7.18-6.96 (m, 3H), 4.37 (br dd, J=4.8, 10.1 Hz, 1H), 4.03 (d, J=2.8 Hz, 3H), 3.99-3.92 (m, 1H), 3.83-3.78 (m, 1H), 3.69-3.66 (m, 1H), 3.65-3.52 (m, 9H), 2.74-2.57 (m, 2H), 2.54 (s, 3H), 2.44-2.32 (m, 5H), 2.29-2.21 (m, 2H), 2.19-2.13 (m, 1H), 2.08-1.94 (m, 4H), 1.68 (br d, J=5.6 Hz, 4H), 1.34 (d, J=10.0 Hz, 3H)

### tert-butyl 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

### Step 1: tert-butyl 6-bromo-4-nitro-pyridine-2-carboxylate

To a solution of 6-bromo-4-nitro-pyridine-2-carboxylic acid (5 g, 20.24 mmol, 1 eq) in t-BuOH (50 mL) and pyridine (12.81 g, 161.94 mmol, 13.07 mL, 8 eq) was added TosCl (11.58 g, 60.73 mmol, 3 eq) at 0° C. The mixture was stirred at 25° C. for 2 hr. TLC indicated starting material was consumed completely and one major new spot with lower polarity was detected (PE:EtOAc=3:1, Rf=0.4). The reaction mixture was quenched with NaHCO₃ (10 mL) and then concentrated under reduced pressure to give a residue. The residue was diluted with H2O 40 mL and extracted with ethyl acetate (50 mL×3). The combined organic layers were washed with brine (20 mL×3), dried over anhydrous sodium sulfate, filtered and concentrated under reduced pressure to give a residue. The residue was purified by flash silica gel chromatography (ISCO®; 12 g SepaFlash® Silica Flash Column, Eluent of 0-12% Ethyl acetate/Petroleum ethergradient @70 mL/min, PE: EtOAc=3:1, Rf=0.4) to give compound tert-butyl 6-bromo-4-nitro-pyridine-2-carboxylate (4.54 g, 73% yield) as a yellow solid.

LC-MS [ESI, M-56+1]: 249.0.

1H NMR (400 MHz, CHLOROFORM-d) 6=8.61 (d, J=1.6 Hz, 1H), 8.35 (d, J=1.6 Hz, 1H), 1.65 (s, 9H)

### Step 2: tert-butyl (5R)-7-(6-tert-butoxycarbonyl-4-nitro-2-pyridyl)-2,7-diazaspiro[4.4]nonane-2-carboxylate

To a solution of tert-butyl 6-bromo-4-nitro-pyridine-2-carboxylate (1.5 g, 4.95 mmol, 1 eq) in dioxane (20 mL) was added tert-butyl (5R)-2,7-diazaspiro[4.4]nonane-2-carboxylate (1.12 g, 4.95 mmol, 1 eq), Cs₂CO₃ (4.03 g, 12.37 mmol, 2.5 eq) and 1,3-bis[2,6-bis(1-propylbutyl)phenyl]-4,5-dichloro-2H-imidazol-1-ium-2-ide; 3-chloropyridine; dichloropalladium (481.40 mg, 494.87 mol, 0.1 eq). The resulting mixture was stirred at 100° C. for 12 hr. LCMS showed starting material consumed. The reaction mixture was filtered and concentrated under reduced pressure to give a residue. The residue was purified by flash silica gel chromatography (ISCO®; 20 g SepaFlash® Silica Flash Column, Eluent of 0˜22% Ethyl acetate/Petroleum ethergradient @70 mL/min, PE: EtOAc=3:1, Rf=0.4) to give compound tert-butyl (5R)-7-(6-tert-butoxycarbonyl-4-nitro-2-pyridyl)-2,7-diazaspiro[4.4]nonane-2-carboxylate (1.84 g, 81% yield) as a yellow solid.

LC-MS [ESI, M-56]: 449.1.

1H NMR (400 MHz, CHLOROFORM-d) 6=7.85 (s, 1H), 7.17 (s, 1H), 3.77-3.26 (m, 8H), 2.07-2.03 (m, 2H), 2.01-1.88 (m, 2H), 1.63 (s, 9H), 1.47 (br d, J=5.2 Hz, 9H)

### Step 3: tert-butyl 6-[(5R)-2,7-diazaspiro[4.4]nonan-2-yl]-4-nitro-pyridine-2-carboxylate

To a solution of tert-butyl (5R)-7-(6-tert-butoxycarbonyl-4-nitro-2-pyridyl)-2,7-diazaspiro[4.4]nonane-2-carboxylate (1.84 g, 4.10 mmol, 1 eq) in DCM (18 mL) was added TFA (9.21 g, 80.77 mmol, 6 mL, 19.69 eq). The mixture was stirred at 0° C. for 0.5 h. LCMS showed starting material consumed. The reaction mixture was adjusted pH to 8 by Na₂CO₃ (10 mL) and diluted with H2O 20 mL and extracted with DCM (30 mL×3). The combined organic layers were washed with brine 30 mL (10 mL×3), dried over anhydrous sodium sulfate, filtered and concentrated under reduced pressure to give a residue tert-butyl 6-[(5R)-2,7-diazaspiro[4.4]nonan-2-yl]-4-nitro-pyridine-2-carboxylate (1.2 g, 81% yield) as a yellow solid

LC-MS [ESI, M-56]: 349.1.

### Step 4: tert-butyl 6-[(5R)-2-[8-methyl-6-[(3R)-3-methyl-3-phenyl-pyrrolidin-1-yl]-[1,2,4]triazolo[1,5-a]pyridine2-carbonyl]-2,7-diazaspiro[4.4]nonan-7-yl]-4-nitro-pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (1.1 g, 3.27 mmol, 1 eq) in DMF (12 mL) was added tert-butyl 6-[(5R)-2,7-diazaspiro[4.4]nonan-2-yl]-4-nitropyridine-2-carboxylate (1.14 g, 3.27 mmol, 1.00 eq) and HATU (1.87 g, 4.91 mmol, 1.5 eq). After addition, the mixture was stirred at 25° C. for 30 min, and then DIEA (1.27 g, 9.81 mmol, 1.71 mL, 3 eq) was added. The resulting mixture was stirred at 25° C. for 12 hr. LCMS showed 4% of starting material remained. Several new peaks were shown on LCMS and 68% of desired compound was detected. The reaction mixture was diluted with H₂O (20 mL) and extracted with ethyl acetate (30 mL×3). The combined organic layers were washed with brine (10 mL×3), dried over anhydrous sodium sulfate, filtered and concentrated under reduced pressure to give a residue. The residue was purified by flash silica gel chromatography (ISCO®; 12 g SepaFlash® Silica Flash Column, Eluent of 0˜10% NH₃-MeOH/DCM ethergradient @70 mL/min, DCM: MeOH=20:1, Rf=0.3) to give compound tert-butyl 6-[(5R)-2-[8-methyl-6-[(3R)-3-methyl-3-phenyl-pyrrolidin-1-yl]-[1,2,4]triazolo[1,5-a]pyridine2-carbonyl]-2,7-diazaspiro[4.4]nonan-7-yl]-4-nitro-pyridine-2-carboxylate (1.9 g, 85% yield) as a red solid.

LC-MS [ESI, M-56]: 667.5.

1H NMR (400 MHz, CHLOROFORM-d) 6=7.85 (dd, J=1.2, 4.8 Hz, 1H), 7.65 (br d, J=12.0 Hz, 1H), 7.41-7.28 (m, 4H), 7.27-7.21 (m, 1H), 7.20-7.13 (m, 1H), 7.01 (br d, J=4.0 Hz, 1H), 4.22 (t, J=7.2 Hz, 1H), 4.06-4.01 (m, 1H), 3.95-3.86 (m, 1H), 3.84-3.36 (m, 9H), 2.96 (s, 1H), 2.89 (s, 1H), 2.64 (d, J=7.6 Hz, 3H), 2.43-2.32 (m, 1H), 2.30-2.22 (m, 1H), 2.12-2.07 (m, 2H), 1.63 (d, J=3.2 Hz, 9H), 1.46 (d, J=7.6 Hz, 3H)

### Step 5: tert-butyl 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of tert-butyl 6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxylate (500 mg, 749.89 mol, 1 eq) in EtOAc (30 mL) was added Pd/C (79.80 mg, 74.99 mol, 10% purity, 0.1 eq) under N₂ atmosphere. The suspension was degassed and purged with H₂ for 3 times. The mixture was stirred under H₂ (50 Psi) at 30° C. for 12 h. LCMS showed starting material consumed completely. One main peak was shown on LCMS and 96% of desired compound was detected. The reaction mixture was concentrated under reduced pressure to give a residue tert-butyl 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (450 mg, 91% yield) as a red solid.

LC-MS [ESI, M-56+1]: 637.5.

1H NMR (400 MHz, DMSO-d₆) δ=7.91 (br d, J=11.2 Hz, 1H), 7.46-7.16 (m, 6H), 6.69-6.50 (m, 1H), 5.85 (br d, J=8.0 Hz, 1H), 5.66 (br d, J=16.8 Hz, 1H), 3.93 (br t, J=6.8 Hz, 1H), 3.82-3.73 (m, 1H), 3.66 (br t, J=6.8 Hz, 1H), 3.62-3.46 (m, 4H), 3.46-3.37 (m, 3H), 3.32-3.24 (m, 2H), 2.54 (s, 2H), 2.30-2.18 (m, 2H), 1.99 (s, 3H), 1.96-1.90 (m, 2H), 1.49 (d, J=5.6 Hz, 9H), 1.34 (d, J=8.4 Hz, 3H)

### Example 21

### 4-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

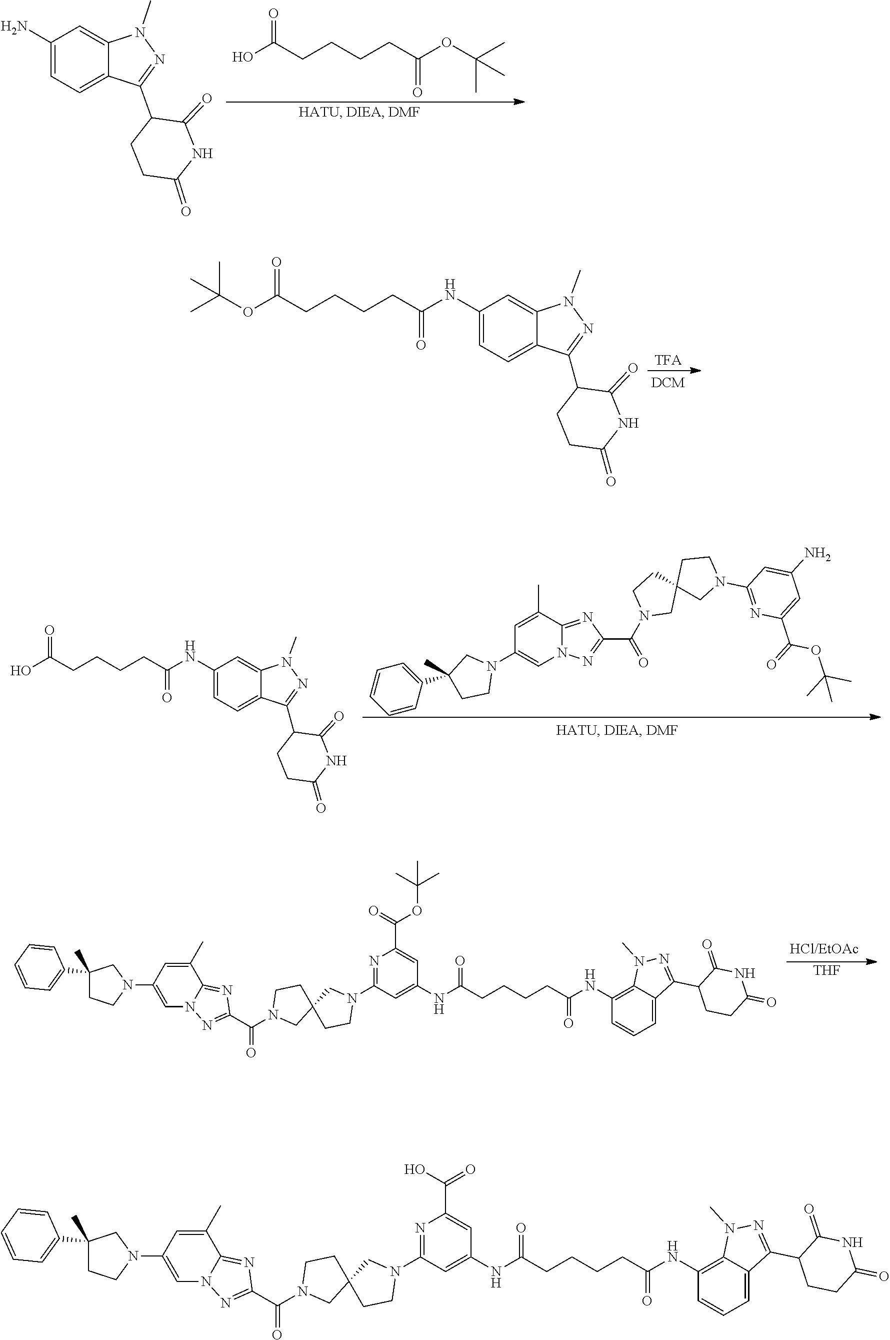

### Step 1: tert-butyl 6-[[3-(2,6-dioxo-3-piperidyl)-1-methyl-indazol-6-yl]amino]-6-oxo-hexanoate

To a solution of 3-(6-amino-1-methyl-indazol-3-yl)piperidine-2,6-dione (300 mg, 1.16 mmol, 1 eq) in DMF (4 mL) was added 6-tert-butoxy-6-oxo-hexanoic acid (352.38 mg, 1.74 mmol, 1.5 eq) and DIEA (225.18 mg, 1.74 mmol, 303.48 μL, 1.5 eq). After addition, the mixture was stirred at 25° C. for 5 min, and then HATU (662.48 mg, 1.74 mmol, 1.5 eq) was added. The resulting mixture was stirred at 25° C. for 55 min. LCMS showed starting material consumed. One main peak was shown on LCMS and 92% of desired compound was detected. The reaction mixture was diluted with H2O 10 mL and extracted with ethyl acetate (15 mL×3). The combined organic layers were washed with brine (10 mL×3), dried over anhydrous sodium sulfate, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ethergradient @60 mL/min, PE: EtOAc=0:1, Rf=0.3) to give compound tert-butyl 6-[[3-(2,6-dioxo-3-piperidyl)-1-methyl-indazol-6-yl]amino]-6-oxo-hexanoate (527 mg, 98% yield) as a white oil.

LC-MS [ESI, M+1]: 443.2.

¹H NMR (400 MHz, CHLOROFORM-d) 6=8.19 (s, 1H), 8.05 (br d, J=13.2 Hz, 1H), 7.85 (br s, 1H), 7.56 (d, J=8.4 Hz, 1H), 6.86 (br d, J=8.4 Hz, 1H), 4.27 (br t, J=6.0 Hz, 1H), 3.98 (s, 3H), 3.06-2.96 (m, 2H), 2.74-2.63 (m, 1H), 2.58-2.47 (m, 1H), 2.44 (br t, J=6.8 Hz, 2H), 2.31 (br t, J=6.8 Hz, 2H), 1.81-1.77 (m, 2H), 1.74-1.66 (m, 2H), 1.47 (s, 9H)

### Step 2: 6-[[3-(2,6-dioxo-3-piperidyl)-1-methyl-indazol-6-yl]amino]-6-oxo-hexanoic acid

To a solution of tert-butyl 6-[[3-(2,6-dioxo-3-piperidyl)-1-methyl-indazol-6-yl]amino]-6-oxo-hexanoate (320 mg, 723.15 mol, 1 eq) in DCM (2 mL) was added TFA (2.46 g, 21.54 mmol, 1.60 mL, 29.79 eq). The mixture was stirred at 25° C. for 0.5 hr. LCMS showed 13% of starting material remained. Several new peaks were shown on LCMS and 83% of desired compound was detected. The reaction mixture was concentrated under reduced pressure to give a residue. The crude product was purified by reversed-phase HPLC (0.1% FA condition) to give compound 6-[[3-(2,6-dioxo-3-piperidyl)-1-methyl-indazol-6-yl]amino]-6-oxo-hexanoic acid (170 mg, 61% yield) as a white solid.

LC-MS [ESI, M+1]: 387.2.

### Step 3: tert-butyl 4-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of 6-[[3-(2,6-dioxo-3-piperidyl)-1-methyl-indazol-6-yl]amino]-6-oxo-hexanoic acid (24.27 mg, 62.82 mol, 1.00 eq) in Pyridine (0.5 mL) was added tert-butyl 4-amino-6-[(5R)-2-[8-methyl-6-[(3R)-3-methyl-3-phenylpyrrolidin-1-yl]-[1,2,4]triazolo[1,5-a]pyridine-2-carbonyl]-2,7-diazaspiro[4.4]nonan-7-yl]pyridine-2-carboxylate (40 mg, 62.82 mol, 1 eq). Then POCl3 (164.50 mg, 1.07 mmol, 0.1 mL, 17.08 eq) was added at 0° C. The mixture was stirred at 0° C. for 0.5 h. LCMS showed starting material consumed. Several new peaks were shown on LCMS and 66% of desired compound was detected. The reaction mixture was quenched with H₂O (0.5 mL) and then diluted with H2O 5 mL and extracted with DCM (10 mL×3). The combined organic layers were washed with brine (3 mL×3), dried over anhydrous sodium sulfate, filtered and concentrated under reduced pressure to give a residue. The residue was purified by prep-HPLC (FA condition, column: CD01-Phenomenex luna C18 150*25*10 um; mobile phase: [water(FA)-ACN]; gradient: 37%-67% B over 10 min) to give compound tert-butyl 4-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (15.15 mg, 22% yield) as a yellow solid.

LC-MS [ESI, M+1]: 1005.6.

¹H NMR (400 MHz, DMSO-d₆) δ=10.88 (s, 1H), 10.32-10.01 (m, 2H), 8.33 (s, 1H), 8.08 (br s, 1H), 7.91 (br d, J=17.2 Hz, 1H), 7.60 (dd, J=1.6, 8.4 Hz, 1H), 7.41-7.20 (m, 7H), 7.15-7.05 (m, 2H), 4.30 (br dd, J=4.7, 9.2 Hz, 1H), 3.93 (br s, 1H), 3.89 (br d, J=2.4 Hz, 3H), 3.83-3.76 (m, 1H), 3.66 (br t, J=6.8 Hz, 1H), 3.63-3.43 (m, 7H), 3.40 (br s, 2H), 2.68-2.59 (m, 2H), 2.54 (s, 3H), 2.37 (br d, J=6.0 Hz, 5H), 2.28-2.21 (m, 2H), 2.16 (br dd, J=5.6, 12.8 Hz, 1H), 2.05-1.92 (m, 4H), 1.64 (br d, J=3.6 Hz, 4H), 1.51 (br d, J=5.6 Hz, 9H), 1.34 (br d, J=11.2 Hz, 3H)

### Step 4: 4-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of tert-butyl 4-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (32 mg, 31.84 mol, 1 eq) in THE (0.5 mL) was added HCl (6 M, 0.3 mL, 56.54 eq) in EtOAc (1.2 mL). The mixture was stirred at 25° C. for 1 hr. LCMS showed starting material consumed. Several new peaks were shown on LCMS and 97% of desired compound was detected. The reaction mixture was concentrated under reduced pressure to give a residue. The residue was purified by prep-HPLC (FA condition, column: CD01-Phenomenex luna C18 150×25×10 um; mobile phase: [water(FA)-ACN]; gradient:32%-62% B over 10 min) to give compound 4-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (2.55 mg, 8% yield) as a yellow solid.

LC-MS [ESI, M+1]: 949.4.

¹H NMR (400 MHz, DMSO-d₆) δ=10.88 (s, 1H), 10.20 (br d, J=9.6 Hz, 1H), 10.10 (br d, J=1.2 Hz, 1H), 8.08 (br s, 1H), 7.91 (br d, J=15.2 Hz, 1H), 7.60 (dd, J=2.4, 8.6 Hz, 1H), 7.42-7.19 (m, 7H), 7.14-6.98 (m, 2H), 4.30 (br dd, J=5.6, 8.8 Hz, 1H), 3.94 (br t, J=6.8 Hz, 1H), 3.89 (d, J=2.8 Hz, 3H), 3.81-3.76 (m, 1H), 3.69-3.65 (m, 1H), 3.62-3.53 (m, 4H), 3.52-3.46 (m, 5H), 2.68-2.60 (m, 2H), 2.57-2.52 (m, 3H), 2.40-2.30 (m, 5H), 2.28-2.21 (m, 2H), 2.16 (br dd, J=5.2, 12.6 Hz, 1H), 2.05-1.90 (m, 4H), 1.64 (br s, 4H), 1.34 (br d, J=11.2 Hz, 3H)

### Example 22

### 4-amino-N-[5-[[3-(2,6-dioxo-3-piperidyl)-1-methyl-indazol-6-yl]amino]-5-oxo-pentyl]-6-[(5R)-2-[8-methyl-6-[(3R)-3-methyl-3-phenyl-pyrrolidin-1-yl][1,2,4]triazolo[1,5-a]pyridine-2-carbonyl]-2,7-diazaspiro[4.4]nonan-7-yl]pyridine-2-carboxamide

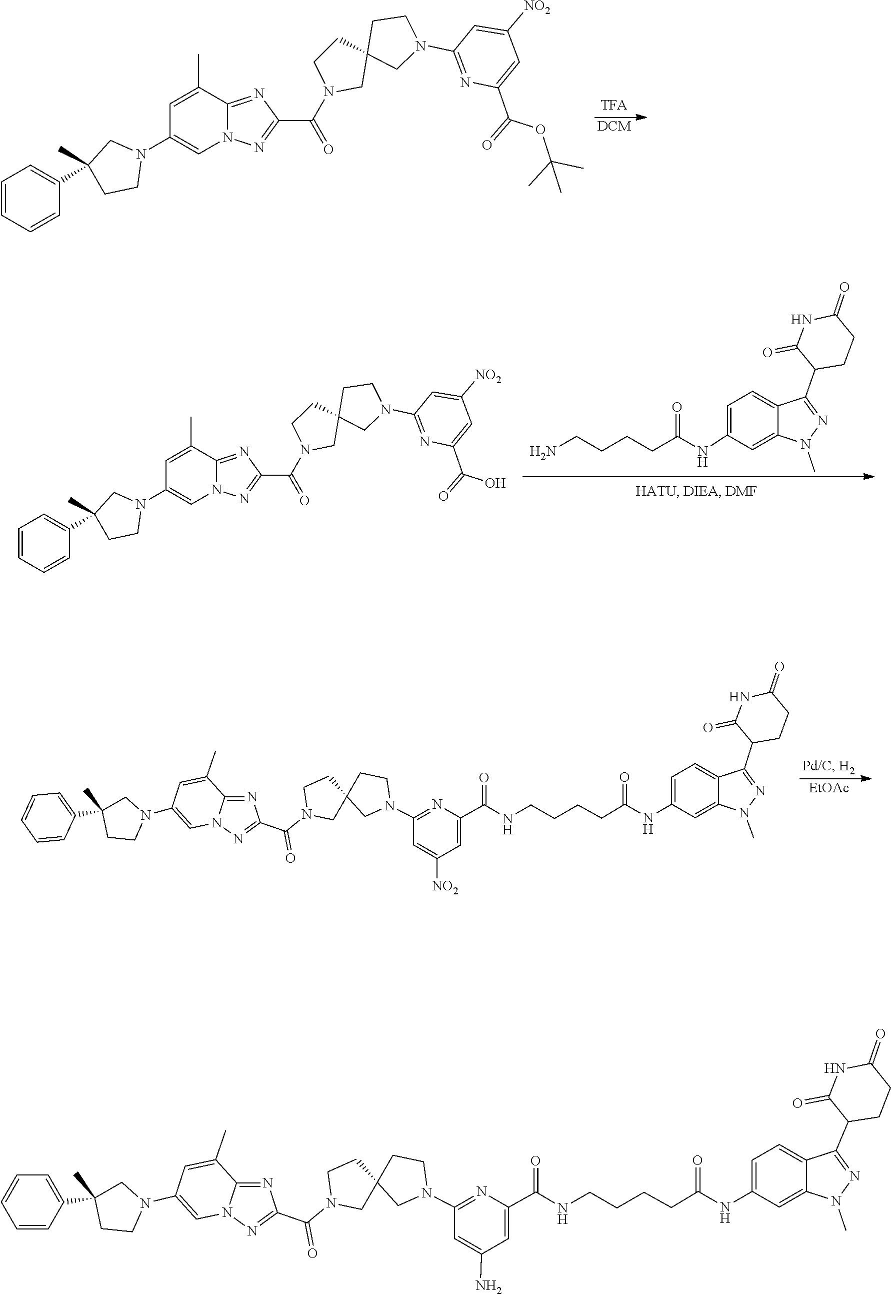

### Step 1: 6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxylic acid

To a solution of tert-butyl 6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxylate (60 mg, 89.99 mol, 1 eq) in DCM (0.5 mL) was added TFA (12 M, 0.3 mL, 40.01 eq). The mixture was stirred at 25° C. for 1 h. LCMS showed starting material consumed. One main peak was shown on LCMS and 95% of desired compound was detected. The reaction mixture was concentrated under reduced pressure to give a residue 6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxylic acid (62 mg, 91% yield, TFA) as a yellow oil.

LC-MS [ESI, M+1]: 611.1.

### Step 2: N-[5-[[3-(2,6-dioxo-3-piperidyl)-1-methyl-indazol-6-yl]amino]-5-oxo-pentyl]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxamide

To a solution of 6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxylic acid (55.20 mg, 76.17 mol, 1 eq, TFA) in DMF (1 mL) was added 5-amino-N-[3-(2,6-dioxo-3-piperidyl)-1-methyl-indazol-6-yl]pentanamide (30 mg, 76.17 mol, 1 eq, HCl) and HATU (28.96 mg, 76.17 mol, 1 eq). After addition, the mixture was stirred at 25° C. for 5 min, and then DIEA (9.84 mg, 76.17 mol, 13.27 μL, 1 eq) was added. The resulting mixture was stirred at 25° C. for 25 min. LCMS showed starting material consumed. Several new peaks were shown on LCMS and 61% of desired compound was detected. The reaction mixture was diluted with H2O 5 mL and extracted with ethyl acetate (10 mL×3). The combined organic layers were washed with brine (5 mL×3), dried over anhydrous sodium sulfate, filtered and concentrated under reduced pressure to give a residue. The residue was purified by prep-HPLC (FA condition; column: CD01-Phenomenex luna C18 150×25×10 um; mobile phase: [water(FA)-ACN]; gradient:45%-75% B over 8 min) to give compound N-[5-[[3-(2,6-dioxo-3-piperidyl)-1-methyl-indazol-6-yl]amino]-5-oxo-pentyl]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxamide (35 mg, 48% yield) as a yellow solid.

LC-MS [ESI, M+1]: 950.6.

### Step 3: 4-amino-N-[5-[[3-(2,6-dioxo-3-piperidyl)-1-methyl-indazol-6-yl]amino]-5-oxo-pentyl]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxamide

To a solution of N-[5-[[3-(2,6-dioxo-3-piperidyl)-1-methyl-indazol-6-yl]amino]-5-oxo-pentyl]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-4-nitro-pyridine-2-carboxamide (30 mg, 31.58 mol, 1 eq) in THE (15 mL) was added Pd/C (3.36 mg, 3.16 mol, 10% purity, 0.1 eq) under N₂ atmosphere. The suspension was degassed and purged with H₂ for 3 times. The mixture was stirred under H₂ (63.65 g, 31.58 mol, 1 eq) (50 Psi.) at 25° C. for 1 hr. LCMS showed starting material consumed. Several new peaks were shown on LCMS and 62% of desired compound was detected. The reaction mixture was concentrated under reduced pressure to give a residue. The residue was purified by prep-HPLC (FA condition, column: CD01-Phenomenex luna C18 150×25×10 um; mobile phase: [water(FA)-ACN]; gradient: 24%-54% B over 10 min) to gine compound 4-amino-N-[5-[[3-(2,6-dioxo-3-piperidyl)-1-methyl-indazol-6-yl]amino]-5-oxo-pentyl]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxamide (2.8 mg, 9% yield) as a white solid.

LC-MS [ESI, M+1]: 920.4.

¹H NMR (400 MHz, DMSO-d₆) δ=10.87 (s, 1H), 10.07 (s, 1H), 8.35 (br dd, J=6.0, 12.7 Hz, 2H), 8.07 (s, 1H), 7.90 (br d, J=6.4 Hz, 1H), 7.59 (d, J=8.8 Hz, 1H), 7.45-7.15 (m, 6H), 7.07 (d, J=8.8 Hz, 1H), 6.76-6.56 (m, 1H), 5.92-5.81 (m, 2H), 5.67-5.58 (m, 1H), 4.36-4.23 (m, 1H), 4.03-3.91 (m, 1H), 3.89 (d, J=1.2 Hz, 3H), 3.81-3.75 (m, 1H), 3.69-3.64 (m, 1H), 3.62-3.55 (m, 2H), 3.54-3.40 (m, 9H), 2.68-2.60 (m, 2H), 2.53 (br s, 3H), 2.41-2.32 (m, 3H), 2.28-2.21 (m, 2H), 2.20-2.12 (m, 1H), 2.03-1.88 (m, 4H), 1.68-1.50 (m, 4H), 1.34 (d, J=9.2 Hz, 3H)

### Example 23

### tert-butyl 4-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

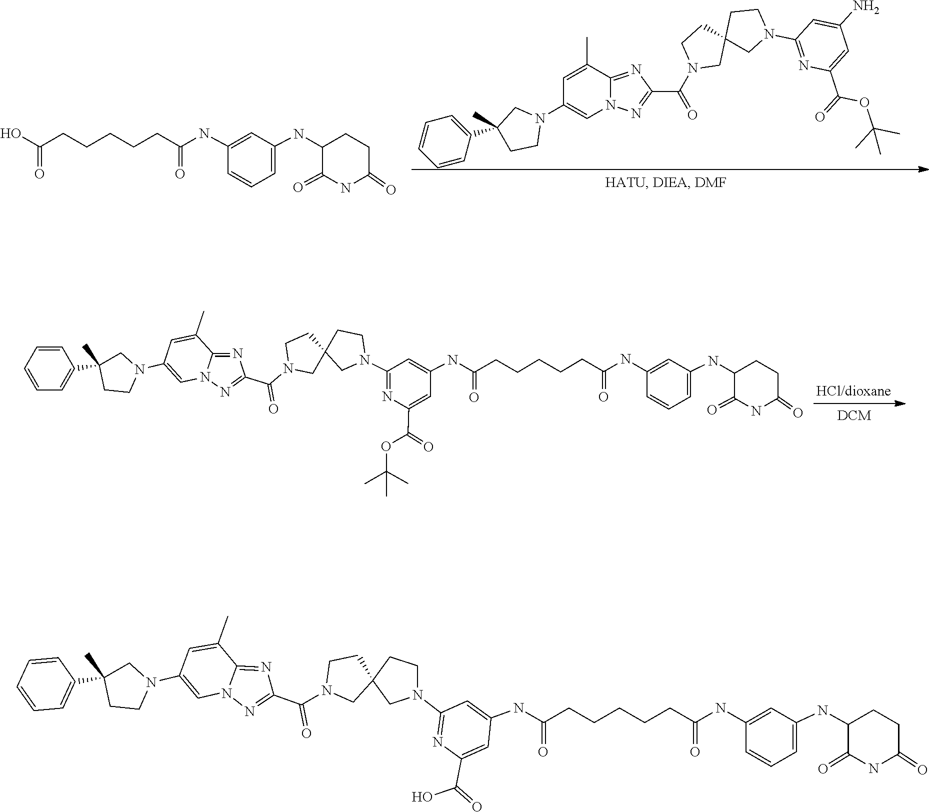

### Step 1: tert-butyl 4-(7-((3-((2,6-dioxopiperidin-3-yl)amino)phenyl)amino)-7-oxoheptanamido)-6-((R)-7-(8-methyl-6-((R)-3-methyl-3-phenylpyrrolidin-1-yl)-[1,2,4]triazolo[1,5-a]pyridine-2-carbonyl)-2,7-diazaspiro[4.4]nonan-2-yl)picolinate

To a solution of 7-[3-[(2,6-dioxo-3-piperidyl)amino]anilino]-7-oxo-heptanoic acid (43.73 mg, 109.93 mol, 1 eq, HCl) and tert-butyl 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (70 mg, 109.93 mol, 1 eq) in Pyridine (0.5 mL) was added POCl3 (16.86 mg, 109.93 mol, 10.25 μL, 1 eq) at 0° C. The mixture was stirred at 0° C. for 0.1 h. LCMS showed starting material was consumed completely and the desired mass was detected. The reaction mixture was quenched by H₂O (0.5 mL). The reaction mixture was concentrated under the reduced pressure to give the crude product The residue was purified by prep-HPLC (column: CD01-Phenomenex luna C18 150*25*10 um; mobile phase:[Water(FA)-MeCN]; gradient:35%-65% B over 8 min) and lyophilized to give tert-butyl 4-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (30 mg, 27% yield) as white solid.

LC-MS [ESI, M+1]: 491.0.

### Step 2: 4-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine2-carboxylic acid

To a solution of tert-butyl 4-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (30 mg, 30.61 μmol, 1 eq) in THF (1 mL) was added HCl/EtOAc (6M, 3.00 mL, 588.10 eq). The mixture was stirred at 25° C. for 1 h. LCMS showed starting material was consumed completely and the desired mass was detected. The reaction mixture was concentrated under the reduced pressure to give the crude product. The residue was purified by prep-HPLC (column: CD01-Phenomenex luna C18 150*25*10 um; mobile phase: [Water(FA)-MeCN]; gradient:2832%-5862% B over 8 min) and lyophilized to give 4-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine2-carboxylic acid (13.95 mg, 46% yield) as yellow solid.

LC-MS [ESI, M+1]: 462.9.

¹H NMR (400 MHz, DMSO-d₆) δ=10.77 (br s, 1H), 10.17 (br d, J=9.2 Hz, 1H), 9.60 (br s, 1H), 7.91 (br d, J=15.2 Hz, 1H), 7.42-7.31 (m, 5H), 7.30-7.19 (m, 2H), 7.06 (br d, J=18.6 Hz, 1H), 7.01-6.91 (m, 2H), 6.77 (br d, J=7.2 Hz, 1H), 6.35 (br d, J=8.0 Hz, 1H), 5.83 (br d, J=7.6 Hz, 1H), 4.29-4.18 (m, 1H), 3.95 (br s, 1H), 3.79 (br d, J=5.2 Hz, 1H), 3.70-3.54 (m, 4H), 3.50 (br d, J=9.2 Hz, 4H), 3.42 (br d, J=1.2 Hz, 2H), 2.77-2.67 (m, 1H), 2.64-2.51 (m, 4H), 2.36-2.19 (m, 6H), 2.10-1.85 (m, 6H), 1.59 (br s, 4H), 1.34 (br d, J=9.2 Hz, 5H)

### Example 24

### 3-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

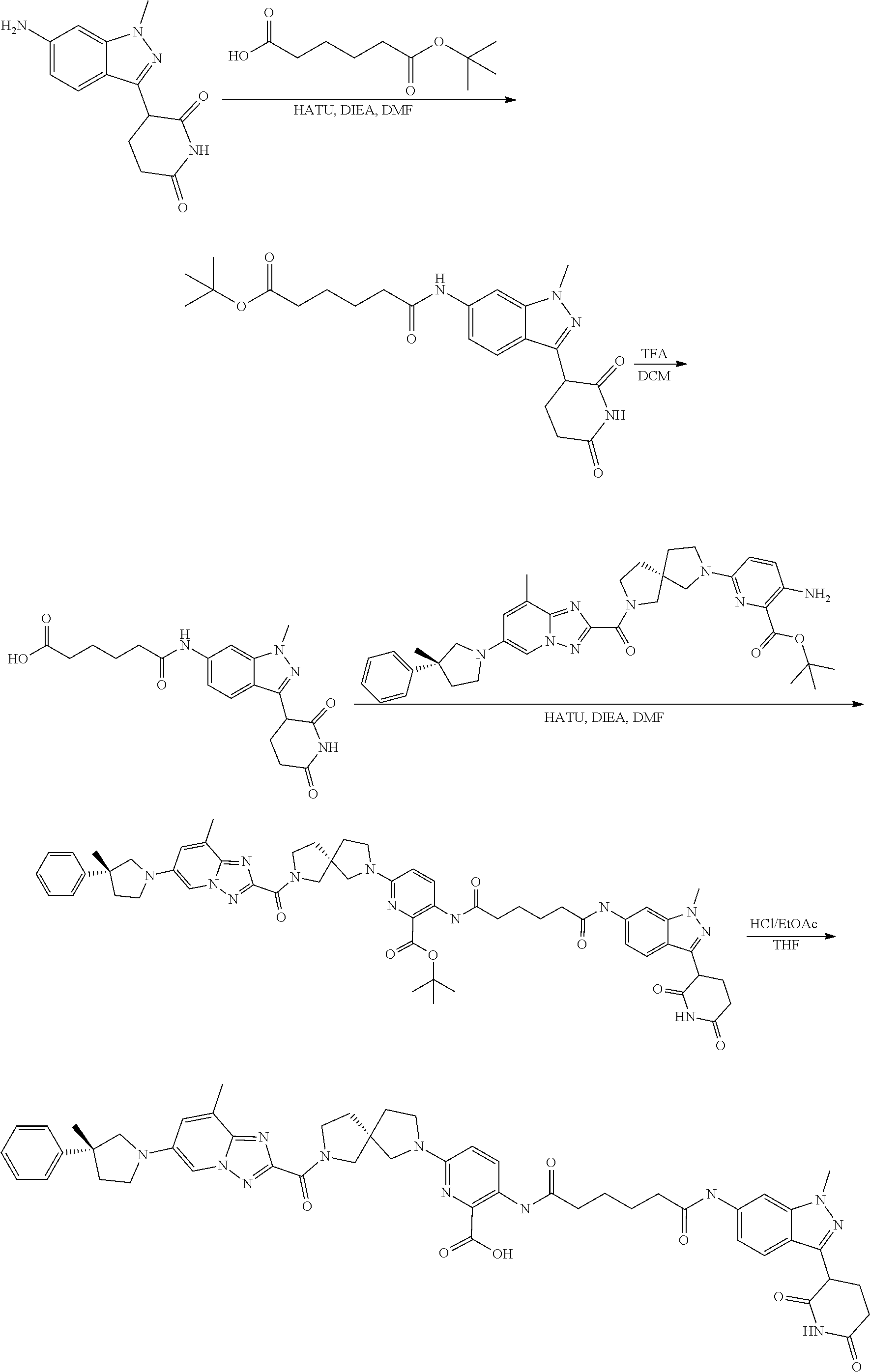

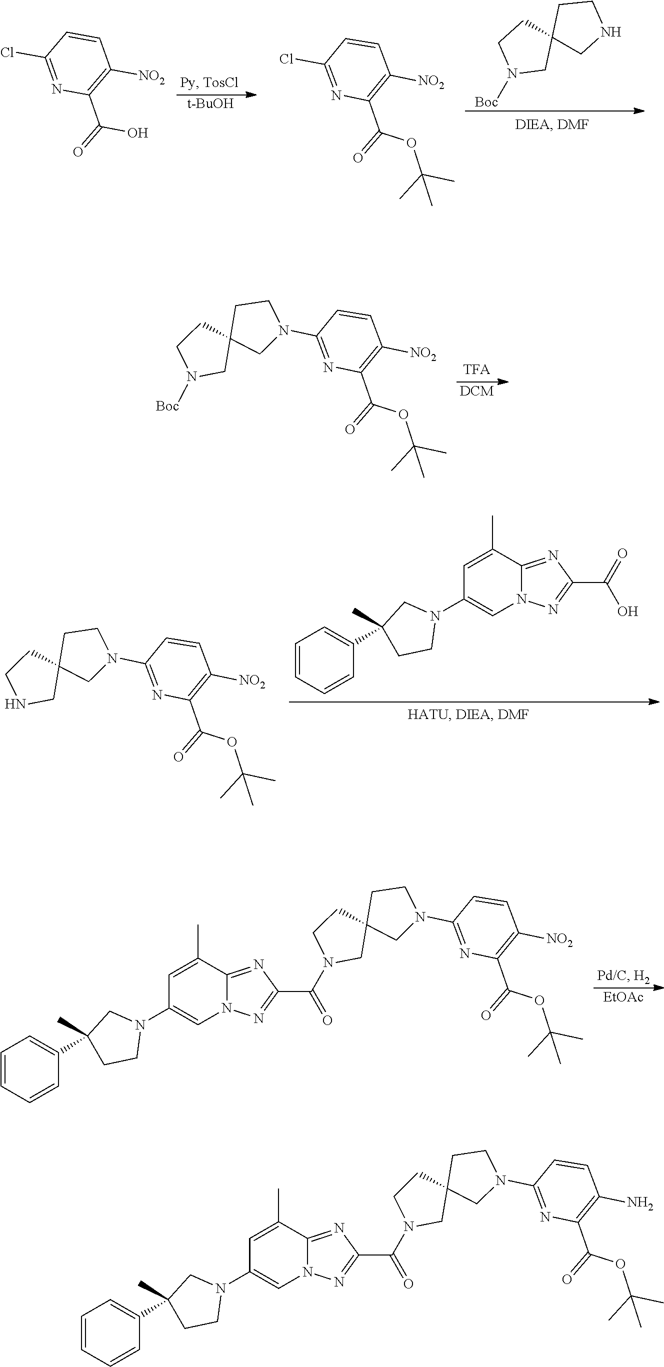

### Step 1: tert-butyl 6-[[3-(2,6-dioxo-3-piperidyl)-1-methyl-indazol-6-yl]amino]-6-oxo-hexanoate

To a solution of 3-(6-amino-1-methyl-indazol-3-yl)piperidine-2,6-dione (300 mg, 1.16 mmol, 1 eq) in DMF (4 mL) was added 6-tert-butoxy-6-oxo-hexanoic acid (352.38 mg, 1.74 mmol, 1.5 eq) and DIEA (225.18 mg, 1.74 mmol, 303.48 μL, 1.5 eq). After addition, the mixture was stirred at 25° C. for 5 min, and then HATU (662.48 mg, 1.74 mmol, 1.5 eq) was added. The resulting mixture was stirred at 25° C. for 55 min. LCMS showed starting material consumed. One main peak was shown on LCMS and 92% of desired compound was detected. The reaction mixture was diluted with H2O 10 mL and extracted with ethyl acetate (15 mL×3). The combined organic layers were washed with brine 30 mL (10 mL×3), dried over anhydrous sodium sulfate, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ethergradient @60 mL/min, PE: EtOAc=0:1, Rf=0.3) to give compound tert-butyl 6-[[3-(2,6-dioxo-3-piperidyl)-1-methyl-indazol-6-yl]amino]-6-oxo-hexanoate (527 mg, 98% yield) as a white oil.

LC-MS [ESI, M+1]: 443.2.

1H NMR (400 MHz, CHLOROFORM-d) 6=8.19 (s, 1H), 8.05 (br d, J=13.2 Hz, 1H), 7.85 (br s, 1H), 7.56 (d, J=8.8 Hz, 1H), 6.86 (br d, J=8.8 Hz, 1H), 4.27 (br t, J=6.0 Hz, 1H), 3.98 (s, 3H), 3.06-2.96 (m, 2H), 2.74-2.63 (m, 1H), 2.58-2.47 (m, 1H), 2.44 (br t, J=6.8 Hz, 2H), 2.31 (br t, J=6.8 Hz, 2H), 1.81-1.77 (m, 2H), 1.74-1.66 (m, 2H), 1.47 (s, 9H)

### Step 2: 6-[[3-(2,6-dioxo-3-piperidyl)-1-methyl-indazol-6-yl]amino]-6-oxo-hexanoic acid

To a solution of tert-butyl 6-[[3-(2,6-dioxo-3-piperidyl)-1-methyl-indazol-6-yl]amino]-6-oxo-hexanoate (100 mg, 225.99 mol, 1 eq) in DCM (0.5 mL) was added TFA (460.50 mg, 4.04 mmol, 0.3 mL, 17.87 eq). The mixture was stirred at 25° C. for 0.5 h. LCMS showed starting material consumed. One main peak was shown on LCMS and 96% of desired compound was detected. The reaction mixture was concentrated under reduced pressure to give a residue 6-[[3-(2,6-dioxo-3-piperidyl)-1-methyl-indazol-6-yl]amino]-6-oxo-hexanoic acid (100 mg, 85% yield, TFA) as a yellow oil.

LC-MS [ESI, M+1]: 387.0

### Step 3: tert-butyl 3-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of 6-[[3-(2,6-dioxo-3-piperidyl)-1-methyl-indazol-6-yl]amino]-6-oxo-hexanoic acid (70.73 mg, 141.34 mol, 1.5 eq, TFA) in DMF (1 mL) was added tert-butyl 3-amino-6-[(5R)-2-[8-methyl-6-[(3R)-3-methyl-3-phenylpyrrolidin-1-yl]-[1,2,4]triazolo[1,5-a]pyridine-2-carbonyl]-2,7-diazaspiro[4.4]nonan-7-yl]pyridine-2-carboxylate (60 mg, 94.22 mol, 1 eq) and DIEA (60.89 mg, 471.12 mol, 82.06 μL, 5 eq). After addition, the mixture was stirred at 25° C. for 5 min, and then HATU (53.74 mg, 141.34 mol, 1.5 eq) was added. The resulting mixture was stirred at 25° C. for 25 min. LCMS showed starting material consumed. Several new peaks were shown on LCMS and 63% of desired compound was detected. The reaction mixture was diluted with H2O 5 mL and extracted with DCM (10 mL×3). The combined organic layers were washed with brine (3 mL×3), dried over anhydrous sodium sulfate, filtered and concentrated under reduced pressure to give a residue. The residue was purified by prep-HPLC (FA condition; column: CD01-Phenomenex luna C18 150*25*10 um; mobile phase: [water(FA)-ACN]; gradient:61%-991O % B over 10 min) to give compound tert-butyl 3-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (29.93 mg, 29% yield) as a yellow solid.

LC-MS [ESI, M+1]: 1005.5.

1H NMR (400 MHz, DMSO-d₆) δ=10.88 (s, 1H), 10.11 (br s, 1H), 9.63 (s, 1H), 8.40-8.31 (m, 1H), 8.08 (s, 1H), 7.91 (br d, J=9.2 Hz, 1H), 7.75 (dd, J=5.6, 8.4 Hz, 1H), 7.60 (br d, J=8.4 Hz, 1H), 7.43-7.19 (m, 6H), 7.08 (br d, J=8.8 Hz, 1H), 6.63 (dd, J=9.2, 14.8 Hz, 1H), 4.31 (dd, J=4.8, 9.2 Hz, 1H), 3.95 (br t, J=6.8 Hz, 1H), 3.90 (d, J=2.4 Hz, 3H), 3.80 (s, 1H), 3.67 (br t, J=6.4 Hz, 1H), 3.63-3.56 (m, 2H), 3.54-3.47 (m, 4H), 3.42 (br s, 3H), 2.69-2.59 (m, 2H), 2.58-2.51 (m, 3H), 2.41-2.29 (m, 5H), 2.28-2.22 (m, 2H), 2.17 (br dd, J=5.2, 13.2 Hz, 1H), 2.05-1.90 (m, 4H), 1.65 (br s, 4H), 1.47 (d, J=5.2 Hz, 9H), 1.34 (d, J=9.6 Hz, 3H)

### Step 4: 3-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of tert-butyl 3-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (24 mg, 23.88 mol, 1 eq) in THE (0.5 mL) was added HCl (6 M, 0.3 mL, 75.39 eq) in EtOAc (1.2 mL). The mixture was stirred at 30° C. for 1 h. LCMS showed starting material consumed. Several new peaks were shown on LCMS and 87% of desired compound was detected. The reaction mixture was concentrated under reduced pressure to give a residue. The residue was purified by prep-HPLC (FA condition, column: CD01-Phenomenex luna C18 150×25×10 um; mobile phase: [water(FA)-ACN]; gradient:35%-65% B over 10 min) to give compound 3-[[6-[[3-(2,6-dioxo-3-piperidyl)-1-methyl-indazol-6-yl]amino]-6-oxo-hexanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (11.84 mg, 52% yield) as a yellow solid.

LC-MS [ESI, M+1]: 949.5.

1H NMR (400 MHz, DMSO-d₆) δ=10.88 (s, 1H), 10.79-10.62 (m, 1H), 10.13 (s, 1H), 8.49-8.33 (m, 1H), 8.09 (s, 1H), 7.91 (br d, J=12.4 Hz, 1H), 7.60 (br d, J=8.4 Hz, 1H), 7.46-7.26 (m, 5H), 7.23 (q, J=6.8 Hz, 1H), 7.10 (br d, J=8.8 Hz, 1H), 6.73 (br dd, J=9.2, 16.4 Hz, 1H), 4.30 (dd, J=5.2, 9.2 Hz, 1H), 3.95 (br t, J=7.2 Hz, 1H), 3.90 (d, J=2.8 Hz, 3H), 3.83-3.75 (m, 1H), 3.67 (br t, J=6.8 Hz, 1H), 3.63-3.38 (m, 9H), 2.69-2.58 (m, 2H), 2.54 (s, 3H), 2.42-2.30 (m, 5H), 2.28-2.21 (m, 2H), 2.16 (br dd, J=5.2, 13.2 Hz, 1H), 2.08-1.91 (m, 4H), 1.66 (br d, J=2.8 Hz, 4H), 1.34 (d, J=10.0 Hz, 3H)

### tert-butyl 3-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

### Step 1: tert-butyl 6-chloro-3-nitro-pyridine-2-carboxylate

To a solution of 6-chloro-3-nitro-pyridine-2-carboxylic acid (4.5 g, 22.22 mmol, 1 eq) in t-BuOH (45 mL) and Pyridine (14.06 g, 177.73 mmol, 14.35 mL, 8 eq) was added TosCl (12.71 g, 66.65 mmol, 3 eq) at 0° C. The mixture was stirred at 25° C. for 2 h. TLC indicated starting material was consumed completely and one new spot formed. The reaction was clean according to TLC (PE: EtOAc=5:1, Rf=0.3). The reaction mixture was quenched with NaHCO₃ (10 mL) and then concentrated under reduced pressure to give a residue. The residue was diluted with H2O 20 mL and extracted with ethyl acetate (40 mL×3). The combined organic layers were washed with brine (20 mL×3), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜10% EA/PE @70 mL/min, PE: EtOAc=5:1, Rf=0.3) to give compound tert-butyl 6-chloro-3-nitro-pyridine-2-carboxylate (4.16 g, 72% yield) as a yellow solid.

1H NMR (400 MHz, CHLOROFORM-d) 6=8.35 (d, J=8.4 Hz, 1H), 7.58 (d, J=8.4 Hz, 1H), 1.63 (s, 9H)

### Step 2: tert-butyl (5R)-7-(6-tert-butoxycarbonyl-5-nitro-2-pyridyl)-2,7-diazaspiro[4.4]nonane-2-carboxylate

To a solution of tert-butyl 6-chloro-3-nitro-pyridine-2-carboxylate (1.5 g, 5.80 mmol, 1 eq) in DMF (15 mL) was added tert-butyl (5R)-2,7-diazaspiro[4.4]nonane-2-carboxylate (1.31 g, 5.80 mmol, 1 eq) and DIEA (2.25 g, 17.40 mmol, 3.03 mL, 3 eq). The mixture was stirred at 100° C. for 12 h. LCMS showed starting material consumed. One main peak was shown on LCMS and 97% of desired compound was detected. The reaction mixture was diluted with H2O 20 mL and extracted with ethyl acetate (30 mL×3). The combined organic layers were washed with brine (20 mL×3), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜22% Ethyl acetate/Petroleum ethergradient @70 mL/min, PE: EtOAc=2:1, Rf=0.4) to give compound tert-butyl (5R)-7-(6-tert-butoxycarbonyl-5-nitro-2-pyridyl)-2,7-diazaspiro[4.4]nonane-2-carboxylate (1.83 g, 69% yield) as a yellow solid.

LC-MS [ESI, M-56+1]: 203.0.

1H NMR (400 MHz, CHLOROFORM-d) 6=8.19 (d, J=9.2 Hz, 1H), 6.34 (br d, J=7.2 Hz, 1H), 3.96-3.62 (m, 2H), 3.60-3.25 (m, 6H), 2.05 (s, 2H), 1.93 (br t, J=6.4 Hz, 2H), 1.63 (s, 9H), 1.47 (s, 9H)

### Step 3: tert-butyl 6-[(5R)-2,7-diazaspiro[4.4]nonan-2-yl]-3-nitro-pyridine-2-carboxylate

To a solution of tert-butyl (5R)-7-(6-tert-butoxycarbonyl-5-nitro-2-pyridyl)-2,7-diazaspiro[4.4]nonane-2-carboxylate (1.83 g, 4.08 mmol, 1 eq) in DCM (18 mL) was added TFA (12 M, 6 mL, 17.65 eq). The mixture was stirred at 0° C. for 0.5 h. LCMS showed starting material consumed. One main peak was shown on LCMS and 96% of desired compound was detected. The reaction mixture was adjusted pH to 8 by Na₂CO₃ (10 mL) and diluted with H₂O 30 mL and extracted with DCM (50 mL×3). The combined organic layers were washed with brine (15 mL×3), dried over anhydrous sodium sulfate, filtered and concentrated under reduced pressure to give a residue tert-butyl 6-[(5R)-2,7-diazaspiro[4.4]nonan-2-yl]-3-nitro-pyridine-2-carboxylate (1.19 g, 81% yield) as a yellow solid.

LC-MS [ESI, M+1]: 349.0.

### Step 4: tert-butyl 6-[(5R)-2-[8-methyl-6-[(3R)-3-methyl-3-phenyl-pyrrolidin-1-yl]-[1,2,4]triazolo[1,5-a]pyridine2-carbonyl]-2,7-diazaspiro[4.4]nonan-7-yl]-3-nitro-pyridine-2-carboxylate

To a solution of 8-methyl-6-[(3R)-3-methyl-3-phenyl-pyrrolidin-1-yl]-[1,2,4]triazolo[1,5-a]pyridine-2-carboxylic acid (1.15 g, 3.42 mmol, 1 eq) in DMF (12 mL) was added dropwise HATU (1.95 g, 5.13 mmol, 1.5 eq) and tert-butyl 6-[(5R)-2,7-diazaspiro[4.4]nonan-2-yl]-3-nitro-pyridine-2-carboxylate (1.19 g, 3.42 mmol, 1 eq). After addition, the mixture was stirred at 25° C. for 30 min, and then DIEA (1.33 g, 10.26 mmol, 1.79 mL, 3 eq) was added. The resulting mixture was stirred at 25° C. for 12 h. LCMS showed 9% of starting material remained. Several new peaks were shown on LCMS and 63% of desired compound was detected. The reaction mixture was diluted with H2O 20 mL and extracted with EA (30 mL×3). The combined organic layers were washed with brine (10 mL×3), dried over anhydrous sodium sulfate, filtered and concentrated under reduced pressure to give a residue. The residue was purified by flash silica gel chromatography (ISCO®; 12 g SepaFlash® Silica Flash Column, Eluent of 0˜10% NH₃-MeOH/DCM ethergradient @70 mL/min, DCM: MeOH=20:1, Rf=0.3) to give compound tert-butyl 6-[(5R)-2-[8-methyl-6-[(3R)-3-methyl-3-phenyl-pyrrolidin-1-yl]-[1,2,4]triazolo[1,5-a]pyridine2-carbonyl]-2,7-diazaspiro[4.4]nonan-7-yl]-3-nitro-pyridine-2-carboxylate (1.79 g, 77% yield) as a yellow solid.

LC-MS [ESI, M-56+1]: 667.6.

1H NMR (400 MHz, CHLOROFORM-d) 6=8.17 (br d, J=9.2 Hz, 1H), 7.64 (br d, J=11.2 Hz, 1H), 7.42-7.27 (m, 4H), 7.27-7.21 (m, 1H), 7.01 (br d, J=6.4 Hz, 1H), 6.34 (br d, J=9.2 Hz, 1H), 4.29-4.16 (m, 1H), 4.02 (br s, 1H), 3.96-3.68 (m, 4H), 3.65-3.33 (m, 6H), 2.96 (s, 1H), 2.89 (s, 1H), 2.63 (d, J=6.4 Hz, 3H), 2.43-2.32 (m, 1H), 2.30-2.21 (m, 1H), 2.08 (br d, J=7.6 Hz, 2H), 1.70-1.56 (m, 9H), 1.45 (d, J=6.8 Hz, 3H)

### Step 5: tert-butyl 3-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of tert-butyl 6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]-3-nitro-pyridine-2-carboxylate (500.00 mg, 749.89 mol, 1 eq) in EtOAc (30 mL) was added PtO2 (17.03 mg, 74.99 mol, 0.1 eq) under N₂ atmosphere. The suspension was degassed and purged with H₂ for 3 times. The mixture was stirred under H₂ (50 Psi) at 30° C. for 12 hr. LCMS showed 2% of starting material remained. Several new peaks were shown on LCMS and 88% of desired compound was detected. The reaction mixture was concentrated under reduced pressure to give a residue. The residue was purified by prep-HPLC (neutral condition; column: CD07-Daisogel SP-100-8-ODS-PK 150×25×10 um; mobile phase: [water(NH₄HCO₃)-ACN]; gradient: 54%-84% B over 10 min) to give compound tert-butyl 3-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (107.75 mg, 22% yield) as a yellow solid.

LC-MS [ESI, M-56+1]: 637.3.

1H NMR (400 MHz, DMSO-d₆) δ=7.91 (br d, J=7.6 Hz, 1H), 7.45-7.27 (m, 5H), 7.22 (br s, 1H), 7.11 (dd, J=9.2, 10.8 Hz, 1H), 6.66 (dd, J=8.8, 15.6 Hz, 1H), 5.79 (br s, 2H), 4.01-3.85 (m, 1H), 3.77 (s, 1H), 3.68-3.35 (m, 10H), 2.54 (s, 3H), 2.33-2.20 (m, 2H), 2.05-1.85 (m, 4H), 1.51 (d, J=5.6 Hz, 9H), 1.35 (d, J=8.4 Hz, 3H)

### Example 25

### 3-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine2-carboxylic acid

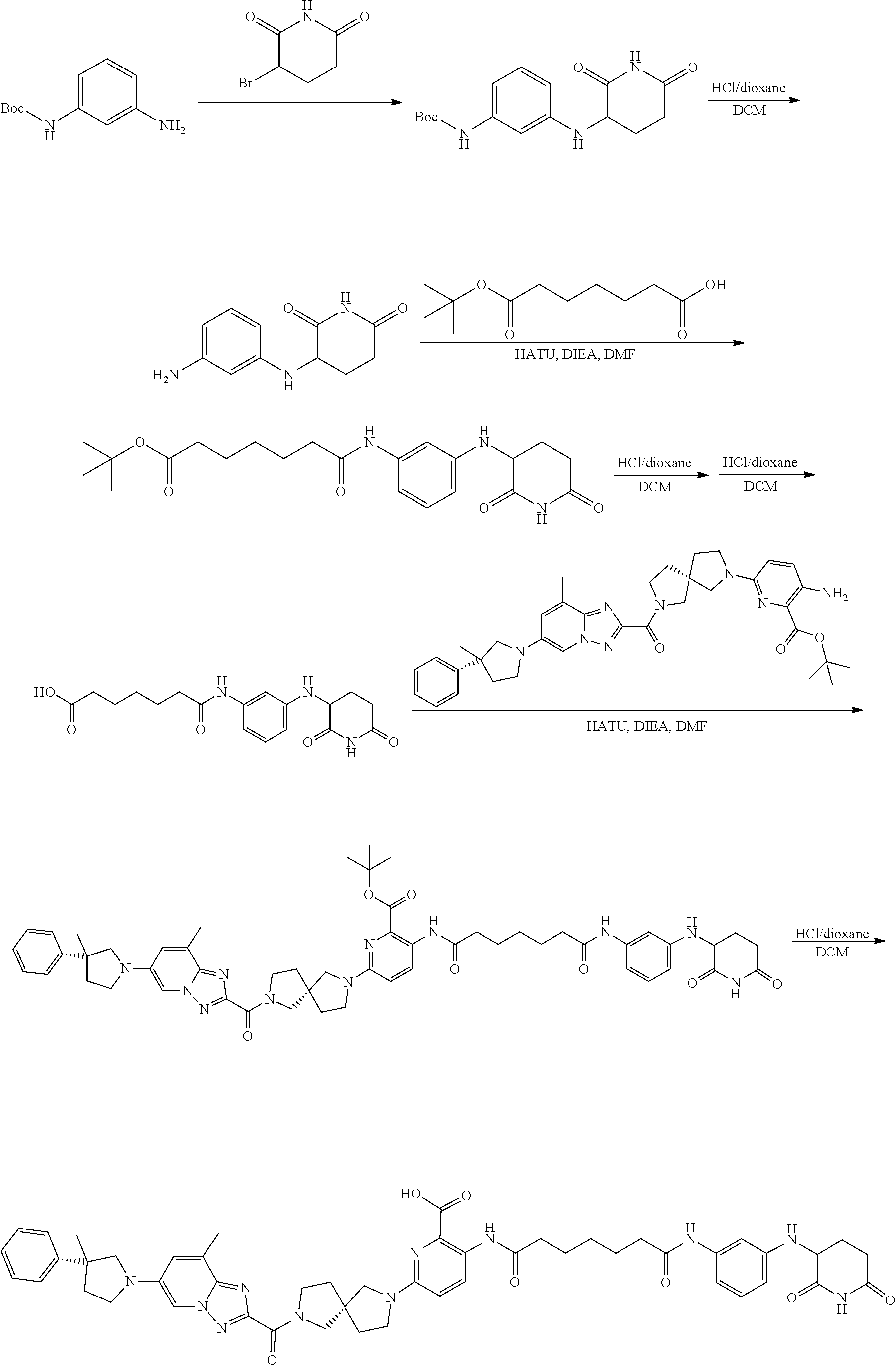

### Step 1: tert-butyl N-[3-[(2,6-dioxo-3-piperidyl)amino]phenyl]carbamate

To a solution of 3-bromopiperidine-2,6-dione (10 g, 52.08 mmol, 1 eq) in DMF (100 mL) was added tert-butyl N-(3-aminophenyl)carbamate (10.85 g, 52.08 mmol, 1 eq). The mixture was stirred at 30° C. for 12 h. LCMS showed starting material was consumed completely and the desired compound was detected. The reaction mixture was added water, then was extracted with EtOAc (200 mL×3). The combined organic phase was washed with brine (30 mL), dried with anhydrous Na₂SO₄, filtered and concentrated in vacuum to give a residue, the residue was purified by prep-HPLC (FA) and lyophilized to give tert-butyl N-[3-[(2,6-dioxo-3-piperidyl)amino]phenyl]carbamate (5.1 g, 31% yield) a green solid.

LC-MS [ESI, M-56]: 264.1.

¹H NMR (400 MHz, DMSO-d₆) δ=10.77 (s, 1H), 9.03 (s, 1H), 6.92 (t, J=8.0 Hz, 1H), 6.85 (s, 1H), 6.65 (br d, J=8.0 Hz, 1H), 6.29 (dd, J=1.2, 8.1 Hz, 1H), 5.79 (d, J=7.6 Hz, 1H), 4.25-4.16 (m, 1H), 2.78-2.65 (m, 1H), 2.63-2.54 (m, 1H), 2.10 (br dd, J=4.4, 8.8 Hz, 1H), 1.87 (br dd, J=4.4, 12.4 Hz, 1H).

### Step 2: 3-(3-aminoanilino)piperidine-2,6-dione

To a solution of tert-butyl N-[3-[(2,6-dioxo-3-piperidyl)amino]phenyl]carbamate (800 mg, 2.51 mmol, 1 eq) in DCM (10 mL) was added HCl (12 M, 1.87 mL, 8.93 eq). The mixture was stirred at 25° C. for 1 h. LCMS showed starting material was consumed completely and the desired compound was detected. The reaction mixture was filtered and the filtrate was concentrated under the reduced pressure to give 3-(3-aminoanilino)piperidine-2,6-dione (800 mg, crude, HCl) as a green solid.

### Step 3: tert-butyl 7-[3-[(2,6-dioxo-3-piperidyl)amino]anilino]-7-oxo-heptanoate

To a solution of 7-tert-butoxy-7-oxo-heptanoic acid (676.65 mg, 3.13 mmol, 1 eq) in DMF (8 mL) was added HATU (2.38 g, 6.26 mmol, 2 eq) and then was added 3-(3-aminoanilino)piperidine-2,6-dione (800 mg, 3.13 mmol, 1 eq, HCl) and DIEA (1.21 g, 9.39 mmol, 1.63 mL, 3 eq) after 0.1 h. The mixture was stirred at 25° C. for 1 h. LCMS showed starting material was consumed completely and the desired mass was detected. The reaction mixture was diluted with water (30 mL), then was extracted with ethyl acetate (30 mL×3). The combined organic phase was washed with brine (30 mL), dried with anhydrous Na₂SO₄, filtered and concentrated in vacuum to give a residue. The residue was purified by prep-HPLC (FA) and lyophilized to give tert-butyl 7-[3-[(2,6-dioxo-3-piperidyl)amino]anilino]-7-oxo-heptanoate (900 mg, 69% yield) as white solid.

LC-MS [ESI, M −55]: 362.0.

¹H NMR (400 MHz, CHLOROFORM-d) 6=8.36 (br d, J=1.6 Hz, 1H), 7.38 (br s, 2H), 7.18-7.03 (m, 1H), 6.65 (br d, J=3.6 Hz, 1H), 6.45 (br d, J=5.6 Hz, 1H), 4.12 (br d, J=9.2 Hz, 1H), 2.90-2.69 (m, 2H), 2.55 (br d, J=10.4 Hz, 1H), 2.39-2.31 (m, 2H), 2.26-2.20 (m, 2H), 1.89 (br d, J=8.8 Hz, 1H), 1.79-1.69 (m, 2H), 1.65-1.59 (m, 2H), 1.44 (s, 11H).

### Step 4: 7-[3-[(2,6-dioxo-3-piperidyl)amino]anilino]-7-oxo-heptanoic acid

To a solution of tert-butyl 7-[3-[(2,6-dioxo-3-piperidyl)amino]anilino]-7-oxo-heptanoate (200 mg, 479.04 mol, 1 eq) in DCM (2 mL) was added HCl/dioxane (2 M, 2.86 mL, 11.93 eq). The mixture was stirred at 25° C. for 1 h. LCMS showed starting material was consumed completely and the desired mass was detected. The reaction mixture was concentrated under the reduced pressure to give 7-[3-[(2,6-dioxo-3-piperidyl)amino]anilino]-7-oxo-heptanoic acid (220 mg, crude, HCl) as white solid.

LC-MS [ESI, M+1]: 362.1.

### Step 5: tert-butyl 3-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of 7-[3-[(2,6-dioxo-3-piperidyl)amino]anilino]-7-oxo-heptanoic acid (31.24 mg, 78.52 mol, 1 eq, HCl) in DMF (1 mL) was added HATU (59.71 mg, 157.04 mol, 2 eq) and then was added DIEA (30.44 mg, 235.56 mol, 41.03 μL, 3 eq) and tert-butyl 3-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (50 mg, 78.52 mol, 1 eq) after 0.1 h. The mixture was stirred at 25° C. for 1 h. LCMS showed starting material was consumed completely and the desired compound was detected. The reaction mixture was concentrated under the reduced pressure to give a residue. The residue was purified by prep-HPLC (column: CD01-Phenomenex luna C18 150*25*10 um; mobile phase: [water(FA)-ACN]; gradient:53%-83% B over 12 min) and lyophilized to give tert-butyl 3-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (20 mg, 25.99% yield) as white solid.

LC-MS [ESI, M/2+1]: 491.0.

### Step 6: 3-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine2-carboxylic acid

To a solution of tert-butyl 3-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (15 mg, 15.30 mol, 1 eq) in DCM (0.5 mL) was added HCl/EtOAc (6 M, 1.5 mL, 588.10 eq). The mixture was stirred at 25° C. for 1 h. LCMS showed starting material was consumed and one main peak with desired mass was detected. The reaction mixture was concentrated under the reduced pressure to give the crude product to give a residue, the residue was purified by prep-HPLC (column: CD01-Phenomenex luna C18 150*25*10 um; mobile phase: [water(FA)-ACN]; gradient:34%-64% B over 8 min) and lyophilized to give 3-[[7-[3-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine2-carboxylic acid (2.36 mg, 16% yield) as white solid.

LC-MS [ESI, M+1]: 924.6.

¹H NMR (400 MHz, DMSO-d₆) δ=10.77 (s, 1H), 9.85 (br d, J=2.0 Hz, 1H), 8.47 (br t, J=9.6 Hz, 1H), 8.15 (s, 1H), 7.91 (br d, J=11.6 Hz, 1H), 7.42-7.20 (m, 5H), 7.05 (br d, J=1.2 Hz, 1H), 6.99-6.91 (m, 1H), 6.85 (br d, J=7.2 Hz, 1H), 6.65-6.49 (m, 1H), 6.35 (br d, J=8.8 Hz, 1H), 5.79 (br d, J=6.8 Hz, 1H), 4.34-4.20 (m, 1H), 3.95 (br t, J=6.8 Hz, 1H), 3.79 (br s, 1H), 3.67 (br t, J=7.2 Hz, 1H), 3.63-3.55 (m, 2H), 3.53-3.41 (m, 7H), 2.85-2.75 (m, 1H), 2.63-2.57 (m, 1H), 2.54 (s, 3H), 2.30-2.20 (m, 6H), 2.15-2.08 (m, 1H), 2.06-1.93 (m, 4H), 1.88 (br s, 1H), 1.61 (br s, 4H), 1.34 (br d, J=10.0 Hz, 5H).

### Example 26

### 3-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

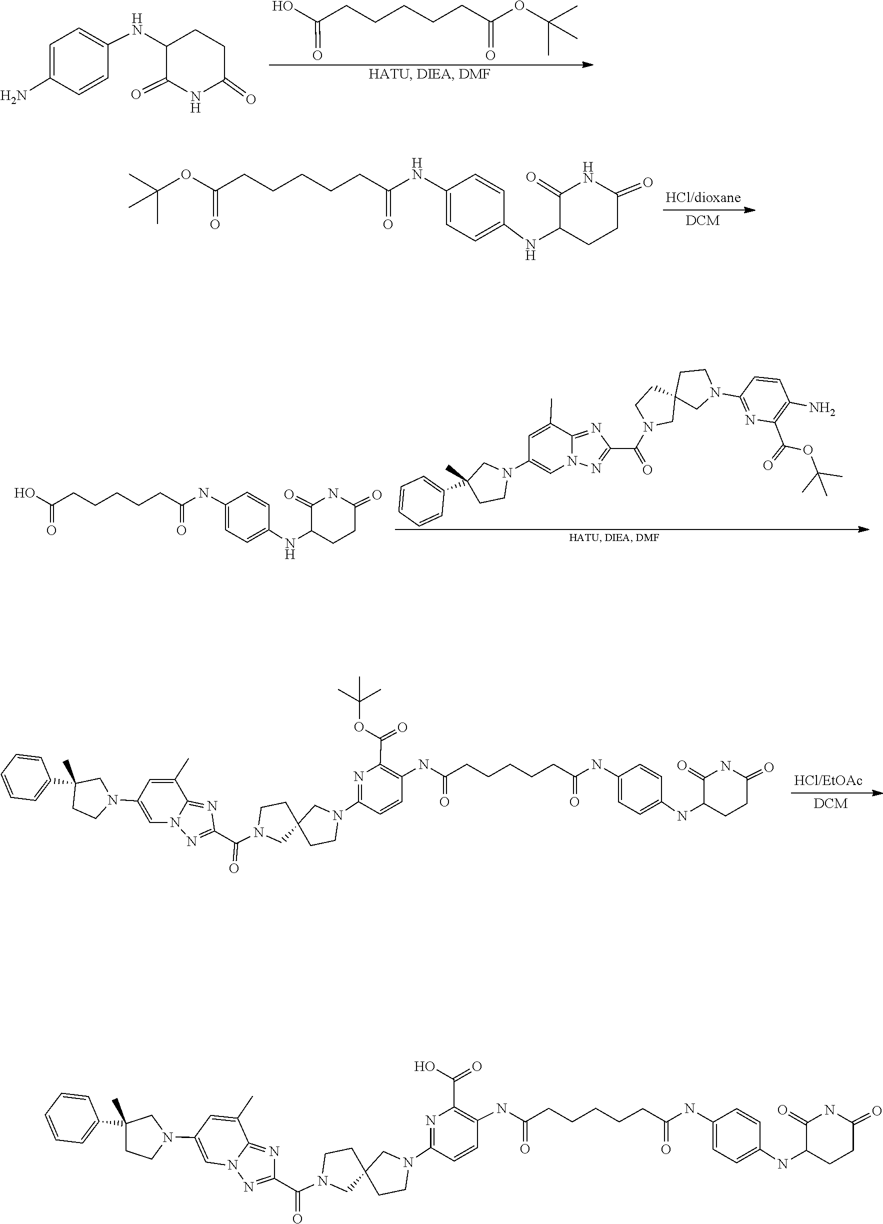

### Step 1: tert-butyl 7-[4-[(2,6-dioxo-3-piperidyl)amino]anilino]-7-oxo-heptanoate

To a solution of 7-tert-butoxy-7-oxo-heptanoic acid (700.33 mg, 3.24 mmol, 1.03 eq) in DMF (8 mL) was added HATU (2.38 g, 6.26 mmol, 2 eq) at 0° C. After addition, the mixture was stirred at this temperature for 0.5 h and then 3-(4-aminoanilino)piperidine-2,6-dione (800 mg, 3.13 mmol, 1 eq, HCl) and DIEA (2.02 g, 15.64 mmol, 2.72 mL, 5 eq) was added at 0° C. The resulting mixture was stirred at 25° C. for 1 h. LCMS showed starting material was consumed completely and the desired compound was detected. The reaction mixture was quenched by water (10 mL) and extracted with EtOAc (10 mL×3), the combined organic layers were washed with brine (20 mL×3), dried over anhydrous Na₂SO₄, filtered and dried to give a residue. The residue was purified by flash silica gel chromatography (ISCO®; 40 g SepaFlash® Silica Flash Column, Eluent of 0˜70% EtOAc: PE @100 mL/min) to give tert-butyl 7-[4-[(2,6-dioxo-3-piperidyl)amino]anilino]-7-oxo-heptanoate (800 mg, 61%) as a blue solid.

LC-MS [ESI, M]: 418.2

¹H NMR (400 MHz, DMSO-d₆) δ=10.77 (s, 1H), 9.47 (s, 1H), 7.27 (d, J=8.8 Hz, 2H), 6.60 (d, J=8.8 Hz, 2H), 5.63 (d, J=7.6 Hz, 1H), 4.32-4.18 (m, 1H), 2.80-2.67 (m, 1H), 2.63-2.54 (m, 1H), 2.19 (td, J=7.2, 12.2 Hz, 4H), 2.11 (qd, J=4.4, 13.2 Hz, 1H), 1.85 (dq, J=4.8, 12.0 Hz, 1H), 1.61-1.45 (m, 4H), 1.38 (s, 9H), 1.33-1.21 (m, 2H)

### Step 2: 7-[4-[(2,6-dioxo-3-piperidyl)amino]anilino]-7-oxo-heptanoic acid

To a solution of tert-butyl 7-[4-[(2,6-dioxo-3-piperidyl)amino]anilino]-7-oxo-heptanoate (400 mg, 958.09 mol, 1 eq) in DCM (4 mL) was added HCl/dioxane (2 M, 4 mL, 8.35 eq). The mixture was stirred at 25° C. for 0.5 h. LCMS showed starting material was consumed and the desired mass was detected. The reaction solution was concentrated under the reduced pressure to give compound 7-[4-[(2,6-dioxo-3-piperidyl)amino]anilino]-7-oxo-heptanoic acid (380 mg, crude, HCl) as a green solid

LC-MS [ESI, M+1]: 362.1.

### Step 3: tert-butyl 3-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of 7-[4-[(2,6-dioxo-3-piperidyl)amino]anilino]-7-oxo-heptanoic acid (22.49 mg, 56.53 mol, 1 eq, HCl) in DMF (1 mL) was added HATU (42.99 mg, 113.07 mol, 2 eq) at 0° C. After addition, the mixture was stirred at this temperature for 0.5 h, and then tert-butyl 3-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (36.00 mg, 56.53 mol, 1 eq) and DIEA (36.53 mg, 282.67 mol, 49.23 μL, 5 eq) was added at 25° C. The resulting mixture was stirred at 25° C. for 1 h. LCMS showed starting material was consumed and the desired mass was detected. The reaction solution was filtered and the filtrate was concentrated under the reduced pressure to give the residue. The residue was purified by prep-HPLC (column: CD01-Phenomenex luna C18 150*25*10 um; mobile phase: [water(FA)-ACN]; gradient:54%-84% B over 10 min.) and lyophilized to give tert-butyl 3-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (15 mg, 25%) as a white solid

LC-MS [ESI, M+1]:980.6.

¹H NMR (400 MHz, DMSO-d₆) δ=10.78 (s, 1H), 9.58 (s, 1H), 9.49 (br s, 1H), 7.92 (br d, J=6.8 Hz, 1H), 7.73 (dd, J=5.6, 8.8 Hz, 1H), 7.43-7.18 (m, 9H), 6.65-6.57 (m, 3H), 4.26 (br dd, J=4.0, 11.2 Hz, 1H), 3.95 (br t, J=6.8 Hz, 1H), 3.80 (s, 1H), 3.67 (br t, J=6.8 Hz, 1H), 3.58 (br dd, J=3.6, 8.8 Hz, 2H), 3.54-3.45 (m, 7H), 2.78-2.69 (m, 1H), 2.61-2.52 (m, 4H), 2.33-2.21 (m, 6H), 2.10 (td, J=4.4, 8.4 Hz, 1H), 2.05-1.93 (m, 4H), 1.91-1.79 (m, 1H), 1.59 (br d, J=6.8 Hz, 4H), 1.49 (d, J=5.2 Hz, 9H), 1.35 (br d, J=8.4 Hz, 5H)

### Step 4: 3-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of tert-butyl 3-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (15 mg, 15.30 mol, 1 eq) in THF (1 mL) was added HCl/EtOAc (6 M, 4.50 mL, 1764.29 eq). The mixture was stirred at 25° C. for 1 h. LCMS showed starting material was consumed and one main peak with desired mass was detected. The mixture was filtered. The residue was purified by prep-HPLC (column: CD01-Phenomenex luna C18 150*25*10 um; mobile phase: [water(FA)-ACN]; gradient:30%-60% B over 10 min.) and lyophilized to give compound 3-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (12.34 mg, 86.98%) as a yellow solid.

LC-MS [ESI, M]: 924.6.

¹H NMR (400 MHz, DMSO-d₆) δ=10.78 (s, 1H), 10.46 (br s, 1H), 9.51 (s, 1H), 8.38 (br t, J=9.6 Hz, 1H), 7.88 (br d, J=13.2 Hz, 1H), 7.44-7.16 (m, 8H), 6.74 (br dd, J=9.2, 17.2 Hz, 1H), 6.60 (br d, J=8.0 Hz, 2H), 5.63 (br s, 1H), 4.29-4.21 (m, 1H), 3.95 (br s, 1H), 3.79 (br d, J=2.0 Hz, 1H), 3.66 (br d, J=6.8 Hz, 1H), 3.61-3.52 (m, 9H), 2.74 (ddd, J=5.2, 12.0, 17.6 Hz, 1H), 2.63-2.51 (m, 4H), 2.37-2.28 (m, 2H), 2.28-2.18 (m, 4H), 2.14-1.93 (m, 5H), 1.85 (dq, J=4.4, 12.0 Hz, 1H), 1.59 (br d, J=6.4 Hz, 4H), 1.33 (br d, J=9.6 Hz, 5H)

### Example 27

### 8-(aminomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide

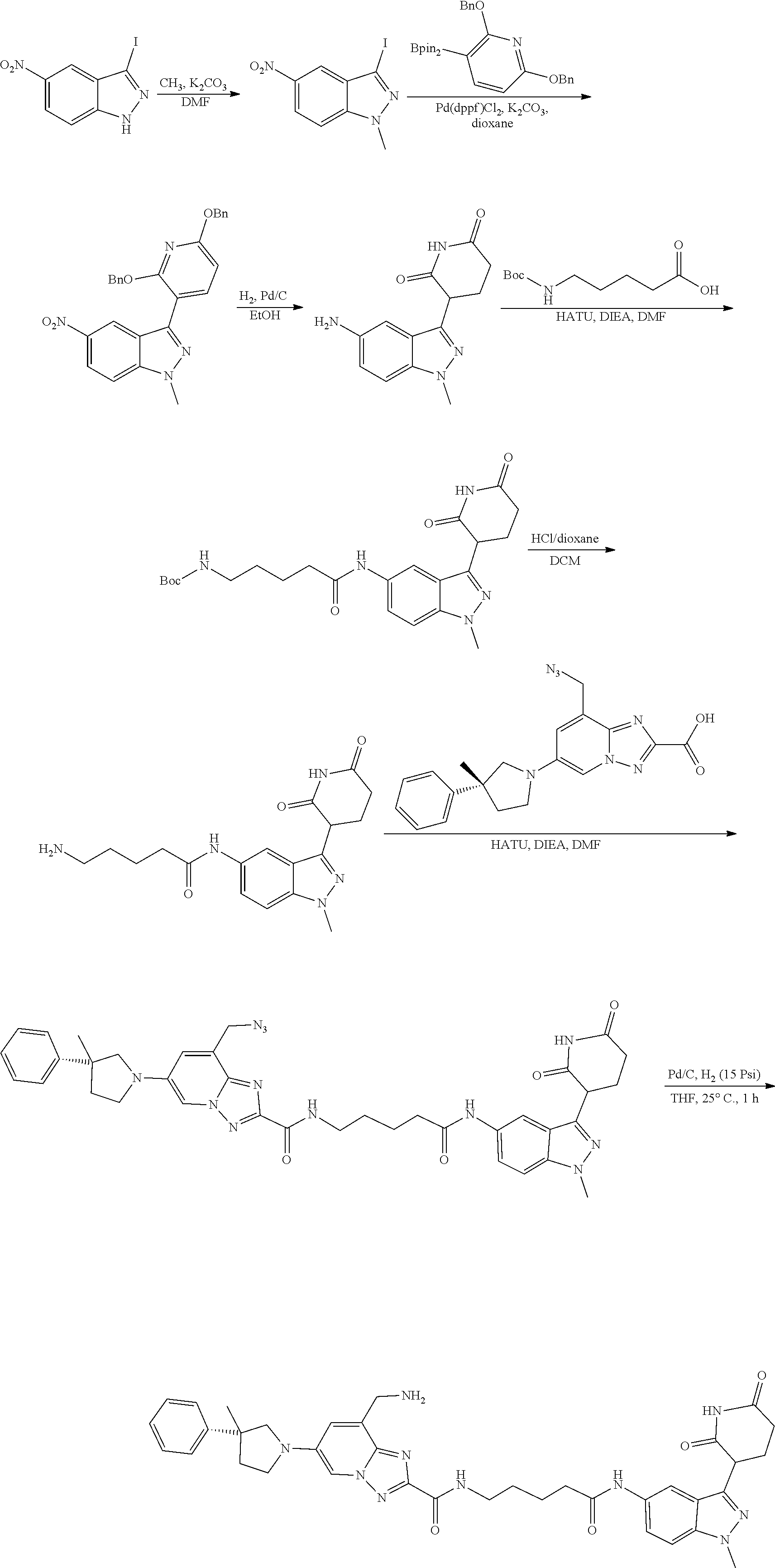

### Step 1: 3-iodo-1-methyl-5-nitro-1H-indazole

To a solution of 3-iodo-5-nitro-1H-indazole (1.00 g, 3.46 mmol, 1 eq) and K₂CO₃ (478 mg, 3.46 mmol, 1 eq) in DMF (10 mL) was added CH₃I (982 mg, 6.92 mmol, 2 eq) at 0° C. The mixture was stirred at 25° C. for 1 h. LCMS showed starting material was consumed and the desired mass was detected. The reaction was quenched by ice water (5 mL) slowly and then extracted with ethyl acetate (10 mL×3). The combined organic phase was washed with brine (10 mL), dried, filtered and concentrated in vacuum to give the residue, which was purified by column chromatography (SiO₂, Petroleum ether/Ethyl acetate=3:1, Rf=0.36) to give 3-iodo-1-methyl-5-nitro-1H-indazole (400 mg, 38% yield) as a yellow solid

LC-MS [ESI, M+1]: 304.0.

¹H NMR (400 MHz, CDCl₃) δ=8.50 (d, J=2.0 Hz, 1H), 8.34 (dd, J=2.0, 9.2 Hz, 1H), 7.46 (d, J=9.2 Hz, 1H), 4.18 (s, 3H)

### Step 2: 3-(2,6-bis(benzyloxy)pyridin-3-yl)-1-methyl-5-nitro-1H-indazole

A mixture of 3-iodo-1-methyl-5-nitro-indazole (400 mg, 1.32 mmol, 1 eq), 2,6-dibenzyloxy-3-(4,4,5,5-tetramethyl-1,3,2-dioxaborolan-2-yl)pyridine (551 mg, 1.32 mmol, 1 eq), cyclopentyl(diphenyl)phosphane; dichloromethane; dichloropalladium; iron (54 mg, 65.99 mol, 0.05 eq), Cs₂CO₃ (860 mg, 2.64 mmol, 2 eq) in dioxane (5 mL) and H₂O (1 mL) was degassed and purged with N₂ for 3 times, and then the mixture was stirred at 100° C. for 12 h under N₂ atmosphere. LCMS showed starting material was consumed and the desired mass was detected. The mixture was concentrated to give the residue, which was purified by column chromatography (SiO₂, Petroleum ether/Ethyl acetate=3:1, Rf=0.25) to give 3-(2,6-bis(benzyloxy)pyridin-3-yl)-1-methyl-5-nitro-1H-indazole (510 mg, 83% yield) as a yellow solid

LC-MS [ESI, M+23]: 467.2.

¹H NMR (400 MHz, CDCl₃) δ=8.85 (d, J=2.0 Hz, 1H), 8.24 (dd, J=2.0, 9.2 Hz, 1H), 7.95 (d, J=8.0 Hz, 1H), 7.53-7.45 (m, 2H), 7.45-7.30 (m, 6H), 7.29-7.23 (m, 3H), 6.57 (d, J=8.0 Hz, 1H), 5.47 (d, J=5.6 Hz, 4H), 4.15 (s, 3H)

### Step 3: 3-(5-amino-1-methyl-1H-indazol-3-yl)piperidine-2,6-dione

To a solution of 3-(2,6-dibenzyloxy-3-pyridyl)-1-methyl-5-nitro-indazole (510 mg, 1.09 mmol, 1 eq) in THE (10 mL) was added Pd/C (116.35 mg, 109.33 mol, 10% purity, 0.1 eq), Pd(OH)₂ (77 mg, 109.33 mol, 20% purity, 0.1 eq), AcOH (131 mg, 2.19 mmol, 2 eq) under N₂ atmosphere. The mixture was stirred under H₂ (50 Psi) at 55° C. for 12 h. LCMS showed starting material was consumed and the desired mass was detected. The reaction mixture was filtered and the filtrate was concentrated under the reduced pressure to give 3-(5-amino-1-methyl-1H-indazol-3-yl)piperidine-2,6-dione (350 mg, crude) as a yellow solid.

LC-MS [ESI, M+1]: 259.1.

### Step 4: tert-butyl (5-((3-(2,6-dioxopiperidin-3-yl)-1-methyl-1H-indazol-5-yl)amino)-5-oxopentyl)carbamate

To a solution of 3-(5-amino-1-methyl-1H-indazol-3-yl)piperidine-2,6-dione (340 mg, 1.32 mmol, 1 eq) and 5-(tert-butoxycarbonylamino)pentanoic acid (429 mg, 1.97 mmol, 1.5 eq) in DMF (3 mL) was added HATU (1 g, 2.63 mmol, 2 eq), and the mixture was stirred at 25° C. for 0.5 h, then DIEA (851 mg, 6.58 mmol, 5 eq) was added, the mixture was stirred at 25° C. for 1 h. LCMS showed starting material was consumed and the desired mass was detected. The reaction mixture was filtered and the filtrate was concentrated under the reduced pressure to give the residue, which was purified by prep-HPLC (NH₄HCO₃ condition column: CD06-Waters Xbidge C18 150*40*10 um; mobile phase: [water(NH₄HCO₃)-ACN]; gradient: 20%-50% B over 12 min) to give tert-butyl (5-((3-(2,6-dioxopiperidin-3-yl)-1-methyl-1H-indazol-5-yl)amino)-5-oxopentyl)carbamate (210 mg, 35% yield) as a red solid.

LC-MS [ESI, M-100+1]: 358.2

¹H NMR (400 MHz, CDCl₃) δ=8.04 (s, 2H), 7.75 (s, 1H), 7.42 (dd, J=1.6, 8.8 Hz, 1H), 7.30 (d, J=9.2 Hz, 1H), 4.69 (s, 1H), 4.29 (dd, J=5.2, 7.6 Hz, 1H), 4.01 (s, 3H), 3.20 (d, J=5.6 Hz, 2H), 2.97-2.88 (m, 1H), 2.67 (ddd, J=5.2, 7.7, 17.8 Hz, 1H), 2.56-2.47 (m, 1H), 2.46-2.32 (m, 3H), 1.83-1.76 (m, 2H), 1.61-1.57 (m, 2H), 1.45 (s, 9H)

### Step 5: 5-amino-N-(3-(2,6-dioxopiperidin-3-yl)-1-methyl-1H-indazol-5-yl)pentanamide

To a solution of tert-butyl (5-((3-(2,6-dioxopiperidin-3-yl)-1-methyl-1H-indazol-5-yl)amino)-5-oxopentyl)carbamate (100 mg, 218.57 mol, 1 eq) in DCM (2 mL) was added HCl (6 M, 1 mL, 27.45 eq), the mixture was stirred at 25° C. for 1 h. LCMS showed starting material was consumed and the desired mass was detected. The reaction mixture was concentrated under the reduced pressure to give 5-amino-N-(3-(2,6-dioxopiperidin-3-yl)-1-methyl-1H-indazol-5-yl)pentanamide (87 mg, crude, HCl) as a brown solid.

LC-MS [ESI, M+1]: 358.1.

### Step 6: 8-(azidomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide

To a solution of 5-amino-N-(3-(2,6-dioxopiperidin-3-yl)-1-methyl-1H-indazol-5-yl)pentanamide (87 mg, 262 mol, 0.9 eq) and 8-(azidomethyl)-6-[(3R)-3-methyl-3-phenyl-pyrrolidin-1-yl]-[1,2,4]triazolo[1,5-a]pyridine-2-carboxylic acid (110 mg, 290 mol, 1 eq) in DMF (2 mL) was added HATU (221 mg, 583 mol, 2 eq) and DIEA (113 mg, 874 mol, 3 eq). The mixture was stirred at 25° C. for 10 min. LCMS showed starting material was consumed and the desired mass was detected. The residue was purified by prep—HPLC (column: CD02-Waters Xbidge BEH C18 150*25*10 um; mobile phase: [water(NH₄HCO₃)-ACN]; gradient: 36%-66% B over 10 min) to give 8-(azidomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide (54.38 mg, 26% yield) as a green solid.

LC-MS [ESI, M+1]: 717.3.

¹H NMR (400 MHz, CDCl₃) δ=8.43 (s, 1H), 8.20 (s, 1H), 8.05 (s, 1H), 7.74 (s, 1H), 7.56 (d, J=7.2 Hz, 2H), 7.40-7.35 (m, 2H), 7.34-7.30 (m, 2H), 7.28 (s, 1H), 7.27-7.24 (m, 1H), 7.20 (s, 1H), 4.78 (s, 2H), 4.30 (dd, J=5.2, 7.2 Hz, 1H), 3.97 (s, 3H), 3.64-3.48 (m, 5H), 3.43 (dt, J=3.6, 8.8 Hz, 1H), 2.88 (ddd, J=4.8, 8.0, 17.6 Hz, 1H), 2.64 (ddd, J=4.8, 8.0, 17.6 Hz, 1H), 2.54-2.24 (m, 6H), 1.82 (dd, J=6.8, 14.0 Hz, 2H), 1.78-1.72 (m, 2H), 1.47 (s, 3H).

### Step 7: 8-(aminomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide

To a solution of 8-(azidomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide (40 mg, 55 mol, 1 eq) in THE (2 mL) was added Pd/C (11 mg, 11 mol, 10% purity, 0.2 eq) under H₂ (15 psi), the mixture was stirred at 25° C. for 2 h. LCMS showed starting material was consumed and the desired mass was detected. The mixture was filtered and concentrated to give the residue, which was purified by prep—HPLC (column: CD06-Waters Xbidge C18 150*40*10 um; mobile phase: [water(HCl)-ACN]; gradient:16%-46% B over 11 min) to give 8-(aminomethyl)-N-[5-[[3-(2,6-dioxo-3-piperidyl)-1-methyl-indazol-5-yl]amino]-5-oxo-pentyl]-6-[(3R)-3-methyl-3-phenyl-pyrrolidin-1-yl]-[1,2,4]triazolo[1,5-a]pyridine-2-carboxamide (4.21 mg, 11% yield) as a yellow solid.

LC-MS [ESI, M+1]: 691.4.

¹H NMR (400 MHz, DMSO-d₆) δ=10.89 (s, 1H), 9.98 (s, 1H), 8.81-8.72 (m, 1H), 8.65 (s, 3H), 8.06 (d, J=16.4 Hz, 2H), 7.72 (s, 1H), 7.57-7.44 (m, 2H), 7.42-7.30 (m, 4H), 7.25 (d, J=7.2 Hz, 1H), 4.40 (d, J=5.6 Hz, 2H), 4.34-4.26 (m, 1H), 3.96 (s, 3H), 3.63-3.51 (m, 6H), 2.74-2.58 (m, 2H), 2.40-2.27 (m, 5H), 2.19 (d, J=5.6 Hz, 1H), 1.62 (s, 4H), 1.37 (s, 3H).

### Example 28

### 4-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

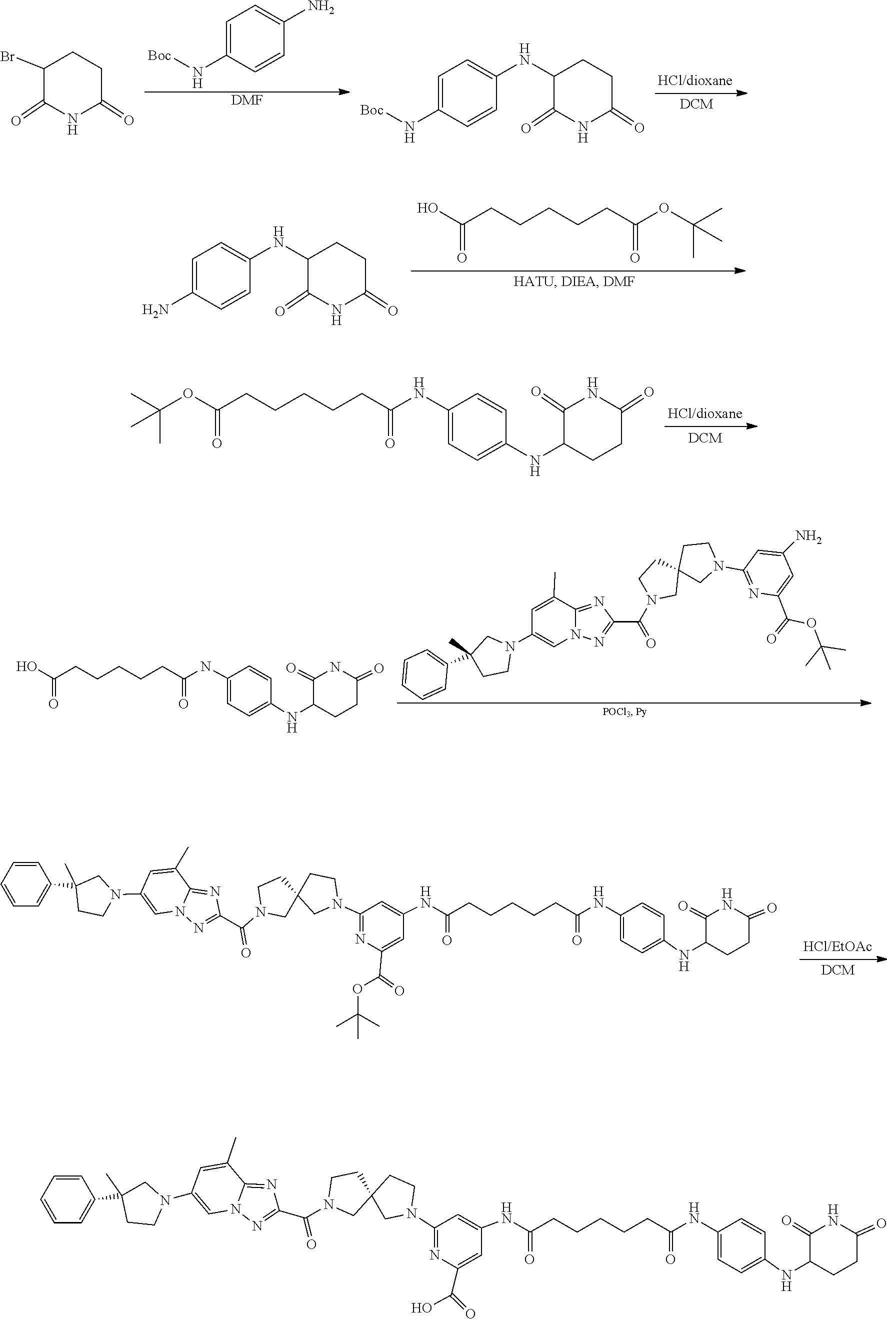

### Step 1: tert-butyl N-14-[(2,6-dioxo-3-piperidyl)amino]phenyl]carbamate

A mixture of 3-bromopiperidine-2,6-dione (1.00 g, 5.21 mmol, 1 eq), tert-butyl N-(4-aminophenyl)carbamate (1 g, 5.21 mmol, 1 eq) in DMF (10 mL) was degassed and purged with N₂ for 3 times, and then the mixture was stirred at 25° C. for 2 h under N₂ atmosphere. LCMS showed starting material was consumed and the desired mass was detected. The reaction solution was concentrated under the reduced pressure to give the residue, which was purified by prep-HPLC (column:120 g Flash Column Welch Ultimate XB_C18 20-40 m; 120 A; mobile phase: [water(FA)-ACN]; B %: 5%-42%, 25 min) to give tert-butyl N-[4-[(2,6-dioxo-3-piperidyl)amino]phenyl]carbamate (680 mg, 41% yield) as a gray solid.

LC-MS [ESI, M]: 320.2.

¹H NMR (400 MHz, DMSO-d₆) δ=10.76 (s, 1H), 8.84 (s, 1H), 7.14 (d, J=6.8 Hz, 2H), 6.59 (d, J=8.8 Hz, 2H), 5.55 (d, J=6.4 Hz, 1H), 4.32-4.15 (m, 1H), 2.81-2.67 (m, 1H), 2.60 (s, 1H), 2.11 (td, J=4.0, 8.4 Hz, 1H), 1.85 (m, 1H), 1.45 (s, 9H).

### Step 2: 3-(4-aminoanilino)piperidine-2,6-dione

To a solution of tert-butyl N-[4-[(2,6-dioxo-3-piperidyl)amino]phenyl]carbamate (680 mg, 2.13 mmol, 1 eq) in DCM (4 mL) was added HCl/dioxane (2 M, 6 mL, 5.64 eq). The mixture was stirred at 20° C. for 0.5 h. LCMS showed starting material was consumed and the desired mass was detected. The reaction solution was concentrated under the reduced pressure to give compound 3-(4-aminoanilino)piperidine-2,6-dione (544 mg, crude, HCl) as a violet solid.

LC-MS [ESI, M+1]: 220.1.

¹H NMR (400 MHz, DMSO-d₆) δ=10.80 (s, 1H), 9.99 (s, 3H), 7.11 (d, J=8.8 Hz, 2H), 6.74 (d, J=8.8 Hz, 2H), 4.35 (dd, J=4.8, 11.6 Hz, 1H), 2.80-2.67 (m, 1H), 2.63-2.55 (m, 1H), 2.13-2.02 (m, 1H), 1.96-1.83 (m, 1H).

### Step 3: tert-butyl 7-[4-[(2,6-dioxo-3-piperidyl)amino]anilino]-7-oxo-heptanoate

To a solution of 7-tert-butoxy-7-oxo-heptanoic acid (700 mg, 3.24 mmol, 1 eq) in DMF (8 mL) was added HATU (2.38 g, 6.26 mmol, 2 eq) at 0° C. After addition, the mixture was stirred at this temperature for 0.5 h and then 3-(4-aminoanilino)piperidine-2,6-dione (800 mg, 3.13 mmol, 1 eq, HCl) and DIEA (2 g, 15 mmol, 5 eq) was added at 0° C. The resulting mixture was stirred at 25° C. for 1 h. LCMS showed starting material was consumed and the desired mass was detected. The reaction mixture was quenched by water (30 mL) and extracted with EtOAc (40 mL×3), the combined organic layers were washed with brine (20 mL×3), dried o, filtered and dried to give the residue, which was purified by flash silica gel chromatography (ISCO®; 40 g SepaFlash® Silica Flash Column, Eluent of 0˜70% EtOAc: PE @100 mL/min) to give tert-butyl 7-[4-[(2,6-dioxo-3-piperidyl)amino]anilino]-7-oxo-heptanoate (800 mg, 61%) as a blue solid.

LC-MS [ESI, M]: 418.2

¹H NMR (400 MHz, DMSO-d₆) δ=10.77 (s, 1H), 9.47 (s, 1H), 7.27 (d, J=8.8 Hz, 2H), 6.60 (d, J=8.8 Hz, 2H), 5.63 (d, J=7.6 Hz, 1H), 4.32-4.18 (m, 1H), 2.80-2.67 (m, 1H), 2.63-2.54 (m, 1H), 2.19 (td, J=7.2, 12.2 Hz, 4H), 2.11 (qd, J=4.4, 13.2 Hz, 1H), 1.85 (dq, J=4.8, 12.0 Hz, 1H), 1.61-1.45 (m, 4H), 1.38 (s, 9H), 1.33-1.21 (m, 2H).

### Step 4: 7-[4-[(2,6-dioxo-3-piperidyl)amino]anilino]-7-oxo-heptanoic acid

To a solution of tert-butyl 7-[4-[(2,6-dioxo-3-piperidyl)amino]anilino]-7-oxo-heptanoate (400 mg, 958 mol, 1 eq) in DCM (4 mL) was added HCl/dioxane (2 M, 4 mL, 8.35 eq). The mixture was stirred at 25° C. for 0.5 h. LCMS showed starting material was consumed and the desired mass was detected. The reaction solution was concentrated under the reduced pressure to give 7-[4-[(2,6-dioxo-3-piperidyl)amino]anilino]-7-oxo-heptanoic acid (380 mg, crude, HCl) as a green solid

LC-MS [ESI, M+1]: 362.1.

### Step 5: tert-butyl 4-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate

To a solution of 7-[4-[(2,6-dioxo-3-piperidyl)amino]anilino]-7-oxo-heptanoic acid (30 mg, 70 mol, 1.1 eq, HCl) and tert-butyl 4-amino-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (40 mg, 62 mol, 1 eq) in Py (0.5 mL) was added a solution of POCl3 (10 mg, 63 mol, 1 eq) in DCM (0.1 mL) dropwise at 0° C. The mixture was stirred at 0° C. for 1 h. LCMS showed starting material was consumed and the desired mass was detected. The reaction was quenched by addition of 1 mL of water and then extracted by diethyl ether (3×1 mL), dried, filtered and concentrated to give the residue, which was purified by prep-HPLC (column: CD01-Phenomenex luna C18 150*25*10 um; mobile phase: [water(FA)-ACN]; gradient:37%-67% B over 10 min.) to give tert-butyl 4-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (15 mg, 23%) as a yellow solid

LC-MS [ESI, M]: 980.6

¹H NMR (400 MHz, DMSO-d₆) δ=10.77 (s, 1H), 10.17 (d, J=9.2 Hz, 1H), 9.49 (s, 1H), 8.40 (s, 1H), 7.91 (d, J=15.6 Hz, 1H), 7.43-7.32 (m, 4H), 7.32-7.19 (m, 5H), 7.15-7.04 (m, 1H), 6.59 (d, J=8.0 Hz, 2H), 5.63 (d, J=7.6 Hz, 1H), 4.30-4.17 (m, 1H), 4.00-3.88 (m, 1H), 3.83-3.75 (m, 1H), 3.69-3.64 (m, 1H), 3.63-3.56 (m, 2H), 3.55-3.44 (m, 7H), 2.78-2.66 (m, 1H), 2.63-2.51 (m, 4H), 2.36-2.29 (m, 2H), 2.28-2.17 (m, 4H), 2.10 (dd, J=4.8, 13.6 Hz, 1H), 2.06-1.91 (m, 4H), 1.90-1.79 (m, 1H), 1.59 (br s, 4H), 1.51 (d, J=5.6 Hz, 9H), 1.34 (d, J=9.6 Hz, 5H).

### Step 6: 4-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid

To a solution of tert-butyl 4-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylate (10 mg, 10.20 mol, 1 eq) in THE (1 mL) was added HCl/EtOAc (6 M, 3 mL). The mixture was stirred at 25° C. for 1 h. LCMS showed starting material was consumed and the desired mass was detected. The reaction solution was filtered and the filtrate was concentrated under the reduced pressure to give the residue, which was purified by prep-HPLC (column: CD01-Phenomenex luna C18 150*25*10 um; mobile phase: [water(FA)-ACN]; gradient:33%-63% B over 10 min.) to give 4-[[7-[4-[(2,6-dioxo-3-piperidyl)amino]anilino]-7-oxo-heptanoyl]amino]-6-[(5R)-2-[8-methyl-6-[(3R)-3-methyl-3-phenyl-pyrrolidin-1-yl]-[1,2,4]triazolo[1,5-a]pyridine-2-carbonyl]-2,7-diazaspiro[4.4]nonan-7-yl]pyridine-2-carboxylic acid (3.29 mg, 33% yield) as a gray solid.

LC-MS [ESI, M+1]:924.6.

¹H NMR (400 MHz, DMSO-d₆) δ=10.77 (s, 1H), 10.16 (d, J=8.4 Hz, 1H), 9.50 (d, J=2.0 Hz, 1H), 7.90 (d, J=14.8 Hz, 1H), 7.44-7.17 (m, 9H), 7.04 (d, J=19.2 Hz, 1H), 6.59 (d, J=7.6 Hz, 2H), 5.63 (d, J=7.6 Hz, 1H), 4.30-4.17 (m, 1H), 3.94 (t, J=6.8 Hz, 1H), 3.79 (d, J=4.0 Hz, 1H), 3.68-3.65 (m, 1H), 3.61-3.50 (m, 9H), 2.75-2.67 (m, 1H), 2.62-2.51 (m, 4H), 2.32 (q, J=7.6 Hz, 2H), 2.28-2.18 (m, 4H), 2.13-2.06 (m, 1H), 2.06-1.91 (m, 4H), 1.84 (dq, J=4.4, 12.0 Hz, 1H), 1.65-1.52 (m, 4H), 1.38-1.25 (m, 5H)

### Example 29

### Differential Scanning Fluorimetry Assay

The stabilization effects the compounds have on the immunoglobulin light chain protein was quantitively measured using a thermal shift assay. Recombinant amyloidogenic full length light chain protein WIL-FL T46L was produced in E. coli, purified, and used in the assay. The Thermal Shift Protein Stability Kit from Biotium was used and the assay was done in 96-well Plates following the manufacturer's instructions. The protein and compounds were incubated at RT for 30 min before adding the dye and subjected to the thermal shift assay. The thermal stability (Tm) of each sample was then determined using standard curve fitting methods and compared to vehicle. Data are expressed as temperature shift: [Tm]_{compound}−[Tm]vehicle.

### Results

The compounds provided herein induce shifts in the ranges below:

$$0 - 0.5 ° C . = *$$

$$> 0.5 - 1 ° C . = **$$

$$> 1 - 1.5 ° C . = \text{***}$$

$$> 1.5 - 2.5 ° C . = \text{****}$$

$$> 2.5 ° C . = \text{*****}$$

Results for the compounds provided herein are shown in the Table below.

| Ex. No. | DSF |
|---|---|
| 1 | *** |
| 2 | ***** |
| 3 | ***** |
| 4 | ***** |
| 5 | ***** |
| 6 | **** |
| 7 | ***** |
| 8 | |
| 9 | ***** |
| 10 | **** |
| 11 | **** |
| 12 | **** |
| 13 | ***** |
| 14 | **** |
| 15 | ***** |
| 16 | ** |
| 17 | ***** |
| 18 | ***** |
| 19 | ***** |
| 20 | ***** |
| 21 | ***** |
| 22 | **** |
| 23 | ***** |
| 24 | ***** |
| 24A | ***** |
| 25 | ***** |
| 26 | ***** |
| 27 | ***** |
| 28 | ***** |

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

1. A compound of Formula I: 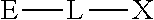
or a pharmaceutically acceptable derivative thereof, wherein E is an E3 ligase ligand or a moiety that activates the N-degron pathway; L is a bond or a divalent chemical linker; and X is a moiety that stabilizes immunoglobulin light chains.

2. The compound of claim 1 that has any one of Formulae II-IX.

3. The compound of claim 1, wherein L is 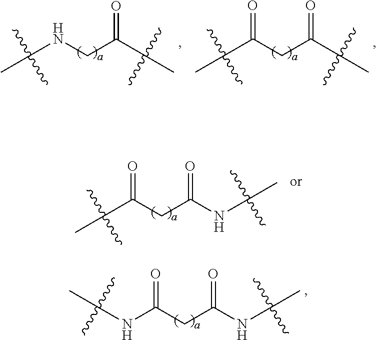
wherein a in an integer from 2 to 14.

4. The compound of claim 1, wherein E is

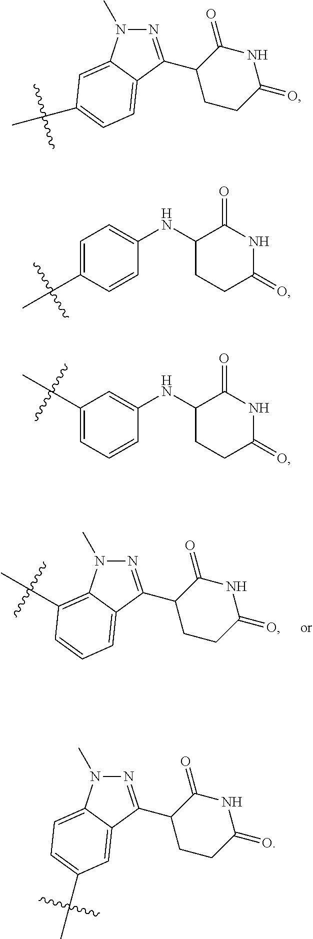 [SMILES: *c1ccc2c(C3CCC(=O)NC3=O)nn(C)c2c1 ; *c1ccc(NC2CCC(=O)NC2=O)cc1 ; *c1cccc(NC2CCC(=O)NC2=O)c1 ; *c1cccc2c(C3CCC(=O)NC3=O)nn(C)c12 ; *c1ccc2c(c1)c(C1CCC(=O)NC1=O)nn2C]

5. The compound of claim 1, wherein the compound is disclosed in Table 1 herein or in any one of Examples 1-28 herein.

6. A pharmaceutical composition, comprising the compound of claim 1 and a pharmaceutically acceptable carrier.

7. A method of treating light chain amyloidosis, comprising administering to a subject the compound of claim 1.

8. A method of stabilizing immunoglobulin light chains, comprising contacting the immunoglobulin light chains with the compound of claim 1.

9. The method of claim 8, wherein the immunoglobulin light chains are stabilized in a native conformation thereof.

10. The method of claim 8, wherein the immunoglobulin light chains are dimers.

11. A method of preventing or lessening immunoglobulin light chain misfolding and/or endoproteolysis, comprising contacting the immunoglobulin light chains with the compound of claim 1.

12. A method of maintenance therapy upon recurrence of light chain amyloidosis following primary treatment, comprising administering to a subject the compound of claim 1.

13. The method of claim 7, further comprising administering to the subject a second active agent selected from proteasome inhibitors (e.g., bortezomib, ixazomib, carfilzomib), alkylating agents (e.g., bendamustine, melphalan, cyclophosphamide), steroids (e.g., dexamethasone), immunomodulatory agents (e.g., thalidomide, lenalidomide, pomalidomide), an anti-CD38 antibody (e.g., daratumumab, isatuximab), an anti-CD20 antibody (e.g., rituximab), an anti-IL-6 antibody (e.g., siltuximab), a UPR activator (e.g., an ATF-6 activator), an antibody-drug-conjugate (e.g., belantamab mafodotin, STI-6129), an agent that promotes amyloid deposit clearance (e.g., CAEL-101, birtamimab), an anti-thymocyte antibody (e.g., Thymoglobulin®, Atgam®), atacicept or an anti-amyloid antibody.

14. The method of claim 7, further comprising stem cell transplant therapy.

15. The method of claim 13, wherein the second active agent is a plasma cell-directed therapy.
